(19) United States
(12) Patent Application Publication
(10) Pub. No.: US 2026/0240878 A1
(43) Pub. Date: Aug. 20, 2026

(54) PTP1B/TC-PTP DUAL INHIBITORS AND PROTEIN DEGRADERS

(71) Applicant: Purdue Research Foundation, West Lafayette, IN (US)

(72) Inventors: Zhong-Yin Zhang, West Lafayette, IN (US); Jiajun Dong, Jupiter, FL (US); Jinmin Miao, West Lafayette, IN (US); Yiming Miao, West Lafayette, IN (US)

(21) Appl. No.: 19/154,182

(22) PCT Filed: Dec. 8, 2023
(86) PCT No.: PCT/US2023/083261

§ 371 Date: Aug. 6, 2025

Related U.S. Application Data
(60) Provisional application No. 63/443,582, filed on Feb. 6, 2023.

(51) Int. Cl. A61K 31/662; A61K 31/675; A61P 3/04; A61P 3/10; A61P 35/00; C07F 9/38; C07F 9/6558

(52) U.S. Cl. CPC A61K 31/662; A61K 31/675; A61P 3/04; A61P 3/10; A61P 35/00; C07F 9/3882; C07F 9/65583

(57) ABSTRACT

Compounds that are dual inhibitors and dual degraders of protein-tyrosine phosphatase 1B (PTP1B) and T-cell-PTP (TC-PTP): compositions comprising the same: and their use for treating diseases or conditions mediated by PTP1B and TC-PTP.

### CROSS-REFERENCE TO RELATED APPLICATION

This application claims priority to U.S. provisional patent application No. 63/443,582, which was filed Feb. 6, 2023, and which is hereby incorporated by reference in its entirety.

### STATEMENT OF GOVERNMENT SUPPORT

This invention was made with government support under CA069202 awarded by the National Institutes of Health. The government has certain rights in the invention.

### TECHNICAL FIELD

The present disclosure relates to dual protein-tyrosine phosphatase 1B (PTP1B) and T-cell-PTP (TC-PTP) inhibitors and PTP1B and TC-PTP protein degraders and their use in the treatment of cancer and other diseases.

### BACKGROUND

This section introduces aspects that may help facilitate a better understanding of the disclosure. Accordingly, these statements are to be read in this light and are not to be construed as admissions about what is or is not prior art.

Protein tyrosine phosphorylation is a crucial post-translational modification controlled by protein tyrosine kinases (PTKs) and protein tyrosine phosphatases (PTPs) and plays a vital role in regulating essential cellular functions. Given that numerous human diseases are associated with aberrant protein tyrosine phosphorylation, both PTKs and PTPs are highly sought-after targets for drug discovery. Among members of the PTP family, protein-tyrosine phosphatase 1B (PTP1B, also called PTPN1) and T-cell protein tyrosine phosphatase (TC-PTP, also called PTPN2) are the two most closely related cytosolic PTPs, sharing over 72% amino acid sequence identity between their catalytic domains. Despite their structural similarity, PTP1B and TC-PTP are known to play non-redundant and synergistic roles in coordinating several important signaling pathways.

PTP1B and TC-PTP have long been known to function in concert in regulating both insulin and leptin-mediated cellular processes. PTP1B negatively regulates the amplitude of insulin action by the dephosphorylating insulin receptor and insulin receptor substrate 1, while TC-PTP catalyzes insulin receptor dephosphorylation to limit the duration of insulin signaling. PTP1B and TC-PTP also attenuate leptin signaling by bringing about the dephosphorylation of JAK2 and STAT3, respectively.

PTP1B and TC-PTP play non-redundant roles in attenuating IFN-γ signaling. Elimination of PTP1B increases JAK2 phosphorylation and enhances IFN-γ mediated STAT1 activation, while TC-PTP removal from tumor cells augments IFN-γ signaling and antigen presentation as a result of increased phosphorylation of JAK1 and its downstream effector STAT1. Interestingly, PTP1B and TC-PTP also serve distinct functions as negative regulators of T cell activation. To this end, PTP1B deletion in T cells promotes antigen-induced expansion and cytotoxicity of CD8⁺ T cells against solid tumors through increased JAK2/STAT5 phosphorylation. Genetic ablation of TC-PTP in T cells increases the expansion and survival of CD8⁺ T cells and promotes the activation of CD8⁺ T cells through amplifying LCK and STAT5 phosphorylation.

Based on the roles of PTP1B and TC-PTP in cellular signaling, targeting both enzymes concurrently may produce synergistic effects for a number of therapeutic applications, including Type II diabetes, obesity, and anti-cancer immunotherapies. However, combination therapies are prone to elicit complex pharmacokinetics/pharmacodynamics, unanticipated drug-drug interactions, toxicity, and/or patient compliance problems. Polypharmacology, the design or use of multi-targeting compounds that act on two or more selected targets, has gained considerable interest in drug discovery owing to the increasing appreciation of the complexity of multifactorial human diseases (Anighoro, A. et al., 2014, J. Med. Chem. 57, 7874-7887). Compared to single-targeting drugs or a combination of multiple drugs, a polypharmacological agent or multi-targeted ligand offers a valuable alternative with several advantages, such as superior therapeutic effects, reduced risk of drug-drug interactions, more predictable pharmacokinetic/pharmacodynamic profiles, and simplification of the treatment regimen.

Thus, there is an unmet need for PTP1B/TC-PTP dual inhibitors and PTP1B/TC-PTP dual degraders having physical and pharmacological properties that allow them to be used in therapeutic applications for treating disease. It is an object of the present disclosure to provide such compounds. This and other objects and advantages, as well as inventive features, will be apparent from the detailed description provided herein.

### SUMMARY

Provided is a compound of formula (I), or a pharmaceutically acceptable salt, hydrate, tautomer, or stereoisomer thereof:

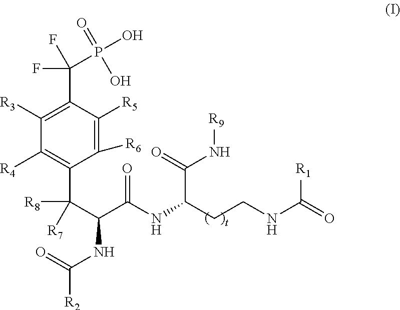

wherein
each R₁ and R₂ independently is a residue of a carboxylic acid, or a pharmaceutically acceptable salt thereof, wherein R₁ and R₂ can be the same or different;
R₃, R₄, R₅, R₆, R₇, and R₈ each independently are selected from the group consisting of hydrogen, deuterium, halogen, hydroxy. C₁-C₆ alkyl, C₃-C₆ cycloalkyl. C₁-C₆ alkylene, 4-6 membered heterocyclyl, and —C₁-C₆alkylene-4-6 membered heterocyclyl, wherein C₁-C₆ alkyl, C₃-C₆ cycloalkyl, C₁-C₆ alkylene, 4-6 membered heterocyclyl, and —C₁-C₆alkylene-4-6 membered heterocyclyl group optionally substituted on one or more available carbons by one or more substituents each independently selected from deuterium, halogen, hydroxy, C═O, C₁-C₆ alkyl, C₃-C₆ alkenyl, C₃-C₆ alkynyl, C₃-C₆ cycloalkyl. —C₁-C₆-alkylene-C₃-C₆ cycloalkyl, C₁-C₆ alkyl-S(O)₂—, C₃-C₆ cycloalkyl-S(O)₂—, C₁-C₆ alkyl-C(O)—, C₁-C₆ alkoxy-C(O)—, —NH—C(O)—Rₐ, and —C(O)—NH—Rₐ, wherein Rₐ is C₁-C₆ alkyl, C₃-C₆ cycloalkyl, C₁-C₆ alkylene, 4-6 membered heterocyclyl, and —C₁-C₆ alkylene-4-6 membered heterocyclyl optionally substituted on one or more available carbons by one, or more substituents each independently selected from deuterium, halogen, hydroxy, C═O, C₁-C₆ alkyl, C₃-C₆ alkenyl, C₃-C₆ alkynyl, C₃-C₆ cycloalkyl, —C₁-C₆-alkylene-C₃-C₆cycloalkyl, C₁-C₆alkyl-S(O)₂—, C₁-C₆cycloalkyl-S(O)₂—, C₁-C₆ alkyl-C(O)—, C₁-C₆ alkoxy-C(O)—, —NH—C(O)—R_{b}, and —C(O)—NH—R_{b}, wherein R_{b} is independently selected from deuterium, halogen, hydroxy, C═O, C₁-C₆ alkyl, C₃-C₆ alkenyl, C₃-C₆ alkynyl, C₃-C₆ cycloalkyl, —C₁-C₆-alkylene-C₃-C₆ cycloalkyl, C₁-C₆ alkyl-S(O)₂—, C₃-C₆ cycloalkyl-S(O)₂—, C₁-C₆ alkyl-C(O)—, and C₁-C₆alkoxy-C(O)—;
t is 0 to 6;
R₉ is H or a group represented by formula L-B, wherein L is a linker selected from the group consisting of:

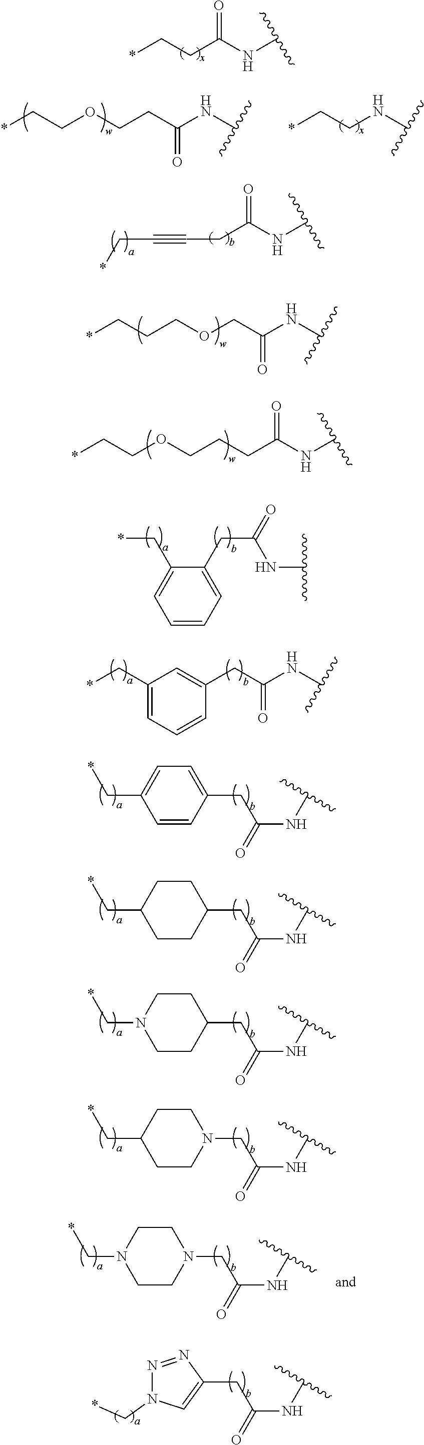

wherein w is 1-5, x is 1-15, and each a and b is independently 0-3;
the bond designated with “ [symbol] ” is attached to B; the bond designated with represents the point of attachment of R₉; and
B is selected from the group B₁, B₂, and B₃, wherein B₁ is represented by a structure:

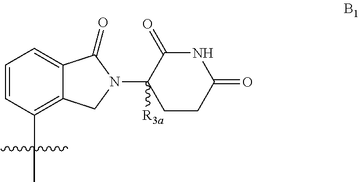

wherein R₃ₐ is fluoro, hydrogen, or deuterium;
B₂ is represented by a structure:

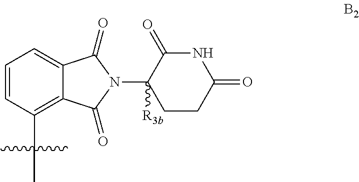

wherein R_{3b} is fluoro, hydrogen, or deuterium; and
B₃ is represented by a structure:

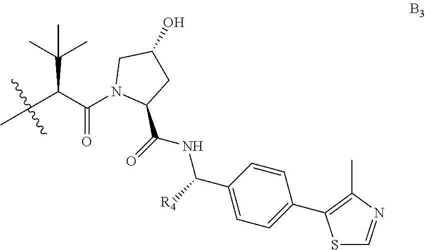

wherein R₄ is methyl, hydrogen, or deuterium.

Further provided is a compound of formula (II), or a pharmaceutically acceptable salt, hydrate, tautomer, and stereoisomer thereof:

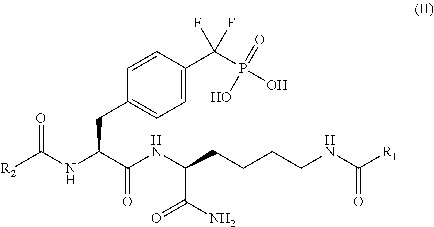

wherein R₁ and R₂ are as defined as for formula (I) above.

In some embodiments, R₁ or R₂ is:

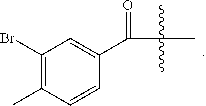 [SMILES: *C(=O)c1ccc(C)c(Br)c1]

In some embodiments, R₁ or R₂ is:

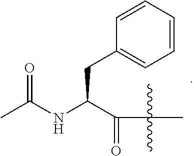 [SMILES: *C(=O)[C@H](Cc1ccccc1)NC(C)=O]

In some embodiments. R₁ or R₂ is:

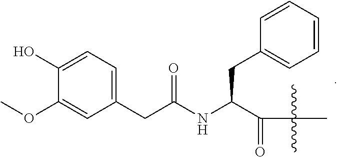 [SMILES: *C(=O)[C@H](Cc1ccccc1)NC(=O)Cc1ccc(O)c(OC)c1]

In some embodiments, the compound of formula (II) is:

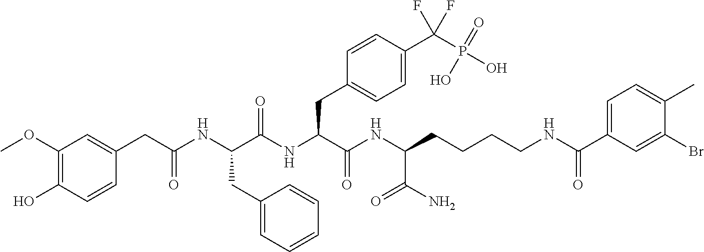

or a pharmaceutically acceptable salt, hydrate, tautomer, or stereoisomer thereof.

Further provided is a compound of formula (III), or a pharmaceutically acceptable salt, hydrate, tautomer, or stereoisomer thereof:

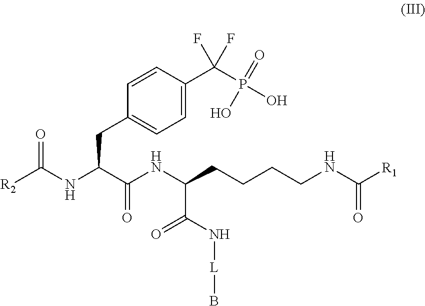

wherein R₁, R₂, L, and B are as defined as for formula (I) above.

In some embodiments, the B is B₃:

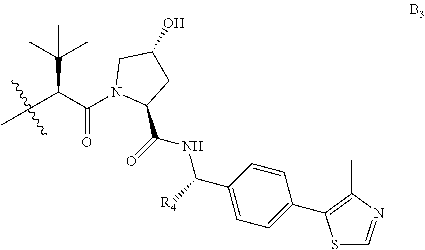

wherein R₄ is methyl, hydrogen, or deuterium.

In some embodiments, the R₁ or R₂ is selected from:

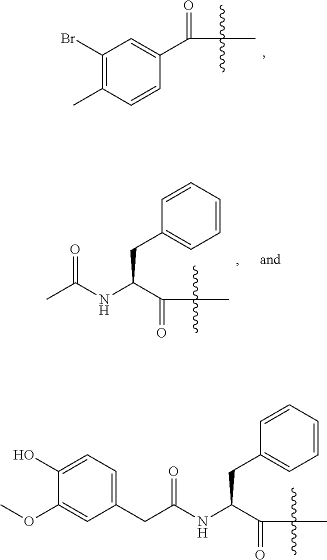 [SMILES: *C(=O)c1ccc(C)c(Br)c1 ; *C(=O)[C@H](Cc1ccccc1)NC(C)=O ; *C(=O)[C@H](Cc1ccccc1)NC(=O)Cc1ccc(O)c(OC)c1]

In some embodiments, the linker L is selected from:

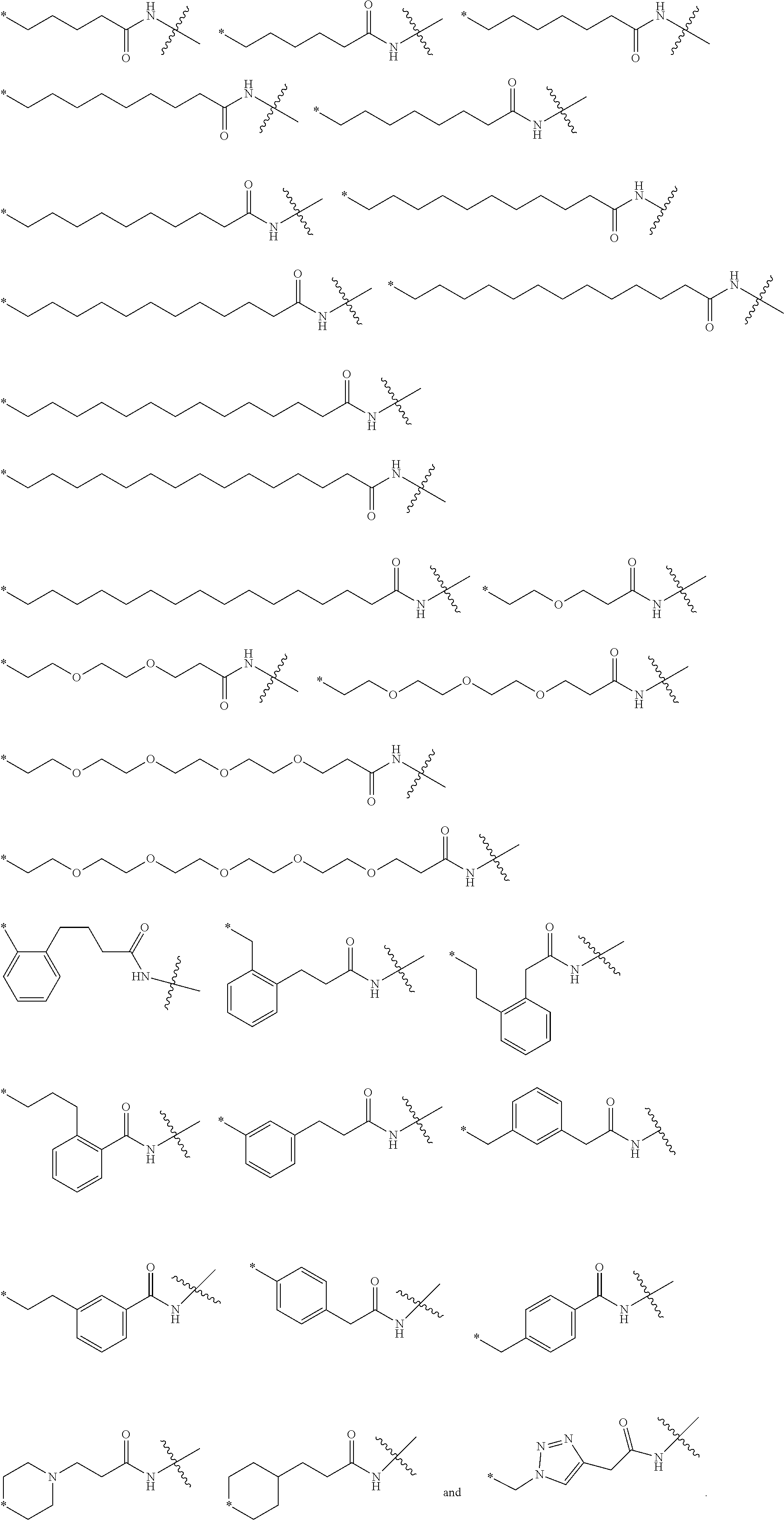

In some embodiments, the carboxylic acid in the compound of formula (I), (II) and (III) is represented by a formula RCOOH, wherein R is selected from a group consisting of hydrogen, an aliphatic group, or an aromatic group, wherein the aliphatic group is saturated or unsaturated, and wherein the aliphatic group or the aromatic group is substituted with C₁-C₂ alkyl, C₁-C₂₄ alkenyl, C₁-C₂₄ alkynyl, haloalkyl, haloalkenyl, heteroalkyl, cycloalkyl, cycloalkenyl, heterocycloalkyl, heterocycloalkenyl, aryl, heteroaryl, cycloalkylalkyl, heterocycloalkylalkyl, arylalkyl, heteroarylalkyl, arylalkenyl, cycloalkylheteroalkyl, heterocycloalkylheteroalkyl, heteroarylheteroalkyl, arylheteroalkyl, and acyl, wherein R group is optionally substituted with at least one of the groups selected from C₁-C₂₄ alkyl, hydroxy, alkoxy, cyano, halo, nitro, aryl, amino, C₁-C₂₆ alkenyl, C₁-C₂₄ alkynyl, haloalkyl, haloalkenyl, heteroalkyl, cycloalkyl, cycloalkenyl, heterocycloalkyl, heterocycloalkenyl, aryl, heteroaryl, cycloalkylalkyl, heterocycloalkylalkyl, arylalkyl, heteroarylalkyl, arylalkenyl, cycloalkylheteroalkyl, heterocycloalkylheteroalkyl, heteroarylheteroalkyl, arylheteroalkyl, and acyl.

In some embodiments, the carboxylic acid is selected from the group consisting of 3-dimethylaminobenzoic acid, 2-(2-cyanophenylthio)benzoic acid, 2-(4-chlorobenzoyl)benzoic acid, (−)-2-oxo-4-thiazolidine-carboxylic acid, (−)-N-acetylneuraminic acid, (+)-6-methoxy-α-methyl-2-naphthaleneacetic acid, (+)-carbobenzyloxy-D-proline, (+)-menthoxyacetic acid, (±)-2-(2-chlorophenoxy)propionic acid, (±)-1-methyl-2-cyclohexene-1-carboxylic acid, (1-naphthoxy)acetic acid, (IR)-(Ia,2b,3a)-(+)-3-methyl-2-nitromethyl-5-oxocyclopentaneacetic acid, (1R,4R)-7,7-dimethyl-2-oxobicyclo[2.2.1]heptane-1-carboxylic acid, (1S)-(+)-camphanic acid, (1S,3R,4S,5R)-1,3,4,5-tetrahydroxycyclohexanecarboxylic acid, (2,4-di-tert-pentylphenoxyl)acetic acid, (2-naphthoxy)acetic acid, (2-pyrimidylthio)acetic acid, (4-carboxybutyl)triphenyl-phosphonium bromide, (4-chlorophenylthio)acetic acid, (4-methylphenoxy)acetic acid, (α, α, α-trofluoro-m-tolyl)acetic acid, (E)-2-((4-hydroxyphenyl)diazenyl)benzoic acid, (E)-2-methyl-3-(2,4,5-trimethoxyphenyl)acrylic acid, (methylthio)acetic acid, (R)-(−)-2-hydroxy-4-phenylbutyric acid, (R)-(−)-3-chloromandelic acid, (R)-(−)-hexahydromandelic acid, (R)-(+)-2-pyrrolidone-5-carboxylic acid, (R)-(+)-citronellic acid, (R)-2-(l-phenylethylcarbamoyl)benzoic acid, (R)-2-hydroxy-2-phenylacetic acid, (R)-3,3,3-trifluoro-2-methoxy-2-phenylpropanoic acid, (R)-6-hydroxy-2,5,7,8-tetramethylchroman-2-carboxylic acid, (S)-(−)-indoline-2-carboxylic acid, (S)-(+)-2-oxo-4-phenyl-3-oxazolidineacetic acid, (S)-(+)-5-oxo-2-tetrahydro-furancarboxylic acid, (S)-(+)-hexahydromandelic acid, (S)-(+)-N-[1-(1-naphthyl)-ethyl]-phthalamic acid, (S)-(+)-O-acetylmandelic acid, (S)-2-(1-phenylethylcarbamoyl)benzoic acid, (S)-2-(4-isobutylphenyl)propanoic acid, (S)-2-(phenylcarbamoyloxy)propanoic acid, (S)-3-(benzyloxycarbonyl)-2-oxoimidazolidine-4-carboxylic acid, (S)-3,3,3-trifluoro-2-methoxy-2-phenylpropanoic acid, (S)-3,3,3-trifluoro-2-methoxy-2-phenylpropanoic acid, (S)-6-methoxy-2,5,7,8-tetramethylchroman-2-carboxylic acid, (trimethylsilyl)acetic acid, (Z)-2-cyano-3-(3-hydroxyphenyl)acrylic acid, 1-(4-chlorophenyl)-1-cyclopentanecarboxylic acid, 1-(tert-butyl)hydrocinnamic acid, 1,2-phenylenedioxydiacetic acid, 1,4-dihydro-2-methylbenzoic acid, 1,4-dihydroxy-2-naphthoic acid, 10-hydroxydecanoic acid, 10-undecynoic acid, 1-admantanecarboxylic acid, 1-cyano-1-cyclopropane-carboxylic acid, 1-hydroxy-2-naphthoic acid, 1-isoquinolinecarboxylic acid, 1-methyl-(1S,2R)-(+)-cis-1,2,3,6-tetrahydrophthalate, 1-methyl-1-cyclohexane-carboxylic acid, 1-methyl-1H-indole-2-carboxylic acid, 1-methyl-2-pyrrolecarboxylic acid, 1-methylcyclopropane-carboxylic acid, 1-naphthoic acid, 1-phenyl-1-cyclopentane-carboxylic acid, 1-phenyl-1-cyclopropane-carboxylic acid, 1-pyreneacetic acid, 1-pyrenebutyric acid, 1-pyrenecarboxylic acid, 2-((1R,2R,3R,4S)-3-hydroxy-4,7,7-trimethylbicyclo[2.2.1]heptan-2-yl)acetic acid, 2-((benzyloxycarbonyl)(methyl)amino)-2-methylpropanoic acid, 2-(2, (trifluoromethyl)phenyl)acetic acid, 2-(2,4,5-trichlorophenoxy)-propionic acid, 2-(2,4-dichlorophenoxy)-propionic acid, 2-(3,5-dinitrobenzamido)-2-phenylacetic acid, 2-(3,5-dinitrobenzamido)-4-methylpentanoic acid, 2-(3-chlorophenoxy)propionic acid, 2-(4-(trifluoromethyl)phenyl)acetic acid, 2-(4-chloro-3-nitrobenzoyl)-benzoic acid, 2-(4-chlorophenoxy)-2-methyl-propionic acid, 2-(4-chlorophenoxy)propionic acid, 2-(4-fluorobenzoyl)benzoic acid, 2-(4-hydroxy-3-methoxyphenyl)acetic acid, 2-(4-hydroxyphenoxy)-propionic acid, 2-(4-isobutylphenyl)propanoic acid, 2-(4-nitrophenyl)propionic acid, 2-(benzyloxycarbonylamino)-3-(1 H-indol-3-yl)propanoic acid, 2-(trifluoromethyl)acrylic acid, 2-(trifluoromethyl)benzoic acid, 2-(trifluoromethyl)cinnamic acid, 2,2,3,3-tetramethyl-cyclopropanecarboxylic acid, 2,2-bis(hydroxymethyl)-propionic acid, 2,3,4,5,6-pentafluoro-cinnamic acid, 2,3,4,5,6-pentafluorophenoxy acetic acid, 2,3,4,5,6-pentafluorophenyl-acetic acid, 2,3,4,5-tetrafluorobenzoic acid, 2,3,4-trifluorocinnamic acid, 2,3,4-trihydroxybenzoic acid, 2,3,4-trimethoxybenzoic acid, 2,3,5,6-tetrafluoro-4-hydroxy-benzoic acid hydrate, 2,3,5,6-tetrafluorobenzoic acid, 2,3,5,6-tetrafluoro-p-toluic acid, 2,3,5-triiodobenzoic acid, 2,3,6-trifluorobenzoic acid, 2,3-dichlorobenzoic acid, 2,3-difluorobenzoic acid, 2,3-dihydroxybenzoic acid, 2,3-dimethylbenzoic acid, 2,4,5-trichlorophenoxyacetic acid, 2,4,5-trimethoxybenzoic acid, 2,4,6-trichlorobenzoic acid, 2,4,6-trifluorobenzoic acid, 2,4,6-trihydroxybenzoic acid monohydrate, 2,4,6-trimethylbenzoic acid, 2,4-bis(trifluoromethyl)-benzoic acid, 2,4-dichloro-5-fluorobenzoic acid, 2,4-dichloro-5-sulfamoyl-benzoic acid, 2,4-dichlorobenzoic acid, 2,4-dichlorophenylacetic acid, 2,4-difluorobenzoic acid, 2,4-difluorophenylacetic acid, 2,4-dihydroxybenzoic acid, 2,4-dimethylbenzoic acid, 2,4-dinitrobenzoic acid, 2,4-dinitrophenylacetic acid, 2,4-hexadienoic acid, 2,5-bis(trifluoromethyl)-benzoic acid, 2,5-dichlorobenzoic acid, 2,5-difluorobenzoic acid, 2,5-difluorophenylacetic acid, 2,5-dihydroxybenzoic acid, 2,5-dihydroxyphenylacetic acid, 2,5-dimethoxybenzoic acid, 2,5-dimethoxycinnamic acid, 2,6-dichloro-3-nitrobenzoic acid, 2,6-difluorobenzoic acid, 2,6-difluorophenylacetic acid, 2,6-dihydroxybenzoic acid, 2,6-dimethoxynicotinic acid, 2,6-dimethylbenzoic acid, 2,6-heptadienoic acid, 2-[4-(dibutylamino)-2-hydroxy-benzoyl]benzoic acid, 2-bibenzylcarboxylic acid, 2-biphenylcarboxylic acid, 2-bromo-3-nitrobenzoic acid, 2-bromo-4,5-dimethoxybenzoic acid, 2-bromo-5-methoxybenzoic acid, 2-bromo-5-nitrobenzoic acid, 2-bromoacrylic acid, 2-bromophenylacetic acid, 2-chloro-3-nitrobenzoic acid, 2-chloro-4,5-difluorobenzoic acid, 2-chloro-4-fluorobenzoic acid, 2-chloro-5-(methylthio)-benzoic acid, 2-chloro-5-(trifluoro-methyl)benzoic acid, 2-chloro-5-nitrobenzoic acid, 2-chloro-5-nitrocinnamic acid, 2-chloro-6-fluorobenzoic acid, 2-chloro-6-fluorophenylacetic acid, 2-chloro-6-methylnicotinic acid, 2-chlorobenzoic acid, 2-chloronicotinic acid, 2-chlorophenylacetic acid, 2-chloropropionic acid, 2-ethoxy-1-naphthoic acid, 2-ethoxybenzoic acid, 2-ethyl-2-hydroxybutyric acid, 2-ethylbutyric acid, 2-ethylhexanoic acid, 2-ethylthio-2,2-diphenyl-acetic acid, 2-fluoro-3-(trifluoromethyl)-benzoic acid, 2-fluoro-4-(trifluoromethyl)-benzoic acid, 2-fluoro-5-methylbenzoic acid, 2-fluoro-5-nitrobenzoic acid, 2-fluoro-6-(trifluoromethyl)-benzoic acid, 2-fluorobenzoic acid, 2-fluorocinnamic acid, 2-fluorophenylacetic acid, 2-hydroxy-3-isopropyl-6-methylbenzoic acid, 2-hydroxy-3-isopropylbenzoic acid, 2-hydroxy-3-methylbutyric acid, 2-hydroxy-6-isopropyl-3-methylbenzoic acid, 2-hydroxycaproic acid, 2-hydroxyhippuric acid, 2-hydroxyisobutyric acid, 2-hydroxynicotinic acid, 2-hydroxyphenylacetic acid, 2-iodobenzoic acid, 2-mercaptonicotinic acid, 2-methoxy-2-phenylacetic acid, 2-methoxy-4-(methylthio)-benzoic acid, 2-methoxy-4-nitrobenzoic acid, 2-methoxyphenylacetic acid, 2-methyl-1-cyclohexane-carboxylic acid (cis and trans), 2-methyl-3-nitrobenzoic acid, 2-methyl-3-phenylpropanoic acid, 2-methyl-4-oxo-4-phenylbutyric acid, 2-methy-6-nitrobenzoic acid, 2-methylbutyric acid, 2-methylcinnamic acid, 2-methylcyclopropane-carboxylic acid (cis and trans), 2-methylhexanoic acid, 2-methylhippuric acid, 2-methylhydrocinnamic acid, 2-methylvaleric acid, 2-naphthoic acid, 2-naphthylacetic acid, 2-nitro-4-(trifluoromethyl)benzoic acid, 2-nitrobenzoic acid, 2-norbornaneacetic acid, 2-oxo-6-pentyl-2H-pyran-3-carboxylic acid, 2-phenoxybenzoic acid, 2-phenoxybutyric acid, 2-phenoxypropionic acid, 2-propylpentanoic acid, 2-quinoxalinecarboxylic acid, 2-thiopheneacetic acid, 2-thiopheneacetic acid, 2-thiopheneglyoxylic acid, 3-(2-hydroxyphenyl)propionic acid, 3-(2-thienyl)acrylic acid, 3-(3,4,5-trimethoxyphenyl)-propionic acid, 3-(3,4-dimethoxyphenyl)-propionic acid, 3-(3-hydroxy-2,4,6-triiodophenyl)pentanoic acid, 3-(3-hydroxyphenyl)-propionic acid, 3-(3-methoxyphenyl)propionic acid, 3-(4-chlorobenzoyl)propionic acid, 3-(4-fluorobenzoyl)propionic acid, 3-(4-hydroxyphenyl)propionic acid, 3-(phenylsulfonyl)propionic acid, 3-(trifluoromethyl)cinnamic acid, 3-(trimethylsilyl)propynoic acid, 3,3,3-triphenylpropionic acid, 3,4-(methylenedioxy)cinnamic acid, 3,4-(methylenedioxy)phenyl-acetic acid, 3,4-dichlorobenzoic acid, 3,4-dichlorophenoxyacetic acid, 3,4-diethoxybenzoic acid, 3,4-difluorobenzoic acid, 3,4-dihydroxybenzoic acid, 3,4-dihydroxyhydrocinnamic acid, 3,4-dihydroxyphenylacetic acid, 3,5,6-trichlorosalicylic acid, 3,5-bis(trifluoromethyl)-phenyl acetic acid, 3,5-dibromobenzoic acid, 3,5-dichlorosalicylic acid, 3,5-difluorocinnamic acid, 3,5-dihydroxy-2-naphthoic acid, 3,5-dinitrobenzoic acid, 3,5-dinitro-o-toluic acid, 3,5-dinitro-p-toluic acid, 3,5-dinitrosalicyclic acid, 3,5-di-tert-butyl-4-hydroxy-benzoic acid, 3,5-di-tert-butylbenzoic acid, 3,7-dihydroxy-2-naphthoic acid, 3, thiopheneacetic acid, 3-benzoyl-2-pyridine-carboxylic acid, 3-benzoylbenzoic acid, 3-bromo-4-fluorobenzoic acid (95%), 3-bromo-4-methylbenzoic acid, 3-bromo-5-iodobenzoic acid, 3-bromobenzoic acid, 3-bromocinnamic acid, 3-carboxy-proxyl, 3-chloro-2-nitrobenzoic acid, 3-chloro-4-fluorobenzoic acid, 3-chloro-4-hydroxyphenyl-acetic acid, 3-chlorosalicylic acid, 3-cyanobenzoic acid, 3-fluoro-2-methylbenzoic acid, 3-fluoro-4-hydroxy-phenylacetic acid, 3-fluoro-4-methoxybenzoic acid, 3-fluorophenylacetic acid, 3-furoic acid, 3-hydroxy-2-naphthoic acid, 3-hydroxy-2-quinoxaline-carboxylic acid, 3-hydroxy-4-methoxybenzoic acid, 3-hydroxy-4-methoxy-cinnamic acid, 3-hydroxy-4-nitrobenzoic acid, 3-hydroxybenzoic acid, 3-hydroxybutyric acid, 3-hydroxyphenylacetic acid, 3-indolebutyric acid, 3-indoleglyoxylic acid, 3-indolepropionic acid, 3-iodo-4-methylbenzoic acid, 3-iodobenzoic acid, 3-isoquinolinecarboxylic acid hydrate, 3-methoxy-4-nitrobenzoic acid, 3-methoxycyclohexane-carboxylic acid (cis and trans), 3-methyl-2-phenylvaleric acid, 3-methylhippuric acid, 3-methylindene-2-carboxylic acid, 3-methylsalicylic acid, 3-methyl valeric acid, 3-nitrobenzoic acid, 3-nitrophenylacetic acid, 3-nitropropionic acid, 3-noradamantanecarboxylic acid, 3-oxo-1-indancarboxylic acid, 3-phenoxybenzoic acid, 3-phenylbutyric acid, 3-p-tolylpropanoic acid, 3-thiophenecarboxylic acid, 4-(1,3-dioxoisoindolin-2-yl)-2-hydroxybutanoic acid, 4-(2,4,5-trichlorophenoxy)-butyric acid, 4-(2,4-dichlorophenoxy)-butyric acid, 4-(2,4-di-tert-pentylphenoxy)butyric acid, 4-(2-phenoxyethoxy)benzoic acid, 4-(3,4-dimethoxyphenyl)-butyric acid, 4-(4-methoxyphenyl)butyric acid, 4-(4-nitrophenyl)butyric acid, a-(diethylamino)benzoic acid, 4-(dimethylamino)cinnamic acid, 4-(dimethylamino)phenyl-acetic acid, 4-(ethylthio)benzoic acid, 4-(hydroxymethyl)benzoic acid, 4-(methylsulfonyl)benzoic acid, 4-(methylthio)benzoic acid, 4-(methylthio)phenylacetic acid, 4-(trifluoromethoxy)benzoic acid, 4′-(trifluoromethyl)biphenyl-2-carboxylic acid, α-(trifluoromethyl)mandelic acid, 4,4,4-trifluoro-3-methyl-2-butenoic acid, 4,4-bis(4-hydroxyphenyl)-valeric acid, 4,5-dimethoxy-2-nitrobenzoic acid, 4,6-dioxoheptanoic acid, 4-[4-(2-carboxybenzoyl)-phenyl]butyric acid, 4-acetamidobenzoic acid, α-acetylbenzoic acid, 4-acetylphenoxyacetic acid, 4-benzyloxy-3-methoxyphenyl-acetic acid, 4-biphenylacetic acid, 4-bromo-3,5-dihydroxy-benzoic acid, 4-bromobenzoic acid, 4-bromocinnamic acid, 4-bromophenylacetic acid, 4-butoxybenzoic acid, α-butoxyphenylacetic acid, 4-butylbenzoic acid, 4-chloro-2,5-difluorobenzoic acid, 4-chloro-3-sulfamoylbenzoic acid, 4-chlorobenzoic acid, 4-chloro-o-tolyloxyacetic acid, a-chlorophenylacetic acid, 4-chlorosalicylic acid, 4-ethoxycarbonyloxy-3,5-dimethoxybenzoic acid, 4-ethoxyphenylacetic acid, 4-ethylbenzoic acid, 4′-ethylbiphenyl-4-carboxylic acid, 4-fluorenecarboxylic acid, 4-fluoro-1-naphthoic acid, α-fluoro-2-(trifluoromethyl)-benzoic acid, 4-fluoro-3-nitrobenzoic acid, 4-fluorobenzoic acid, 4-fluorobenzoic acid, 4-fluorocinnamic acid, 4-fluorophenoxyacetic acid, α-heptyloxybenzoic acid, 4-hexylbenzoic acid, 4-hexyloxybenzoic acid, 4-hydroxy-3-(morpholino-methyl)benzoic acid hydrate, 4-hydroxy-3,5-dinitrobenzoic acid, 4-hydroxy-3-methoxy-benzoic acid, 4-hydroxy-3-methoxy-mandelic acid, 4-hydroxy-3-nitrobenzoic acid, 4-hydroxy-3-nitrophenylacetic acid, 4-hydroxybenzoic acid, 4′-hydroxybiphenyl-4-carboxylic acid, 4-hydroxyphenylacetic acid, 4-hydroxyphenylacetic acid, α-hydroxyphenylpyruvic acid, 4-iodobenzoic acid, 4-isopropoxybenzoic acid, 4-methoxy-3-nitrobenzoic acid, 4-methoxycyclohexane-carboxylic acid, 4-methoxysalicylic acid, α-methyl-1-cyclohexane-carboxylic acid (cis and trans), 4-methyl-3-nitrobenzoic acid, α-methylhippuric acid, 4-methylsalicylic acid, 4-methylvaleric acid, 4-nitro-3-pyrazolecarboxylic acid, 4-nitrohippuric acid, 4-nonyloxybenzoic acid, 4-octylbenoic acid, 4-oxo-4H-1-benzopyran-2-carboxylic acid, 4-oxo-6-phenyl-5-hexenoic acid, α-pentenoic acid, 4-pentylbenzoic acid, 4-pentylbicyclo[2.2.2]octane-1-carboxylic acid, α-pentyloxybenzoic acid, 4-pentynoic acid, 4-phenylbutyric acid, 4-propoxybenzoic acid, 4-propylbenzoic acid, 4-pyrazolecarboxylic acid, 4-tert-butylbenzoic acid, 4-tert-butylcyclohexanecarboxylic acid, 4-vinylbenzoic acid, 5-(4-chlorophenyl)-2-furoic acid, 5,6-dichloronicotinic acid, 5-bromo-2,4-dihydroxybenzoic acid, 5-fluoro-2-methylbenzoic acid, 5-fluoroindole-2-carboxylic acid, 5-fluorosalicylic acid, 5-hydantoinacetic acid, 5-hydroxy-2-indole-carboxylic acid, 5-methoxy-1-indanone-3-acetic acid, 5-methoxy-2-methyl-3-indoleacetic acid, 5-methoxy-2-nitrobenzoic acid, 5-methoxysalicylic acid, 5-methyl-2-nitrobenzoic acid, 5-methyl-2-pyrazine-carboxylic acid, 5-nitro-2-furoic acid, 5-nitro-3-pyrazolecarboxylic acid, 5-phenyl valeric acid, 6-(carbobenzyloxyamino)-caproic acid, 6-acetamidohexnoic acid, 6-bromohexanoic acid, 6-chloronicotinic acid, 6-hydroxy-2,5,7,8-tetramethylchroman-2-carboxylic acid, 6-methylchromone-2-carboxylic acid, 6-methylnicotinic acid, 6-nitrocaproic acid, 6-oxoheptaoic acid, 6-phenylhexanoic acid, 7-(carboxymethyoxy)-4-methylcoumarin, 7-hydroxycoumarin-4-acetic acid, 7-methoxy-2-benzofuran-carboxylic acid, 7-methoxycoumarin-4-acetic acid, 7-oxoctanoic acid, 9-anthracenecarboxylic acid, 9-fluoreneacetic acid, 9-fluorenone-1-carboxylic acid, α, α, α-trifluoro-m-toluic acid, α-acetamidocinnamic acid, abietic acid, acetic acid, acetyl-L-asparagine, acetylsalicyclic acid, α-cyano-4-hydroxycinnamic acid, adipic acid monoethyl ester, α-hydroxyhippuric acid, anthranilic acid, anti-3-oxotricyclo[2.2.1.02,6]heptane-7-carboxylic acid, α-phenylcyclopentaneacetic acid, α-phenyl-o-toluic acid, atrolactic acid, benzilic acid, benzotriazole-5-carboxylic acid, benzoylformic acid, bis(4-chlorophenyl)acetic acid, carbobenzyloxy-DL-alanine, carbobenzyloxy-L-alanine, carbobenzyloxy-1-glutamine, carbobenzyloxy-L-valine, cis-2-methoxycinnamic acid, crotonic acid, cyclohexanebutyric acid, cyclohexanecarboxylic acid, cyclohexanepentanoic acid, cyclohexanepropionic acid, cyclopentylacetic acid, D,L-3,4-dihydroxymandelic acid, D-3-phenyllactic acid, decanoic acid, dicyclohexylacetic acid, diethylphosphonoacetic acid, dikegulac hydrate, diphenylacetic acid, fumaric acid monoethyl ester, fusaric acid, gallic acid, geranic acid, glycolic acid, heptadecafluorononanoic acid, heptanoic acid, hexanoic acid, hippuric acid, hydrocinnamic acid, indole-3-carboxylic acid, indole-4-carboxylic acid, isovaleric acid, L-3-phenyl lactic acid, lauric acid, L-lactic acid (85%), maleamic acid, methoxyacetic acid, mono-(1R)-(−)-menthyl phthalate, mono-(1S)-(+)-menthyl phthalate, mono-methyl cis-5-norbornene-endo-2,3-dicarboxylate, mono-methyl phthalate, mono-methylterephthalate, N-(2-furoyl)glycine, n-(3,5-dinitrobenzoyl)-DL-a-phenylglycine, N-(3-indolylacetyl)-L-alanine, N-(3-indolylacetyl)-L-isoleucine, N-(3-indolylacetyl)-L-leucine, N-(3-indolylacetyl)-L-phenylalanine, N-(3-indolylacetyl)-L-valine, N-(carbobenzyloxy)-1-phenyl-alanine, N,N-diethyl-3,6-difluoro-phthalamic acid, N—[(R)—I-(1-naphthyl)ethyl]-phthalamic acid, N-[5-(trifluoromethyl)-2-pyridyl]-L-valine, N-acetyl-4-fluoro-DL-phenylalanine, N-acetyl-DL-tryptophan, N-acetyl-1-leucine, N-acetyl-L-methionine, N-acetyl-L-phenylalanine, N-acetyl-1-tyrosine, N-benzoyl-(2R,3S)-3-phenyl-isoserine, N-benzoyl-L-threonine, N-carbobenzyloxy-2-methyl-alanine, N-carbobenzyloxy-L-glutamic acid 1-methyl ester, N-carbobenzyloxy-L-isoleucine, N-carbobenzyloxy-L-Leucine, N-carbobenzyloxy-1-threonine, N-ethoxycarbonyl-1-phenylalanine, nonanoic acid, N-p-tosylglycine, N-p-tosyl-L-phenylalanine, o-anisic acid, p-anisic acid, pentafluorobenzoic acid, phenoxyacetic acid, phenylacetic acid, podocarpic acid, pyruvic acid, rhodanine-3-acetic acid, S-(thiobenzoyl)thioglycolic acid, S-benzyl-n-carbobenzyloxy-1-cysteine, sebacic acid monomethyl ester, succinamic acid, succinic 2,2-dimethyl-hydrazide, tetrahydro-2-furoic acid, trans-1-acetyl-4-hydroxy-L-proline, trans-2,3-dimethoxycinnamic acid, trans-2,4-dichlorocinnamic acid, trans-2,4-difluorocinnamic acid, trans-2,5-difluorocinnamic acid, trans-2,6-difluorocinnamic acid, trans-2-chloro-6-fluro-cinnamic acid, trans-2-hexenoic acid, trans-3-(2,3,5,6-tetramethyl-benzoyl)acrylic acid, trans-3-(2,5-dimethylbenzo-yl)-acrylic acid, trans-3-(4-ethoxy-benzoyl)acrylic acid, trans-3-(4-methoxybenzoyl)-acrylic acid, trans-3-(4-methylbenzoyl)-acrylic acid, trans-3,4-difluorocinnamic acid, trans-3-fluorocinnamic acid, trans-3-furanacrylic acid, trans-3-hexenoic acid, trans-4-chloro-3-nitrocinnamic acid, trans-4-hydroxy-3-methoxy-cinnamic acid, trans-4-methyl-1-cyclohexane carboxylic acid, trans-4-pentylcyclohexane carboxylic acid, trans-5-bromo-2-methoxy cinnamic acid, trans-styrylacetic acid, tridecafluoroheptanoic acid, trimethylacetic acid, triphenylacetic acid, valeric acid, and yohimbinic acid mono-hydrate.

In some embodiments, the compound of formula (III) is:

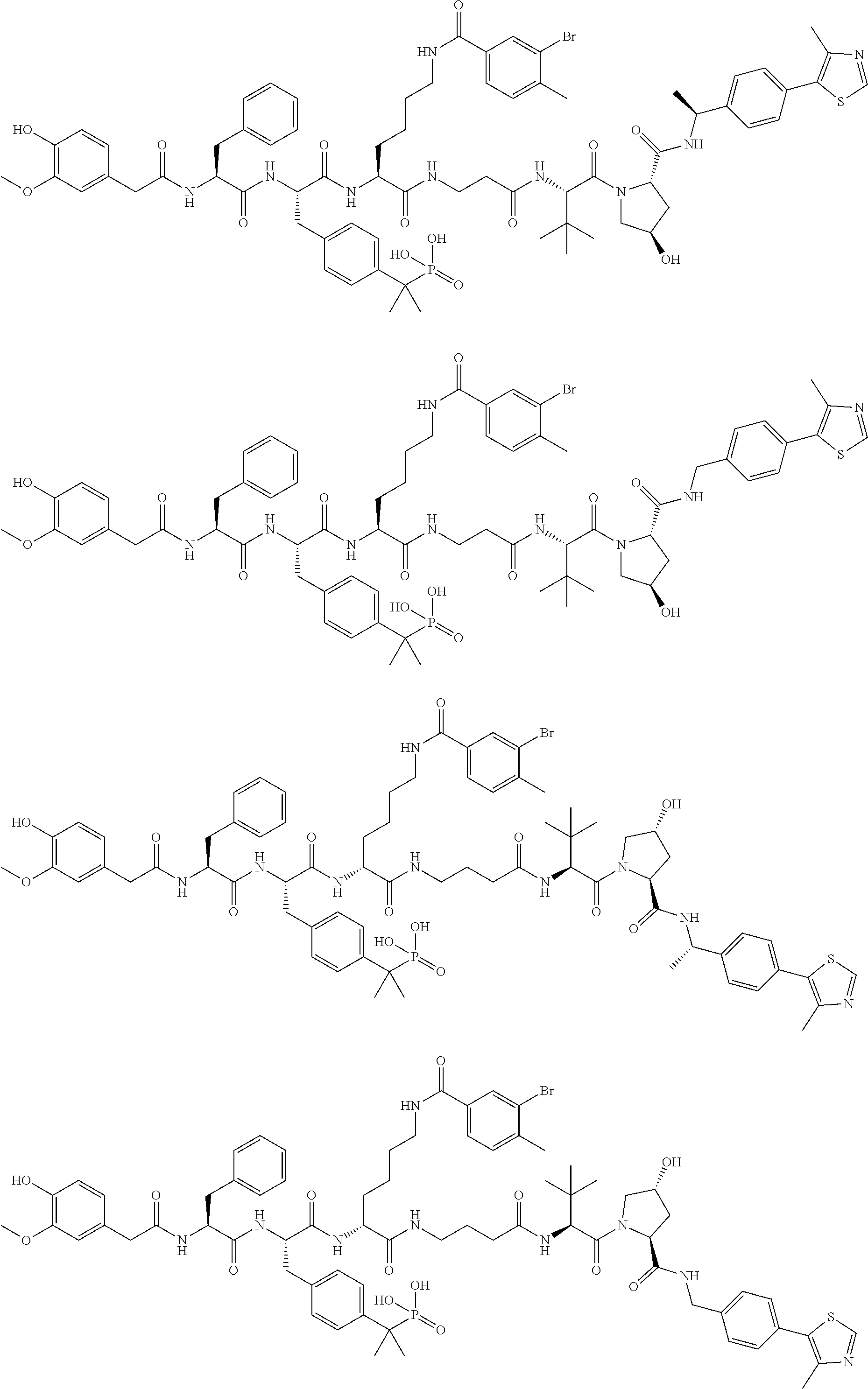

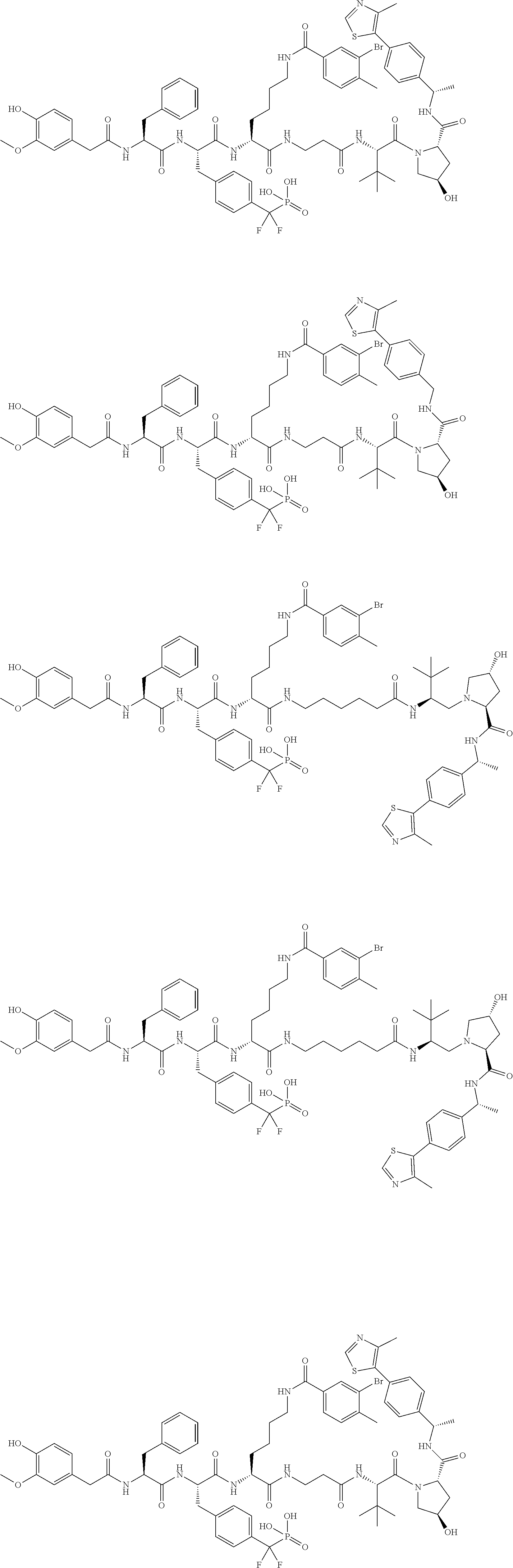

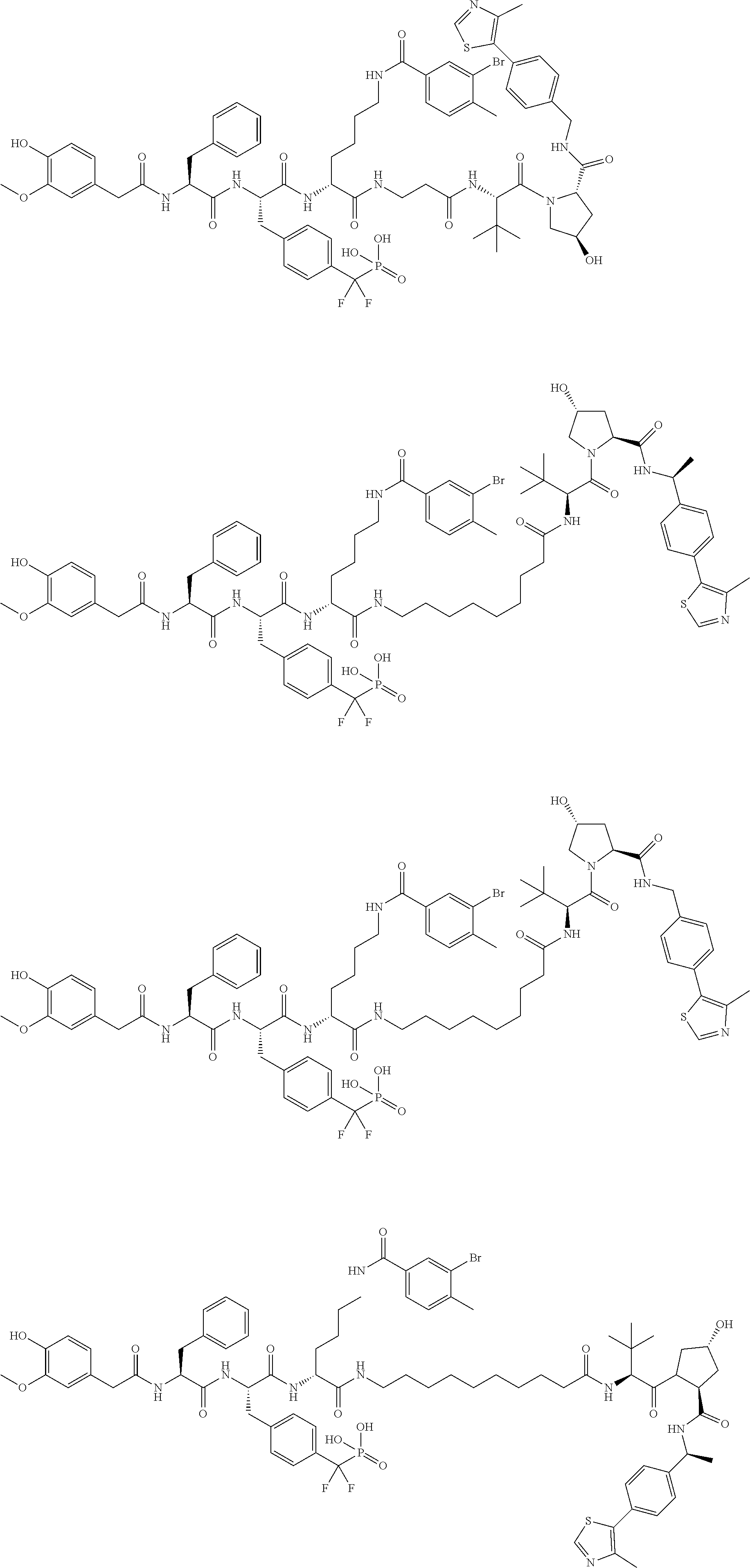

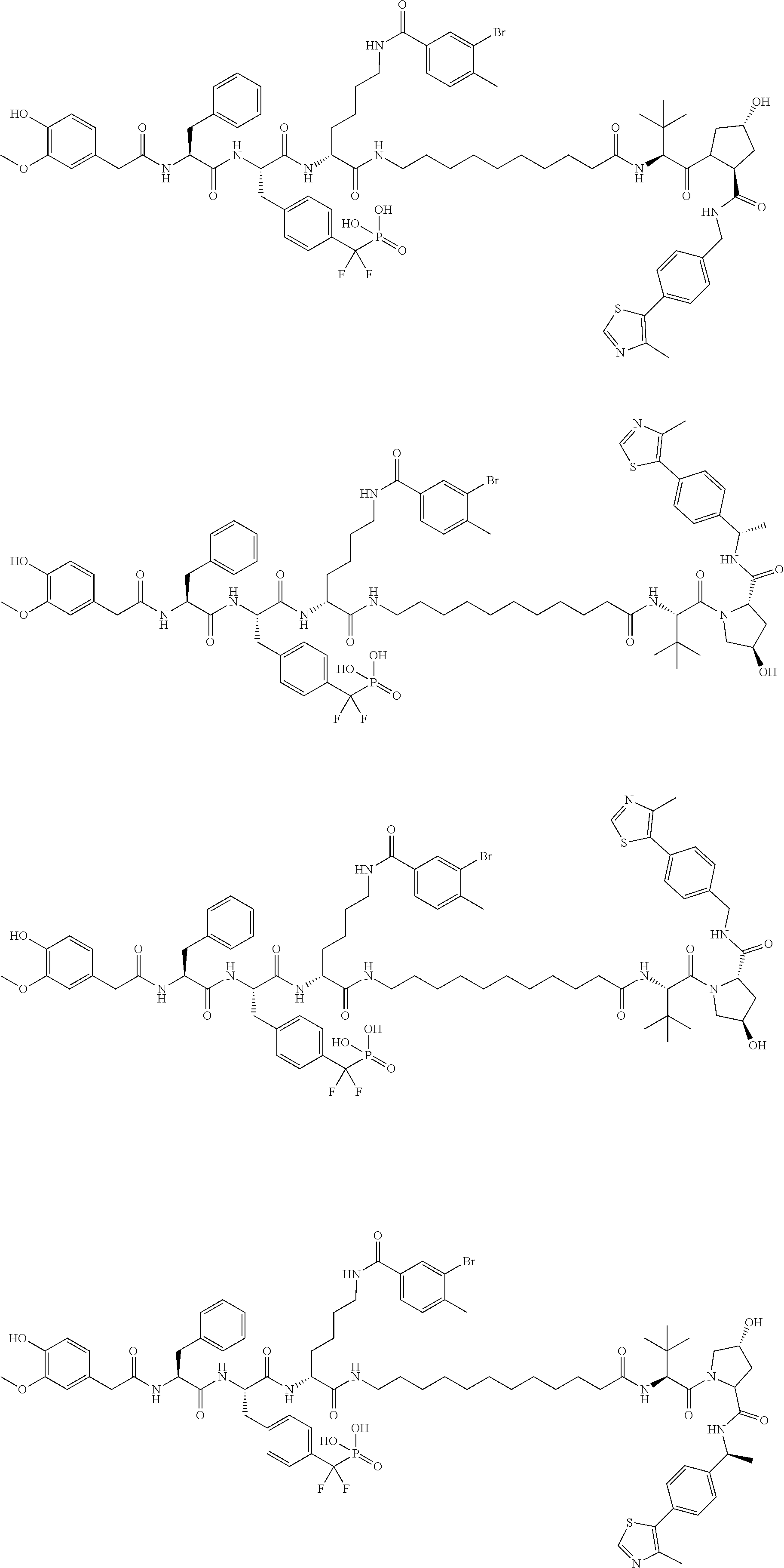

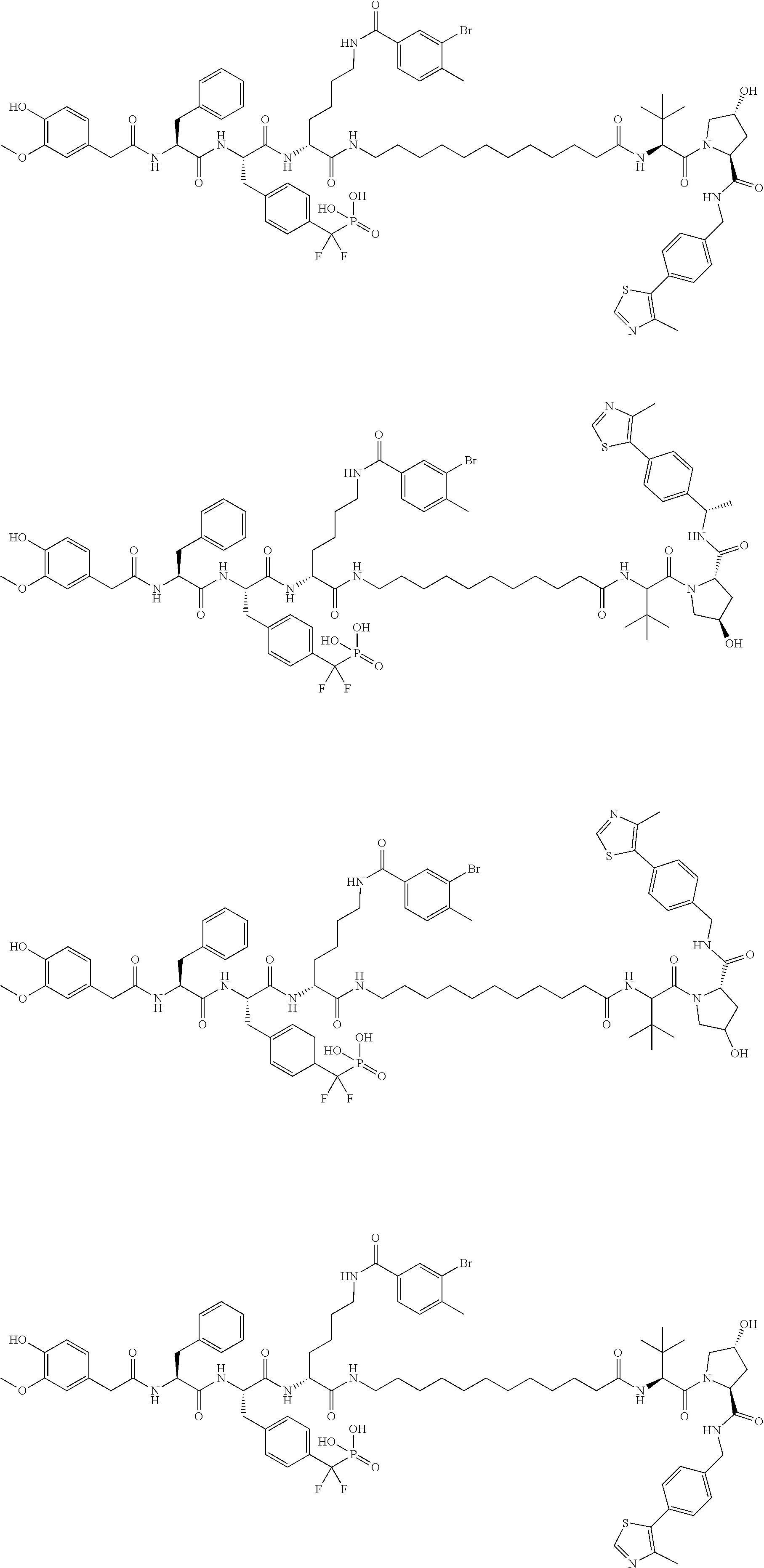

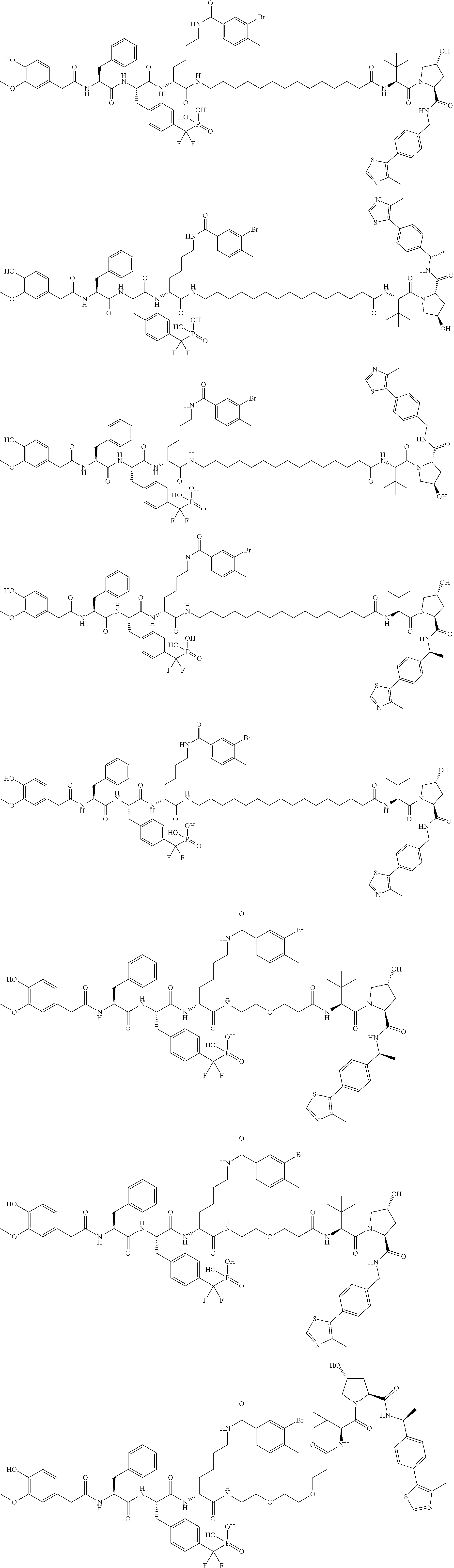

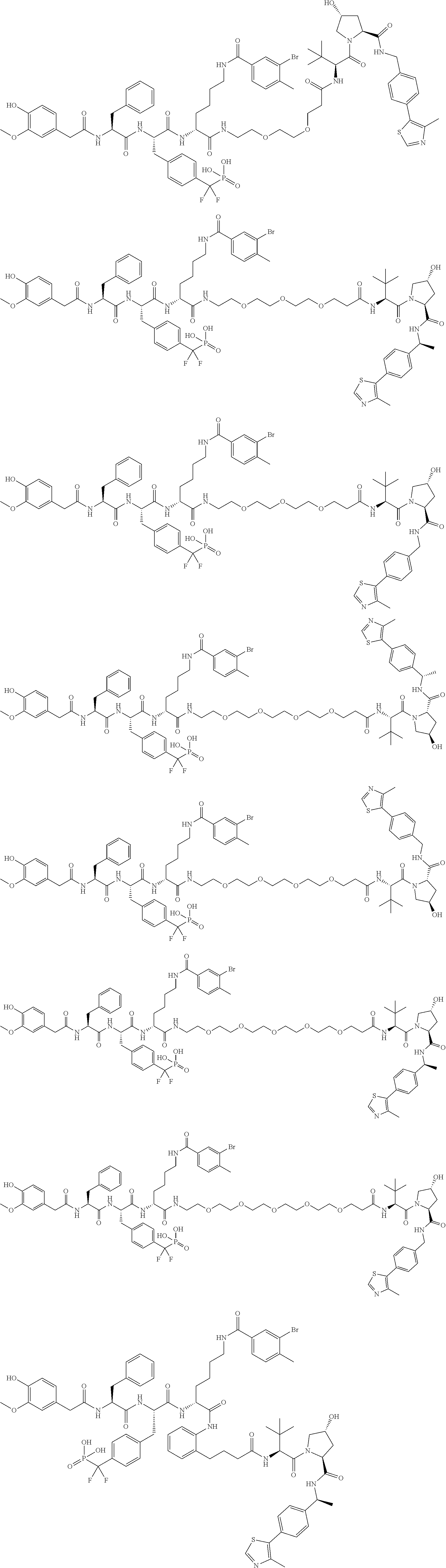

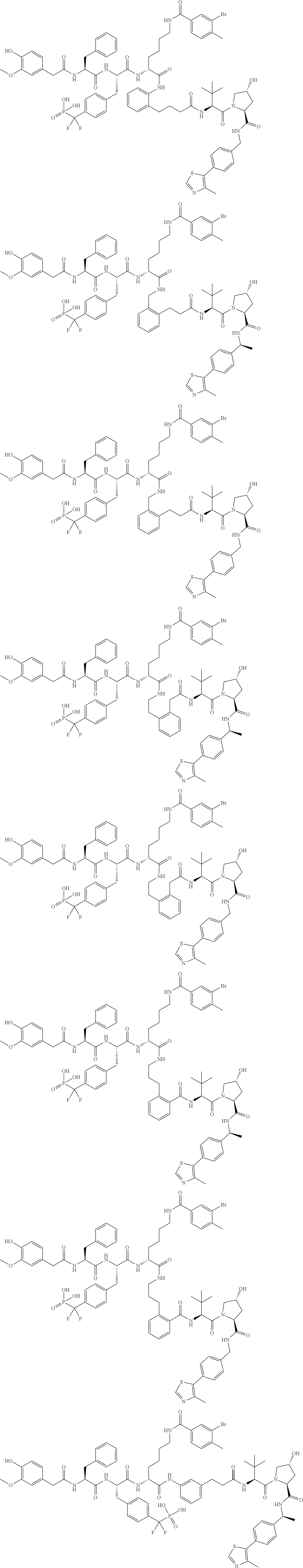

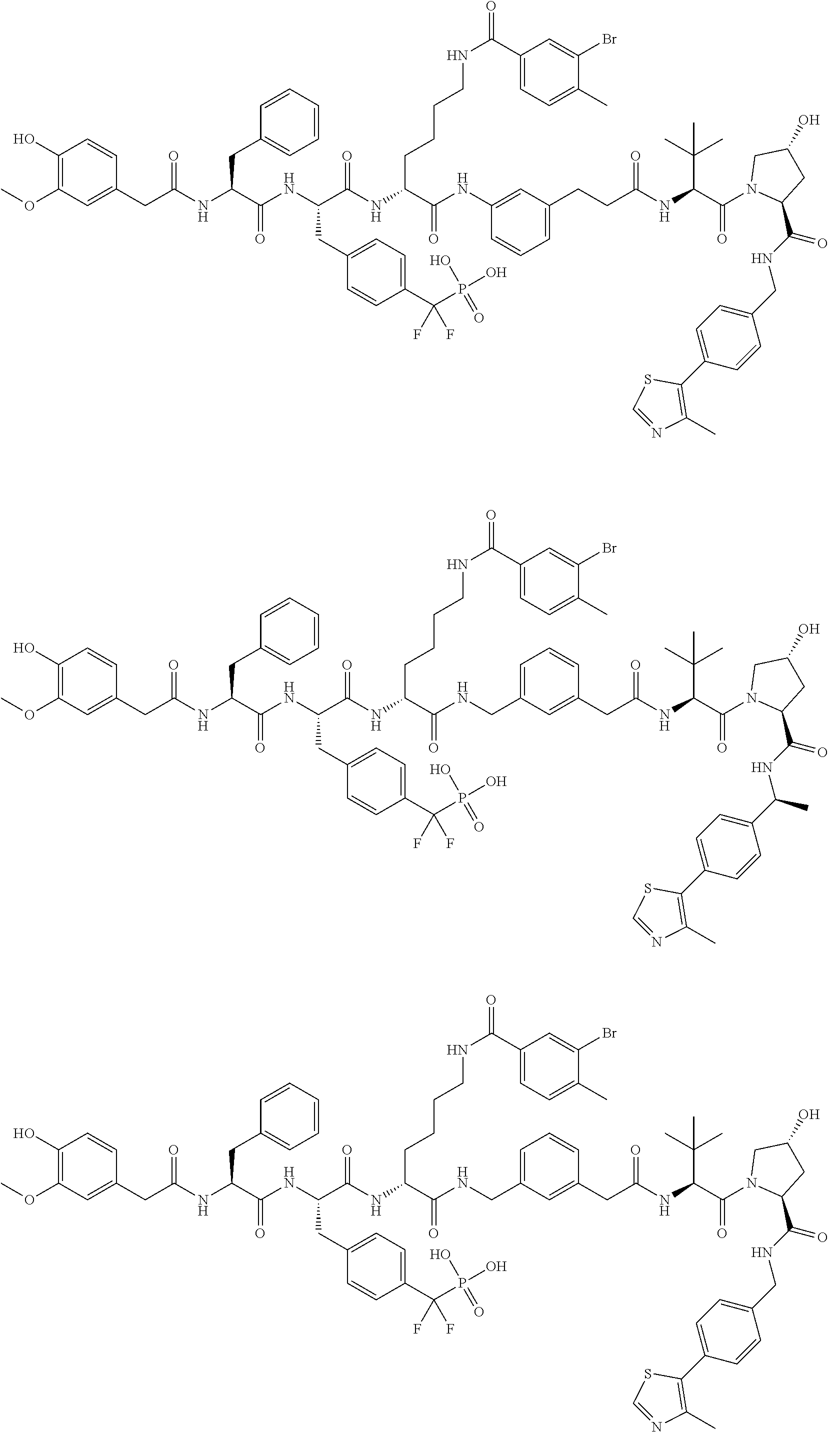

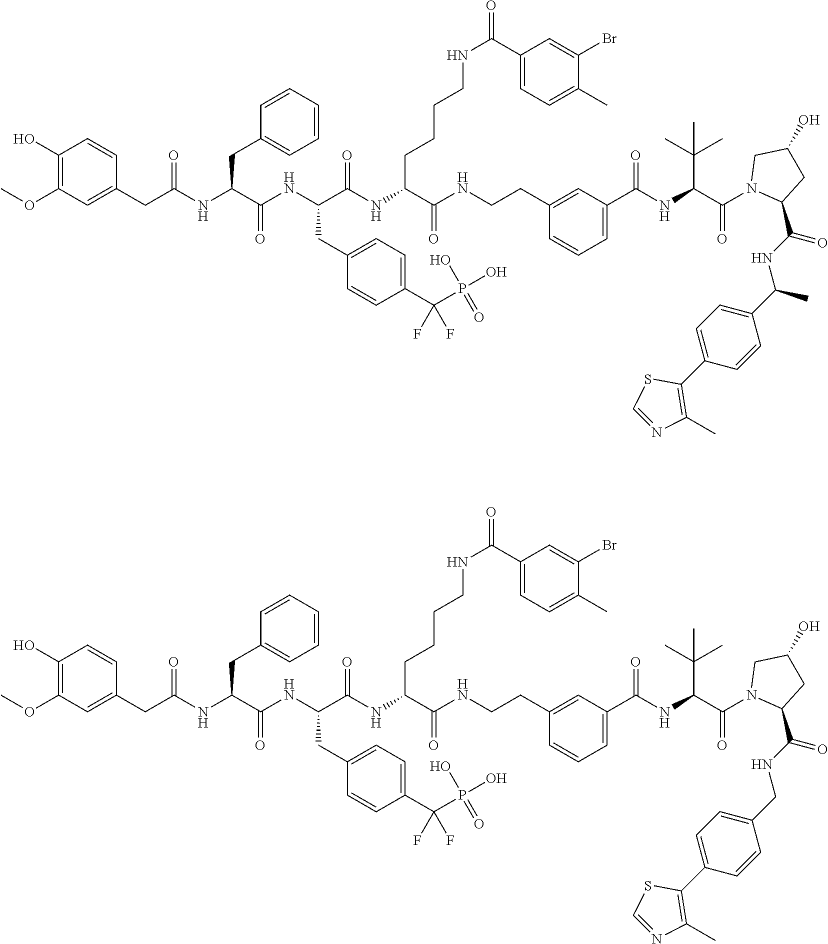 [SMILES: COc1cc(CC(=O)N[C@@H](Cc2ccccc2)C(=O)N[C@@H](Cc2ccc(C(F)(F)P(=O)(O)O)cc2)C(=O)N[C@H](CCCCNC(=O)c2ccc(C)c(Br)c2)C(=O)NCCc2cccc(C(=O)N[C@H](C(=O)N3C[C@H](O)C[C@H]3C(=O)N[C@@H](C)c3ccc(-c4scnc4C)cc3)C(C)(C)C)c2)ccc1O ; COc1cc(CC(=O)N[C@@H](Cc2ccccc2)C(=O)N[C@@H](Cc2ccc(C(F)(F)P(=O)(O)O)cc2)C(=O)N[C@H](CCCCNC(=O)c2ccc(C)c(Br)c2)C(=O)NCCc2cccc(C(=O)N[C@H](C(=O)N3C[C@H](O)C[C@H]3C(=O)NCc3ccc(-c4scnc4C)cc3)C(C)(C)C)c2)ccc1O]

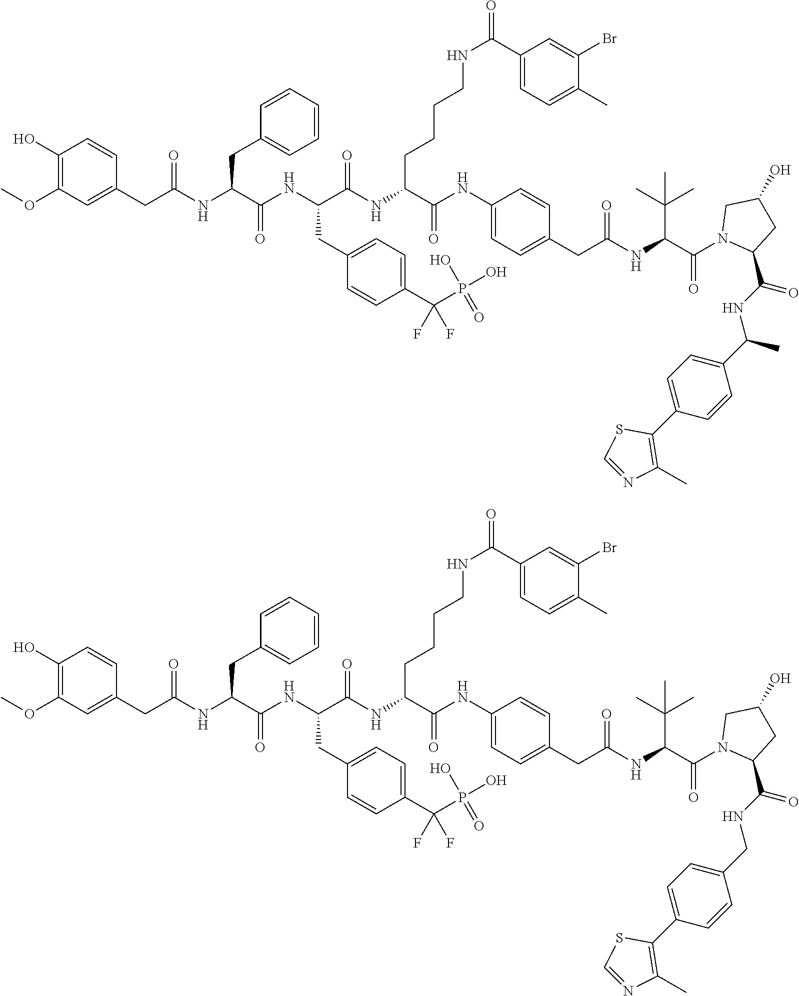 [SMILES: COc1cc(CC(=O)N[C@@H](Cc2ccccc2)C(=O)N[C@@H](Cc2ccc(C(F)(F)P(=O)(O)O)cc2)C(=O)N[C@H](CCCCNC(=O)c2ccc(C)c(Br)c2)C(=O)Nc2ccc(CC(=O)N[C@H](C(=O)N3C[C@H](O)C[C@H]3C(=O)N[C@@H](C)c3ccc(-c4scnc4C)cc3)C(C)(C)C)cc2)ccc1O ; COc1cc(CC(=O)N[C@@H](Cc2ccccc2)C(=O)N[C@@H](Cc2ccc(C(F)(F)P(=O)(O)O)cc2)C(=O)N[C@H](CCCCNC(=O)c2ccc(C)c(Br)c2)C(=O)Nc2ccc(CC(=O)N[C@H](C(=O)N3C[C@H](O)C[C@H]3C(=O)NCc3ccc(-c4scnc4C)cc3)C(C)(C)C)cc2)ccc1O]

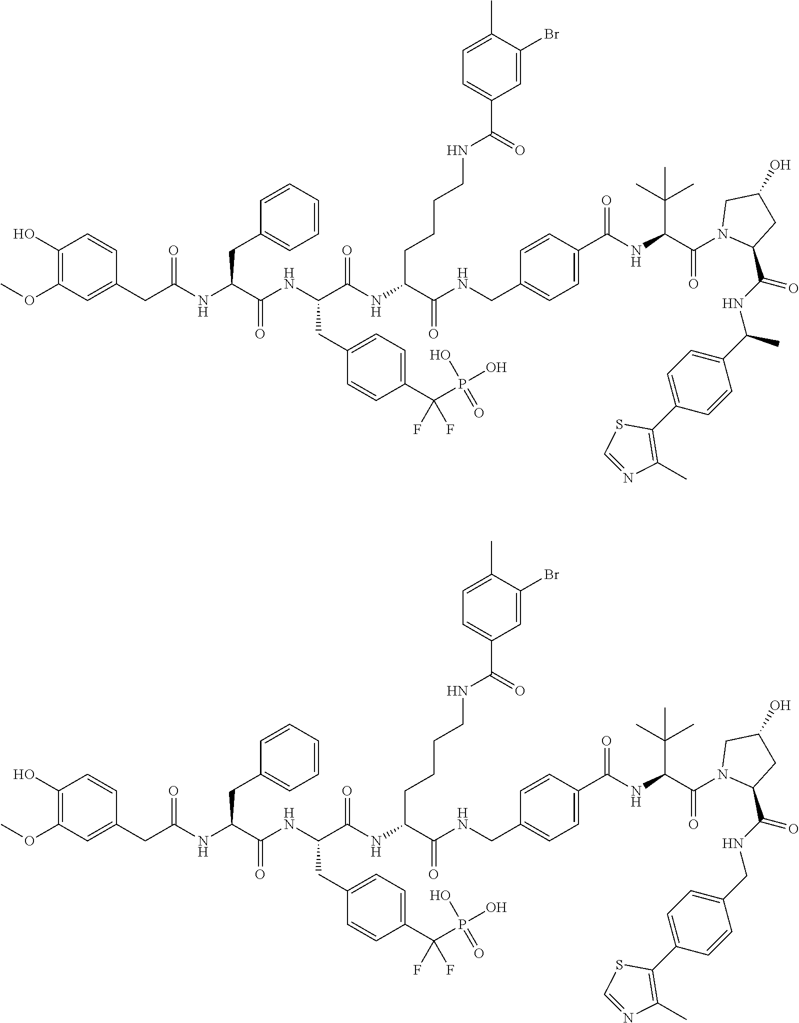 [SMILES: COc1cc(CC(=O)N[C@@H](Cc2ccccc2)C(=O)N[C@@H](Cc2ccc(C(F)(F)P(=O)(O)O)cc2)C(=O)N[C@H](CCCCNC(=O)c2ccc(C)c(Br)c2)C(=O)NCc2ccc(C(=O)N[C@H](C(=O)N3C[C@H](O)C[C@H]3C(=O)N[C@@H](C)c3ccc(-c4scnc4C)cc3)C(C)(C)C)cc2)ccc1O ; COc1cc(CC(=O)N[C@@H](Cc2ccccc2)C(=O)N[C@@H](Cc2ccc(C(F)(F)P(=O)(O)O)cc2)C(=O)N[C@H](CCCCNC(=O)c2ccc(C)c(Br)c2)C(=O)NCc2ccc(C(=O)N[C@H](C(=O)N3C[C@H](O)C[C@H]3C(=O)NCc3ccc(-c4scnc4C)cc3)C(C)(C)C)cc2)ccc1O]

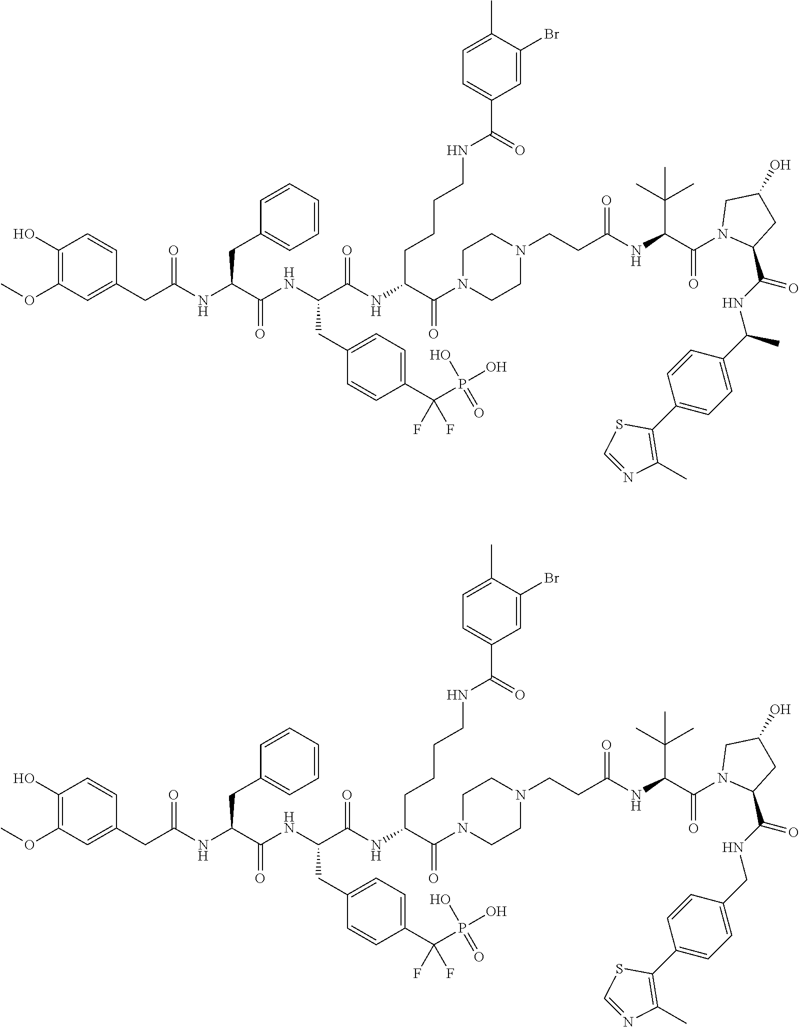 [SMILES: COc1cc(CC(=O)N[C@@H](Cc2ccccc2)C(=O)N[C@@H](Cc2ccc(C(F)(F)P(=O)(O)O)cc2)C(=O)N[C@H](CCCCNC(=O)c2ccc(C)c(Br)c2)C(=O)N2CCN(CCC(=O)N[C@H](C(=O)N3C[C@H](O)C[C@H]3C(=O)N[C@@H](C)c3ccc(-c4scnc4C)cc3)C(C)(C)C)CC2)ccc1O ; COc1cc(CC(=O)N[C@@H](Cc2ccccc2)C(=O)N[C@@H](Cc2ccc(C(F)(F)P(=O)(O)O)cc2)C(=O)N[C@H](CCCCNC(=O)c2ccc(C)c(Br)c2)C(=O)N2CCN(CCC(=O)N[C@H](C(=O)N3C[C@H](O)C[C@H]3C(=O)NCc3ccc(-c4scnc4C)cc3)C(C)(C)C)CC2)ccc1O]

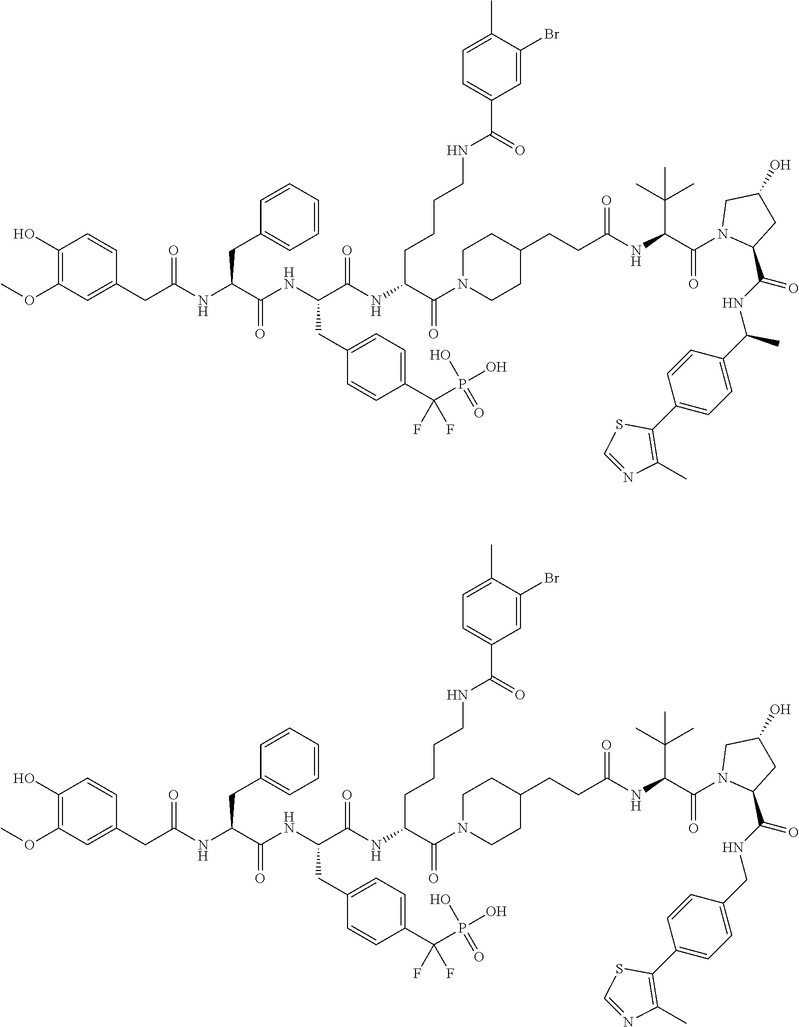 [SMILES: COc1cc(CC(=O)N[C@@H](Cc2ccccc2)C(=O)N[C@@H](Cc2ccc(C(F)(F)P(=O)(O)O)cc2)C(=O)N[C@H](CCCCNC(=O)c2ccc(C)c(Br)c2)C(=O)N2CCC(CCC(=O)N[C@H](C(=O)N3C[C@H](O)C[C@H]3C(=O)N[C@@H](C)c3ccc(-c4scnc4C)cc3)C(C)(C)C)CC2)ccc1O ; COc1cc(CC(=O)N[C@@H](Cc2ccccc2)C(=O)N[C@@H](Cc2ccc(C(F)(F)P(=O)(O)O)cc2)C(=O)N[C@H](CCCCNC(=O)c2ccc(C)c(Br)c2)C(=O)N2CCC(CCC(=O)N[C@H](C(=O)N3C[C@H](O)C[C@H]3C(=O)NCc3ccc(-c4scnc4C)cc3)C(C)(C)C)CC2)ccc1O]

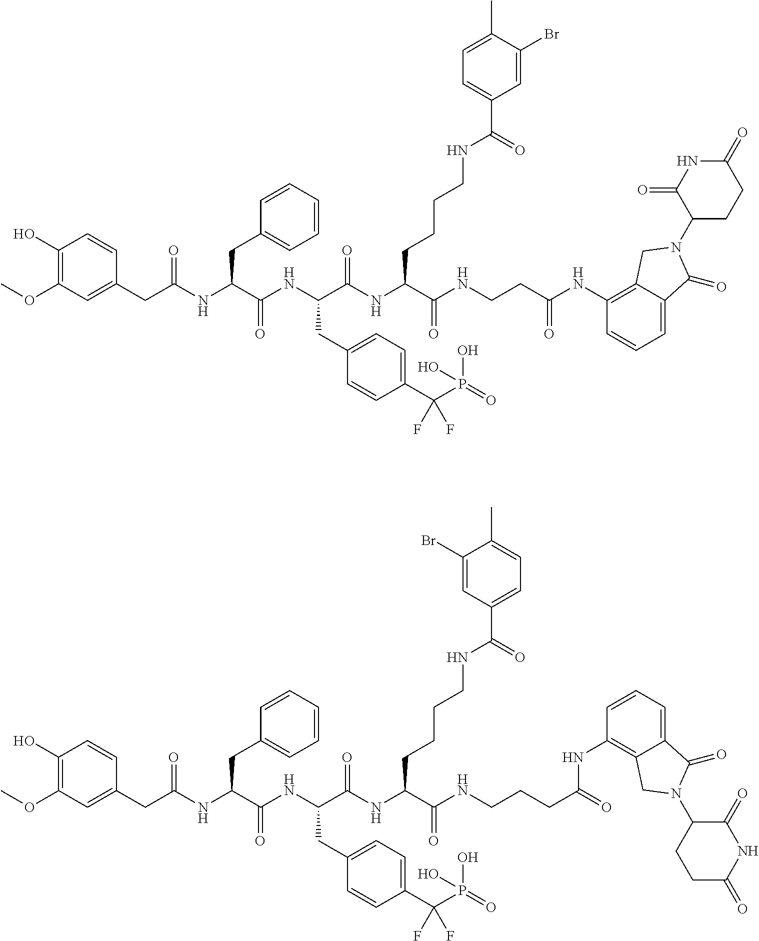 [SMILES: COc1cc(CC(=O)N[C@@H](Cc2ccccc2)C(=O)N[C@@H](Cc2ccc(C(F)(F)P(=O)(O)O)cc2)C(=O)N[C@@H](CCCCNC(=O)c2ccc(C)c(Br)c2)C(=O)NCCC(=O)Nc2cccc3c2CN(C2CCC(=O)NC2=O)C3=O)ccc1O ; COc1cc(CC(=O)N[C@@H](Cc2ccccc2)C(=O)N[C@@H](Cc2ccc(C(F)(F)P(=O)(O)O)cc2)C(=O)N[C@@H](CCCCNC(=O)c2ccc(C)c(Br)c2)C(=O)NCCCC(=O)Nc2cccc3c2CN(C2CCC(=O)NC2=O)C3=O)ccc1O]

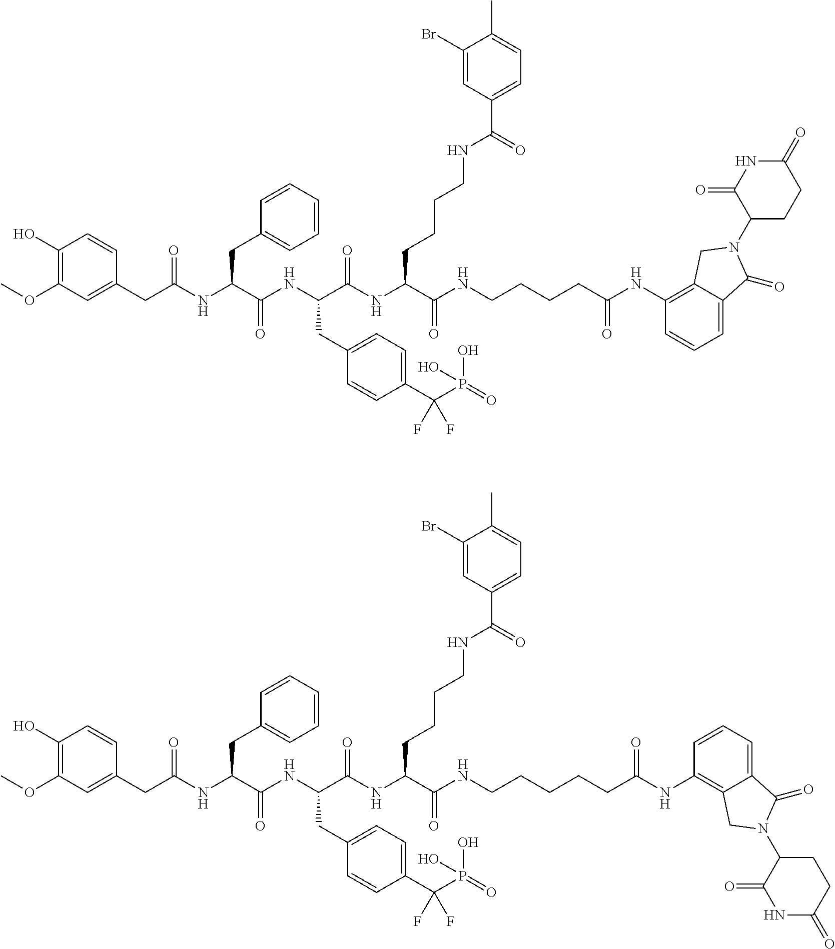 [SMILES: COc1cc(CC(=O)N[C@@H](Cc2ccccc2)C(=O)N[C@@H](Cc2ccc(C(F)(F)P(=O)(O)O)cc2)C(=O)N[C@@H](CCCCNC(=O)c2ccc(C)c(Br)c2)C(=O)NCCCCC(=O)Nc2cccc3c2CN(C2CCC(=O)NC2=O)C3=O)ccc1O ; COc1cc(CC(=O)N[C@@H](Cc2ccccc2)C(=O)N[C@@H](Cc2ccc(C(F)(F)P(=O)(O)O)cc2)C(=O)N[C@@H](CCCCNC(=O)c2ccc(C)c(Br)c2)C(=O)NCCCCCC(=O)Nc2cccc3c2CN(C2CCC(=O)NC2=O)C3=O)ccc1O]

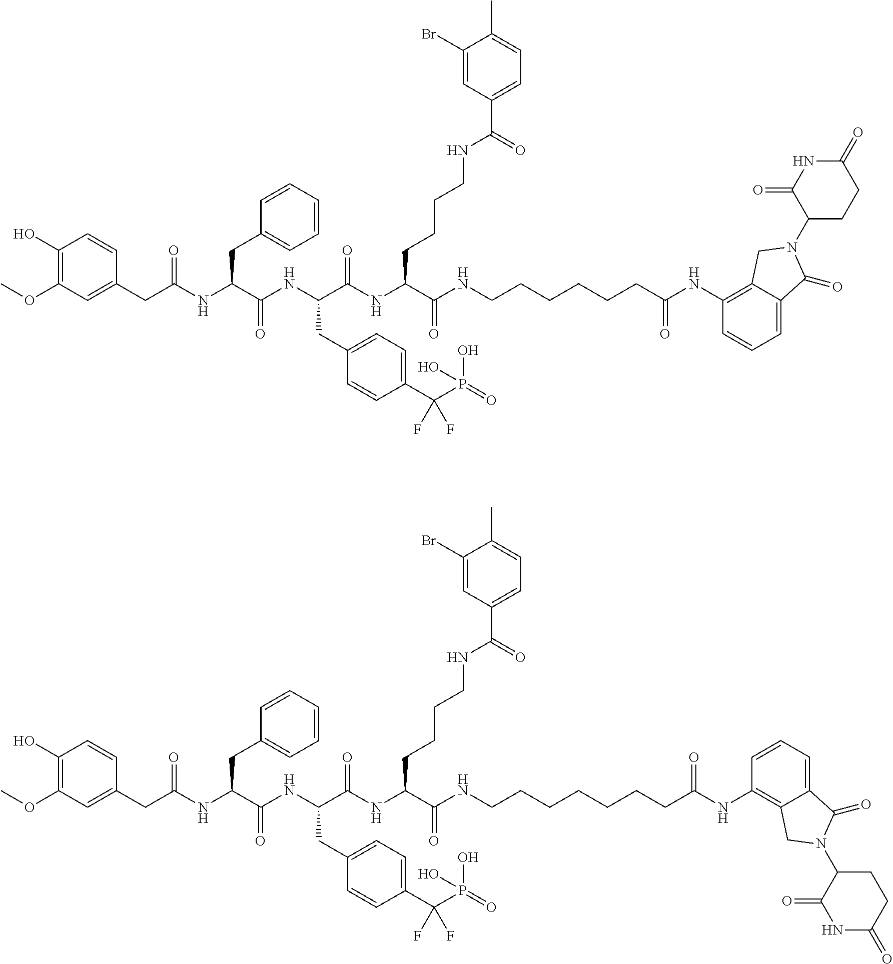 [SMILES: COc1cc(CC(=O)N[C@@H](Cc2ccccc2)C(=O)N[C@@H](Cc2ccc(C(F)(F)P(=O)(O)O)cc2)C(=O)N[C@@H](CCCCNC(=O)c2ccc(C)c(Br)c2)C(=O)NCCCCCCC(=O)Nc2cccc3c2CN(C2CCC(=O)NC2=O)C3=O)ccc1O ; COc1cc(CC(=O)N[C@@H](Cc2ccccc2)C(=O)N[C@@H](Cc2ccc(C(F)(F)P(=O)(O)O)cc2)C(=O)N[C@@H](CCCCNC(=O)c2ccc(C)c(Br)c2)C(=O)NCCCCCCCC(=O)Nc2cccc3c2CN(C2CCC(=O)NC2=O)C3=O)ccc1O]

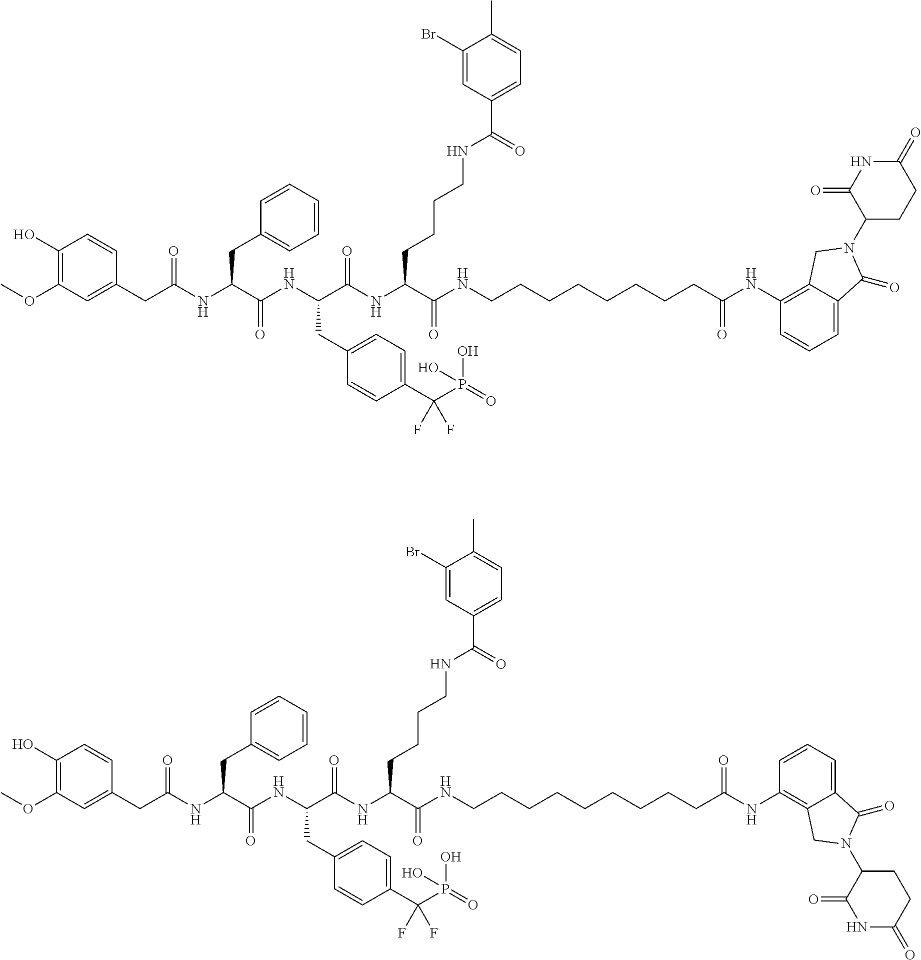 [SMILES: COc1cc(CC(=O)N[C@@H](Cc2ccccc2)C(=O)N[C@@H](Cc2ccc(C(F)(F)P(=O)(O)O)cc2)C(=O)N[C@@H](CCCCNC(=O)c2ccc(C)c(Br)c2)C(=O)NCCCCCCCCC(=O)Nc2cccc3c2CN(C2CCC(=O)NC2=O)C3=O)ccc1O ; COc1cc(CC(=O)N[C@@H](Cc2ccccc2)C(=O)N[C@@H](Cc2ccc(C(F)(F)P(=O)(O)O)cc2)C(=O)N[C@@H](CCCCNC(=O)c2ccc(C)c(Br)c2)C(=O)NCCCCCCCCCC(=O)Nc2cccc3c2CN(C2CCC(=O)NC2=O)C3=O)ccc1O]

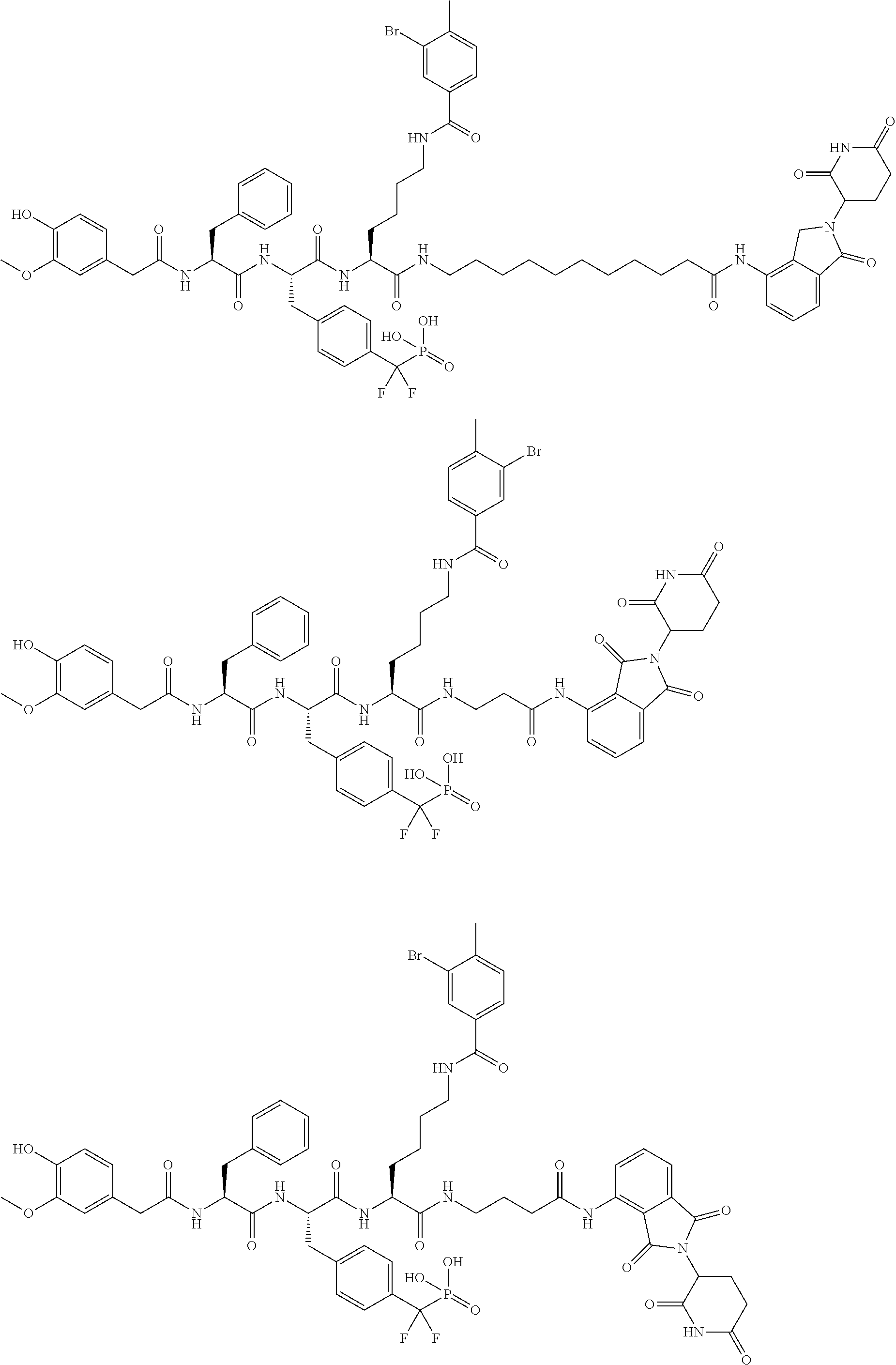

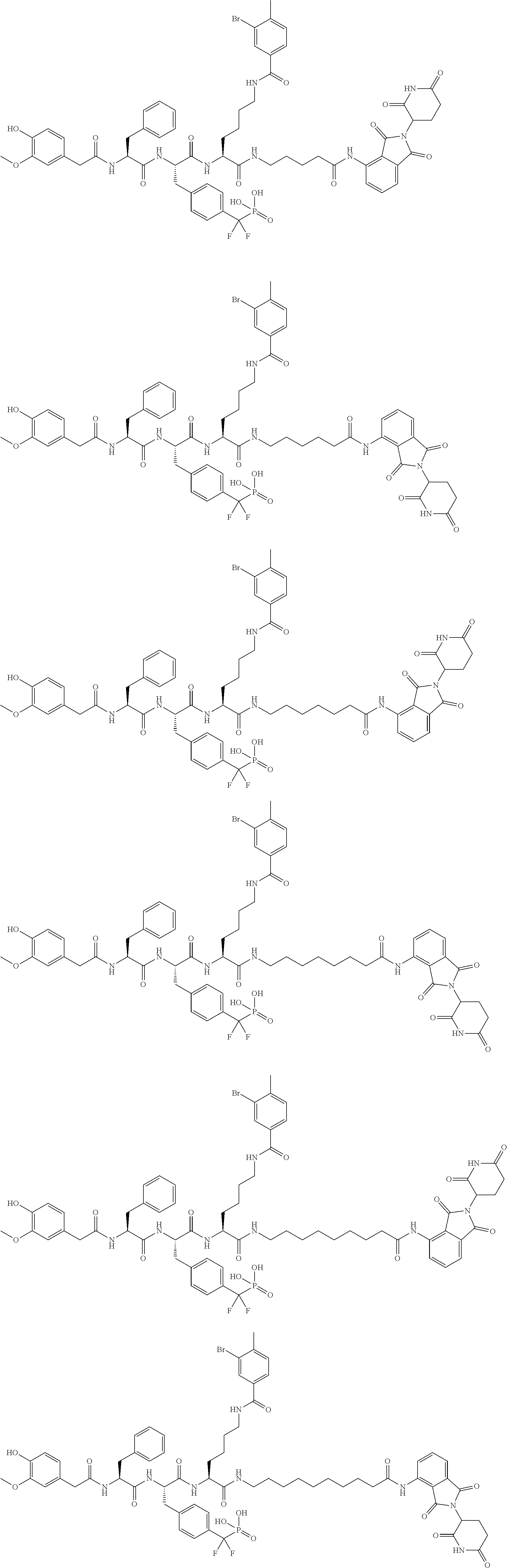

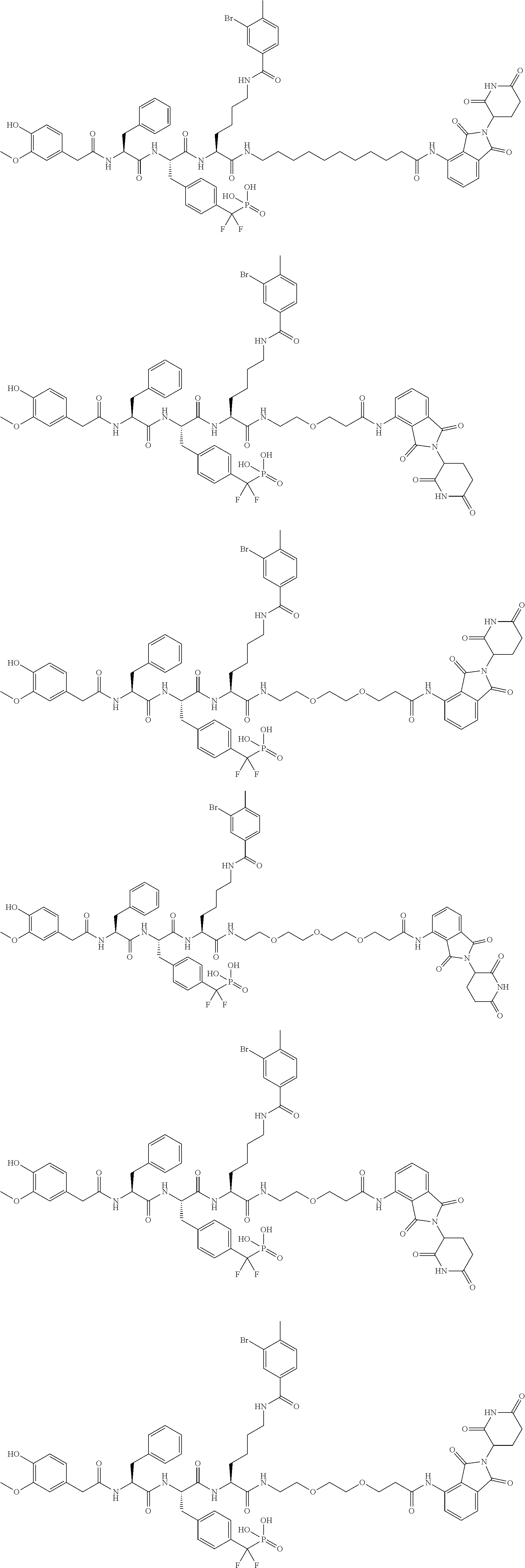

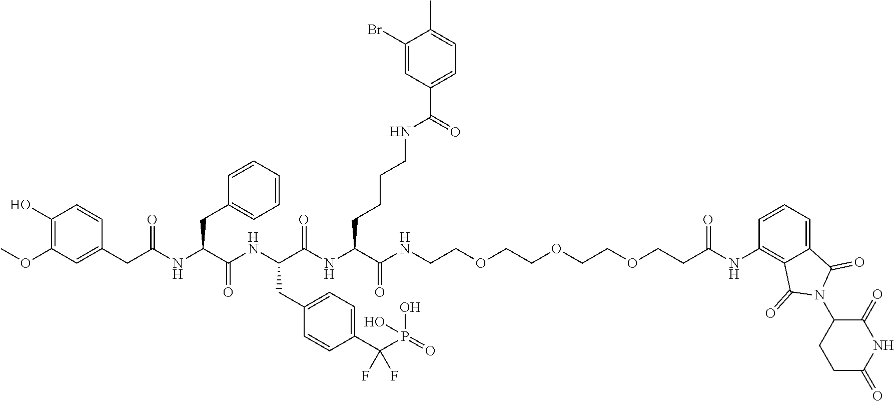 [SMILES: COc1cc(CC(=O)N[C@@H](Cc2ccccc2)C(=O)N[C@@H](Cc2ccc(C(F)(F)P(=O)(O)O)cc2)C(=O)N[C@@H](CCCCNC(=O)c2ccc(C)c(Br)c2)C(=O)NCCOCCOCCOCCC(=O)Nc2cccc3c2C(=O)N(C2CCC(=O)NC2=O)C3=O)ccc1O]

or pharmaceutically acceptable salt, hydrate, tautomer, and stereoisomer thereof.

Further provided is a pharmaceutical composition comprising one or more compounds of formula (I) or a pharmaceutically acceptable salt, hydrate, tautomer, and stereoisomer thereof, and a pharmaceutically acceptable carrier or excipient. Still further provided is a pharmaceutical composition comprising one or more compounds of formula (II) or a pharmaceutically acceptable salt, hydrate, tautomer, and stereoisomer thereof, and a pharmaceutically acceptable carrier or excipient. Still further provided is a pharmaceutical composition comprising one or more compounds of formula (III) or a pharmaceutically acceptable salt, hydrate, tautomer, and stereoisomer thereof, and a pharmaceutically acceptable carrier or excipient.

Still further provided is a method of treating or preventing cancer in a patient, wherein the method comprises inhibiting dual protein-tyrosine phosphatase 1B (PTP1B) and T-cell-PTP (TC-PTP) inhibitors by administering to the patient in need thereof an effective amount of a compound of formula (I) or (II) or a pharmaceutically acceptable salt, hydrate, tautomer, and stereoisomer of either of the foregoing, or a pharmaceutical composition comprising the compound and a pharmaceutically acceptable carrier or excipient, whereupon the cancer in the patient is treated or prevented.

Still further provided is a method of treating or preventing cancer in a patient, wherein the method comprises degrading dual PTP1B and TC-PTP proteins by administering to the patient in need thereof an effective amount of a compound of formula (I) or (III) or a pharmaceutically acceptable salt, hydrate, tautomer, and stereoisomer of either of the foregoing, or a pharmaceutical composition comprising the compound and a pharmaceutically acceptable carrier or excipient, whereupon the cancer in the patient is treated or prevented.

In some embodiments, the cancer is colon cancer, lung adenocarcinoma, squamous cell carcinoma, and melanoma. The compound is administered orally.

Still further provided is a method of treating or preventing type II diabetes in a patient, wherein the method comprises inhibiting dual PTP1B and TC-PTP inhibitors by administering to the patient in need thereof an effective amount of a compound of formula (I) or (II) or a pharmaceutically acceptable salt, hydrate, tautomer, and stereoisomer of either of the foregoing, or a pharmaceutical composition comprising the compound and a pharmaceutically acceptable carrier or excipient, whereupon the type 11 diabetes in the patient is treated or prevented.

Still further provided is a method of treating or preventing type II diabetes in a patient, wherein the method comprises degrading dual PTP1B and TC-PTP proteins by administering to the patient in need thereof an effective amount of a compound of formula (I) or (III) or a pharmaceutically acceptable salt, hydrate, tautomer, and stereoisomer of either of the foregoing, or a pharmaceutical composition comprising the compound and a pharmaceutically acceptable carrier or excipient, whereupon the type II diabetes in the patient is treated or prevented.

Still further provided is a method of treating or preventing obesity in a patient, wherein the method comprises inhibiting dual PTP1B and TC-PTP inhibitors by administering to the patient in need thereof an effective amount of a compound of formula (I) or (II) of or a pharmaceutically acceptable salt, hydrate, tautomer, and stereoisomer of either of the foregoing, or a pharmaceutical composition comprising the compound and a pharmaceutically acceptable carrier or excipient, whereupon the obesity in the patient is treated or prevented.

Still further provided is a method of treating or preventing obesity in a patient, wherein the method comprises degrading dual PTP1B and TC-PTP proteins by administering to the patient in need thereof an effective amount of a compound of formula (I) or (III) or a pharmaceutically acceptable salt, hydrate, tautomer, and stereoisomer of either of the foregoing, or a pharmaceutical composition comprising the compound and a pharmaceutically acceptable carrier or excipient, whereupon the obesity in the patient is treated or prevented.

Provided is a method of inhibiting or degrading dual PTP1B and TC-PTP in a patient, wherein the method comprises administering to the patient in need thereof an effective amount of a compound of formulae (I), (II) or (III) or a pharmaceutical composition comprising the same, whereupon dual PTP1B and TC-PTP in the patient is inhibited or degraded. In some embodiments, the patient has colon cancer, lung adenocarcinoma, squamous cell carcinoma, or melanoma. In some embodiments, the patient has type II diabetes. In some embodiments, the patient is obese.

Provided is the use of a compound of formula (I) in the treatment of a disease or condition that can be treated by inhibiting or degrading dual PTP1B or TC-PTP proteins. Further provided is the use of a compound of formula (II) in the treatment of a disease or condition that can be treated by inhibiting dual PTP1B or TC-PTP proteins. Still further provided is the use of a compound of formula (III) in the treatment of a disease or condition that can be treated by degrading dual PTP1B or TC-PTP proteins. In some embodiments, the disease or condition that can be treated is cancer, type II diabetes, or obesity.

### BRIEF DESCRIPTION OF THE DRAWINGS

The present disclosure will be more readily understood from the detailed description of embodiments presented below considered in conjunction with the attached drawings of which:

FIG. 1, shows that the compound of Example 7 is a potent and selective protein-tyrosine phosphatase 1B (PTP1B)/T-cell-PTP (TC-PTP) dual competitive inhibitor. (A) shows the effect of the compound of Example 7 on PTP1B-catalyzed pNPP hydrolysis. (B) shows the effect of the compound of Example 7 on TC-PTP catalyzed pNPP hydrolysis. The Lineweaver-Burk plot displayed the characteristic intersecting line pattern consistent with competitive inhibition. The concentrations of the compound of Example 7 were 0 (●), 5 (▪), and 10 nM (▴), respectively. The compound of Example 7 inhibits PTP1B and TC-PTP with Ki values of 2.4 and 3.6 nM, respectively. (C) shows the selectivity of the compound of Example 7 over a panel of 12 mammalian PTPs.

FIG. 2A shows a western blot of whole cell lysates from indicated cell lines that were treated with compound 47 for 16 hours (4 hours when specified) at the indicated concentration and stimulated with 10 ng/ml IFN-γ for 15 mins.

FIG. 2 B shows a western blot of whole cell lysates from indicated cell lines that were treated with compound 47 for 16 hours (4 hours when specified) at the indicated concentration and stimulated with 10 ng/ml IFN-γ for 15 mins.

FIG. 3A shows the quantification of PTP1B and TC-PTP levels after compound 47 induces PTP1B and TC-PTP degradation. The GAPDH protein was used as the loading control. Compound 47 induces PTP1B and TC-PTP degradation in all tested cell lines with low nanomolar DC₅₀ₛ.

FIG. 3B shows the quantification of pSTAT1 level after compound 47 upregulates STAT1 phosphorylation in multiple cell lines. The GAPDH protein was used as the loading control. Compound 47 treatment enhances pSTAT1 level in all cell lines. The changes in pSTAT1 level were positively correlated with TC-PTP/PTP1B degradation among all cell lines.

FIG. 4 shows MC38 cells were treated with DMSO or 500 nM compound 47 for 16 hours for PTP1B and TC-PTP degradation and then stimulated with mouse IFN-γ for 48 hours to induce MHC-I expression. Mouse MHC-I complex was stained with mouse H2K(b)/H2D(b) antibody and measured by flow cytometry. Compound 47-treated MC38 cells exhibited elevated expression of MHC-I.

FIG. 5 shows compound 47 effectively depletes PTP1B and TC-PTP protein in mouse MC38 syngeneic tumor and suppresses xenograft tumor growth. (A) shows mice blood concentration of compound 47 over time after single-dose administration of 25 (▪) or 50 (●) mg/kg compound 47 intraperitoneal (i.p.) injection. (B) shows MC38 tumor growth curve among ten days of treatment of 25 (▪) and 50 mg/kg (▴) compound 47 or saline (e) showing compound 47 significantly inhibits MC38 tumor growth. (C) shows bodyweight change of mice over ten days treatment period. (D) shows immunoblots of MC38 tumor extraction from mice treated with 25 mg/kg compound 47, 50 mg/kg compound 47, or saline showing PTP1B and TC-PTP degradation after compound 47 treatment in vivo. (E and F) shows image and quantification of mouse CD8α immunohistochemistry (IHC) staining of MC38 tumor slides from mice treated with 25 mg/kg compound 47 or saline. The tumor from compound 47 treated mice exhibits a high level of CD8-T cell infiltration. Quantification was performed based on six representative images from six sections.

FIG. 6A shows compound 47 is a dual PTP1B/TC-PTP PROTAC. The figure shows a mechanistic investigation of PTP1B/TC-PTP degradation induced by compound 47 in HEK293 cells. Cells were pre-treated with indicated concentration of MLN4924 (prevents ubiquitination), MG132 (blocks proteasome activity), lenalidomide (prevents CRBN binding) or (S,R,S)-AHPC-Me (prevents VHL binding) followed by 4 hours of treatment with compound 47 at 100 nM showing compound 47-mediated PTP1B/TC-PTP degradation depends on the ubiquitination-proteasome pathway. No degradation was observed when cells were treated with 100 nM cis-compound 47 (inactivated degrader) for 4 hours.

FIG. 6B-6C shows immunofluorescences of PTP1B and TC-PTP with U2OS cells treated with DMSO and 1 μM compound 47 for 3 hours and 24 hours. Compound 47 degraded cytoplasm-localized PTP1B and both nucleus- and cytoplasm-localized TC-PTP. MG132 (20 μM) was used along with compound 47 to block PTP1B and TC-PTP degradation.

FIG. 7 shows proteomic analysis showing the specificity of compound 47 for PTP1B degradation when HEK293 cells were treated with DMSO or 100 nM compound 47 for 4 hours.

FIG. 8 shows immunoblots of whole cell lysates from wild-type, PTP1B-deleted, or TC-PTP-deleted MEF cells that were treated with 0.5 μM compound 47 for 16 hours and stimulated with 20 ng/ml mouse IFN-γ for 15 mins. Deletion of TC-PTP or PTP1B abolished the compound 47-induced phosphorylation of the TC-PTP substrate JAK1 or the PTP1B substrate JAK2. Thus, compound 47 efficiently amplifies cellular IFN-7 signaling by degrading PTP1B and TC-PTP. U2OS cells were treated with DMSO or 0.2 μM compound 47 for 16 hours and stimulated with 20 ng/ml IFN-γ for 30 minutes. Immunofluorescence showing compound 47 dramatically enhanced IFN-γ mediated STAT1 phosphorylation and nucleus translocation. DAPI was used to stain the cell nucleus.

FIG. 9 shows compound 47 induces PTP1B and TC-PTP degradation in CD8+ naïve T-cells, enhances STAT1 and STAT5 phosphorylation, and promotes CD8⁺ T-cell activation. In (A) purified CD8⁺ naïve T-cells from control (Ptpn2^{fl/fl}) and Lck-Cre; Ptpn2^{fl/fl} mice (n=3/genotype/condition) were incubated for 48 hours with IL-7 in the presence or absence of compound 47 as indicated, and TC-PTP and PTP1B protein levels were monitored by flow cytometry. (B) show vehicle-treated control, TCPTP-deficient Lck-Cre; Ptpn2^{fl/fl} and compound 47-treated control naïve CD8⁺ T-cells were stimulated with plate-bound α-CD3/α-CD28 for 48 hours to promote T-cell activation. Basal STAT-1 (p(Y701) STAT-1) and STAT-5 (p(Y694) STAT-5) phosphorylation were assessed by flow cytometry. (C) cell size (FSC-A) and the T-cell activation markers CD25, CD69 and CD44 (MFI: mean fluorescence intensities) were measured by flow cytometry. (dot (●) represents Ptpn2^{fl/fl}+Vehicle, square (▪) represents Lck-Cre; Ptpn2^{fl/fl}+Vehicle, triangle (▴) represents Ptpn2^{fl/fl}+ compound 47).

FIG. 10 shows pharmaceutical kinetic studies of compound 47 in mice. Plasma concentrations of compound 47 were measured by LCMS after i.p. injection with different formulas at the indicated time.

FIG. 11 shows compound 47 reduces blood glucose levels of high-fat diet-fed (HFD) mice. (A) shows a reduction in blood glucose levels in high-fat diet-fed male mice (~50 g) when injected with 0, 15, or 50 mg/kg of compound 47. Blood glucose levels were measured 24 or 48 hours after injection. No starvation applied. NFD=normal fat diet fed mice. (B) shows blood glucose levels in MC38 synergetic tumor studies. Mice were treated with 25 or 50 mg/kg of compound 47 daily through i.p. injection. Blood glucose levels were measured several times before injection during the experiment.

FIG. 12A shows the western blots technique. Wild-type HEK293 cells were treated with compound 47 or ABBV-CLS-484 for 16 hours, and stimulated with 20 ng/ml IFN-γ to induce JAK-STAT signaling pathway activation. Western blots showed that compound 47 induced TC-PTP degradation and pSTAT1 elevation, while ABBV-CLS-484 also elevated the pSTAT1 level. Compound 47 activates pSTAT1 with superior efficiency compared to ABBV-CLS-484 in HEK293 cells.

FIG. 12B shows quantification of pSTAT1 levels based on western blotting results.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of the claimed invention is thereby intended.

The term “PTP1B/TC-PTP dual inhibitor” refers to a compound that inhibits both protein-tyrosine phosphatase 1B (PTP1B, also called tyrosine-protein phosphatase non-receptor type 1; protein tyrosine phosphatase non-receptor type 1; protein tyrosine phosphatase, placental; PTPN1; EC 3.1.3.48 and PTP-1B) and T-cell protein tyrosine phosphatase (TC-PTP, also called PTPN2; protein tyrosine phosphatase non-receptor type 2; TCELLPTP, T-cell protein-tyrosine phosphatase) proteins.

The term “PTP1B/TC-PTP dual degrader” refers to a compound that degrades both PTP1B and TC-PTP proteins.

The term “protein degrader” or “proteolysis targeting chimera (PROTAC)” refers to a heterobifunctional compound composed of three components: a ligand that binds to a target protein meant for degradation, a linker that can remove specific unwanted proteins, and a protein binding moiety that binds E3 ubiquitin ligase ligand.

The term von Hippel-Lindau (VHL) E3 ligase ligand “(S,R,S)-AHPC” refers to the following compound:

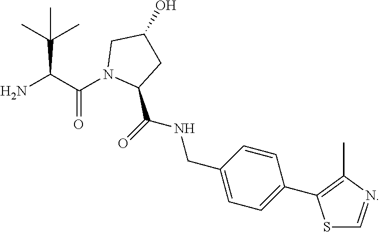 [SMILES: Cc1ncsc1-c1ccc(CNC(=O)[C@@H]2C[C@@H](O)CN2C(=O)[C@@H](N)C(C)(C)C)cc1]

The term VHL E3 ligase ligand “(S,R,S)-AHPC-Me” refers to the following compound:

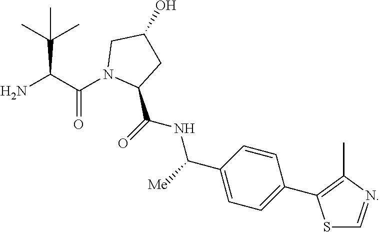 [SMILES: Cc1ncsc1-c1ccc([C@H](C)NC(=O)[C@@H]2C[C@@H](O)CN2C(=O)[C@@H](N)C(C)(C)C)cc1]

PTP1B and TC-PTP play non-redundant negative regulatory roles in T-cell activation, tumor antigen presentation, insulin, and leptin signaling and are potential targets for several therapeutic applications. The compound-mediated PTP1B and TC-PTP degradation depends on both target protein(s) and VHL E3 ligase ligand and is also ubiquitination- and proteasome-dependent. The ubiquitin-proteasome pathway (UPP) is used to induce selective protein degradation, including the use of fusion proteins to ubiquitinate artificially target proteins and synthetic small-molecule probes to induce proteasome-dependent degradation.

The present disclosure provides compounds that are dual inhibitors and dual degraders of PTP1B and TC-PTP.

Provided is a compound of formula (I), or a pharmaceutically acceptable salt, hydrate, tautomer, or stereoisomer thereof:

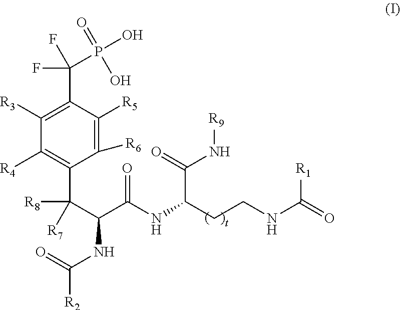

wherein
each R₁ and R₂ independently is a residue of a carboxylic acid, or a pharmaceutically acceptable salt thereof, wherein R₁ and R₂ can be the same or different;
R₃, R₄, R₅, R₆, R₇, and R₈ each independently are selected from the group consisting of hydrogen, deuterium, halogen, hydroxy, C₁-C₆ alkyl, C₃-C₆ cycloalkyl, —C₁-C₆ alkylene, 4-6 membered heterocyclyl, and —C₁-C₆ alkylene-4-6 membered heterocyclyl, wherein C₁-C₆ alkyl, C₃-C₆ cycloalkyl, —C₁-C₆ alkylene, 4-6 membered heterocyclyl, and —C₁-C₆ alkylene-4-6 membered heterocyclyl group optionally substituted on one or more available carbons by one or more substituents each independently selected from deuterium, halogen, hydroxy, C═O, C₁-C₆ alkyl, C₃-C₆ alkenyl, C₃-C₆ alkynyl, C₃-C₆ cycloalkyl, —C₁-C₆-alkylene-C₃-C₆ cycloalkyl, C₁-C₆ alkyl-S(O)₂—, C₃-C₆ cycloalkyl-S(O)₂—, C₁-C₆ alkyl-C(O)—, C₁-C₆ alkoxy-C(O)—, —NH—C(O)—Rₐ, and —C(O)—NH—Rₐ, wherein Rₐ is C₁-C₆ alkyl, C₃-C₆ cycloalkyl, —C₁-C₆ alkylene, 4-6 membered heterocyclyl, and —C₁-C₆ alkylene-4-6 membered heterocyclyl optionally substituted on one or more available carbons by one, or more substituents each independently selected from deuterium, halogen, hydroxy, C═O, C₁-C₆ alkyl, C₃-C₆ alkenyl, C₃-C₆ alkynyl, C₃-C₆ cycloalkyl, —C₁-C₆-alkylene-C₃-C₆ cycloalkyl, C₁-C₆ alkyl-S(O)₂—, C₁-C₆ cycloalkyl-S(O)₂—, C₁-C₆ alkyl-C(O)—, C₁-C₆ alkoxy-C(O)—, —NH—C(O)—R_{b}, and —C(O)—NH—R_{b}, wherein R_{b} is independently selected from deuterium, halogen, hydroxy, C═O, C₁-C₆ alkyl, C₃-C₆ alkenyl, C₃-C₆ alkynyl, C₃-C₆ cycloalkyl, —C₁-C₆-alkylene-C₃-C₆ cycloalkyl, C₁-C₆ alkyl-S(O)₂—, C₃-C₆ cycloalkyl-S(O)₂—, C₁-C₆ alkyl-C(O)—, and C₁-C₆alkoxy-C(O)—;
t is 0 to 6;
R₉ is H or a group represented by the formula L-B, wherein L is a linker selected from the group consisting of:

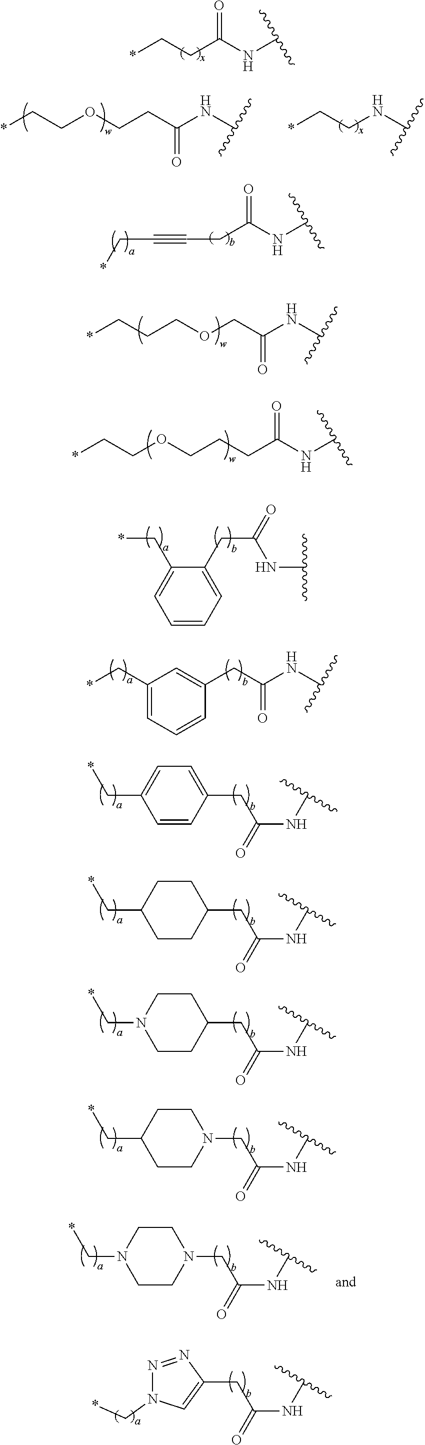

wherein w is 1-5, x is 1-15, and each a and b is independently 0-3;
the bond designated with an “ [symbol] ” is attached to B; the bond designated with “*” represents the point of attachment of R₉; and
B is selected from the group B₁, B₂, and B₃, wherein B₁ is represented by a structure:

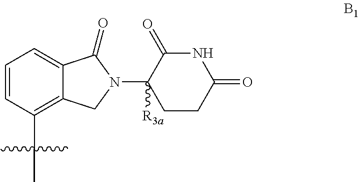

wherein R₃ₐ is fluoro, hydrogen, or deuterium;
B₂ is represented by a structure:

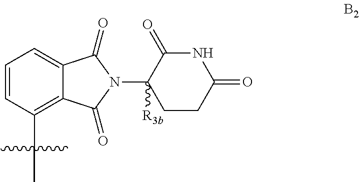

wherein R_{3b} is fluoro, hydrogen, or deuterium; and
B₃ is represented by a structure:

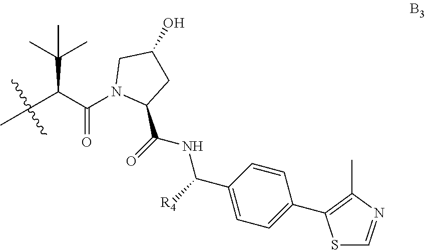

wherein R₄ is methyl, hydrogen, or deuterium.

In some embodiments, the compound of formula (I) can comprise (i) a first ligand represented by a structure:

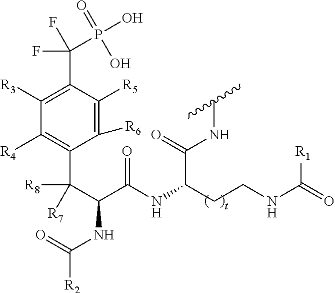

which binds to PTP1B/TC-PTP protein, (ii) a second ligand B, which binds to E3 ligase and is selected from the group B₁, B₂, and B₃ as defined above, and (iii) a linker L which binds the first ligand and the second ligand, wherein L, R₁, R₂, R₃, R₄, R₅, R₆, R₇, R₈ and t are as defined above.

Provided is a compound of formula (II), which is a dual inhibitor of PTP1B/TC-PTP, or a pharmaceutically acceptable salt, hydrate, tautomer, or stereoisomer thereof:

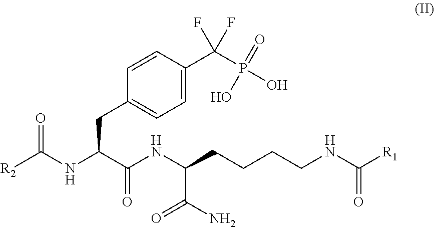

wherein R₁ and R₂ are as defined as for formula (I) above.

In some embodiments, R₁ or R₂ is:

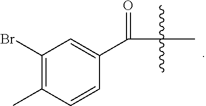 [SMILES: *C(=O)c1ccc(C)c(Br)c1]

In some embodiments, R₁ or R₂ is:

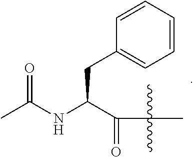 [SMILES: *C(=O)[C@H](Cc1ccccc1)NC(C)=O]

In some embodiments, R₁ or R₂ is:

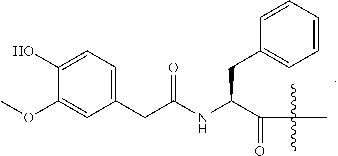 [SMILES: *C(=O)[C@H](Cc1ccccc1)NC(=O)Cc1ccc(O)c(OC)c1]

In some embodiments, the compound of formula (II) is:

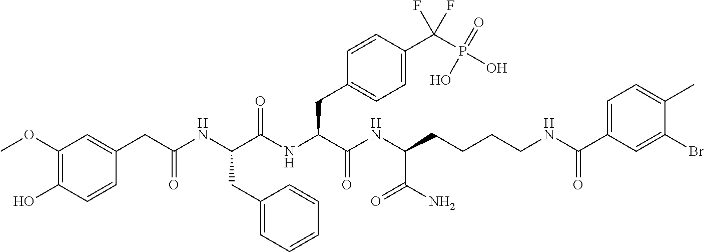

or a pharmaceutically acceptable salt, hydrate, tautomer, or stereoisomer thereof.

In some embodiments, provided are PROTAC compounds comprising (i) a first target protein-binding ligand that binds PTP1B and TC-PTP target proteins, (ii) a linker that tethers both first and second ligands and (iii) a second ligand that binds an E3 ligase that exploits the cell's ubiquitin-proteasome system to achieve selective target protein degradation. They induce the formation of a ternary complex by simultaneously binding to an E3 ligase, PTP1B, and TC-PTP, thereby bringing the PTP1B and TC-PTP into proximity of the E3 ligase for efficient ubiquitination and subsequent proteasome-mediated degradation.

Further provided is a compound of formula (III), which is a PTP1B/TC-PTP dual degrader:

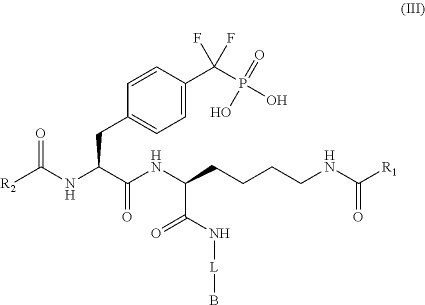

or a pharmaceutically acceptable salt, hydrate, tautomers, or stereoisomer thereof,
wherein R₁, R₂, L, and B are as defined as for formula (I) above.

In some embodiments, the R₁ or R₂ is selected from:

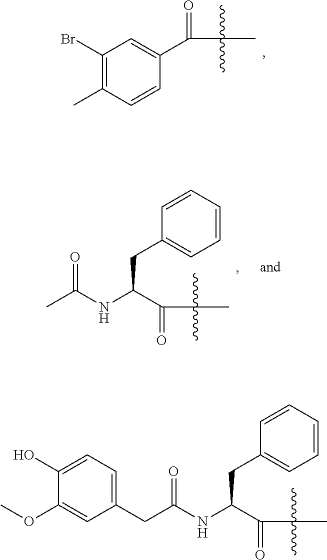 [SMILES: *C(=O)c1ccc(C)c(Br)c1 ; *C(=O)[C@H](Cc1ccccc1)NC(C)=O ; *C(=O)[C@H](Cc1ccccc1)NC(=O)Cc1ccc(O)c(OC)c1]

In some embodiments, the linker L is selected from:

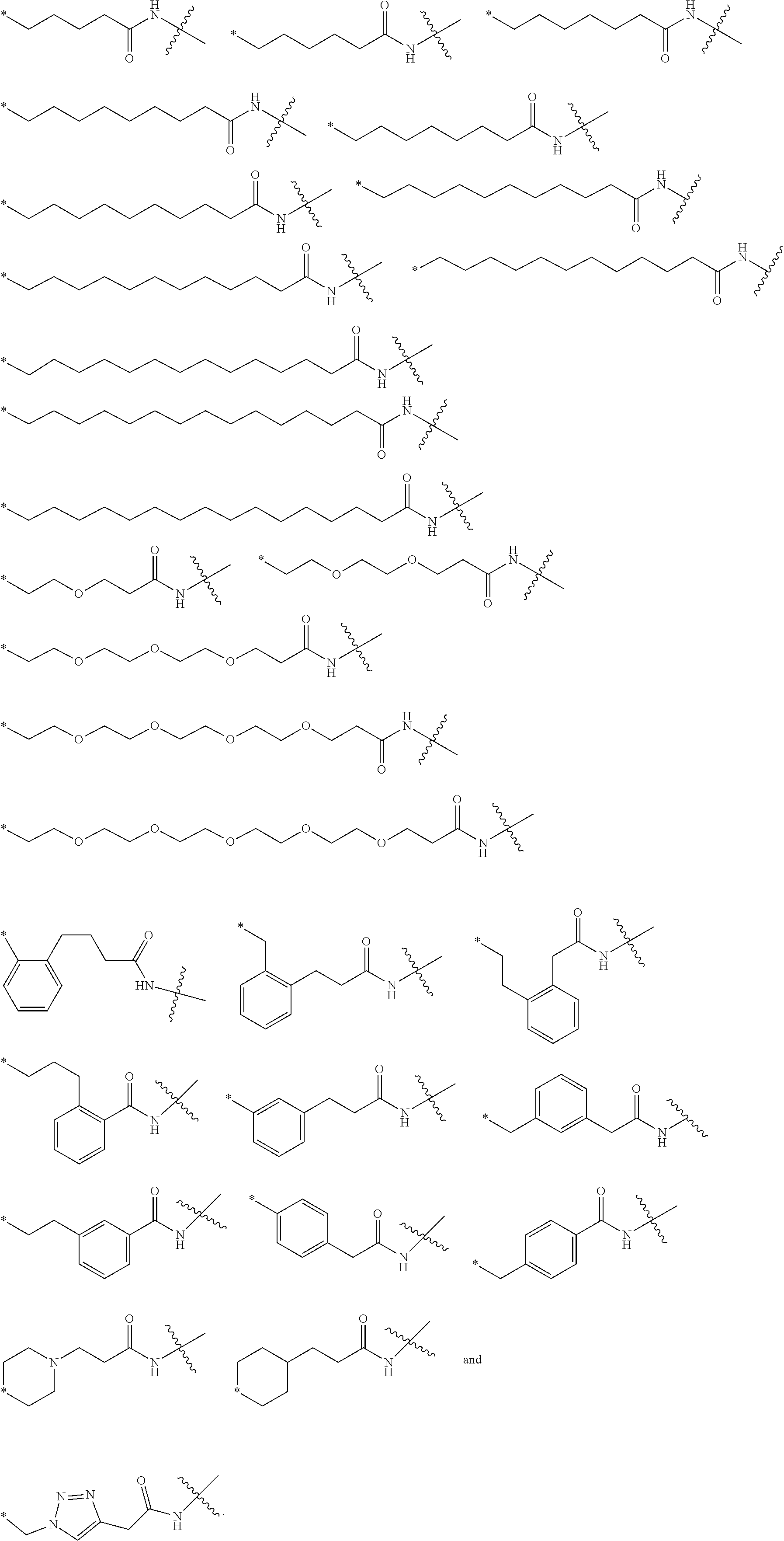

In some embodiments, the carboxylic acid in the compound of formula (I), (II) and (III) is represented by a formula RCOOH, wherein R is selected from a group consisting of hydrogen, an aliphatic group, or an aromatic group, wherein the aliphatic group is saturated or unsaturated, and wherein the aliphatic group or the aromatic group is substituted with C₁-C₂ alkyl, C₁-C₂ alkenyl, C₁-C₂₄ alkynyl, haloalkyl, haloalkenyl, heteroalkyl, cycloalkyl, cycloalkenyl, heterocycloalkyl, heterocycloalkenyl, aryl, heteroaryl, cycloalkylalkyl, heterocycloalkylalkyl, arylalkyl, heteroarylalkyl, arylalkenyl, cycloalkylheteroalkyl, heterocycloalkylheteroalkyl, heteroarylheteroalkyl, arylheteroalkyl, and acyl, wherein R group is optionally substituted with at least one of the groups selected from C₁-C₂₄ alkyl, hydroxy, alkoxy, cyano, halo, nitro, aryl, amino, C₁-C₂₄ alkenyl, C₁-C₂₄ alkynyl, haloalkyl, haloalkenyl, heteroalkyl, cycloalkyl, cycloalkenyl, heterocycloalkyl, heterocycloalkenyl, aryl, heteroaryl, cycloalkylalkyl, heterocycloalkylalkyl, arylalkyl, heteroarylalkyl, arylalkenyl, cycloalkylheteroalkyl, heterocycloalkylheteroalkyl, heteroarylheteroalkyl, arylheteroalkyl, and acyl.

In some embodiments, the carboxylic acid is selected from the group consisting of 3-dimethylaminobenzoic acid, 2-(2-cyanophenylthio)benzoic acid, 2-(4-chlorobenzoyl)benzoic acid, (−)-2-oxo-4-thiazolidine-carboxylic acid, (−)-N-acetylneuraminic acid, (+)-6-methoxy-α-methyl-2-naphthaleneacetic acid, (+)-carbobenzyloxy-D-proline, (+)-menthoxyacetic acid, (±)-2-(2-chlorophenoxy)propionic acid, (±)-1-methyl-2-cyclohexene-1-carboxylic acid, (1-naphthoxy)acetic acid, (IR)-(Ia,2b,3a)-(+)-3-methyl-2-nitromethyl-5-oxocyclopentaneacetic acid, (1R,4R)-7,7-dimethyl-2-oxobicyclo[2.2.1]heptane-1-carboxylic acid, (1S)-(+)-camphanic acid, (1S,3R,4S,5R)-1,3,4,5-tetrahydroxycyclohexanecarboxylic acid, (2,4-di-tert-pentylphenoxyl)acetic acid, (2-naphthoxy)acetic acid, (2-pyrimidylthio)acetic acid, (4-carboxybutyl)triphenyl-phosphonium bromide, (4-chlorophenylthio)acetic acid, (4-methylphenoxy)acetic acid, (α, α, α-trifluoro-m-tolyl)acetic acid, (E)-2-((4-hydroxyphenyl)diazenyl)benzoic acid, (E)-2-methyl-3-(2,4,5-trimethoxyphenyl)acrylic acid, (methylthio)acetic acid, (R)-(−)-2-hydroxy-4-phenylbutyric acid, (R)-(−)-3-chloromandelic acid, (R)-(−)-hexahydromandelic acid, (R)-(+)-2-pyrrolidone-5-carboxylic acid, (R)-(+)-citronellic acid, (R)-2-(1-phenylethylcarbamoyl)benzoic acid, (R)-2-hydroxy-2-phenylacetic acid, (R)-3,3,3-trifluoro-2-methoxy-2-phenylpropanoic acid, (R)-6-hydroxy-2,5,7,8-tetramethylchroman-2-carboxylic acid, (S)-(−)-indoline-2-carboxylic acid, (S)-(+)-2-oxo-4-phenyl-3-oxazolidineacetic acid, (S)-(+)-5-oxo-2-tetrahydro-furancarboxylic acid, (S)-(+)-hexahydromandelic acid, (S)-(+)-N-[1-(1-naphthyl)-ethyl]-phthalamic acid, (S)-(+)-O-acetylmandelic acid, (S)-2-(1-phenylethylcarbamoyl)benzoic acid, (S)-2-(4-isobutylphenyl)propanoic acid, (S)-2-(phenylcarbamoyloxy)propanoic acid, (S)-3-(benzyloxycarbonyl)-2-oxoimidazolidine-4-carboxylic acid, (S)-3,3,3-trifluoro-2-methoxy-2-phenylpropanoic acid, (S)-3,3,3-trifluoro-2-methoxy-2-phenylpropanoic acid, (S)-6-methoxy-2,5,7,8-tetramethylchroman-2-carboxylic acid, (trimethylsilyl)acetic acid, (Z)-2-cyano-3-(3-hydroxyphenyl)acrylic acid, 1-(4-chlorophenyl)-1-cyclopentanecarboxylic acid, 1-(tert-butyl)hydrocinnamic acid, 1,2-phenylenedioxydiacetic acid, 1,4-dihydro-2-methylbenzoic acid, 1,4-dihydroxy-2-naphthoic acid, 10-hydroxydecanoic acid, 10-undecynoic acid, 1-admantanecarboxylic acid, 1-cyano-1-cyclopropane-carboxylic acid, 1-hydroxy-2-naphthoic acid, 1-isoquinolinecarboxylic acid, 1-methyl-(1S,2R)-(+)-cis-1,2,3,6-tetrahydrophthalate, 1-methyl-1-cyclohexane-carboxylic acid, 1-methyl-1H-indole-2-carboxylic acid, 1-methyl-2-pyrrolecarboxylic acid, 1-methylcyclopropane-carboxylic acid, 1-naphthoic acid, 1-phenyl-1-cyclopentane-carboxylic acid, 1-phenyl-1-cyclopropane-carboxylic acid, 1-pyreneacetic acid, 1-pyrenebutyric acid, 1-pyrenecarboxylic acid, 2-((1R,2R,3R,4S)-3-hydroxy-4,7,7-trimethylbicyclo[2.2.1]heptan-2-yl)acetic acid, 2-((benzyloxycarbonyl)(methyl)amino)-2-methylpropanoic acid, 2-(2, (trifluoromethyl)phenyl)acetic acid, 2-(2,4,5-trichlorophenoxy)-propionic acid, 2-(2,4-dichlorophenoxy)-propionic acid, 2-(3,5-dinitrobenzamido)-2-phenylacetic acid, 2-(3,5-dinitrobenzamido)-4-methylpentanoic acid, 2-(3-chlorophenoxy)propionic acid, 2-(4-(trifluoromethyl)phenyl)acetic acid, 2-(4-chloro-3-nitrobenzoyl)-benzoic acid, 2-(4-chlorophenoxy)-2-methyl-propionic acid, 2-(4-chlorophenoxy)propionic acid, 2-(4-fluorobenzoyl)benzoic acid, 2-(4-hydroxy-3-methoxyphenyl)acetic acid, 2-(4-hydroxyphenoxy)-propionic acid, 2-(4-isobutylphenyl)propanoic acid, 2-(4-nitrophenyl)propionic acid, 2-(benzyloxycarbonylamino)-3-(1 H-indol-3-yl)propanoic acid, 2-(trifluoromethyl)acrylic acid, 2-(trifluoromethyl)benzoic acid, 2-(trifluoromethyl)cinnamic acid, 2,2,3,3-tetramethyl-cyclopropanecarboxylic acid, 2,2-bis(hydroxymethyl)-propionic acid, 2,3,4,5,6-pentafluoro-cinnamic acid, 2,3,4,5,6-pentafluorophenoxy acetic acid, 2,3,4,5,6-pentafluorophenyl-acetic acid, 2,3,4,5-tetrafluorobenzoic acid, 2,3,4-trifluorocinnamic acid, 2,3,4-trihydroxybenzoic acid, 2,3,4-trimethoxybenzoic acid, 2,3,5,6-tetrafluoro-4-hydroxy-benzoic acid hydrate, 2,3,5,6-tetrafluorobenzoic acid, 2,3,5,6-tetrafluoro-p-toluic acid, 2,3,5-triiodobenzoic acid, 2,3,6-trifluorobenzoic acid, 2,3-dichlorobenzoic acid, 2,3-difluorobenzoic acid, 2,3-dihydroxybenzoic acid, 2,3-dimethylbenzoic acid, 2,4,5-trichlorophenoxyacetic acid, 2,4,5-trimethoxybenzoic acid, 2,4,6-trichlorobenzoic acid, 2,4,6-trifluorobenzoic acid, 2,4,6-trihydroxybenzoic acid monohydrate, 2,4,6-trimethylbenzoic acid, 2,4-bis(trifluoromethyl)-benzoic acid, 2,4-dichloro-5-fluorobenzoic acid, 2,4-dichloro-5-sulfamoyl-benzoic acid, 2,4-dichlorobenzoic acid, 2,4-dichlorophenylacetic acid, 2,4-difluorobenzoic acid, 2,4-difluorophenylacetic acid, 2,4-dihydroxybenzoic acid, 2,4-dimethylbenzoic acid, 2,4-dinitrobenzoic acid, 2,4-dinitrophenylacetic acid, 2,4-hexadienoic acid, 2,5-bis(trifluoromethyl)-benzoic acid, 2,5-dichlorobenzoic acid, 2,5-difluorobenzoic acid, 2,5-difluorophenylacetic acid, 2,5-dihydroxybenzoic acid, 2,5-dihydroxyphenylacetic acid, 2,5-dimethoxybenzoic acid, 2,5-dimethoxycinnamic acid, 2,6-dichloro-3-nitrobenzoic acid, 2,6-difluorobenzoic acid, 2,6-difluorophenylacetic acid, 2,6-dihydroxybenzoic acid, 2,6-dimethoxynicotinic acid, 2,6-dimethylbenzoic acid, 2,6-heptadienoic acid, 2-[4-(dibutylamino)-2-hydroxy-benzoyl]benzoic acid, 2-bibenzylcarboxylic acid, 2-biphenylcarboxylic acid, 2-bromo-3-nitrobenzoic acid, 2-bromo-4,5-dimethoxybenzoic acid, 2-bromo-5-methoxybenzoic acid, 2-bromo-5-nitrobenzoic acid, 2-bromoacrylic acid, 2-bromophenylacetic acid, 2-chloro-3-nitrobenzoic acid, 2-chloro-4,5-difluorobenzoic acid, 2-chloro-4-fluorobenzoic acid, 2-chloro-5-(methylthio)-benzoic acid, 2-chloro-5-(trifluoro-methyl)benzoic acid, 2-chloro-5-nitrobenzoic acid, 2-chloro-5-nitrocinnamic acid, 2-chloro-6-fluorobenzoic acid, 2-chloro-6-fluorophenylacetic acid, 2-chloro-6-methylnicotinic acid, 2-chlorobenzoic acid, 2-chloronicotinic acid, 2-chlorophenylacetic acid, 2-chloropropionic acid, 2-ethoxy-1-naphthoic acid, 2-ethoxybenzoic acid, 2-ethyl-2-hydroxybutyric acid, 2-ethylbutyric acid, 2-ethylhexanoic acid, 2-ethylthio-2,2-diphenyl-acetic acid, 2-fluoro-3-(trifluoromethyl)-benzoic acid, 2-fluoro-4-(trifluoromethyl)-benzoic acid, 2-fluoro-5-methylbenzoic acid, 2-fluoro-5-nitrobenzoic acid, 2-fluoro-6-(trifluoromethyl)-benzoic acid, 2-fluorobenzoic acid, 2-fluorocinnamic acid, 2-fluorophenylacetic acid, 2-hydroxy-3-isopropyl-6-methylbenzoic acid, 2-hydroxy-3-isopropylbenzoic acid, 2-hydroxy-3-methylbutyric acid, 2-hydroxy-6-isopropyl-3-methylbenzoic acid, 2-hydroxycaproic acid, 2-hydroxyhippuric acid, 2-hydroxyisobutyric acid, 2-hydroxynicotinic acid, 2-hydroxyphenylacetic acid, 2-iodobenzoic acid, 2-mercaptonicotinic acid, 2-methoxy-2-phenylacetic acid, 2-methoxy-4-(methylthio)-benzoic acid, 2-methoxy-4-nitrobenzoic acid, 2-methoxyphenylacetic acid, 2-methyl-1-cyclohexane-carboxylic acid (cis and trans), 2-methyl-3-nitrobenzoic acid, 2-methyl-3-phenylpropanoic acid, 2-methyl-4-oxo-4-phenylbutyric acid, 2-methyl-6-nitrobenzoic acid, 2-methylbutyric acid, 2-methylcinnamic acid, 2-methylcyclopropane-carboxylic acid (cis and trans), 2-methylhexanoic acid, 2-methylhippuric acid, 2-methylhydrocinnamic acid, 2-methylvaleric acid, 2-naphthoic acid, 2-naphthylacetic acid, 2-nitro-4-(trifluoromethyl)benzoic acid, 2-nitrobenzoic acid, 2-norbornaneacetic acid, 2-oxo-6-pentyl-2H-pyran-3-carboxylic acid, 2-phenoxybenzoic acid, 2-phenoxybutyric acid, 2-phenoxypropionic acid, 2-propylpentanoic acid, 2-quinoxalinecarboxylic acid, 2-thiopheneacetic acid, 2-thiopheneacetic acid, 2-thiopheneglyoxylic acid, 3-(2-hydroxyphenyl)propionic acid, 3-(2-thienyl)acrylic acid, 3-(3,4,5-trimethoxyphenyl)-propionic acid, 3-(3,4-dimethoxyphenyl)-propionic acid, 3-(3-hydroxy-2,4,6-triiodophenyl)pentanoic acid, 3-(3-hydroxyphenyl)-propionic acid, 3-(3-methoxyphenyl)propionic acid, 3-(4-chlorobenzoyl)propionic acid, 3-(4-fluorobenzoyl)propionic acid, 3-(4-hydroxyphenyl)propionic acid, 3-(phenylsulfonyl)propionic acid, 3-(trifluoromethyl)cinnamic acid, 3-(trimethylsilyl)propynoic acid, 3,3,3-triphenylpropionic acid, 3,4-(methylenedioxy)cinnamic acid, 3,4-(methylenedioxy)phenyl-acetic acid, 3,4-dichlorobenzoic acid, 3,4-dichlorophenoxyacetic acid, 3,4-diethoxybenzoic acid, 3,4-difluorobenzoic acid, 3,4-dihydroxybenzoic acid, 3,4-dihydroxyhydrocinnamic acid, 3,4-dihydroxyphenylacetic acid, 3,5,6-trichlorosalicylic acid, 3,5-bis(trifluoromethyl)-phenyl acetic acid, 3,5-dibromobenzoic acid, 3,5-dichlorosalicyclic acid, 3,5-difluorocinnamic acid, 3,5-dihydroxy-2-naphthoic acid, 3,5-dinitrobenzoic acid, 3,5-dinitro-o-tuluic acid, 3,5-dinitro-p-toluic acid, 3,5-dinitrosalicyclic acid, 3,5-di-tert-butyl-4-hydroxy-benzoic acid, 3,5-di-tert-butylbenzoic acid, 3,7-dihydroxy-2-naphthoic acid, 3, thiopheneacetic acid, 3-benzoyl-2-pyridine-carboxylic acid, 3-benzoylbenzoic acid, 3-bromo-4-fluorobenzoic acid (95%), 3-bromo-4-methylbenzoic acid, 3-bromo-5-iodobenzoic acid, 3-bromobenzoic acid, 3-bromocinnamic acid, 3-carboxy-proxyl, 3-chloro-2-nitrobenzoic acid, 3-chloro-4-fluorobenzoic acid, 3-chloro-4-hydroxyphenyl-acetic acid, 3-chlorosalicylic acid, 3-cyanobenzoic acid, 3-fluoro-2-methylbenzoic acid, 3-fluoro-4-hydroxy-phenylacetic acid, 3-fluoro-4-methoxybenzoic acid, 3-fluorophenylacetic acid, 3-furoic acid, 3-hydroxy-2-naphthoic acid, 3-hydroxy-2-quinoxaline-carboxylic acid, 3-hydroxy-4-methoxybenzoic acid, 3-hydroxy-4-methoxy-cinnamic acid, 3-hydroxy-4-nitrobenzoic acid, 3-hydroxybenzoic acid, 3-hydroxybutyric acid, 3-hydroxyphenylacetic acid, 3-indolebutyric acid, 3-indoleglyoxylic acid, 3-indolepropionic acid, 3-iodo-4-methylbenzoic acid, 3-iodobenzoic acid, 3-isoquinolinecarboxylic acid hydrate, 3-methoxy-4-nitrobenzoic acid, 3-methoxycyclohexane-carboxylic acid (cis and trans), 3-methyl-2-phenylvaleric acid, 3-methylhippuric acid, 3-methylindene-2-carboxylic acid, 3-methylsalicylic acid, 3-methyl valeric acid, 3-nitrobenzoic acid, 3-nitrophenylacetic acid, 3-nitropropionic acid, 3-noradamantanecarboxylic acid, 3-oxo-1-indancarboxylic acid, 3-phenoxybenzoic acid, 3-phenylbutyric acid, 3-p-tolylpropanoic acid, 3-thiophenecarboxylic acid, 4-(1,3-dioxoisoindolin-2-yl)-2-hydroxybutanoic acid, 4-(2,4,5-trichlorophenoxy)-butyric acid, 4-(2,4-dichlorophenoxy)-butyric acid, 4-(2,4-di-tert-pentylphenoxy)butyric acid, 4-(2-phenoxyethoxy)benzoic acid, 4-(3,4-dimethoxyphenyl)-butyric acid, 4-(4-methoxyphenyl)butyric acid, 4-(4-nitrophenyl)butyric acid, A-(diethylamino)benzoic acid, 4-(dimethylamino)cinnamic acid, 4-(dimethylamino)phenyl-acetic acid, 4-(ethylthio)benzoic acid, 4-(hydroxymethyl)benzoic acid, 4-(methylsulfonyl)benzoic acid, 4-(methylthio)benzoic acid, 4-(methylthio)phenylacetic acid, 4-(trifluoromethoxy)benzoic acid, 4′-(trifluoromethyl)biphenyl-2-carboxylic acid, α-(trifluoromethyl)mandelic acid, 4,4,4-trifluoro-3-methyl-2-butenoic acid, 4,4-bis(4-hydroxyphenyl)-valeric acid, 4,5-dimethoxy-2-nitrobenzoic acid, 4,6-dioxoheptanoic acid, 4-[4-(2-carboxybenzoyl)-phenyl]butyric acid, 4-acetamidobenzoic acid. α-acetylbenzoic acid, 4-acetylphenoxyacetic acid, 4-benzyloxy-3-methoxyphenyl-acetic acid, 4-biphenylacetic acid, 4-bromo-3,5-dihydroxy-benzoic acid, 4-bromobenzoic acid, 4-bromocinnamic acid, 4-bromophenylacetic acid, 4-butoxybenzoic acid, A-butoxyphenylacetic acid, 4-butylbenzoic acid, 4-chloro-2,5-difluorobenzoic acid, 4-chloro-3-sulfamoylbenzoic acid, 4-chlorobenzoic acid, 4-chloro-o-tolyloxyacetic acid, A-chlorophenylacetic acid, 4-chlorosalicylic acid, 4-ethoxycarbonyloxy-3,5-dimethoxybenzoic acid, 4-ethoxyphenylacetic acid, 4-ethylbenzoic acid, 4′-ethylbiphenyl-4-carboxylic acid, 4-fluorenecarboxylic acid, 4-fluoro-1-naphthoic acid, α-fluoro-2-(trifluoromethyl)-benzoic acid, 4-fluoro-3-nitrobenzoic acid, 4-fluorobenzoic acid, 4-fluorobenzoic acid, 4-fluorocinnamic acid, 4-fluorophenoxyacetic acid, α-heptyloxybenzoic acid, 4-hexylbenzoic acid, 4-hexyloxybenzoic acid, 4-hydroxy-3-(morpholino-methyl)benzoic acid hydrate, 4-hydroxy-3,5-dinitrobenzoic acid, 4-hydroxy-3-methoxy-benzoic acid, 4-hydroxy-3-methoxy-mandelic acid, 4-hydroxy-3-nitrobenzoic acid, 4-hydroxy-3-nitrophenylacetic acid, 4-hydroxybenzoic acid, 4′-hydroxybiphenyl-4-carboxylic acid, 4-hydroxyphenylacetic acid, 4-hydroxyphenylacetic acid, α-hydroxyphenylpyruvic acid, 4-iodobenzoic acid, 4-isopropoxybenzoic acid, 4-methoxy-3-nitrobenzoic acid, 4-methoxycyclohexane-carboxylic acid, 4-methoxysalicylic acid, a-methyl-1-cyclohexane-carboxylic acid (cis and trans), 4-methyl-3-nitrobenzoic acid, α-methylhippuric acid, 4-methylsalicylic acid, 4-methylvaleric acid, 4-nitro-3-pyrazolecarboxylic acid, 4-nitrohippuric acid, 4-nonyloxybenzoic acid, 4-octylbenoic acid, 4-oxo-4H-1-benzopyran-2-carboxylic acid, 4-oxo-6-phenyl-5-hexenoic acid, α-pentenoic acid, 4-pentylbenzoic acid, 4-pentylbicyclo[2.2.2]octane-1-carboxylic acid, α-pentyloxybenzoic acid, 4-pentynoic acid, 4-phenylbutyric acid, 4-Propoxybenzoic acid, 4-propylbenzoic acid, 4-pyrazolecarboxylic acid, 4-tert-butylbenzoic acid, 4-tert-butylcyclohexanecarboxylic acid, 4-vinylbenzoic acid, 5-(4-chlorophenyl)-2-furoic acid, 5,6-dichloronicotinic acid, 5-bromo-2,4-dihydroxybenzoic acid, 5-fluoro-2-methylbenzoic acid, 5-fluoroindole-2-carboxylic acid, 5-fluorosalicylic acid, 5-hydantoinacetic acid, 5-hydroxy-2-indole-carboxylic acid, 5-methoxy-1-indanone-3-acetic acid, 5-methoxy-2-methyl-3-indoleacetic acid, 5-methoxy-2-nitrobenzoic acid, 5-methoxysalicylic acid, 5-methyl-2-nitrobenzoic acid, 5-methyl-2-pyrazine-carboxylic acid, 5-nitro-2-furoic acid, 5-nitro-3-pyrazolecarboxylic acid, 5-phenyl valeric acid, 6-(carbobenzyloxyamino)-caproic acid, 6-acetamidohexnoic acid, 6-bromohexanoic acid, 6-chloronicotinic acid, 6-hydroxy-2,5,7,8-tetramethylchroman-2-carboxylic acid, 6-methylchromone-2-carboxylic acid, 6-methylnicotinic acid, 6-nitrocaproic acid, 6-oxoheptaoic acid, 6-phenylhexanoic acid, 7-(carboxymethyoxy)-4-methylcoumarin, 7-hydroxycoumarin-4-acetic acid, 7-methoxy-2-benzofuran-carboxylic acid, 7-methoxycoumarin-4-acetic acid, 7-oxoctanoic acid, 9-anthracenecarboxylic acid, 9-fluoreneacetic acid, 9-fluorenone-1-carboxylic acid, α, α, α-trifluoro-m-toluic acid, α-acetamidocinnamic acid, abietic acid, acetic acid, acetyl-L-asparagine, acetylsalicyclic acid, α-cyano-4-hydroxycinnamic acid, adipic acid monoethyl ester, α-hydroxyhippuric acid, anthranilic acid, anti-3-oxotricyclo[2.2.1.02,6]heptane-7-carboxylic acid, α-phenylcyclopentaneacetic acid, α-phenyl-o-toluic acid, atrolactic acid, benzilic acid, benzotriazole-5-carboxylic acid, benzoylformic acid, bis(4-chlorophenyl)acetic acid, carbobenzyloxy-DL-alanine, carbobenzyloxy-L-alanine, carbobenzyloxy-1-glutamine, carbobenzyloxy-L-valine, cis-2-methoxycinnamic acid, crotonic acid, cyclohexanebutyric acid, cyclohexanecarboxylic acid, cyclohexanepentanoic acid, cyclohexanepropionic acid, cyclopentylacetic acid, D,L-3,4-dihydroxymandelic acid, D-3-phenyllactic acid, decanoic acid, dicyclohexylacetic acid, diethylphosphonoacetic acid, dikegulac hydrate, diphenylacetic acid, fumaric acid monoethyl ester, fusaric acid, gallic acid, geranic acid, glycolic acid, heptadecafluorononanoic acid, heptanoic acid, hexanoic acid, hippuric acid, hydrocinnamic acid, indole-3-carboxylic acid, indole-4-carboxylic acid, isovaleric acid. L-3-phenyl lactic acid, lauric acid, L-lactic acid (85%), maleamic acid, methoxyacetic acid, mono-(1R)-(−)-menthyl phthalate, mono-(1S)-(+)-menthyl phthalate, mono-methyl cis-5-norbornene-endo-2,3-dicarboxylate, mono-methyl phthalate, mono-methylterephthalate, N-(2-furoyl)glycine, n-(3,5-dinitrobenzoyl)-DL-a-phenylglycine, N-(3-indolylacetyl)-L-alanine, N-(3-indolylacetyl)-L-isoleucine, N-(3-indolylacetyl)-L-leucine, N-(3-indolylacetyl)-L-phenylalanine, N-(3-indolylacetyl)-L-valine, N-(carbobenzyloxy)-1-phenyl-alanine, N,N-diethyl-3,6-difluoro-phthalamic acid, N—[(R)—I-(1-naphthyl)ethyl]-phthalamic acid, N-[5-(trifluoromethyl)-2-pyridyl]-L-valine, n-acetyl-4-fluoro-DL-phenylalanine, N-acetyl-DL-tryptophan, n-acetyl-1-leucine, N-acetyl-L-methionine, N-acetyl-L-phenylalanine, N-acetyl-1-tyrosine, N-benzoyl-(2R,3S)-3-phenyl-isoserine. N-benzoyl-L-threonine, N-carbobenzyloxy-2-methyl-alanine, N-carbobenzyloxy-L-glutamic acid 1-methyl ester, N-carbobenzyloxy-L-isoleucine, N-carbobenzyloxy-L-Leucine, N-carbobenzyloxy-1-threonine, N-ethoxycarbonyl-1-phenylalanine, nonanoic acid, N-p-tosylglycine, N-p-tosyl-L-phenylalanine, o-anisic acid, p-anisic acid, pentafluorobenzoic acid, phenoxyacetic acid, phenylacetic acid, podocarpic acid, pyruvic acid, rhodanine-3-acetic acid. S-(thiobenzoyl)thioglycolic acid, S-benzyl-n-carbobenzyloxy-1-cysteine, sebacic acid monomethyl ester, succinamic acid, succinic 2,2-dimethyl-hydrazide, tetrahydro-2-furoic acid, trans-1-acetyl-4-hydroxy-L-proline, trans-2,3-dimethoxycinnamic acid, trans-2,4-dichlorocinnamic acid, trans-2,4-difluorocinnamic acid, trans-2,5-difluorocinnamic acid, trans-2,6-difluorocinnamic acid, trans-2-chloro-6-fluro-cinnamic acid, trans-2-hexenoic acid, trans-3-(2,3,5,6-tetramethyl-benzoyl)acrylic acid, trans-3-(2,5-dimethylbenzo-yl)-acrylic acid, trans-3-(4-ethoxy-benzoyl)acrylic acid, trans-3-(4-methoxybenzoyl)-acrylic acid, trans-3-(4-methylbenzoyl)-acrylic acid, trans-3,4-difluorocinnamic acid, trans-3-fluorocinnamic acid, trans-3-furanacrylic acid, trans-3-hexenoic acid, trans-4-chloro-3-nitrocinnamic acid, trans-4-hydroxy-3-methoxy-cinnamic acid, trans-4-methyl-1-cyclohexane carboxylic acid, trans-4-pentylcyclohexane carboxylic acid, trans-5-bromo-2-methoxy cinnamic acid, trans-styrylacetic acid, tridecafluoroheptanoic acid, trimethylacetic acid, triphenylacetic acid, valeric acid, and yohimbinic acid mono-hydrate.

In some embodiments, the compound of formula (III) is,

| Compound No. | Structure |
|---|---|
| 1 | 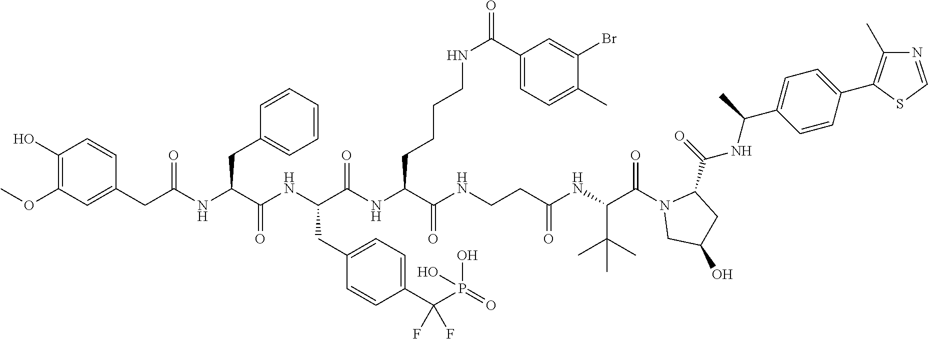 [SMILES: COc1cc(CC(=O)N[C@@H](Cc2ccccc2)C(=O)N[C@@H](Cc2ccc(C(F)(F)P(=O)(O)O)cc2)C(=O)N[C@@H](CCCCNC(=O)c2ccc(C)c(Br)c2)C(=O)NCCC(=O)N[C@H](C(=O)N2C[C@H](O)C[C@H]2C(=O)N[C@@H](C)c2ccc(-c3scnc3C)cc2)C(C)(C)C)ccc1O] |
| 2 | 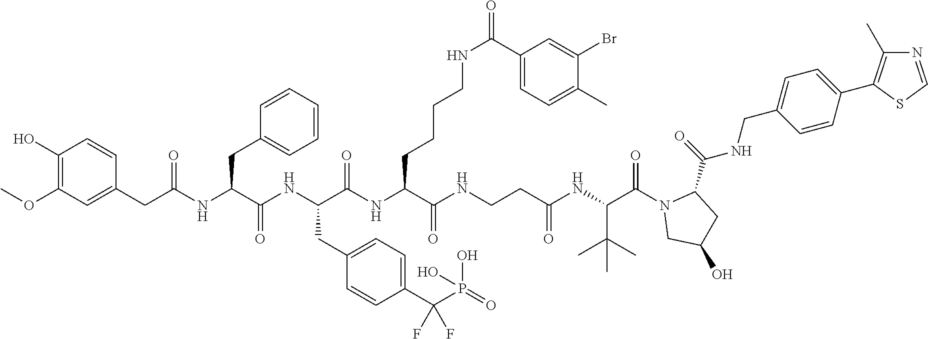 [SMILES: COc1cc(CC(=O)N[C@@H](Cc2ccccc2)C(=O)N[C@@H](Cc2ccc(C(F)(F)P(=O)(O)O)cc2)C(=O)N[C@@H](CCCCNC(=O)c2ccc(C)c(Br)c2)C(=O)NCCC(=O)N[C@H](C(=O)N2C[C@H](O)C[C@H]2C(=O)NCc2ccc(-c3scnc3C)cc2)C(C)(C)C)ccc1O] |
| 3 | 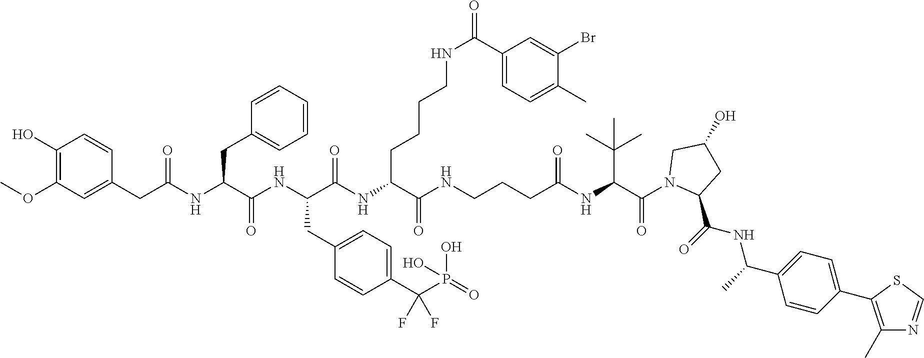 [SMILES: COc1cc(CC(=O)N[C@@H](Cc2ccccc2)C(=O)N[C@@H](Cc2ccc(C(F)(F)P(=O)(O)O)cc2)C(=O)N[C@H](CCCCNC(=O)c2ccc(C)c(Br)c2)C(=O)NCCCC(=O)N[C@H](C(=O)N2C[C@H](O)C[C@H]2C(=O)N[C@@H](C)c2ccc(-c3scnc3C)cc2)C(C)(C)C)ccc1O] |
| 4 | 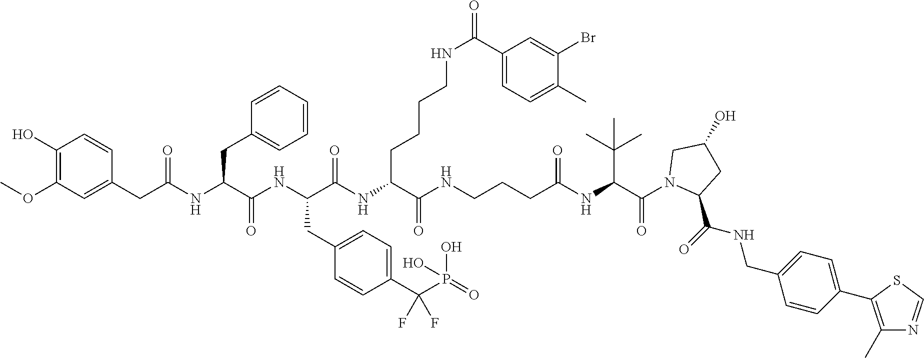 [SMILES: COc1cc(CC(=O)N[C@@H](Cc2ccccc2)C(=O)N[C@@H](Cc2ccc(C(F)(F)P(=O)(O)O)cc2)C(=O)N[C@H](CCCCNC(=O)c2ccc(C)c(Br)c2)C(=O)NCCCC(=O)N[C@H](C(=O)N2C[C@H](O)C[C@H]2C(=O)NCc2ccc(-c3scnc3C)cc2)C(C)(C)C)ccc1O] |
| 5 | 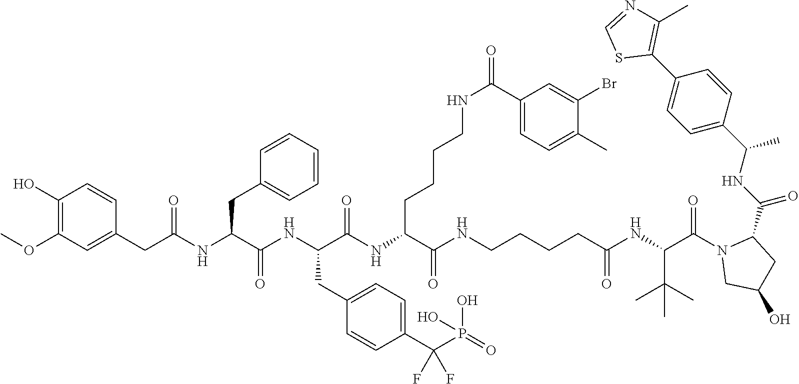 [SMILES: COc1cc(CC(=O)N[C@@H](Cc2ccccc2)C(=O)N[C@@H](Cc2ccc(C(F)(F)P(=O)(O)O)cc2)C(=O)N[C@H](CCCCNC(=O)c2ccc(C)c(Br)c2)C(=O)NCCCCC(=O)N[C@H](C(=O)N2C[C@H](O)C[C@H]2C(=O)N[C@@H](C)c2ccc(-c3scnc3C)cc2)C(C)(C)C)ccc1O] |
| 6 | 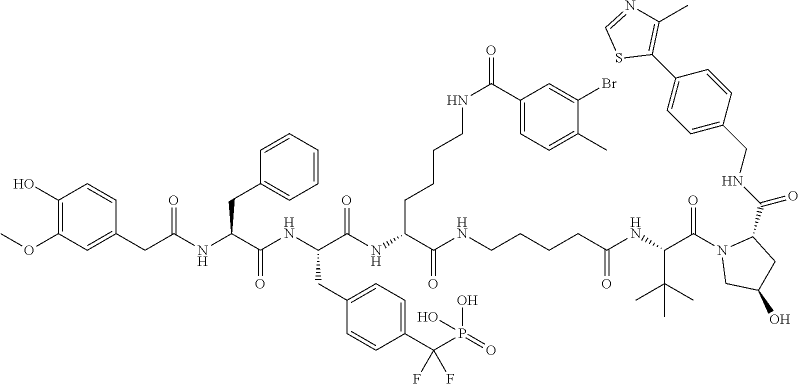 [SMILES: COc1cc(CC(=O)N[C@@H](Cc2ccccc2)C(=O)N[C@@H](Cc2ccc(C(F)(F)P(=O)(O)O)cc2)C(=O)N[C@H](CCCCNC(=O)c2ccc(C)c(Br)c2)C(=O)NCCCCC(=O)N[C@H](C(=O)N2C[C@H](O)C[C@H]2C(=O)NCc2ccc(-c3scnc3C)cc2)C(C)(C)C)ccc1O] |
| 7 | 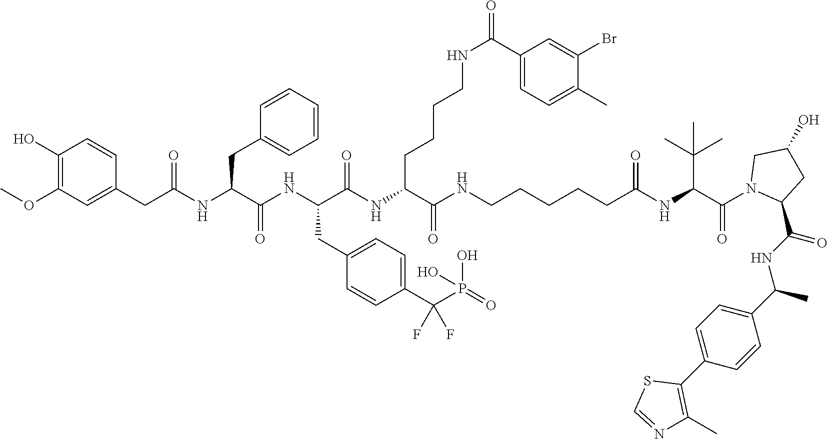 [SMILES: COc1cc(CC(=O)N[C@@H](Cc2ccccc2)C(=O)N[C@@H](Cc2ccc(C(F)(F)P(=O)(O)O)cc2)C(=O)N[C@H](CCCCNC(=O)c2ccc(C)c(Br)c2)C(=O)NCCCCCC(=O)N[C@H](C(=O)N2C[C@H](O)C[C@H]2C(=O)N[C@@H](C)c2ccc(-c3scnc3C)cc2)C(C)(C)C)ccc1O] |
| 8 | 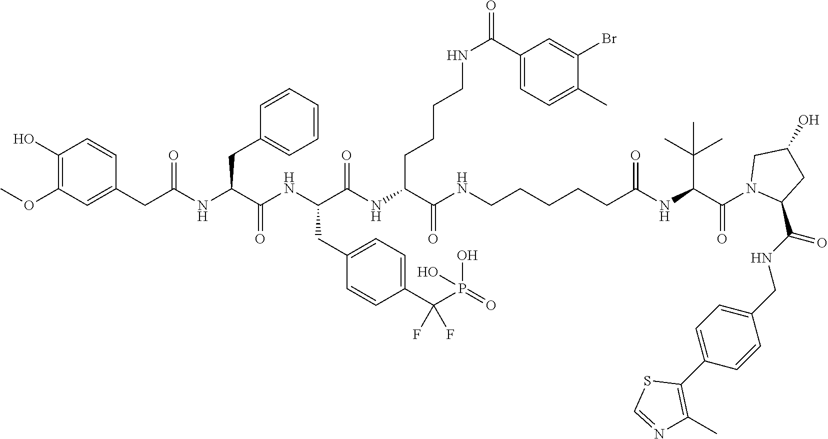 [SMILES: COc1cc(CC(=O)N[C@@H](Cc2ccccc2)C(=O)N[C@@H](Cc2ccc(C(F)(F)P(=O)(O)O)cc2)C(=O)N[C@H](CCCCNC(=O)c2ccc(C)c(Br)c2)C(=O)NCCCCCC(=O)N[C@H](C(=O)N2C[C@H](O)C[C@H]2C(=O)NCc2ccc(-c3scnc3C)cc2)C(C)(C)C)ccc1O] |
| 9 | 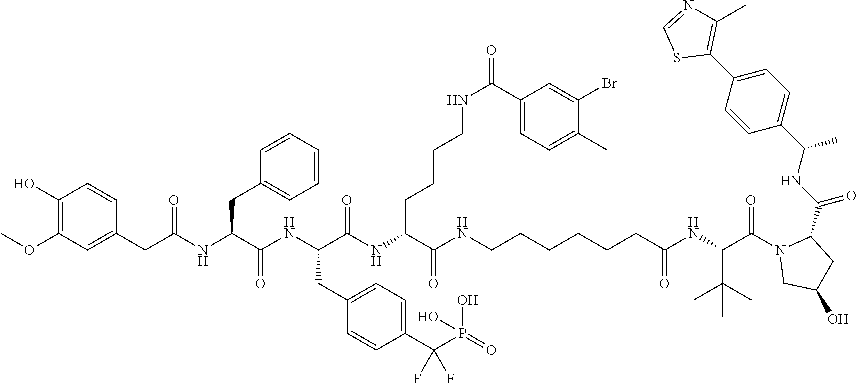 [SMILES: COc1cc(CC(=O)N[C@@H](Cc2ccccc2)C(=O)N[C@@H](Cc2ccc(C(F)(F)P(=O)(O)O)cc2)C(=O)N[C@H](CCCCNC(=O)c2ccc(C)c(Br)c2)C(=O)NCCCCCCC(=O)N[C@H](C(=O)N2C[C@H](O)C[C@H]2C(=O)N[C@@H](C)c2ccc(-c3scnc3C)cc2)C(C)(C)C)ccc1O] |
| 10 | 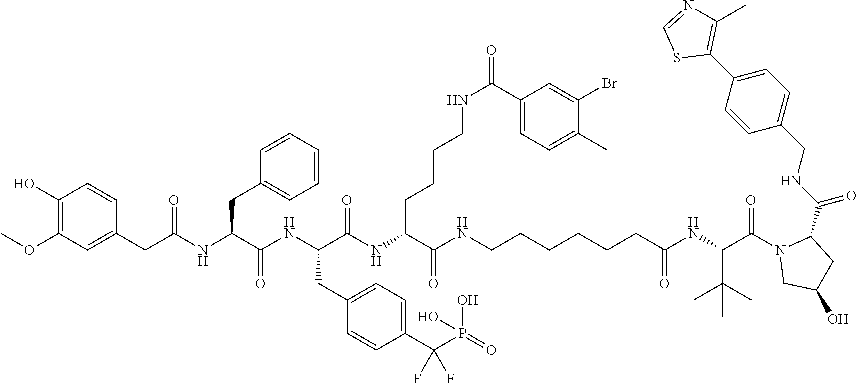 [SMILES: COc1cc(CC(=O)N[C@@H](Cc2ccccc2)C(=O)N[C@@H](Cc2ccc(C(F)(F)P(=O)(O)O)cc2)C(=O)N[C@H](CCCCNC(=O)c2ccc(C)c(Br)c2)C(=O)NCCCCCCC(=O)N[C@H](C(=O)N2C[C@H](O)C[C@H]2C(=O)NCc2ccc(-c3scnc3C)cc2)C(C)(C)C)ccc1O] |
| 11 | 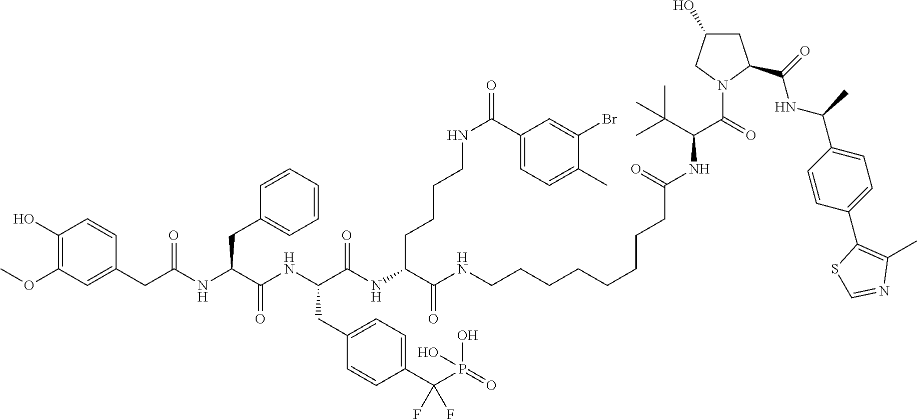 |
| 12 | 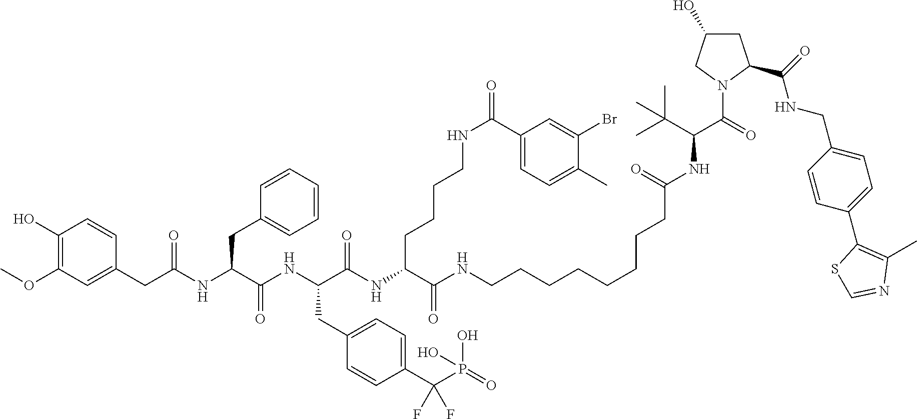 |
| 13 | 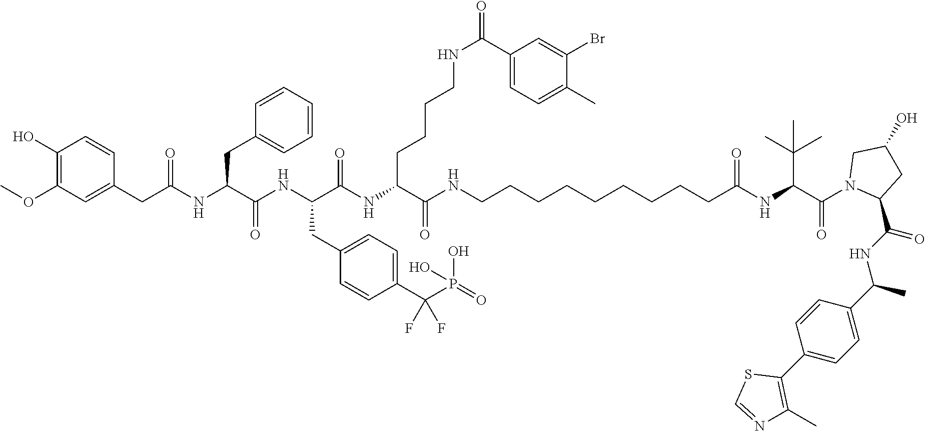 [SMILES: COc1cc(CC(=O)N[C@@H](Cc2ccccc2)C(=O)N[C@@H](Cc2ccc(C(F)(F)P(=O)(O)O)cc2)C(=O)N[C@H](CCCCNC(=O)c2ccc(C)c(Br)c2)C(=O)NCCCCCCCCCC(=O)N[C@H](C(=O)N2C[C@H](O)C[C@H]2C(=O)N[C@@H](C)c2ccc(-c3scnc3C)cc2)C(C)(C)C)ccc1O] |
| 14 | 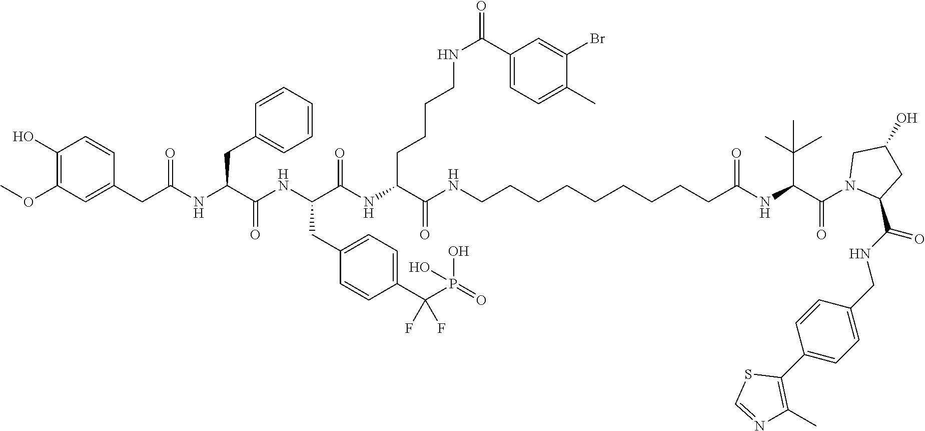 [SMILES: COc1cc(CC(=O)N[C@@H](Cc2ccccc2)C(=O)N[C@@H](Cc2ccc(C(F)(F)P(=O)(O)O)cc2)C(=O)N[C@H](CCCCNC(=O)c2ccc(C)c(Br)c2)C(=O)NCCCCCCCCCC(=O)N[C@H](C(=O)N2C[C@H](O)C[C@H]2C(=O)NCc2ccc(-c3scnc3C)cc2)C(C)(C)C)ccc1O] |
| 15 | 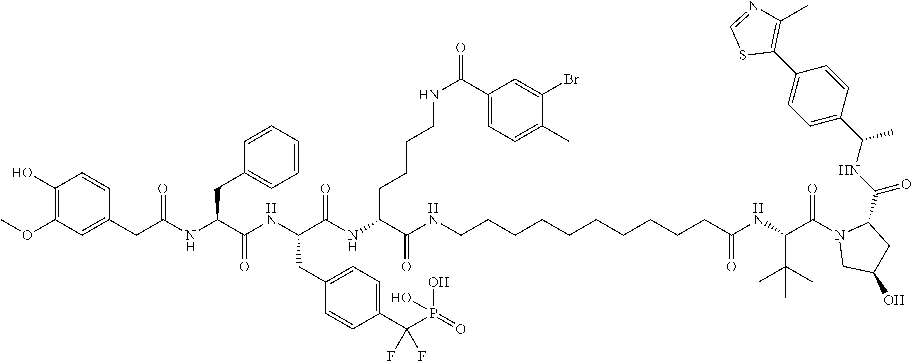 [SMILES: COc1cc(CC(=O)N[C@@H](Cc2ccccc2)C(=O)N[C@@H](Cc2ccc(C(F)(F)P(=O)(O)O)cc2)C(=O)N[C@H](CCCCNC(=O)c2ccc(C)c(Br)c2)C(=O)NCCCCCCCCCCC(=O)N[C@H](C(=O)N2C[C@H](O)C[C@H]2C(=O)N[C@@H](C)c2ccc(-c3scnc3C)cc2)C(C)(C)C)ccc1O] |
| 16 | 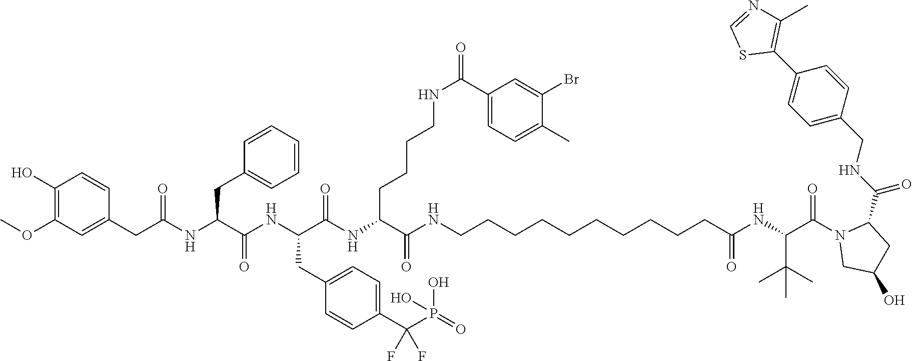 [SMILES: COc1cc(CC(=O)N[C@@H](Cc2ccccc2)C(=O)N[C@@H](Cc2ccc(C(F)(F)P(=O)(O)O)cc2)C(=O)N[C@H](CCCCNC(=O)c2ccc(C)c(Br)c2)C(=O)NCCCCCCCCCCC(=O)N[C@H](C(=O)N2C[C@H](O)C[C@H]2C(=O)NCc2ccc(-c3scnc3C)cc2)C(C)(C)C)ccc1O] |
| 17 | 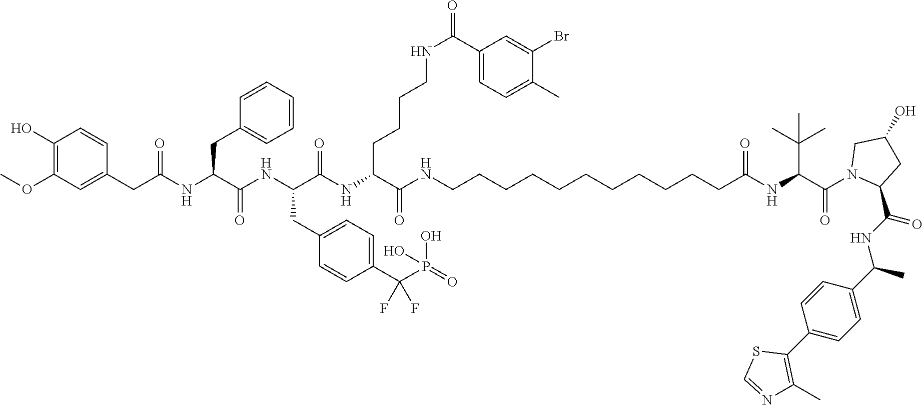 [SMILES: COc1cc(CC(=O)N[C@@H](Cc2ccccc2)C(=O)N[C@@H](Cc2ccc(C(F)(F)P(=O)(O)O)cc2)C(=O)N[C@H](CCCCNC(=O)c2ccc(C)c(Br)c2)C(=O)NCCCCCCCCCCCC(=O)N[C@H](C(=O)N2C[C@H](O)C[C@H]2C(=O)N[C@@H](C)c2ccc(-c3scnc3C)cc2)C(C)(C)C)ccc1O] |
| 18 | 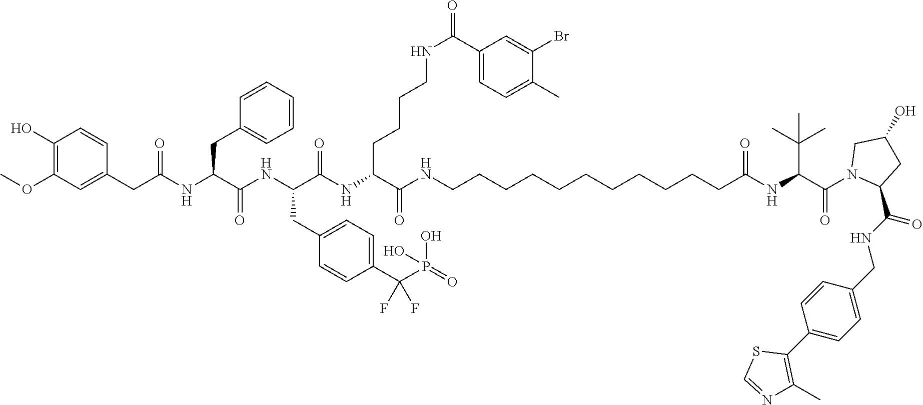 [SMILES: COc1cc(CC(=O)N[C@@H](Cc2ccccc2)C(=O)N[C@@H](Cc2ccc(C(F)(F)P(=O)(O)O)cc2)C(=O)N[C@H](CCCCNC(=O)c2ccc(C)c(Br)c2)C(=O)NCCCCCCCCCCCC(=O)N[C@H](C(=O)N2C[C@H](O)C[C@H]2C(=O)NCc2ccc(-c3scnc3C)cc2)C(C)(C)C)ccc1O] |
| 19 | 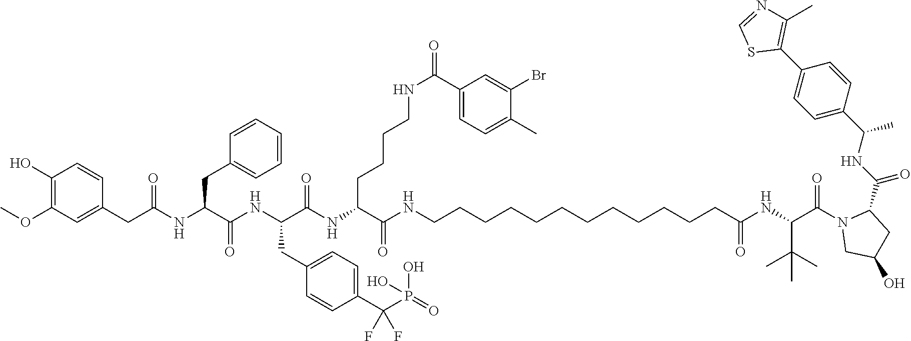 [SMILES: COc1cc(CC(=O)N[C@@H](Cc2ccccc2)C(=O)N[C@@H](Cc2ccc(C(F)(F)P(=O)(O)O)cc2)C(=O)N[C@H](CCCCNC(=O)c2ccc(C)c(Br)c2)C(=O)NCCCCCCCCCCCCC(=O)N[C@H](C(=O)N2C[C@H](O)C[C@H]2C(=O)N[C@@H](C)c2ccc(-c3scnc3C)cc2)C(C)(C)C)ccc1O] |
| 20 | 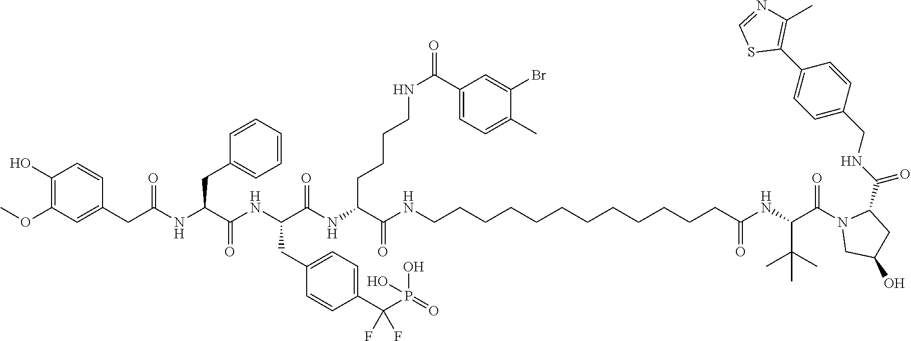 [SMILES: COc1cc(CC(=O)N[C@@H](Cc2ccccc2)C(=O)N[C@@H](Cc2ccc(C(F)(F)P(=O)(O)O)cc2)C(=O)N[C@H](CCCCNC(=O)c2ccc(C)c(Br)c2)C(=O)NCCCCCCCCCCCCC(=O)N[C@H](C(=O)N2C[C@H](O)C[C@H]2C(=O)NCc2ccc(-c3scnc3C)cc2)C(C)(C)C)ccc1O] |
| 21 | 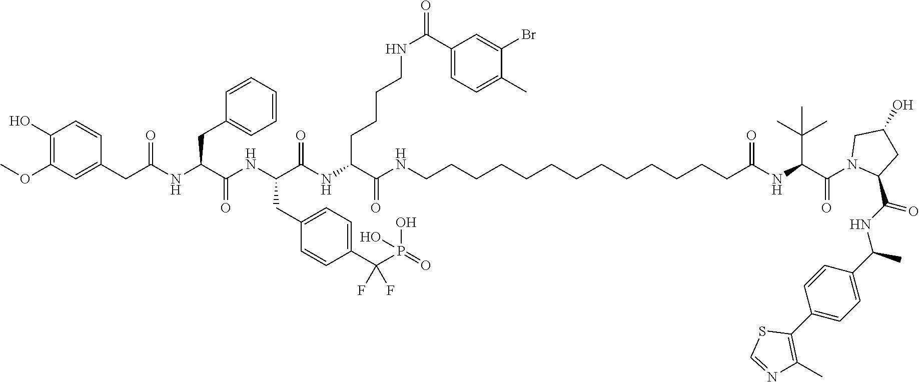 [SMILES: COc1cc(CC(=O)N[C@@H](Cc2ccccc2)C(=O)N[C@@H](Cc2ccc(C(F)(F)P(=O)(O)O)cc2)C(=O)N[C@H](CCCCNC(=O)c2ccc(C)c(Br)c2)C(=O)NCCCCCCCCCCCCCC(=O)N[C@H](C(=O)N2C[C@H](O)C[C@H]2C(=O)N[C@@H](C)c2ccc(-c3scnc3C)cc2)C(C)(C)C)ccc1O] |
| 22 | 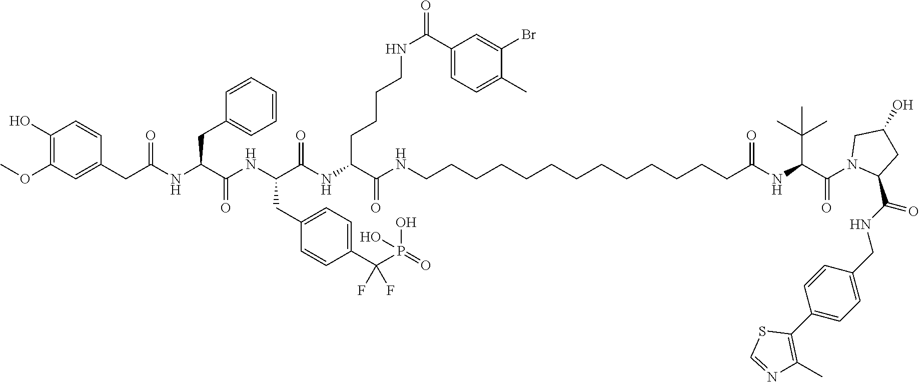 [SMILES: COc1cc(CC(=O)N[C@@H](Cc2ccccc2)C(=O)N[C@@H](Cc2ccc(C(F)(F)P(=O)(O)O)cc2)C(=O)N[C@H](CCCCNC(=O)c2ccc(C)c(Br)c2)C(=O)NCCCCCCCCCCCCCC(=O)N[C@H](C(=O)N2C[C@H](O)C[C@H]2C(=O)NCc2ccc(-c3scnc3C)cc2)C(C)(C)C)ccc1O] |
| 23 | 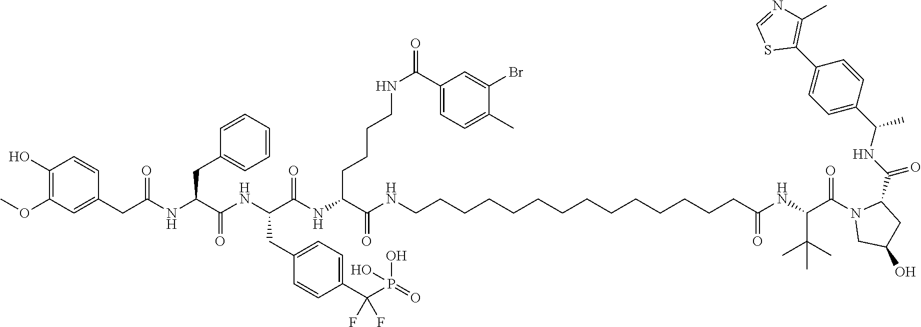 [SMILES: COc1cc(CC(=O)N[C@@H](Cc2ccccc2)C(=O)N[C@@H](Cc2ccc(C(F)(F)P(=O)(O)O)cc2)C(=O)N[C@H](CCCCNC(=O)c2ccc(C)c(Br)c2)C(=O)NCCCCCCCCCCCCCCC(=O)N[C@H](C(=O)N2C[C@H](O)C[C@H]2C(=O)N[C@@H](C)c2ccc(-c3scnc3C)cc2)C(C)(C)C)ccc1O] |
| 24 | 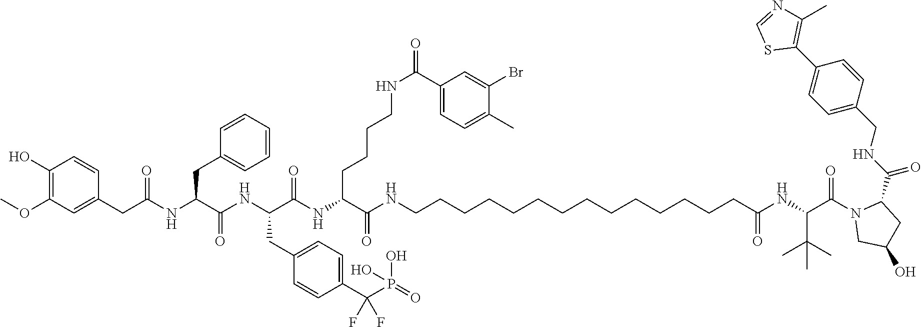 [SMILES: COc1cc(CC(=O)N[C@@H](Cc2ccccc2)C(=O)N[C@@H](Cc2ccc(C(F)(F)P(=O)(O)O)cc2)C(=O)N[C@H](CCCCNC(=O)c2ccc(C)c(Br)c2)C(=O)NCCCCCCCCCCCCCCC(=O)N[C@H](C(=O)N2C[C@H](O)C[C@H]2C(=O)NCc2ccc(-c3scnc3C)cc2)C(C)(C)C)ccc1O] |
| 25 | 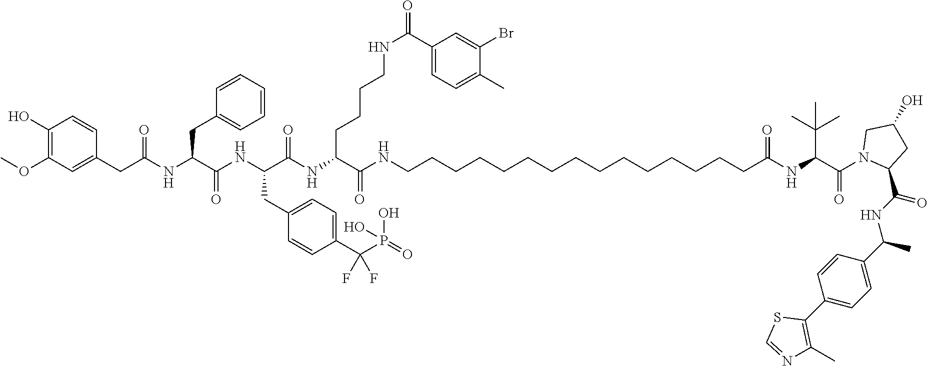 [SMILES: COc1cc(CC(=O)N[C@@H](Cc2ccccc2)C(=O)N[C@@H](Cc2ccc(C(F)(F)P(=O)(O)O)cc2)C(=O)N[C@H](CCCCNC(=O)c2ccc(C)c(Br)c2)C(=O)NCCCCCCCCCCCCCCCC(=O)N[C@H](C(=O)N2C[C@H](O)C[C@H]2C(=O)N[C@@H](C)c2ccc(-c3scnc3C)cc2)C(C)(C)C)ccc1O] |
| 26 | 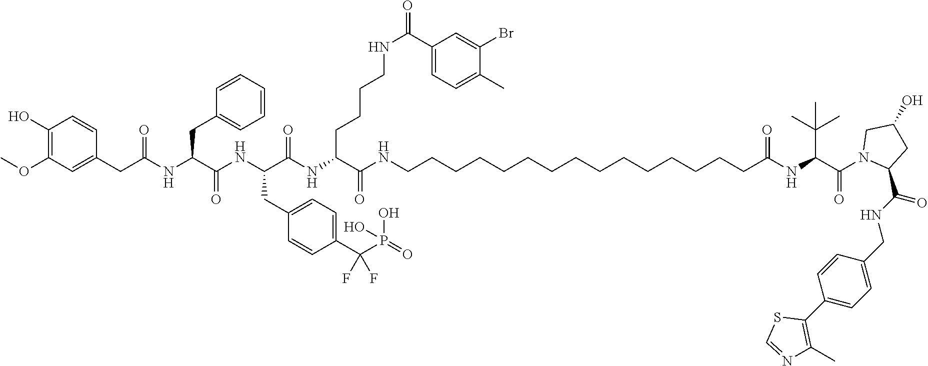 [SMILES: COc1cc(CC(=O)N[C@@H](Cc2ccccc2)C(=O)N[C@@H](Cc2ccc(C(F)(F)P(=O)(O)O)cc2)C(=O)N[C@H](CCCCNC(=O)c2ccc(C)c(Br)c2)C(=O)NCCCCCCCCCCCCCCCC(=O)N[C@H](C(=O)N2C[C@H](O)C[C@H]2C(=O)NCc2ccc(-c3scnc3C)cc2)C(C)(C)C)ccc1O] |
| 27 | 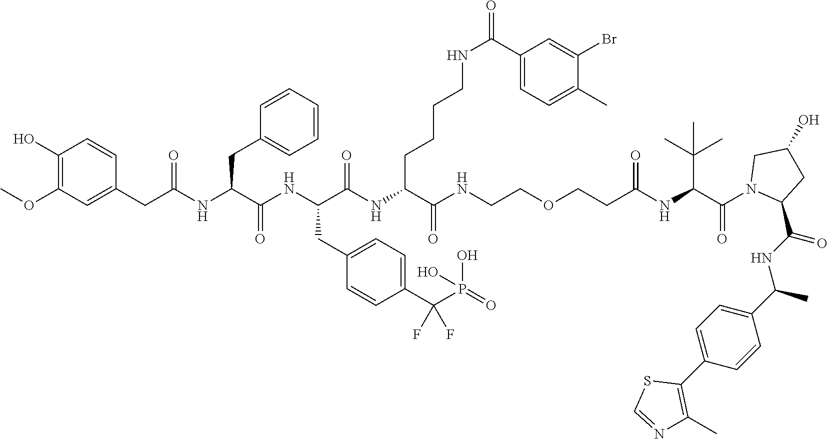 [SMILES: COc1cc(CC(=O)N[C@@H](Cc2ccccc2)C(=O)N[C@@H](Cc2ccc(C(F)(F)P(=O)(O)O)cc2)C(=O)N[C@H](CCCCNC(=O)c2ccc(C)c(Br)c2)C(=O)NCCOCCC(=O)N[C@H](C(=O)N2C[C@H](O)C[C@H]2C(=O)N[C@@H](C)c2ccc(-c3scnc3C)cc2)C(C)(C)C)ccc1O] |
| 28 | 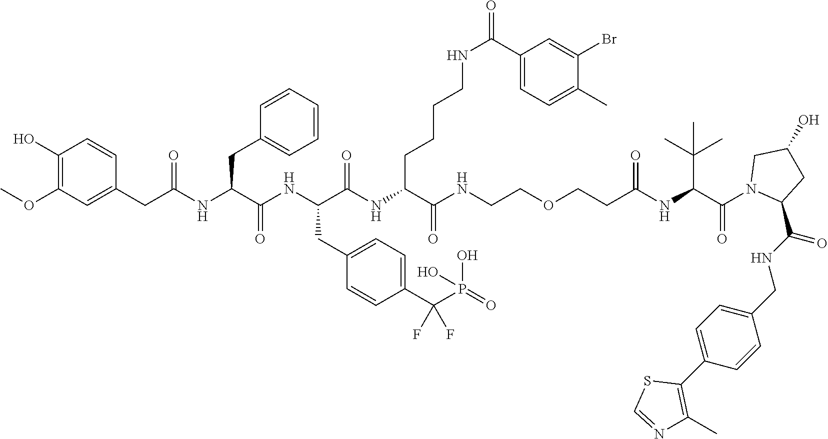 [SMILES: COc1cc(CC(=O)N[C@@H](Cc2ccccc2)C(=O)N[C@@H](Cc2ccc(C(F)(F)P(=O)(O)O)cc2)C(=O)N[C@H](CCCCNC(=O)c2ccc(C)c(Br)c2)C(=O)NCCOCCC(=O)N[C@H](C(=O)N2C[C@H](O)C[C@H]2C(=O)NCc2ccc(-c3scnc3C)cc2)C(C)(C)C)ccc1O] |
| 29 | 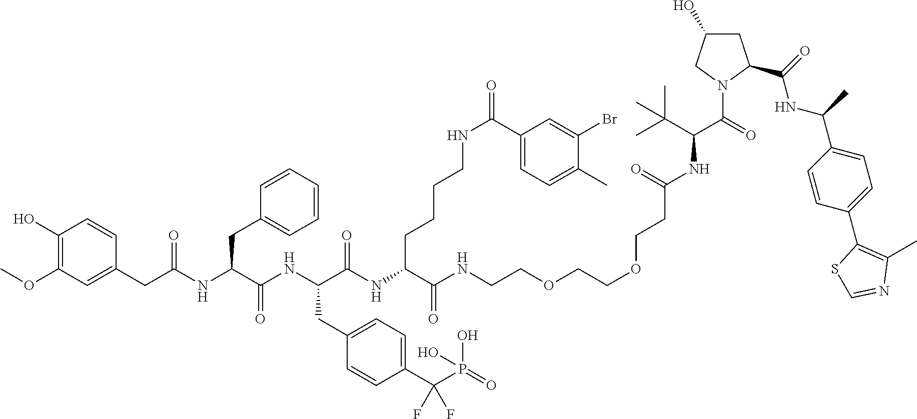 |
| 30 | 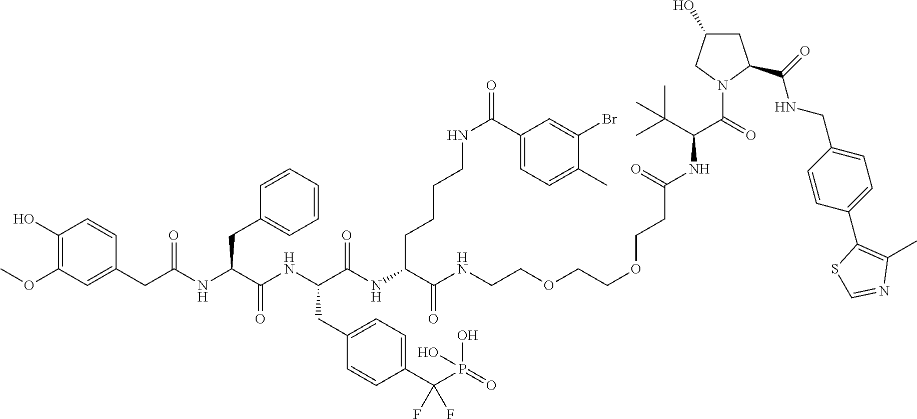 |
| 31 | 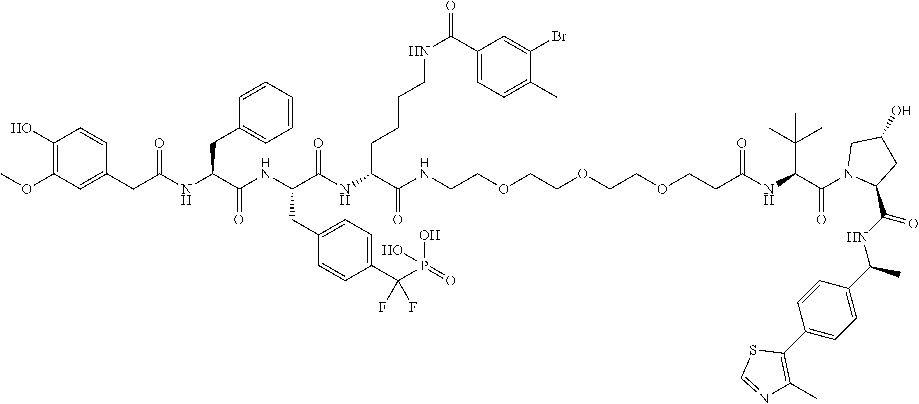 [SMILES: COc1cc(CC(=O)N[C@@H](Cc2ccccc2)C(=O)N[C@@H](Cc2ccc(C(F)(F)P(=O)(O)O)cc2)C(=O)N[C@H](CCCCNC(=O)c2ccc(C)c(Br)c2)C(=O)NCCOCCOCCOCCC(=O)N[C@H](C(=O)N2C[C@H](O)C[C@H]2C(=O)N[C@@H](C)c2ccc(-c3scnc3C)cc2)C(C)(C)C)ccc1O] |
| 32 | 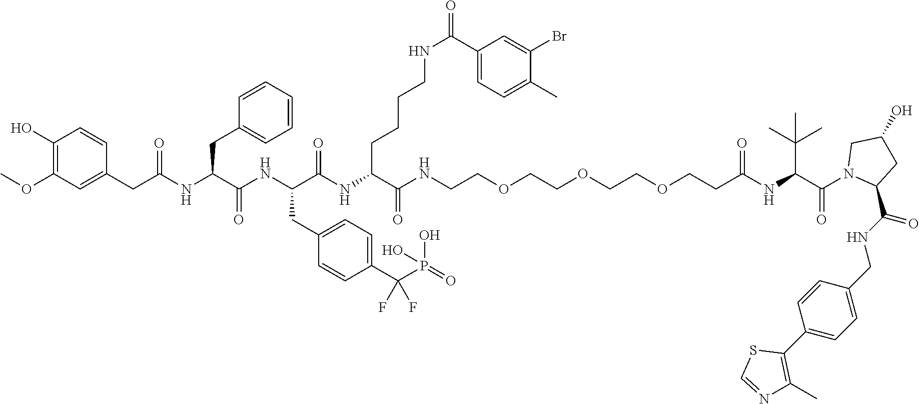 [SMILES: COc1cc(CC(=O)N[C@@H](Cc2ccccc2)C(=O)N[C@@H](Cc2ccc(C(F)(F)P(=O)(O)O)cc2)C(=O)N[C@H](CCCCNC(=O)c2ccc(C)c(Br)c2)C(=O)NCCOCCOCCOCCC(=O)N[C@H](C(=O)N2C[C@H](O)C[C@H]2C(=O)NCc2ccc(-c3scnc3C)cc2)C(C)(C)C)ccc1O] |
| 33 | 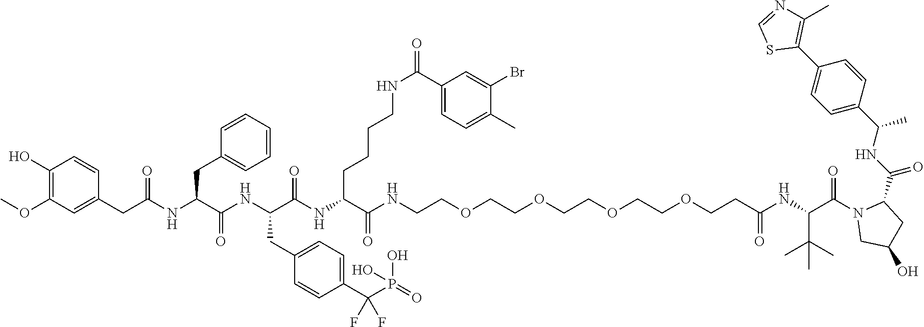 [SMILES: COc1cc(CC(=O)N[C@@H](Cc2ccccc2)C(=O)N[C@@H](Cc2ccc(C(F)(F)P(=O)(O)O)cc2)C(=O)N[C@H](CCCCNC(=O)c2ccc(C)c(Br)c2)C(=O)NCCOCCOCCOCCOCCC(=O)N[C@H](C(=O)N2C[C@H](O)C[C@H]2C(=O)N[C@@H](C)c2ccc(-c3scnc3C)cc2)C(C)(C)C)ccc1O] |
| 34 | 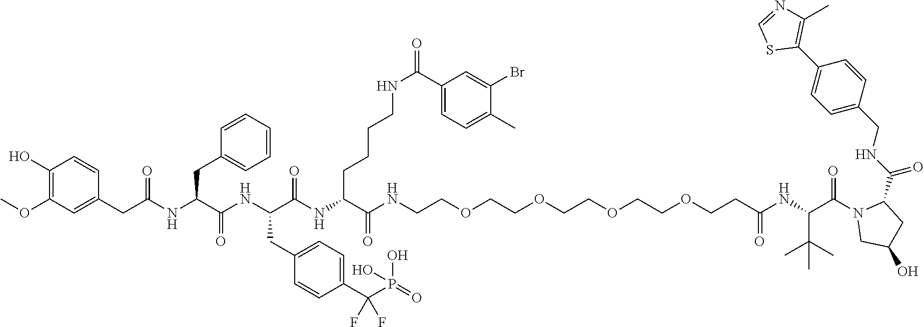 [SMILES: COc1cc(CC(=O)N[C@@H](Cc2ccccc2)C(=O)N[C@@H](Cc2ccc(C(F)(F)P(=O)(O)O)cc2)C(=O)N[C@H](CCCCNC(=O)c2ccc(C)c(Br)c2)C(=O)NCCOCCOCCOCCOCCC(=O)N[C@H](C(=O)N2C[C@H](O)C[C@H]2C(=O)NCc2ccc(-c3scnc3C)cc2)C(C)(C)C)ccc1O] |
| 35 | 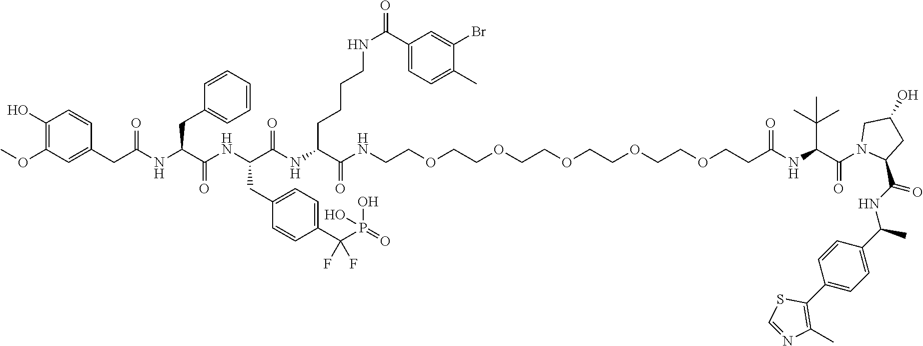 [SMILES: COc1cc(CC(=O)N[C@@H](Cc2ccccc2)C(=O)N[C@@H](Cc2ccc(C(F)(F)P(=O)(O)O)cc2)C(=O)N[C@H](CCCCNC(=O)c2ccc(C)c(Br)c2)C(=O)NCCOCCOCCOCCOCCOCCC(=O)N[C@H](C(=O)N2C[C@H](O)C[C@H]2C(=O)N[C@@H](C)c2ccc(-c3scnc3C)cc2)C(C)(C)C)ccc1O] |
| 36 | 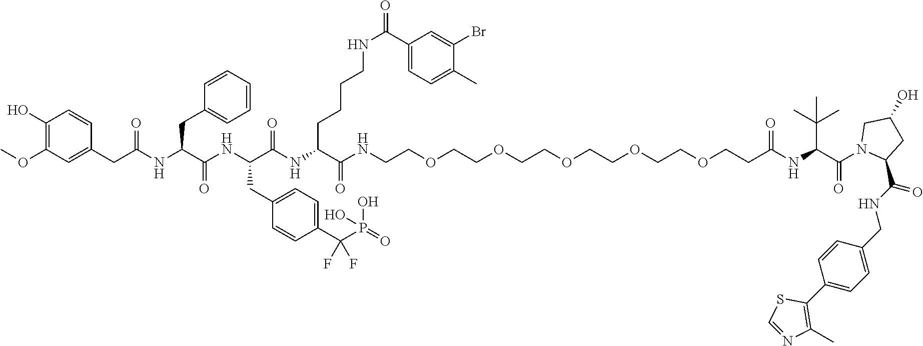 [SMILES: COc1cc(CC(=O)N[C@@H](Cc2ccccc2)C(=O)N[C@@H](Cc2ccc(C(F)(F)P(=O)(O)O)cc2)C(=O)N[C@H](CCCCNC(=O)c2ccc(C)c(Br)c2)C(=O)NCCOCCOCCOCCOCCOCCC(=O)N[C@H](C(=O)N2C[C@H](O)C[C@H]2C(=O)NCc2ccc(-c3scnc3C)cc2)C(C)(C)C)ccc1O] |
| 37 | 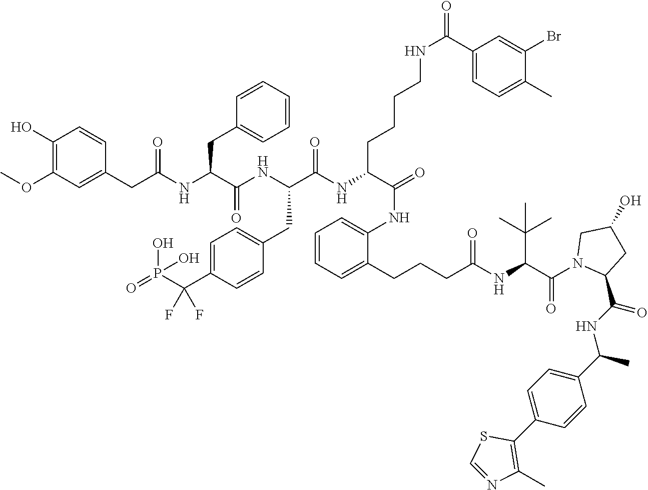 [SMILES: COc1cc(CC(=O)N[C@@H](Cc2ccccc2)C(=O)N[C@@H](Cc2ccc(C(F)(F)P(=O)(O)O)cc2)C(=O)N[C@H](CCCCNC(=O)c2ccc(C)c(Br)c2)C(=O)Nc2ccccc2CCCC(=O)N[C@H](C(=O)N2C[C@H](O)C[C@H]2C(=O)N[C@@H](C)c2ccc(-c3scnc3C)cc2)C(C)(C)C)ccc1O] |
| 38 | 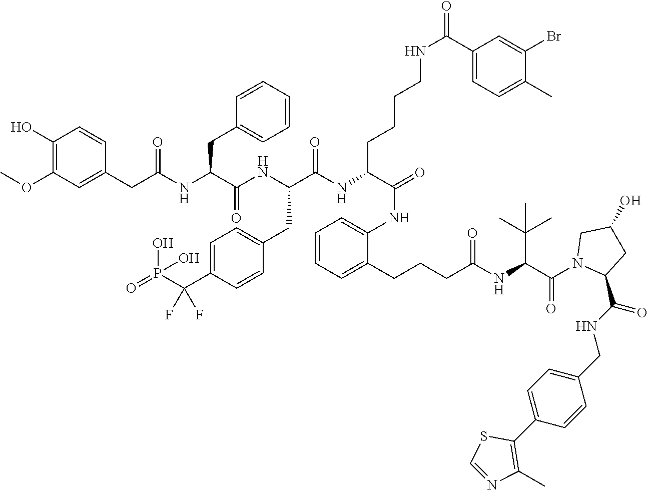 [SMILES: COc1cc(CC(=O)N[C@@H](Cc2ccccc2)C(=O)N[C@@H](Cc2ccc(C(F)(F)P(=O)(O)O)cc2)C(=O)N[C@H](CCCCNC(=O)c2ccc(C)c(Br)c2)C(=O)Nc2ccccc2CCCC(=O)N[C@H](C(=O)N2C[C@H](O)C[C@H]2C(=O)NCc2ccc(-c3scnc3C)cc2)C(C)(C)C)ccc1O] |
| 39 | 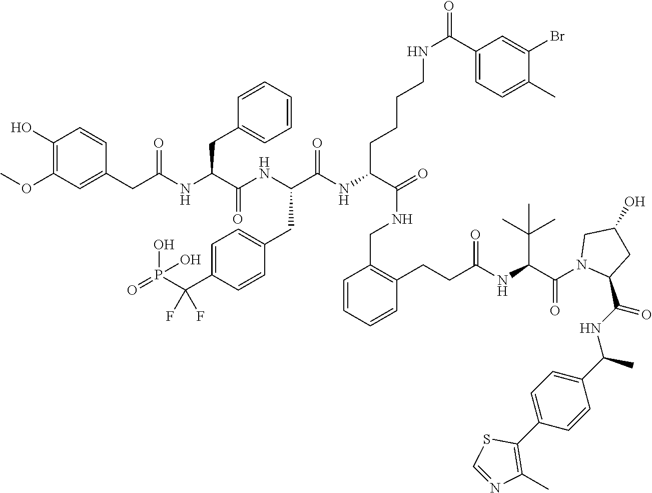 [SMILES: COc1cc(CC(=O)N[C@@H](Cc2ccccc2)C(=O)N[C@@H](Cc2ccc(C(F)(F)P(=O)(O)O)cc2)C(=O)N[C@H](CCCCNC(=O)c2ccc(C)c(Br)c2)C(=O)NCc2ccccc2CCC(=O)N[C@H](C(=O)N2C[C@H](O)C[C@H]2C(=O)N[C@@H](C)c2ccc(-c3scnc3C)cc2)C(C)(C)C)ccc1O] |
| 40 | 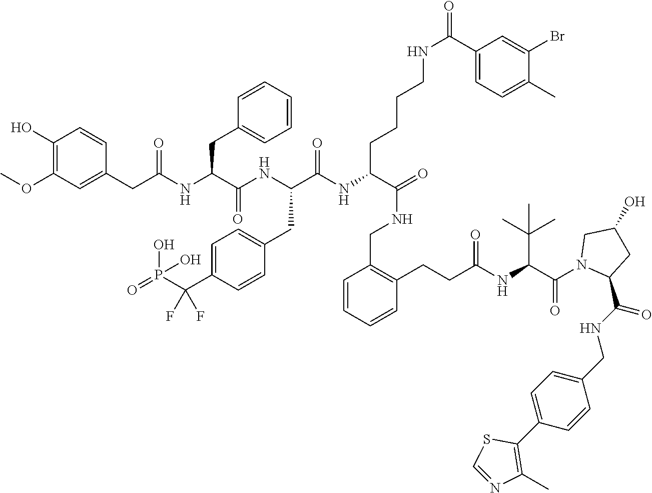 [SMILES: COc1cc(CC(=O)N[C@@H](Cc2ccccc2)C(=O)N[C@@H](Cc2ccc(C(F)(F)P(=O)(O)O)cc2)C(=O)N[C@H](CCCCNC(=O)c2ccc(C)c(Br)c2)C(=O)NCc2ccccc2CCC(=O)N[C@H](C(=O)N2C[C@H](O)C[C@H]2C(=O)NCc2ccc(-c3scnc3C)cc2)C(C)(C)C)ccc1O] |
| 41 | 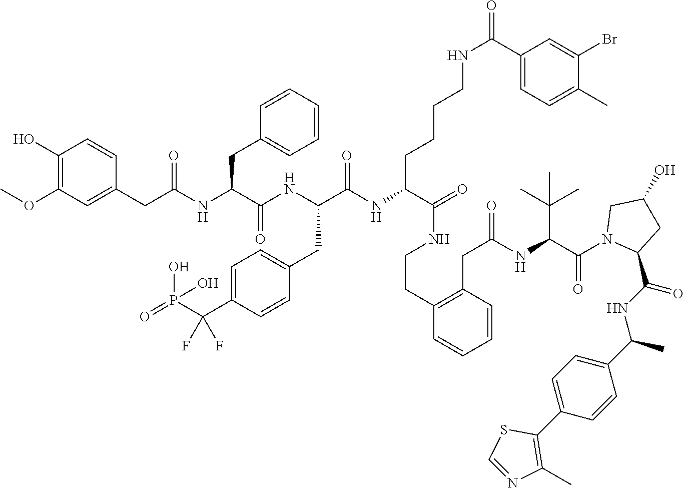 [SMILES: COc1cc(CC(=O)N[C@@H](Cc2ccccc2)C(=O)N[C@@H](Cc2ccc(C(F)(F)P(=O)(O)O)cc2)C(=O)N[C@H](CCCCNC(=O)c2ccc(C)c(Br)c2)C(=O)NCCc2ccccc2CC(=O)N[C@H](C(=O)N2C[C@H](O)C[C@H]2C(=O)N[C@@H](C)c2ccc(-c3scnc3C)cc2)C(C)(C)C)ccc1O] |
| 42 | 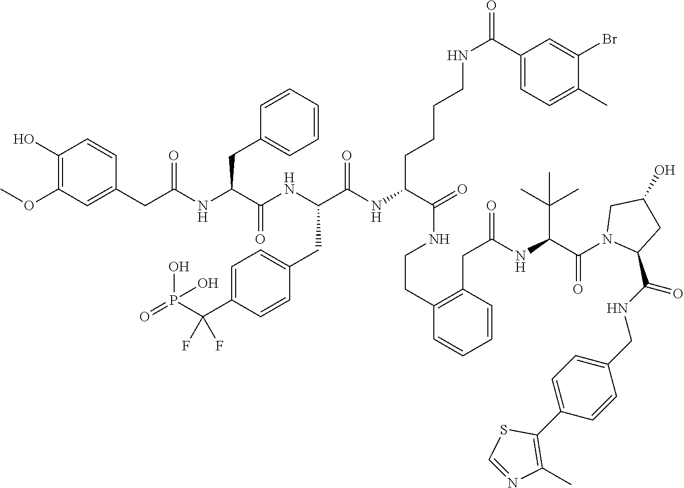 [SMILES: COc1cc(CC(=O)N[C@@H](Cc2ccccc2)C(=O)N[C@@H](Cc2ccc(C(F)(F)P(=O)(O)O)cc2)C(=O)N[C@H](CCCCNC(=O)c2ccc(C)c(Br)c2)C(=O)NCCc2ccccc2CC(=O)N[C@H](C(=O)N2C[C@H](O)C[C@H]2C(=O)NCc2ccc(-c3scnc3C)cc2)C(C)(C)C)ccc1O] |
| 43 | 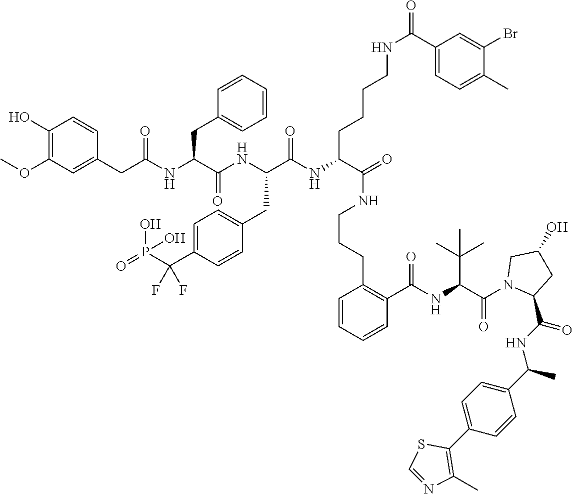 [SMILES: COc1cc(CC(=O)N[C@@H](Cc2ccccc2)C(=O)N[C@@H](Cc2ccc(C(F)(F)P(=O)(O)O)cc2)C(=O)N[C@H](CCCCNC(=O)c2ccc(C)c(Br)c2)C(=O)NCCCc2ccccc2C(=O)N[C@H](C(=O)N2C[C@H](O)C[C@H]2C(=O)N[C@@H](C)c2ccc(-c3scnc3C)cc2)C(C)(C)C)ccc1O] |
| 44 | 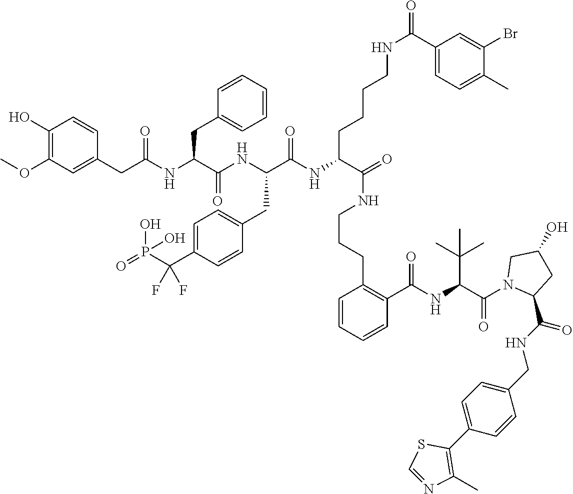 [SMILES: COc1cc(CC(=O)N[C@@H](Cc2ccccc2)C(=O)N[C@@H](Cc2ccc(C(F)(F)P(=O)(O)O)cc2)C(=O)N[C@H](CCCCNC(=O)c2ccc(C)c(Br)c2)C(=O)NCCCc2ccccc2C(=O)N[C@H](C(=O)N2C[C@H](O)C[C@H]2C(=O)NCc2ccc(-c3scnc3C)cc2)C(C)(C)C)ccc1O] |
| 45 | 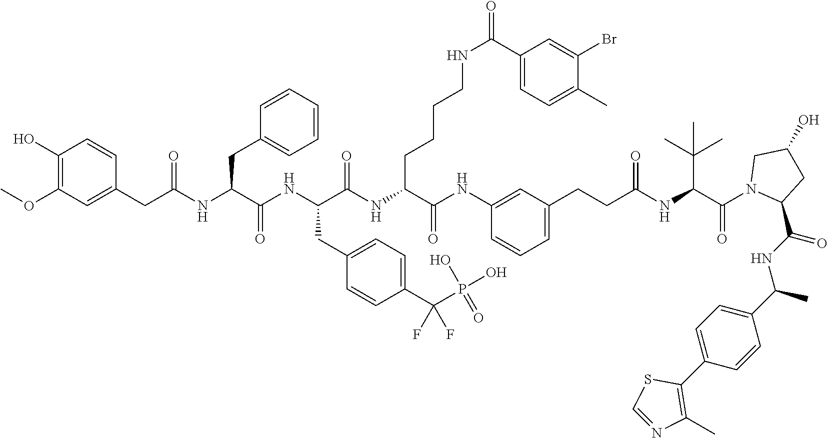 [SMILES: COc1cc(CC(=O)N[C@@H](Cc2ccccc2)C(=O)N[C@@H](Cc2ccc(C(F)(F)P(=O)(O)O)cc2)C(=O)N[C@H](CCCCNC(=O)c2ccc(C)c(Br)c2)C(=O)Nc2cccc(CCC(=O)N[C@H](C(=O)N3C[C@H](O)C[C@H]3C(=O)N[C@@H](C)c3ccc(-c4scnc4C)cc3)C(C)(C)C)c2)ccc1O] |
| 46 | 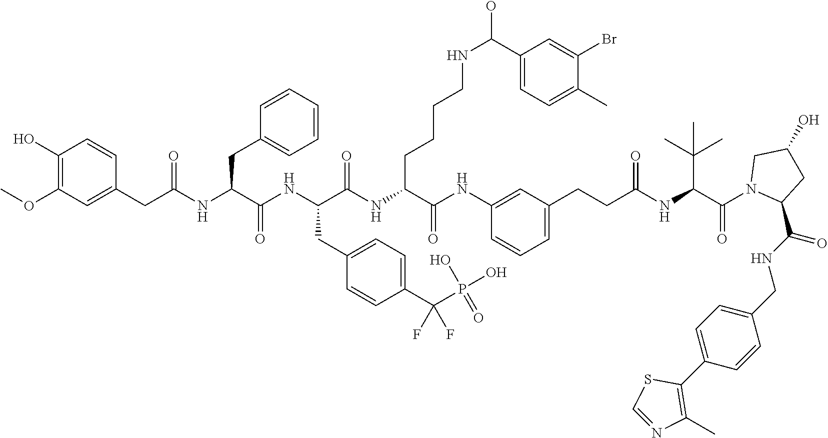 [SMILES: COc1cc(CC(=O)N[C@@H](Cc2ccccc2)C(=O)N[C@@H](Cc2ccc(C(F)(F)P(=O)(O)O)cc2)C(=O)N[C@H](CCCCNC(O)c2ccc(C)c(Br)c2)C(=O)Nc2cccc(CCC(=O)N[C@H](C(=O)N3C[C@H](O)C[C@H]3C(=O)NCc3ccc(-c4scnc4C)cc3)C(C)(C)C)c2)ccc1O] |
| 47 | 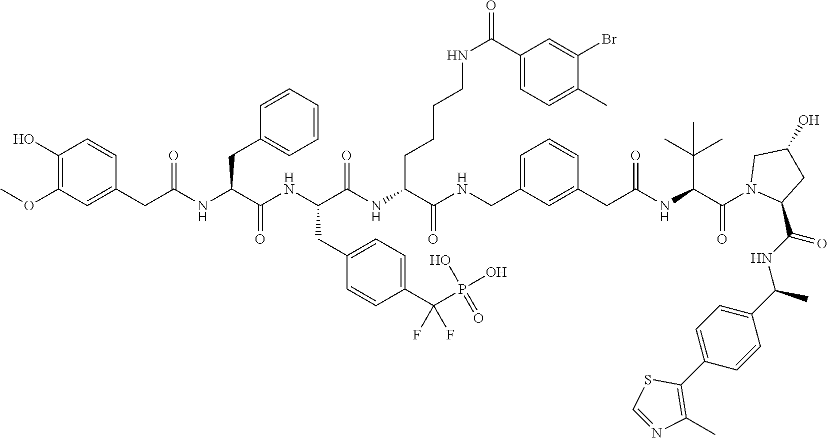 [SMILES: COc1cc(CC(=O)N[C@@H](Cc2ccccc2)C(=O)N[C@@H](Cc2ccc(C(F)(F)P(=O)(O)O)cc2)C(=O)N[C@H](CCCCNC(=O)c2ccc(C)c(Br)c2)C(=O)NCc2cccc(CC(=O)N[C@H](C(=O)N3C[C@H](O)C[C@H]3C(=O)N[C@@H](C)c3ccc(-c4scnc4C)cc3)C(C)(C)C)c2)ccc1O] |
| 48 | 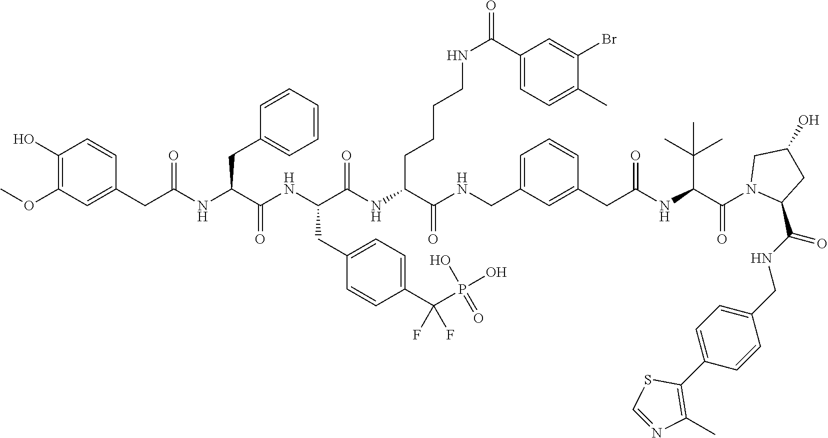 [SMILES: COc1cc(CC(=O)N[C@@H](Cc2ccccc2)C(=O)N[C@@H](Cc2ccc(C(F)(F)P(=O)(O)O)cc2)C(=O)N[C@H](CCCCNC(=O)c2ccc(C)c(Br)c2)C(=O)NCc2cccc(CC(=O)N[C@H](C(=O)N3C[C@H](O)C[C@H]3C(=O)NCc3ccc(-c4scnc4C)cc3)C(C)(C)C)c2)ccc1O] |
| 49 | 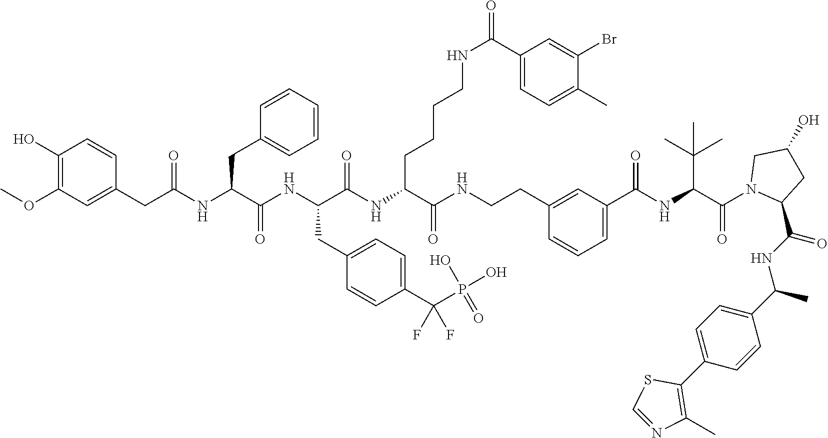 [SMILES: COc1cc(CC(=O)N[C@@H](Cc2ccccc2)C(=O)N[C@@H](Cc2ccc(C(F)(F)P(=O)(O)O)cc2)C(=O)N[C@H](CCCCNC(=O)c2ccc(C)c(Br)c2)C(=O)NCCc2cccc(C(=O)N[C@H](C(=O)N3C[C@H](O)C[C@H]3C(=O)N[C@@H](C)c3ccc(-c4scnc4C)cc3)C(C)(C)C)c2)ccc1O] |
| 50 | 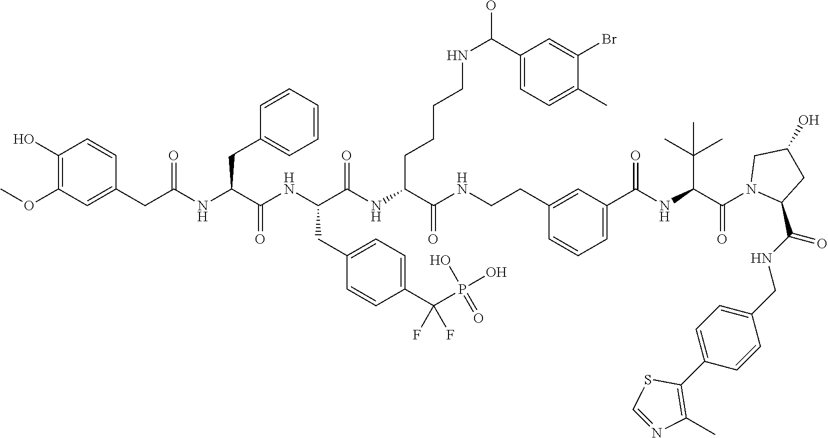 [SMILES: COc1cc(CC(=O)N[C@@H](Cc2ccccc2)C(=O)N[C@@H](Cc2ccc(C(F)(F)P(=O)(O)O)cc2)C(=O)N[C@H](CCCCNC(O)c2ccc(C)c(Br)c2)C(=O)NCCc2cccc(C(=O)N[C@H](C(=O)N3C[C@H](O)C[C@H]3C(=O)NCc3ccc(-c4scnc4C)cc3)C(C)(C)C)c2)ccc1O] |
| 51 | 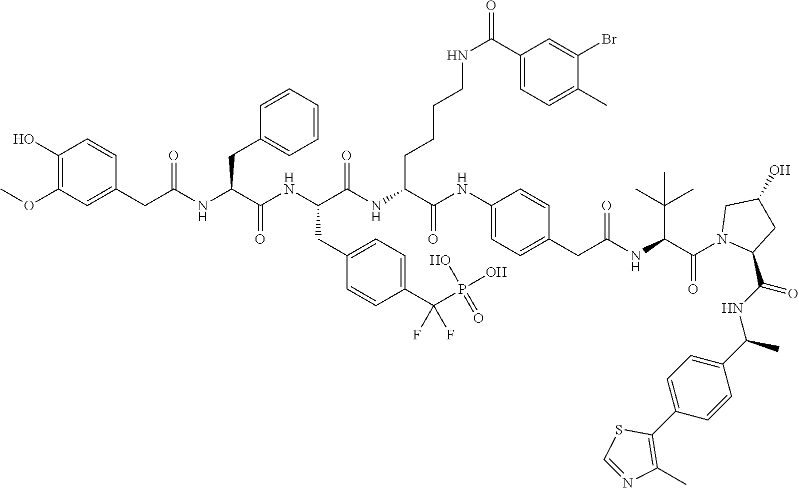 [SMILES: COc1cc(CC(=O)N[C@@H](Cc2ccccc2)C(=O)N[C@@H](Cc2ccc(C(F)(F)P(=O)(O)O)cc2)C(=O)N[C@H](CCCCNC(=O)c2ccc(C)c(Br)c2)C(=O)Nc2ccc(CC(=O)N[C@H](C(=O)N3C[C@H](O)C[C@H]3C(=O)N[C@@H](C)c3ccc(-c4scnc4C)cc3)C(C)(C)C)cc2)ccc1O] |
| 52 | 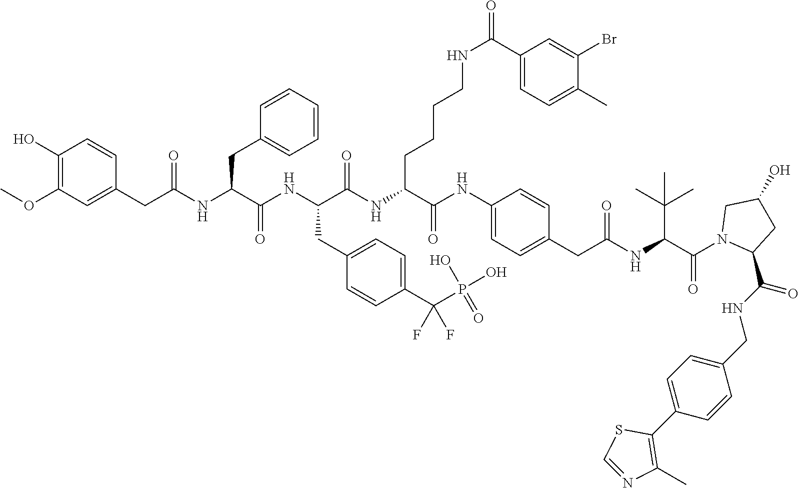 [SMILES: COc1cc(CC(=O)N[C@@H](Cc2ccccc2)C(=O)N[C@@H](Cc2ccc(C(F)(F)P(=O)(O)O)cc2)C(=O)N[C@H](CCCCNC(=O)c2ccc(C)c(Br)c2)C(=O)Nc2ccc(CC(=O)N[C@H](C(=O)N3C[C@H](O)C[C@H]3C(=O)NCc3ccc(-c4scnc4C)cc3)C(C)(C)C)cc2)ccc1O] |
| 53 | 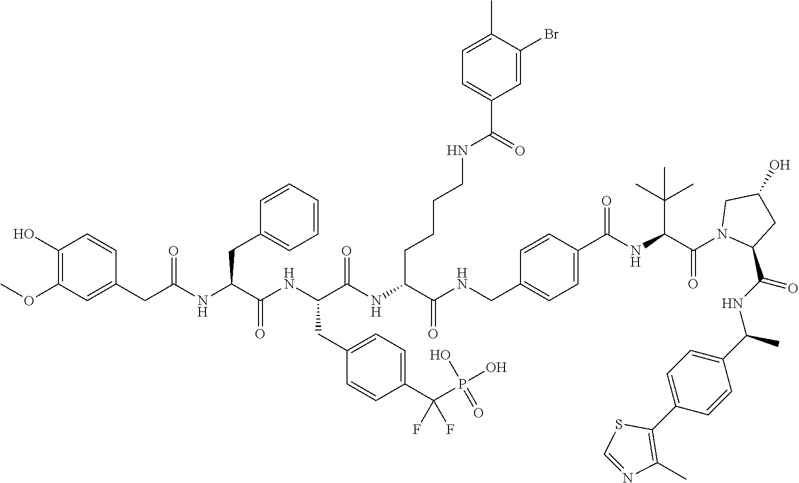 [SMILES: COc1cc(CC(=O)N[C@@H](Cc2ccccc2)C(=O)N[C@@H](Cc2ccc(C(F)(F)P(=O)(O)O)cc2)C(=O)N[C@H](CCCCNC(=O)c2ccc(C)c(Br)c2)C(=O)NCc2ccc(C(=O)N[C@H](C(=O)N3C[C@H](O)C[C@H]3C(=O)N[C@@H](C)c3ccc(-c4scnc4C)cc3)C(C)(C)C)cc2)ccc1O] |
| 54 | 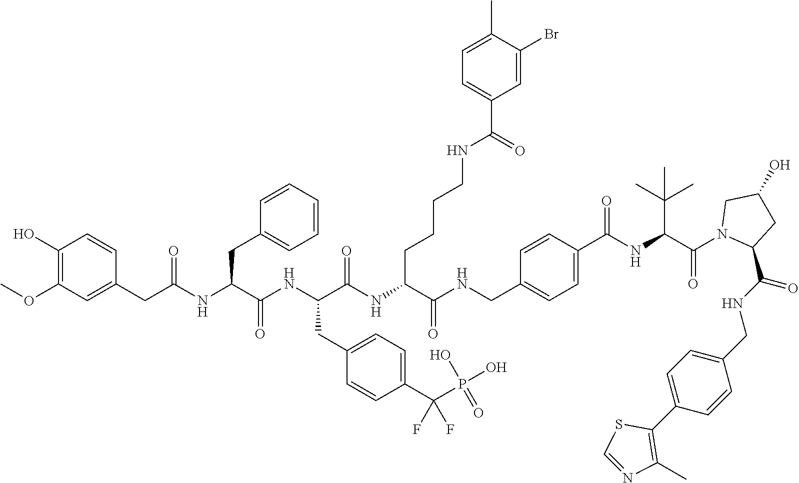 [SMILES: COc1cc(CC(=O)N[C@@H](Cc2ccccc2)C(=O)N[C@@H](Cc2ccc(C(F)(F)P(=O)(O)O)cc2)C(=O)N[C@H](CCCCNC(=O)c2ccc(C)c(Br)c2)C(=O)NCc2ccc(C(=O)N[C@H](C(=O)N3C[C@H](O)C[C@H]3C(=O)NCc3ccc(-c4scnc4C)cc3)C(C)(C)C)cc2)ccc1O] |
| 55 | 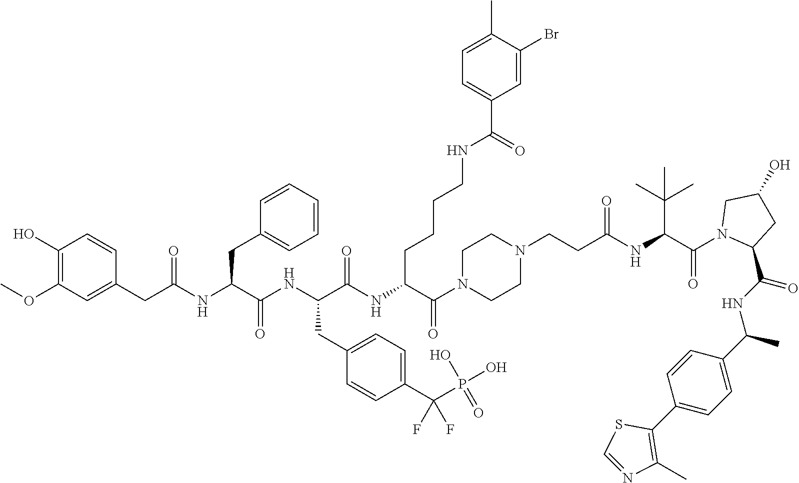 [SMILES: COc1cc(CC(=O)N[C@@H](Cc2ccccc2)C(=O)N[C@@H](Cc2ccc(C(F)(F)P(=O)(O)O)cc2)C(=O)N[C@H](CCCCNC(=O)c2ccc(C)c(Br)c2)C(=O)N2CCN(CCC(=O)N[C@H](C(=O)N3C[C@H](O)C[C@H]3C(=O)N[C@@H](C)c3ccc(-c4scnc4C)cc3)C(C)(C)C)CC2)ccc1O] |
| 56 | 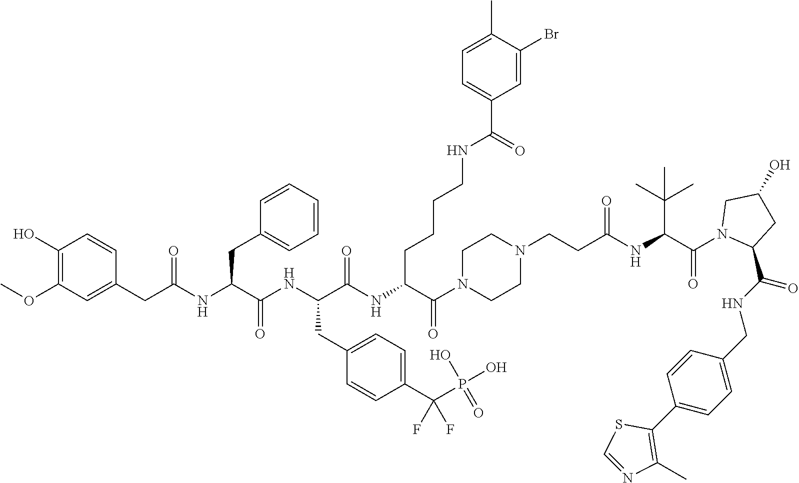 [SMILES: COc1cc(CC(=O)N[C@@H](Cc2ccccc2)C(=O)N[C@@H](Cc2ccc(C(F)(F)P(=O)(O)O)cc2)C(=O)N[C@H](CCCCNC(=O)c2ccc(C)c(Br)c2)C(=O)N2CCN(CCC(=O)N[C@H](C(=O)N3C[C@H](O)C[C@H]3C(=O)NCc3ccc(-c4scnc4C)cc3)C(C)(C)C)CC2)ccc1O] |
| 57 | 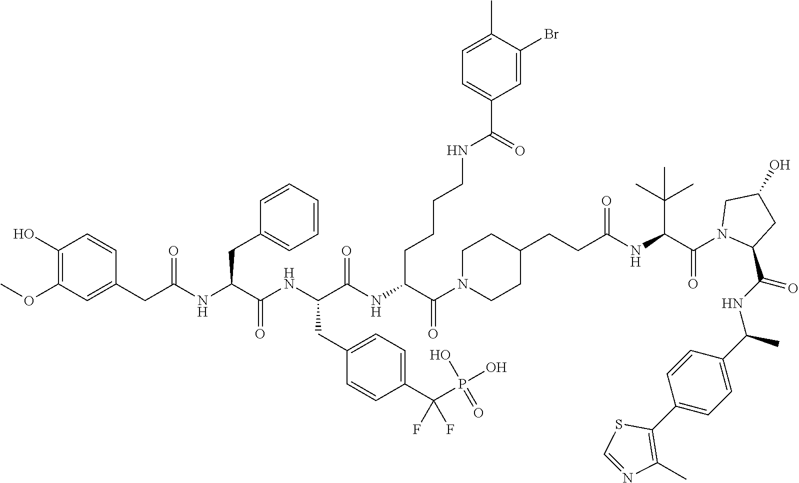 [SMILES: COc1cc(CC(=O)N[C@@H](Cc2ccccc2)C(=O)N[C@@H](Cc2ccc(C(F)(F)P(=O)(O)O)cc2)C(=O)N[C@H](CCCCNC(=O)c2ccc(C)c(Br)c2)C(=O)N2CCC(CCC(=O)N[C@H](C(=O)N3C[C@H](O)C[C@H]3C(=O)N[C@@H](C)c3ccc(-c4scnc4C)cc3)C(C)(C)C)CC2)ccc1O] |
| 58 | 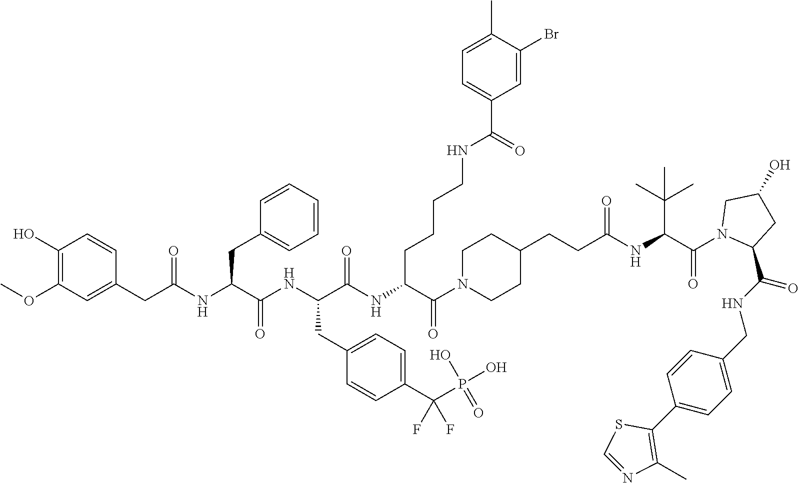 [SMILES: COc1cc(CC(=O)N[C@@H](Cc2ccccc2)C(=O)N[C@@H](Cc2ccc(C(F)(F)P(=O)(O)O)cc2)C(=O)N[C@H](CCCCNC(=O)c2ccc(C)c(Br)c2)C(=O)N2CCC(CCC(=O)N[C@H](C(=O)N3C[C@H](O)C[C@H]3C(=O)NCc3ccc(-c4scnc4C)cc3)C(C)(C)C)CC2)ccc1O] |
| 59 | 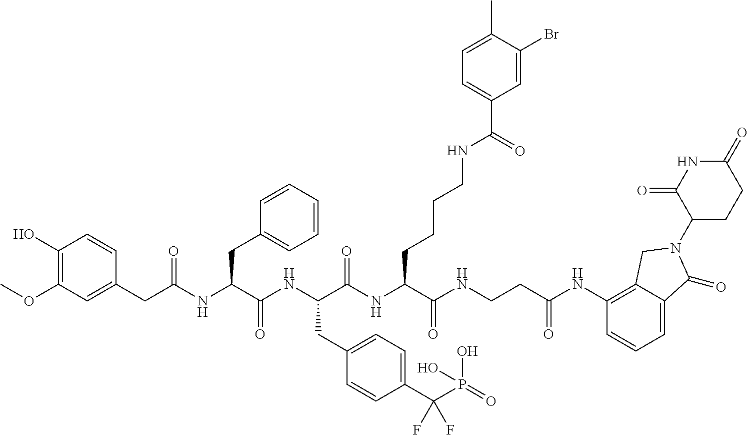 [SMILES: COc1cc(CC(=O)N[C@@H](Cc2ccccc2)C(=O)N[C@@H](Cc2ccc(C(F)(F)P(=O)(O)O)cc2)C(=O)N[C@@H](CCCCNC(=O)c2ccc(C)c(Br)c2)C(=O)NCCC(=O)Nc2cccc3c2CN(C2CCC(=O)NC2=O)C3=O)ccc1O] |
| 60 | 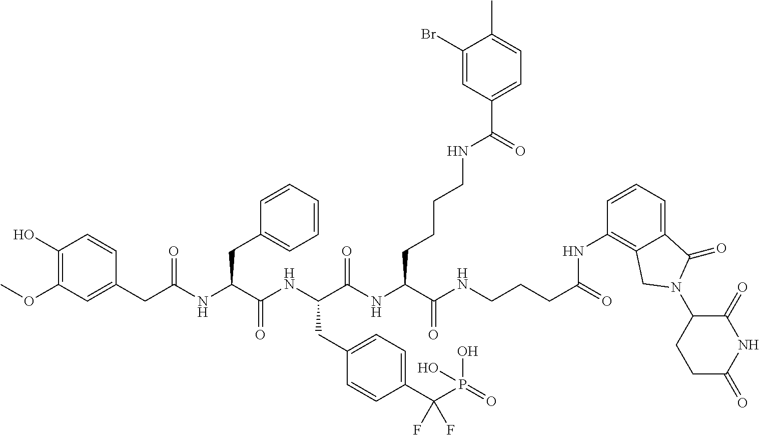 [SMILES: COc1cc(CC(=O)N[C@@H](Cc2ccccc2)C(=O)N[C@@H](Cc2ccc(C(F)(F)P(=O)(O)O)cc2)C(=O)N[C@@H](CCCCNC(=O)c2ccc(C)c(Br)c2)C(=O)NCCCC(=O)Nc2cccc3c2CN(C2CCC(=O)NC2=O)C3=O)ccc1O] |
| 61 | 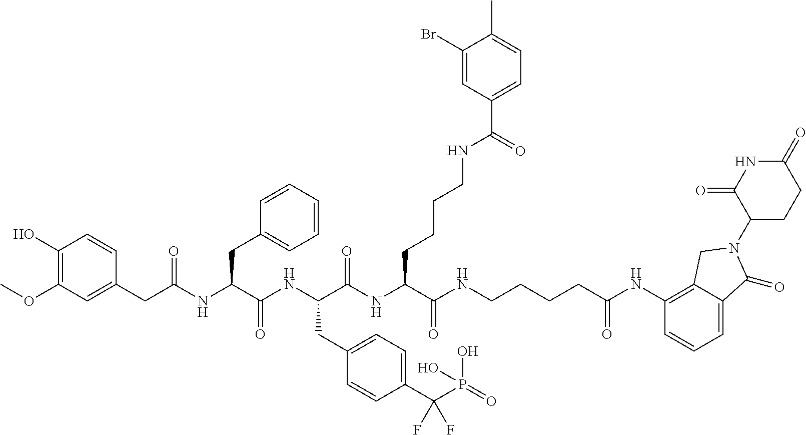 [SMILES: COc1cc(CC(=O)N[C@@H](Cc2ccccc2)C(=O)N[C@@H](Cc2ccc(C(F)(F)P(=O)(O)O)cc2)C(=O)N[C@@H](CCCCNC(=O)c2ccc(C)c(Br)c2)C(=O)NCCCCC(=O)Nc2cccc3c2CN(C2CCC(=O)NC2=O)C3=O)ccc1O] |
| 62 | 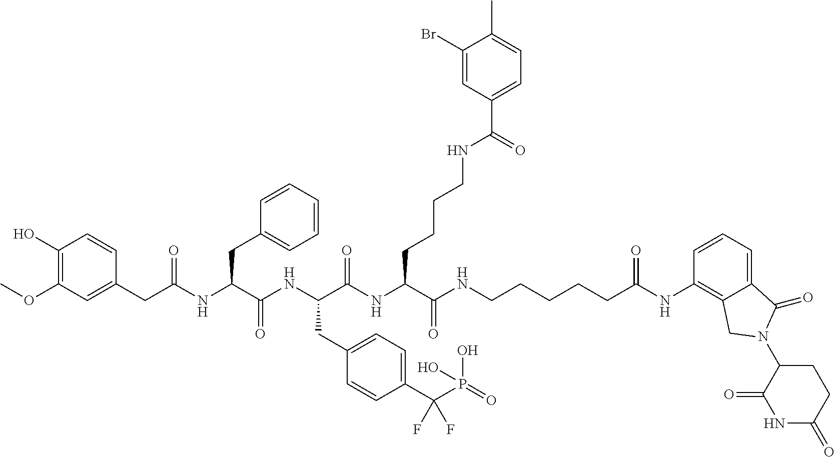 [SMILES: COc1cc(CC(=O)N[C@@H](Cc2ccccc2)C(=O)N[C@@H](Cc2ccc(C(F)(F)P(=O)(O)O)cc2)C(=O)N[C@@H](CCCCNC(=O)c2ccc(C)c(Br)c2)C(=O)NCCCCCC(=O)Nc2cccc3c2CN(C2CCC(=O)NC2=O)C3=O)ccc1O] |
| 63 | 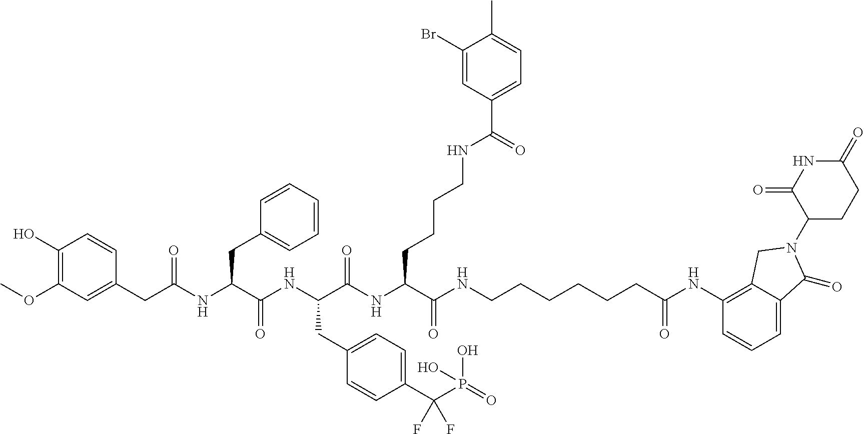 [SMILES: COc1cc(CC(=O)N[C@@H](Cc2ccccc2)C(=O)N[C@@H](Cc2ccc(C(F)(F)P(=O)(O)O)cc2)C(=O)N[C@@H](CCCCNC(=O)c2ccc(C)c(Br)c2)C(=O)NCCCCCCC(=O)Nc2cccc3c2CN(C2CCC(=O)NC2=O)C3=O)ccc1O] |
| 64 | 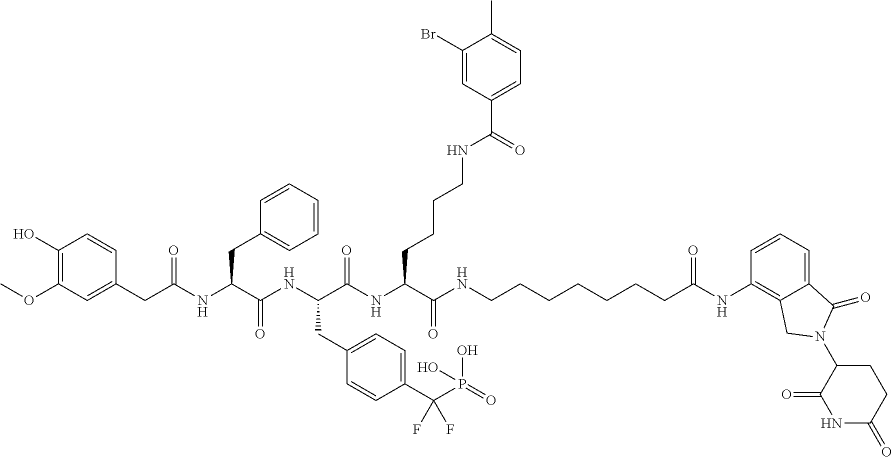 [SMILES: COc1cc(CC(=O)N[C@@H](Cc2ccccc2)C(=O)N[C@@H](Cc2ccc(C(F)(F)P(=O)(O)O)cc2)C(=O)N[C@@H](CCCCNC(=O)c2ccc(C)c(Br)c2)C(=O)NCCCCCCCC(=O)Nc2cccc3c2CN(C2CCC(=O)NC2=O)C3=O)ccc1O] |
| 65 | 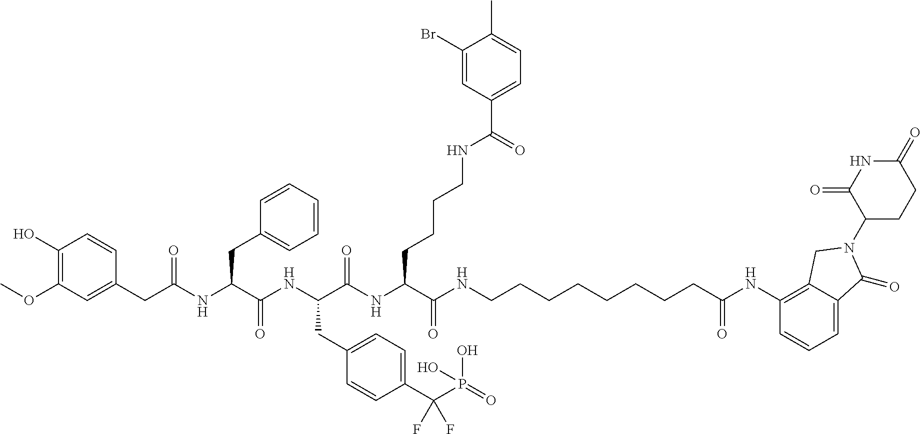 [SMILES: COc1cc(CC(=O)N[C@@H](Cc2ccccc2)C(=O)N[C@@H](Cc2ccc(C(F)(F)P(=O)(O)O)cc2)C(=O)N[C@@H](CCCCNC(=O)c2ccc(C)c(Br)c2)C(=O)NCCCCCCCCC(=O)Nc2cccc3c2CN(C2CCC(=O)NC2=O)C3=O)ccc1O] |
| 66 | 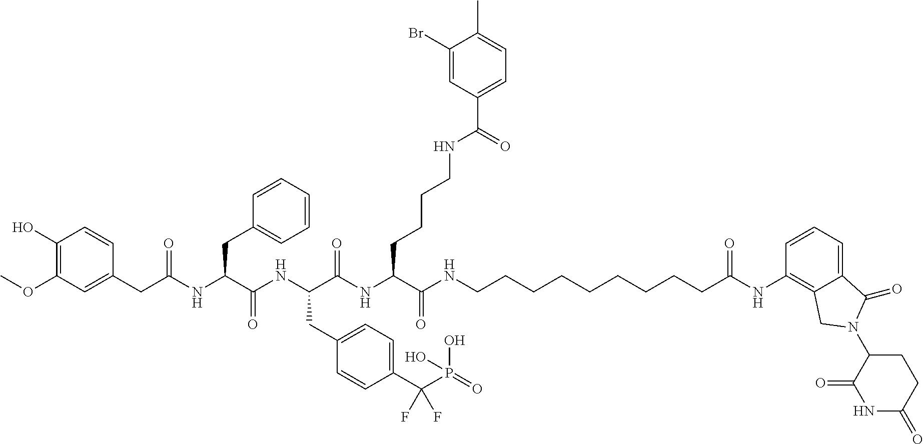 [SMILES: COc1cc(CC(=O)N[C@@H](Cc2ccccc2)C(=O)N[C@@H](Cc2ccc(C(F)(F)P(=O)(O)O)cc2)C(=O)N[C@@H](CCCCNC(=O)c2ccc(C)c(Br)c2)C(=O)NCCCCCCCCCC(=O)Nc2cccc3c2CN(C2CCC(=O)NC2=O)C3=O)ccc1O] |
| 67 | 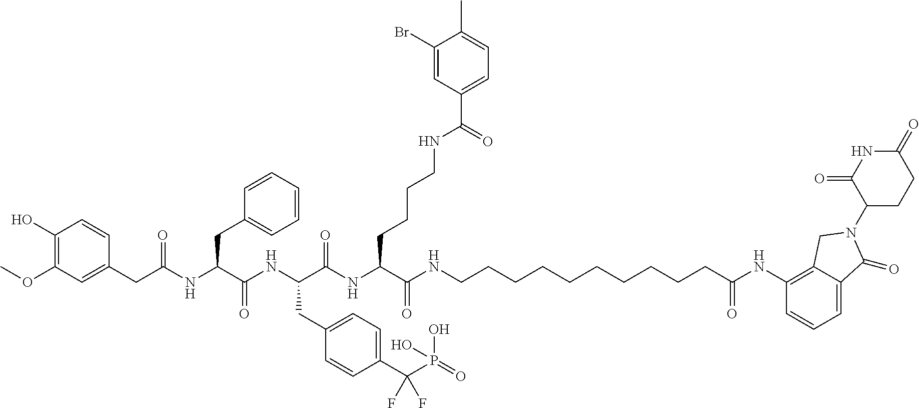 [SMILES: COc1cc(CC(=O)N[C@@H](Cc2ccccc2)C(=O)N[C@@H](Cc2ccc(C(F)(F)P(=O)(O)O)cc2)C(=O)N[C@@H](CCCCNC(=O)c2ccc(C)c(Br)c2)C(=O)NCCCCCCCCCCC(=O)Nc2cccc3c2CN(C2CCC(=O)NC2=O)C3=O)ccc1O] |
| 68 | 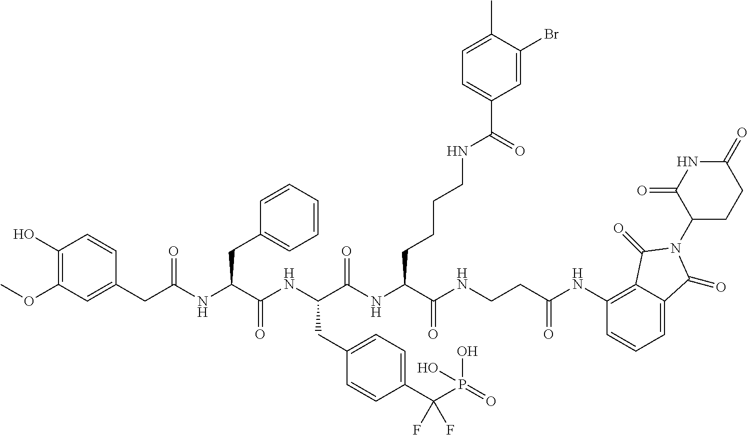 [SMILES: COc1cc(CC(=O)N[C@@H](Cc2ccccc2)C(=O)N[C@@H](Cc2ccc(C(F)(F)P(=O)(O)O)cc2)C(=O)N[C@@H](CCCCNC(=O)c2ccc(C)c(Br)c2)C(=O)NCCC(=O)Nc2cccc3c2C(=O)N(C2CCC(=O)NC2=O)C3=O)ccc1O] |
| 69 | 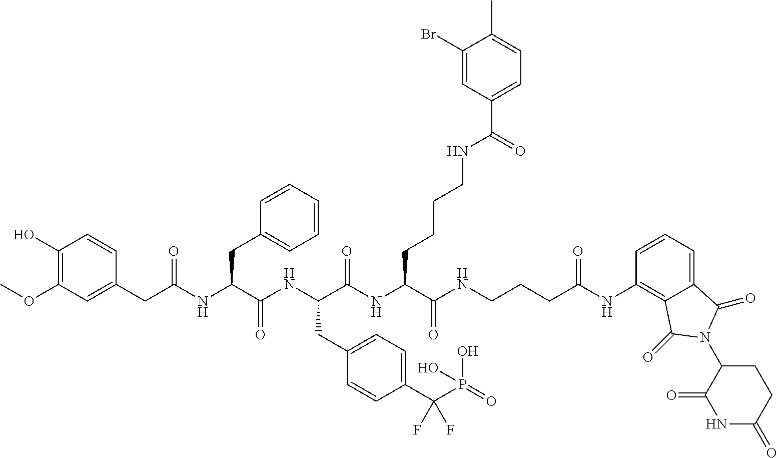 [SMILES: COc1cc(CC(=O)N[C@@H](Cc2ccccc2)C(=O)N[C@@H](Cc2ccc(C(F)(F)P(=O)(O)O)cc2)C(=O)N[C@@H](CCCCNC(=O)c2ccc(C)c(Br)c2)C(=O)NCCCC(=O)Nc2cccc3c2C(=O)N(C2CCC(=O)NC2=O)C3=O)ccc1O] |
| 70 | 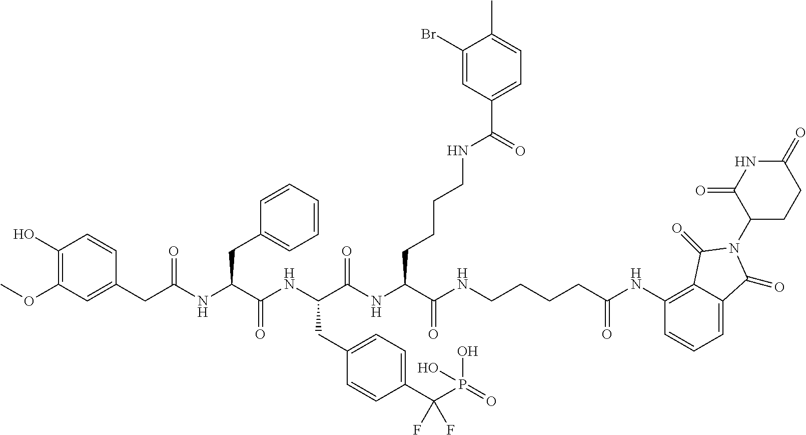 [SMILES: COc1cc(CC(=O)N[C@@H](Cc2ccccc2)C(=O)N[C@@H](Cc2ccc(C(F)(F)P(=O)(O)O)cc2)C(=O)N[C@@H](CCCCNC(=O)c2ccc(C)c(Br)c2)C(=O)NCCCCC(=O)Nc2cccc3c2C(=O)N(C2CCC(=O)NC2=O)C3=O)ccc1O] |
| 71 | 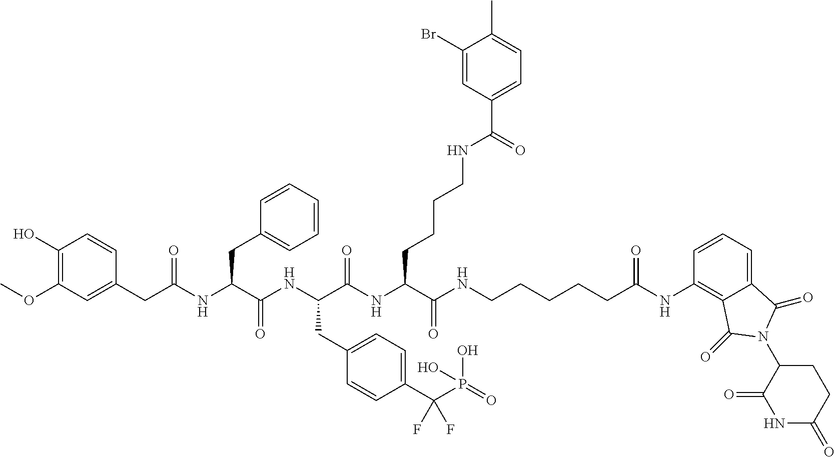 [SMILES: COc1cc(CC(=O)N[C@@H](Cc2ccccc2)C(=O)N[C@@H](Cc2ccc(C(F)(F)P(=O)(O)O)cc2)C(=O)N[C@@H](CCCCNC(=O)c2ccc(C)c(Br)c2)C(=O)NCCCCCC(=O)Nc2cccc3c2C(=O)N(C2CCC(=O)NC2=O)C3=O)ccc1O] |
| 72 | 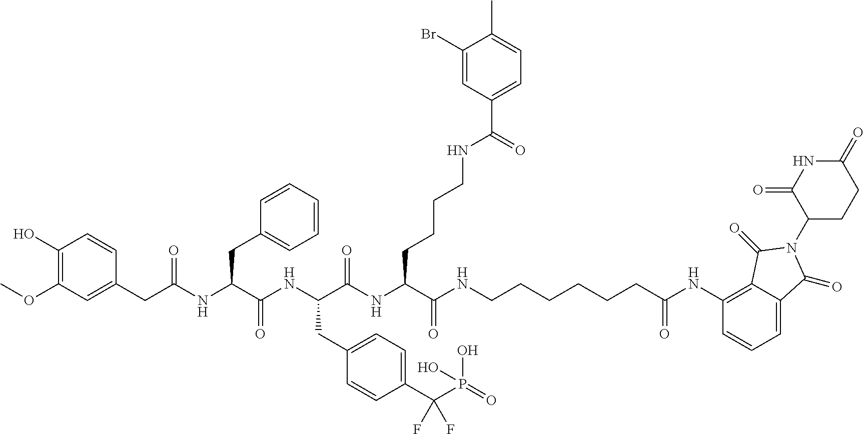 [SMILES: COc1cc(CC(=O)N[C@@H](Cc2ccccc2)C(=O)N[C@@H](Cc2ccc(C(F)(F)P(=O)(O)O)cc2)C(=O)N[C@@H](CCCCNC(=O)c2ccc(C)c(Br)c2)C(=O)NCCCCCCC(=O)Nc2cccc3c2C(=O)N(C2CCC(=O)NC2=O)C3=O)ccc1O] |
| 73 | 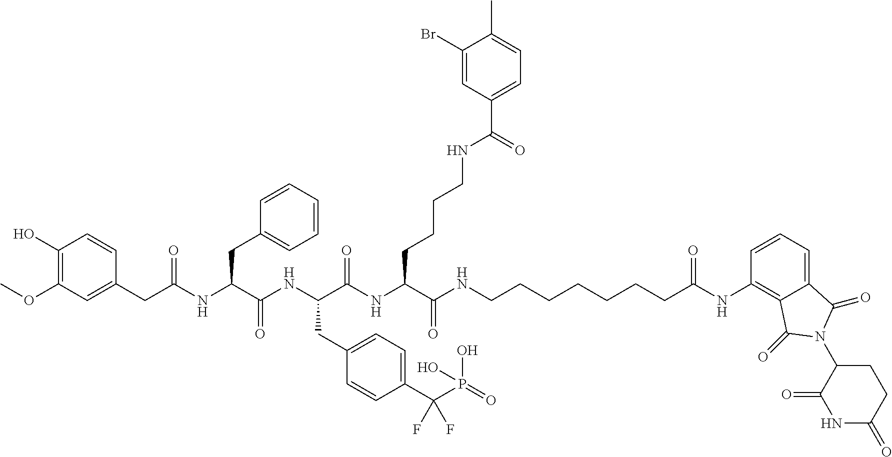 [SMILES: COc1cc(CC(=O)N[C@@H](Cc2ccccc2)C(=O)N[C@@H](Cc2ccc(C(F)(F)P(=O)(O)O)cc2)C(=O)N[C@@H](CCCCNC(=O)c2ccc(C)c(Br)c2)C(=O)NCCCCCCCC(=O)Nc2cccc3c2C(=O)N(C2CCC(=O)NC2=O)C3=O)ccc1O] |
| 74 | 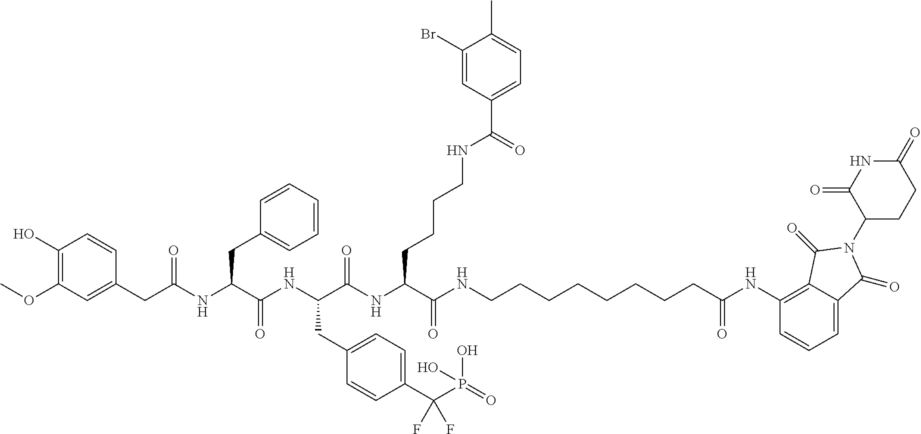 [SMILES: COc1cc(CC(=O)N[C@@H](Cc2ccccc2)C(=O)N[C@@H](Cc2ccc(C(F)(F)P(=O)(O)O)cc2)C(=O)N[C@@H](CCCCNC(=O)c2ccc(C)c(Br)c2)C(=O)NCCCCCCCCC(=O)Nc2cccc3c2C(=O)N(C2CCC(=O)NC2=O)C3=O)ccc1O] |
| 75 | 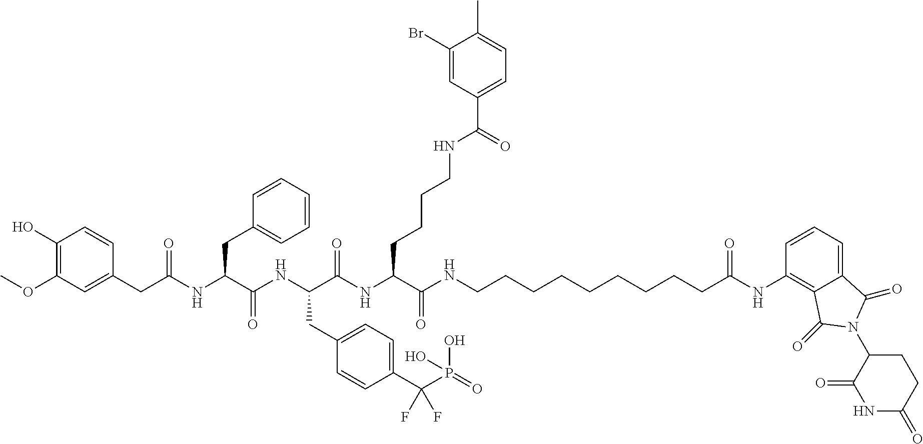 [SMILES: COc1cc(CC(=O)N[C@@H](Cc2ccccc2)C(=O)N[C@@H](Cc2ccc(C(F)(F)P(=O)(O)O)cc2)C(=O)N[C@@H](CCCCNC(=O)c2ccc(C)c(Br)c2)C(=O)NCCCCCCCCCC(=O)Nc2cccc3c2C(=O)N(C2CCC(=O)NC2=O)C3=O)ccc1O] |
| 76 | 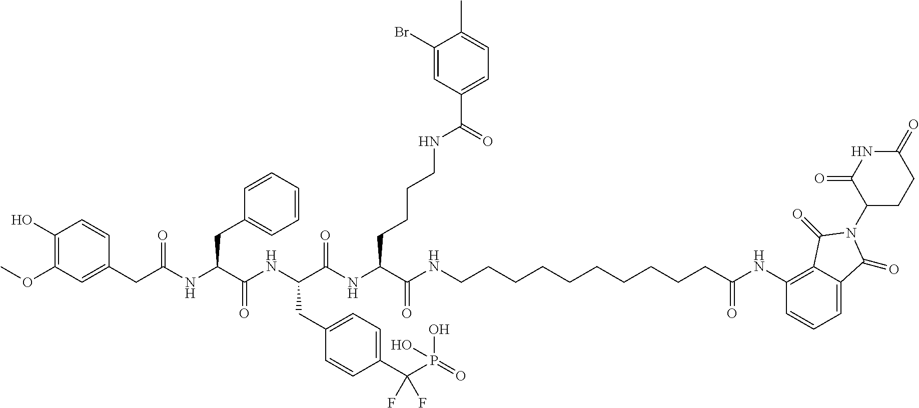 [SMILES: COc1cc(CC(=O)N[C@@H](Cc2ccccc2)C(=O)N[C@@H](Cc2ccc(C(F)(F)P(=O)(O)O)cc2)C(=O)N[C@@H](CCCCNC(=O)c2ccc(C)c(Br)c2)C(=O)NCCCCCCCCCCC(=O)Nc2cccc3c2C(=O)N(C2CCC(=O)NC2=O)C3=O)ccc1O] |
| 77 | 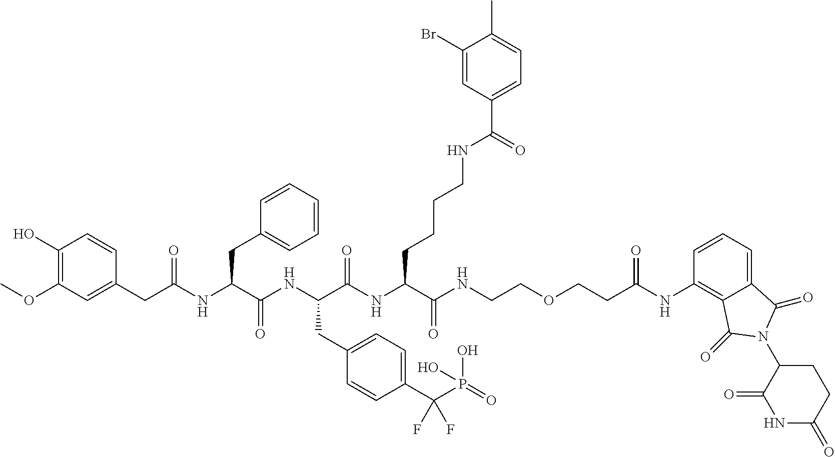 [SMILES: COc1cc(CC(=O)N[C@@H](Cc2ccccc2)C(=O)N[C@@H](Cc2ccc(C(F)(F)P(=O)(O)O)cc2)C(=O)N[C@@H](CCCCNC(=O)c2ccc(C)c(Br)c2)C(=O)NCCOCCC(=O)Nc2cccc3c2C(=O)N(C2CCC(=O)NC2=O)C3=O)ccc1O] |
| 78 | 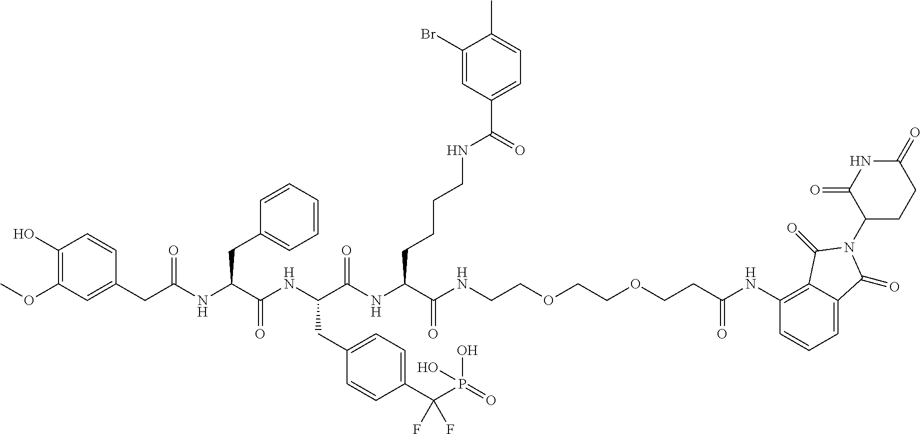 [SMILES: COc1cc(CC(=O)N[C@@H](Cc2ccccc2)C(=O)N[C@@H](Cc2ccc(C(F)(F)P(=O)(O)O)cc2)C(=O)N[C@@H](CCCCNC(=O)c2ccc(C)c(Br)c2)C(=O)NCCOCCOCCC(=O)Nc2cccc3c2C(=O)N(C2CCC(=O)NC2=O)C3=O)ccc1O] |
| 79 | 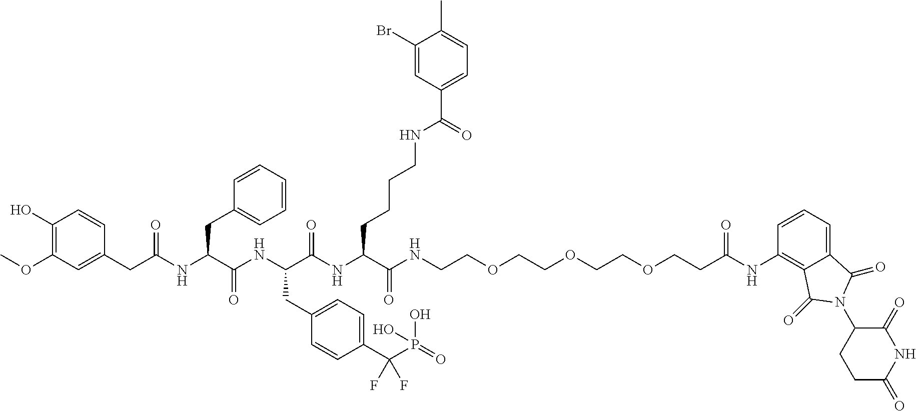 [SMILES: COc1cc(CC(=O)N[C@@H](Cc2ccccc2)C(=O)N[C@@H](Cc2ccc(C(F)(F)P(=O)(O)O)cc2)C(=O)N[C@@H](CCCCNC(=O)c2ccc(C)c(Br)c2)C(=O)NCCOCCOCCOCCC(=O)Nc2cccc3c2C(=O)N(C2CCC(=O)NC2=O)C3=O)ccc1O] |
| 80 | 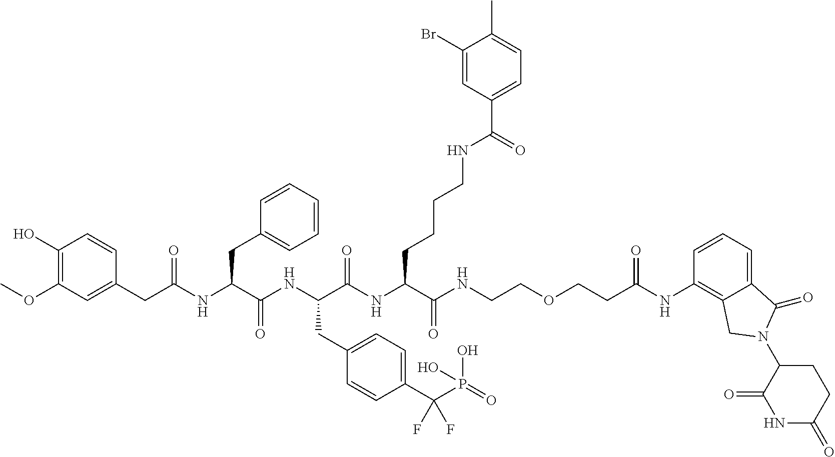 [SMILES: COc1cc(CC(=O)N[C@@H](Cc2ccccc2)C(=O)N[C@@H](Cc2ccc(C(F)(F)P(=O)(O)O)cc2)C(=O)N[C@@H](CCCCNC(=O)c2ccc(C)c(Br)c2)C(=O)NCCOCCC(=O)Nc2cccc3c2CN(C2CCC(=O)NC2=O)C3=O)ccc1O] |
| 81 | 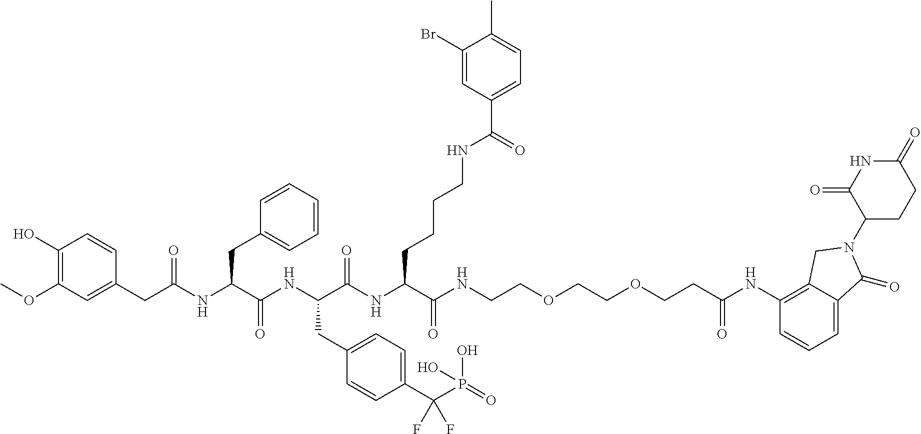 [SMILES: COc1cc(CC(=O)N[C@@H](Cc2ccccc2)C(=O)N[C@@H](Cc2ccc(C(F)(F)P(=O)(O)O)cc2)C(=O)N[C@@H](CCCCNC(=O)c2ccc(C)c(Br)c2)C(=O)NCCOCCOCCC(=O)Nc2cccc3c2CN(C2CCC(=O)NC2=O)C3=O)ccc1O] |
| 82 | 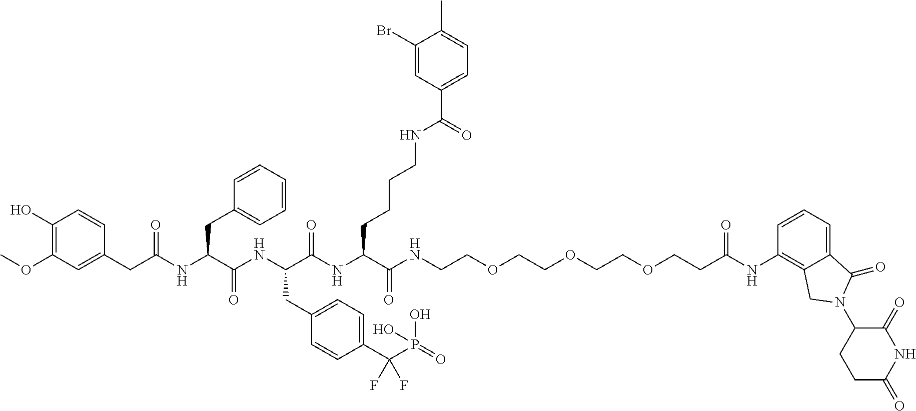 [SMILES: COc1cc(CC(=O)N[C@@H](Cc2ccccc2)C(=O)N[C@@H](Cc2ccc(C(F)(F)P(=O)(O)O)cc2)C(=O)N[C@@H](CCCCNC(=O)c2ccc(C)c(Br)c2)C(=O)NCCOCCOCCOCCC(=O)Nc2cccc3c2CN(C2CCC(=O)NC2=O)C3=O)ccc1O] |
| 83 | 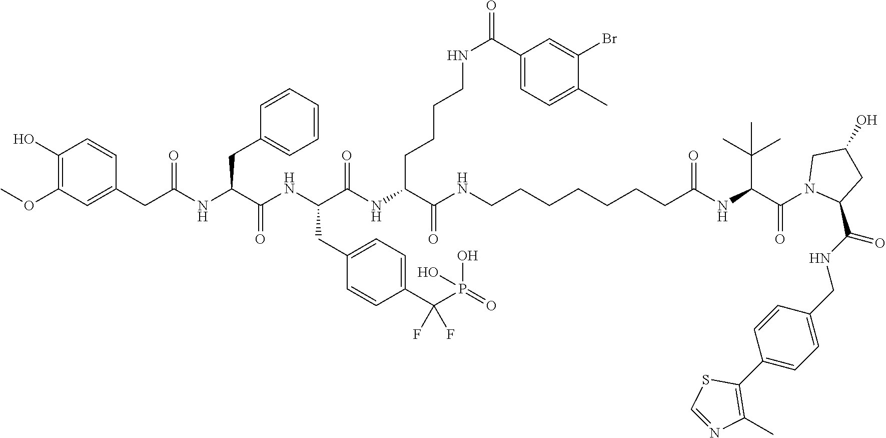 [SMILES: COc1cc(CC(=O)N[C@@H](Cc2ccccc2)C(=O)N[C@@H](Cc2ccc(C(F)(F)P(=O)(O)O)cc2)C(=O)N[C@H](CCCCNC(=O)c2ccc(C)c(Br)c2)C(=O)NCCCCCCCC(=O)N[C@H](C(=O)N2C[C@H](O)C[C@H]2C(=O)NCc2ccc(-c3scnc3C)cc2)C(C)(C)C)ccc1O] |
| 84 | 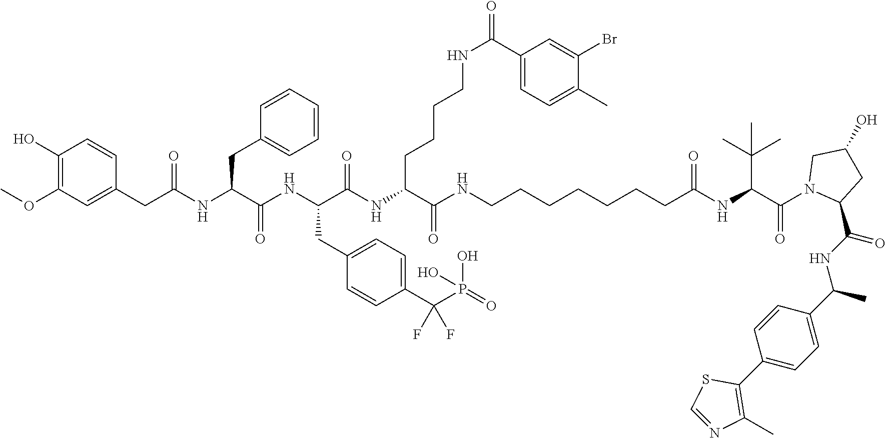 [SMILES: COc1cc(CC(=O)N[C@@H](Cc2ccccc2)C(=O)N[C@@H](Cc2ccc(C(F)(F)P(=O)(O)O)cc2)C(=O)N[C@H](CCCCNC(=O)c2ccc(C)c(Br)c2)C(=O)NCCCCCCCC(=O)N[C@H](C(=O)N2C[C@H](O)C[C@H]2C(=O)N[C@@H](C)c2ccc(-c3scnc3C)cc2)C(C)(C)C)ccc1O] |

or, pharmaceutically acceptable salt, hydrate, tautomer, or stereoisomer thereof.

The compounds of formulae (I), (II), and (III), and intermediates used for the preparation thereof, are enantiomerically enriched, e.g., the enantiomeric excess or “ee” of the compound is about 5% or more as measured by chiral HPLC.

In some embodiments, the ee is about 10%. In some embodiments, the ee is about 20%. In some embodiments, the ee is about 30%. In some embodiments, the ee is about 40%. In some embodiments, the ee is about 50%. In some embodiments, the ee is about 60%. In some embodiments, the ee is about 70%. In some embodiments, the ee is about 80%. In some embodiments, the ee is about 85%. In some embodiments, the ee is about 90%. In some embodiments, the ee is about 91%. In some embodiments, the ee is about 92%. In some embodiments, the ee is about 93%. In some embodiments, the ee is about 94%. In some embodiments, the ee is about 95%. In some embodiments, the ee is about 96%. In some embodiments, the ee is about 97%. In some embodiments, the ee is about 98%. In some embodiments, the ee is about 99%.

In some embodiments. B is the E3 ligase binding moiety of the molecule that can be enantiomerically enriched. In exemplary embodiments, the E3 ligase binding portion of the molecule is racemic. The present disclosure encompasses all possible stereoisomeric compounds, e.g., diastereomeric, forms of compounds of formulae (I), (II), and (III).

Provided are pharmaceutically acceptable salts of the above-described compounds and the preparation and use thereof. Salts can be prepared during the final isolation and purification of the compound or separately by reacting the compound with a suitable acid.

In some embodiments, the salts can be acid addition salts formed with pharmaceutically acceptable acids. Examples of inorganic acids, which can be employed to form pharmaceutically acceptable salts, include, but are not limited to, nitric acid, boric acid, hydrochloric acid, hydrobromic acid, sulfuric acid, and phosphoric acid. Examples of organic acids include, but are not limited to, oxalic acid, maleic acid, succinic acid, and citric acid.

Examples of the salts of the compounds described herein include, but are not limited to, hydrochloride salt, hydrobromide salt, hydroiodide salt, sulfate salt, bisulfate salt, 2-hydroxyethansulfonate salt, phosphate salt, hydrogen phosphate salt, acetate salt, adipate salt, alginate salt, aspartate salt, benzoate salt, bisulfate salt, butyrate salt, camphorate salt, camphorsulfonate salt, digluconate salt, glycerophosphate salt, hemisulfate salt, heptanoate salt, hexanoate salt, formate salt, succinate salt, fumarate salt, maleate salt, ascorbate salt, isethionate salt, salicylate salt, methanesulfonate salt, mesitylenesulfonate salt, naphthylenesulfonate salt, nicotinate salt, 2-naphthalenesulfonate salt, oxalate salt, pamoate salt, pectinate salt, persulfate salt, 3-phenylproprionate salt, picrate salt, pivalate salt, propionate salt, trichloroacetate salt, trifluoroacetate salt, phosphate salt, glutamate salt, bicarbonate salt, paratoluenesulfonate salt, undecanoate salt, lactate salt, citrate salt, tartrate salt, gluconate salt, methanesulfonate salt, ethanedisulfonate salt, benzene sulfonate salt, and p-toluenesulfonate salt.

The amino groups in the compounds can be quaternized with methyl chloride, ethyl chloride, propyl chloride, butyl chloride; methyl bromide, ethyl bromide, propyl bromide, butyl bromide; methyl iodide, ethyl iodide, propyl iodide, butyl iodide; dimethyl sulfate, diethyl sulfate, dibutyl sulfate, diamyl sulfate; decyl chloride, lauryl chloride, myristyl chloride, steryl chloride; decyl bromide, lauryl bromide, myristyl bromide, steryl bromide; decyl iodide, lauryl iodide, myristyl iodide, steryl iodide; and benzyl bromide or phenethyl bromide.

Provided are solvates of the above-described compounds and the preparation and use thereof. Solvates typically do not significantly alter the physiological activity or toxicity of the compounds and, as such, may function as pharmacological equivalents.

The term “solvate” refers to a combination, physical association, and/or solvation of a compound described herein with a solvent molecule such as e.g., a disolvate, monosolvate, or hemisolvate, where the ratio of the solvent molecule to a compound described is about 2:1, about 1:1 or about 1:2, respectively. This physical association involves varying degrees of ionic and covalent bonding, including hydrogen bonding. In certain instances, the solvate can be isolated, such as when one or more solvent molecules are incorporated into the crystal lattice of a crystalline solid. Thus, “solvate” encompasses both solution-phase and isolatable solvates.

Solvated forms of the compounds can be prepared with a pharmaceutically acceptable solvent. Examples of the solvents include, but are not limited to, water, methanol, and ethanol, and it is intended that the disclosure includes both solvated and unsolvated forms of above-described compounds. One type of solvate is a hydrate. A “hydrate” relates to a particular subgroup of solvates where the solvent molecule is water. Solvates typically can function as pharmacological equivalents. The preparation of solvates is known in the art. For example, M. Caira et al., J. Pharmaceut. Sci., 2004, 93(3): 601-611, describes the preparation of solvates of fluconazole with ethyl acetate and with water. Similar preparation of solvates, hemisolvates, hydrates, and the like are described by E. C. van Tonder et al., AAPS Pharm. Sci. Tech., 2004, 5(1): Article 12, and A. L. Bingham et al, 200, Chem. Commun., 603-604. A typical, non-limiting, process of preparing a solvate would involve dissolving a compound in a desired solvent (organic, water, or a mixture thereof) at temperatures above 20° C. to about 25° C. then cooling the solution at a rate sufficient to form crystals, and isolating the crystals by known methods, e.g., filtration. Analytical techniques, such as infrared spectroscopy, can be used to confirm the presence of the solvate in a crystal of the solvate.

The term “pharmaceutically acceptable salt” refers to salts or zwitterionic forms of compounds described herein.

Any reference compounds of the present disclosure appearing herein are intended to include compounds of the present disclosure as well as pharmaceutically acceptable salts or hydrates thereof.

The term “alkyl” refers to substituted or unsubstituted straight-chain and branched alkyl groups and cycloalkyl groups having from 1 to about 20 carbon atoms (C₁-C₂₀), 1 to 12 carbon atoms (C₁-C₁₂), 1 to 8 carbon atoms (C₁-C₈), or, in some embodiments, from 1 to 6 carbon atoms (C₁-C₆). Examples of straight-chain alkyl groups include, but are not limited to, those with from 1 to 8 carbon atoms, such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, tert-butyl, neopentyl, isopentyl, and 2,2-dimethylpropyl groups. The term “alkyl” encompasses n-alkyl, isoalkyl, and anteisoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halogen groups.

The term “alkylene,” by itself or as part of another substituent, means, unless otherwise stated, a divalent radical derived from an alkyl, as exemplified, but not limited by, —CH₂CH₂CH₂CH₂-. Typically, an alkyl (or alkylene) group will have from 1 to 24 carbon atoms, with those groups having 10 or fewer carbon atoms being preferred in various embodiments.

The term “cycloalkyl” refers to substituted or unsubstituted cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments, the number of ring carbon atoms ranges from 3 to 4, 5, 6, or 7. In some embodiments, cycloalkyl groups can have 3 to 6 carbon atoms (C₃-C₆). Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like.

The term “alkenyl” refers to substituted or unsubstituted straight-chain and branched divalent alkenyl and cycloalkenyl groups having from 2 to 20 carbon atoms (C₂-C₂₀), 2 to 12 carbon atoms (C₂-C₁₂), 2 to 8 carbon atoms (C₂-C₈) or, in some embodiments, from 2 to 4 carbon atoms (C₂-C₄) and at least one carbon-carbon double bond. Examples of straight-chain alkenyl groups include those with from 2 to 8 carbon atoms such as —CH═CH—, —CH═CHCH₂—, and the like. Examples of branched alkenyl groups include, but are not limited to, —CH═C(CH₃)— and the like.

The term “alkenylene,” by itself or as part of another substituent, means, unless otherwise stated, a divalent radical derived from an alkene. An alkylene group may be described as, e.g., a 1-6-membered alkylene, wherein the term “membered” refers to the non-hydrogen atoms within the moiety.

The term “alkynyl” refers to an unsaturated monovalent chain of carbon atoms, including at least one triple bond, which may be optionally branched. In various embodiments that include alkynyl, illustrative examples include lower alkynyl, such as C₂-C₆, C₂-C₄ alkynyl, and the like.

The term “alkoxy” refers to an oxygen atom connected to an alkyl group, including a cycloalkyl group, as are defined herein. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can further include double or triple bonds and can also include heteroatoms. For example, an allyloxy group is an alkoxy group within the meaning herein. A methoxyethoxy group is also an alkoxy group within the meaning herein, as is a methylenedioxy group in a context where two adjacent atoms of a structure are substituted therewith.

The term “halogen” is used to describe chemical compounds which contain one or more halogen atoms, such as fluorine, chlorine, bromine, and iodine.

It is understood that each of alkyl, cycloalkyl, alkenyl, cycloalkenyl, alkylene, and heterocycle may be optionally substituted with independently selected groups such as alkyl, haloalkyl, hydroxyalkyl, aminoalkyl, carboxylic acid and derivatives thereof, including esters, amides, and nitrites, hydroxy, alkoxy, acyloxy, amino, alky and dialky-lamino, acylamino, thio, and the like, and combinations thereof.

The term “heterocyclyl” refers to substituted or unsubstituted aromatic and non-aromatic ring compounds containing three or more ring members, of which one or more is a heteroatom such as, but not limited to, B, N, O, and S. Thus, a heterocyclyl can be a cycloheteroalkyl, or a heteroaryl, or if polycyclic, any combination thereof. In some embodiments, heterocyclyl groups include 3 to about 20 ring members, whereas other such groups have 3 to about 15 ring members. In some embodiments, heterocyclyl groups can include 3 to 8 carbon atoms (C₃-C₈), 3 to 6 carbon atoms (C₃-C₆) or 6 to 8 carbon atoms (C₆-C₈).

The term “substituted” refers to a functional group in which one or more hydrogen atoms contained therein are replaced by one or more non-hydrogen atoms. The term “functional group” or “substituent” refers to a group that can be or is substituted onto a molecule. Examples of substituents or functional groups include, but are not limited to, a halo (e.g., F, Cl, Br, and I); an oxygen atom in groups such as hydroxyl groups, alkoxy groups, aryloxy groups, aralkyloxy groups, oxo(carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, azides, hydroxylamines, cyano, nitro groups, N-oxides, hydrazides, and enamines; and other heteroatoms in various other groups.

Non-limiting examples of substituents, which can be bonded to a substituted carbon atom (or other atom, such as nitrogen) include F, Cl, Br, I, OR, OC(O)N(R)₂, CN, NO, NO₂, ONO₂, azido, CF₃, OCF₃, R, O (oxo), S (thiono), C(O), S(O), methylenedioxy, ethylenedioxy, N(R)₂, SR, SOR, SO₂R, SO₂N(R)₂, SO₃R, (CH₂)₀₋₂P(O)OR₂, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)C(O)OR, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, and C(═NOR)R wherein R can be hydrogen or a carbon-based moiety, and wherein the carbon-based moiety can itself be further substituted; for example, where R can be hydrogen, alkyl, acyl, cycloalkyl, aryl, aralkyl, heterocyclyl, heteroaryl, or heteroarylalkyl, any alkyl, acyl, cycloalkyl, aryl, aralkyl, heterocyclyl, heteroaryl, or heteroarylalkyl or R can be independently mono- or multi-substituted; or when two R groups bonded to a nitrogen atom or to adjacent nitrogen atoms can, together with the nitrogen atom or atoms to which they are bonded, form a heterocyclyl, the heterocycle can be mono- or independently multi-substituted.

The terms “optionally substituted” and “optional substituents” indicate that the groups in question are either unsubstituted or substituted with one or more of the substituents specified. When the groups in question are substituted with more than one substituent, the substituents may be the same or different. When used with the terms “independently,” “independently are,” and “independently selected from,” the groups in question may be the same or different. Certain of the herein defined terms may occur more than once in the structure and, upon such occurrence, each term shall be defined independently of the other.

The term “amine” refers to primary, secondary, and tertiary amines having, e.g., the formula N(group)3 wherein each group can independently be H or non-H, such as alkyl, aryl, and the like. Amines include, but are not limited to, R—NH₂, for example, alkylamines, arylamines, alkylarylamines; R₂NH, wherein each R is independently selected, such as dialkylamines, diarylamines, aralkylamines, heterocyclylamines and the like; and R₃N, wherein each R is independently selected, such as trialkylamines, dialkylarylamines, alkyldiarylamines, triarylamines, and the like. The term “amine” also includes ammonium ions.

The term “amino group” refers to a substituent of the form —NH₂, —NHR, —NR₂, —NR₃+, wherein each R is independently selected, and protonated forms of each, except for —NR₃+, which cannot be protonated. Accordingly, any compound substituted with an amino group can be viewed as an amine. An “amino group” can be a primary, secondary, tertiary, or quaternary amino group. An “alkylamino” group includes a monoalkylamino, a dialkylamino, and a trialkylamino group.

The compounds may contain one or more chiral centers, or may otherwise be capable of existing as multiple stereoisomers. In various embodiments, the compounds are not limited to any particular stereochemical requirement, and the compounds, and compositions, methods, uses, and medicaments that include them, may be optically pure or any of a variety of stereoisomeric mixtures, including racemic and other mixtures of enantiomers, other mixtures of diastereomers, and the like. Such mixtures of stereoisomers may include a single stereochemical configuration at one or more chiral centers, while including mixtures of stereochemical configuration at one or more other chiral centers.

Similarly, the compounds described herein may include geometric centers, such as cis, trans, E, and Z double bonds. In various embodiments, the compounds are not limited to any particular geometric isomer requirement, and the compounds, and compositions, methods, uses, and medicaments that include them, may be pure or any of a variety of geometric isomer mixtures. Such mixtures of geometric isomers may include a single configuration at one or more double bonds, while including mixtures of geometry at one or more other double bonds.

The term “compound” as used herein, is meant to include all stereoisomers, geometric isomers, and tautomers of the structures depicted.

In some embodiments, the degrees to which the compound inhibits PTP1B and TC-PTP can be the same. In other embodiments, the compound inhibits PTP1B to a greater extent than it inhibits TC-PTP. In yet other embodiments, the compound inhibits TC-PTP to a greater extent than it inhibits PTP1B. When there is differential inhibition between PTP1B and TC-PTP, the differential inhibition can range from about as little as about 1% (e.g., 1%) to as much as about 80% (e.g., 80%). It can be preferable in various embodiments, and even desirable, for the compound to inhibit PTP1B and TC-PTP at least approximately to the same extent.

In some embodiments, provided is a pharmaceutical composition comprising one or more compounds of formula (I), (II), or (III) or a pharmaceutically acceptable salt, hydrate, tautomer, or stereoisomer of either of the foregoing, and a pharmaceutically acceptable carrier or excipient. The carrier or excipient can vary based on the particular route of administration (see, e.g., Remington's The Science and Practice of Pharmacy, 23rd ed. (2020)).

In some embodiments, a pharmaceutical composition further comprises at least one additional pharmaceutically active agent. Pharmaceutical compositions can be prepared by combining one or more compounds of formula (I), (II), or (III) or a pharmaceutically acceptable salt, hydrate, tautomer, or stereoisomer of either of the foregoing with a pharmaceutically acceptable carrier or excipient and, optionally, one or more additional pharmaceutically active agents.

Provided is a pharmaceutical composition comprising (i) one or more compounds of formula (I), (II), or (III) or a pharmaceutically acceptable salt, hydrate, tautomer, or stereoisomer of either of the foregoing, and (ii) one or more other prophylactic or therapeutic agents, and a pharmaceutically acceptable carrier or excipient.

The compound and one or more other prophylactic or therapeutic agents can be administered as two separate compositions, simultaneously or sequentially, in either order by the same or different routes.

Compounds of the formulae (I). (II), and (III) or a pharmaceutically acceptable salt, hydrate, tautomer, or stereoisomer of either of the foregoing, can inhibit or degrade both PTP1B and TC-PTP. Thus, they can treat or prevent various diseases and conditions associated with PTP1B and TC-PTP. In particular, the compounds are beneficial in treating or preventing a disease or condition wherein inhibition or degradation of both PTP1B and TC-PTP provides a benefit.

Provided is a method of treating or preventing cancer in a patient. The method comprises administering to the patient in need thereof an effective amount of a compound of formula (I), (II), or (III) or a pharmaceutically acceptable salt, hydrate, tautomer, or stereoisomer of either of the foregoing, optionally as a pharmaceutical composition comprising the compound and a pharmaceutically acceptable carrier or excipient, whereupon the cancer in the patient is treated or prevented.

Further provided is a method of treating or preventing cancer in a patient. The method comprises administering to the patient in need thereof an effective amount of (i) a compound of formula (I), (II), or (III) or a pharmaceutically acceptable salt, hydrate, tautomer, or stereoisomer of either of the foregoing, and (ii) one or more other prophylactic or therapeutic agents, optionally as a pharmaceutical composition comprising (i) and (ii) and a pharmaceutically acceptable carrier or excipient. The other prophylactic or therapeutic agent can be selected from drugs known to prevent or treat cancer, e.g., a monoclonal antibody useful in treating a particular cancer.

Examples of cancer include, but are not limited to, colon cancer, lung adenocarcinoma, squamous cell carcinoma, and melanoma.

In some embodiments, the compound of the disclosure can promote weight loss and improve glucose metabolism.

Further provided is a method of treating or preventing type II diabetes. The method comprises administering to a patient in need thereof an effective amount of a compound of formula (I), (II), or (III) or a pharmaceutically acceptable salt, hydrate, tautomer, or stereoisomer of either of the foregoing, optionally as a pharmaceutical composition comprising the compound and a pharmaceutically acceptable carrier or excipient, whereupon the type II diabetes in the patient is treated or prevented. The effective amount of the compound can be optionally administered with one or more other prophylactic or therapeutic agents. The other prophylactic or therapeutic agent can be selected from drugs known to prevent or treat type II diabetes.

Further provided is a method of treating or preventing obesity. The method comprises administering to a patient in need thereof an effective amount of a compound of formula (I), (II), or (III), or a pharmaceutically acceptable salt, hydrate, tautomer, or stereoisomer of either of the foregoing, optionally as a pharmaceutical composition comprising the compound and a pharmaceutically acceptable carrier or excipient, whereupon the obesity in the patient is treated or prevented. The effective amount of the compound can be optionally administered with one or more other prophylactic or therapeutic agents. The other prophylactic or therapeutic agent can be selected from drugs known to prevent or treat obesity.

Since compounds described herein are inhibitors or degraders of PTP1B and TC-PTP protein, a number of diseases and conditions mediated by PTP1B and/or TC-PTP can be treated (e.g., prophylactically or therapeutically) by employing these compounds.

Still further provided is a method of treating or preventing a disease or condition responsive to PTP1B/TC-PTP inhibition or degradation. The method comprises administering to a patient in need thereof an effective amount of an above-described compound or a pharmaceutically acceptable salt, hydrate, tautomer, or stereoisomer thereof, optionally as a pharmaceutical composition comprising the compound and a pharmaceutically acceptable carrier or excipient. The effective amount of the compound can be optionally administered with one or more other prophylactic or therapeutic agents. The other prophylactic or therapeutic agent can be selected from drugs known to prevent or treat the disease or condition.

Still further provided is a method of inhibiting or degrading dual PTP1B and TC-PTP in a patient, wherein the method comprises administering to the patient in need thereof an effective amount of a compound described herein above or a pharmaceutical composition comprising the compound and a pharmaceutically acceptable carrier or excipient, whereupon dual PTP1B and TC-PTP in the patient is inhibited or degraded. In some embodiments, the patient has colon cancer, lung adenocarcinoma, squamous cell carcinoma, or melanoma. In some embodiments, the patient has type II diabetes. In some embodiments, the patient is obese.

The method comprises administering an effective amount of an above-described compound as a neat compound or as a pharmaceutical composition. The compound or pharmaceutical composition can be administered during or after the onset of the disease or condition. Typically, the pharmaceutical compositions are sterile and contain no toxic, carcinogenic, or mutagenic compounds, which would cause an adverse reaction when administered.

Still further provided is the use of a compound described herein above in the treatment of a disease or condition that can be treated by inhibiting or degrading dual PTP1B or TC-PTP proteins. In some embodiments, the disease or condition is cancer, type II diabetes, or obesity.

Still further provided is a kit comprising an above-described compound and, optionally, one or more other prophylactic or therapeutic agents, packaged separately or together, and an insert having instructions for using these active agents.

The other therapeutic agent(s) can be administered simultaneously or sequentially, by the same or a different route, to achieve the desired effect. The compound described herein and one or more other prophylactic or therapeutic agents can be administered from a single composition or two separate compositions, such as by the same or different routes. The prophylactic or therapeutic agent can be administered in an amount to provide its desired prophylactic or therapeutic effect. The effective dosage range for each prophylactic or therapeutic agent is well-known in the art or can be determined in accordance with dosage range-determining methods known to those of ordinary skill in the art, and the therapeutic agent can be administered to a patient in need thereof within such established ranges. The compound described herein, and one or more other prophylactic or therapeutic agents can be administered together as a single unit dose or separately as multi-unit doses, wherein the compound can be administered before the other prophylactic or therapeutic agent (s) or vice versa. One or more doses of the compound and/or one or more doses of the second prophylactic or therapeutic agent can be administered.

The term “effective amount” or “effective dose” refers to an amount of the active ingredient(s) that is(are) sufficient, when administered, to deliver efficaciously the active ingredient(s) for the treatment of a disease or condition of interest to a subject in need thereof. The prophylactically or therapeutically effective amount of such compound will vary depending upon the patient and the disease or condition being treated, the weight and age of the patient, the severity of the disease or condition, the manner of administration, and the like, which can readily be determined by one of ordinary skill in the art. In the case of a cancer or other proliferative disorder, the prophylactically or therapeutically effective amount of the agent may reduce (i.e., inhibit to some extent or stop) unwanted cellular proliferation; reduce the number of cancer cells; reduce the tumor size; inhibit (or stop) cancer cell infiltration into peripheral organs; inhibit (or stop) tumor metastasis; inhibit, e.g., to some extent, tumor growth; and/or relieve, to some extent, one or more of the signs or symptoms associated with the cancer. To the extent the administered compound or composition prevents growth and/or kills existing cancer cells, it may be cytostatic and/or cytotoxic.

The compounds can be typically administered in admixture with a pharmaceutical carrier to give a pharmaceutical composition selected with regard to the intended route of administration and standard pharmaceutical practice. Pharmaceutical compositions can be formulated in a conventional manner using one or more physiologically acceptable carriers comprising excipients and/or auxiliaries that facilitate the processing of the compound. The pharmaceutical compositions can be manufactured, for example, by conventional mixing, dissolving, granulating, dragee-making, emulsifying, encapsulating, entrapping, or lyophilizing processes. Proper formulation is dependent upon the route of administration chosen. When a therapeutically effective amount of a compound described herein is administered orally, the composition typically is in the form of a tablet, capsule, powder, solution, or elixir. When administered in tablet form, the composition additionally can contain a solid carrier, such as a gelatin or an adjuvant. The tablet, capsule, and powder can contain about 0.01% to about 95%, and preferably from about 1% to about 50%, of the compound. When administered in liquid form, a liquid carrier can be added, such as water, petroleum, or oils of animal or plant origin. The liquid form of the composition can further contain the physiological saline solution, dextrose or other saccharide solutions, or glycols. When administered in liquid form, the composition contains about 0.1% to about 90%, and preferably about 1% to about 50%, by weight, of the compound.

The compounds can be administered by any suitable route, for example, oral, buccal, inhalation, sublingual, rectal, vaginal, intracisternal, intrathecal through a lumbar puncture, transurethral, nasal, percutaneous, i.e., transdermal, or parenteral including intravenous, intramuscular, subcutaneous, intracoronary, intradermal, intramammary, intraperitoneal, intraarticular, intrathecal, retrobulbar, intrapulmonary injection and/or surgical implantation at a particular site administration. Parenteral administration can be accomplished using a needle and syringe or using a high-pressure technique.

For oral administration, the compounds can be formulated readily by combining the active compound(s) with pharmaceutically acceptable carriers, excipients, or diluents well-known in the art. Such carriers, excipients, or diluents enable the compounds to be formulated as tablets, pills, powders, dragees, capsules, liquids, gels, syrups, slurries, suspensions, solutions, and the like for oral ingestion by a subject to be treated.

The exact formulation, route of administration, and dosage of a pharmaceutical composition comprising an effective amount of the compound are determined by an individual physician in view of the diagnosed condition or disease. The dosage amount and interval can be adjusted individually to provide levels of the compound that are sufficient to maintain a prophylactic or therapeutic effect.

Toxicity and therapeutic efficacy of the compounds can be determined by standard pharmaceutical procedures in cell cultures or experimental animals, e.g., for determining the maximum tolerated dose (MTD) of a compound, which is defined as the highest dose that causes no toxicity in animals. The therapeutic index is the dose ratio between the maximum tolerated dose and therapeutic effects (e.g., inhibition of tumor growth). The dosage can vary within this range depending upon the dosage form employed and the route of administration utilized. The determination of a therapeutically effective amount is well within the capability of those ordinarily skilled in the art, especially in light of the detailed disclosure provided herein.

A effective amount of a compound required for use in therapy varies with the nature of the condition being treated, the length of time that activity is desired, and the age and the condition of the patient, and ultimately is determined by the attendant physician. Dosage amounts and intervals can be adjusted individually to provide plasma levels of the PTP1B/TC-PTP dual inhibitor or degrader that are sufficient to maintain the desired prophylactic or therapeutic effect. The desired dose can be administered in a single dose or as multiple doses administered at appropriate intervals, for example, as one, two, three, four, or more sub-doses per day. Multiple doses often are desired or required. For example, the compound can be administered at a frequency of four doses delivered as one dose per day at four-day intervals (q4d×4). Four doses delivered as one dose per day at three-day intervals (q3d×4); one dose delivered per day at five-day intervals (qd×5); one dose per week for three weeks (qwk3); five daily doses, with two days rest, and another five daily doses (5/2/5); or, any dose regimen determined to be appropriate for the circumstance.

A compound can be administered in an amount of about 0.005 to about 500 milligrams per dose, about 0.05 to about 250 milligrams per dose, or about 0.5 to about 100 milligrams per dose. For example, the compound can be administered, per dose, in an amount of about 0.005, about 0.05, about 0.5, about 5, about 10, about 20, about 30, about 40, about 50, about 100, about 150, about 200, about 250, about 300, about 350, about 400, about 450, or about 500 milligrams, including all doses between 0.005 and 500 milligrams.

The compositions comprising the compound (s) can be formulated in a unit dosage form, each dosage containing from about 5 to about 1.000 mg (I g), more usually about 100 mg to about 500 mg, of the active ingredient.

The dosage of a composition comprising a compound described herein, can range from about 1 mg/kg to about 200 mg/kg, about 1 mg/kg to about 100 mg/kg, or about 1 mg/kg to about 50 mg/kg. The above dosages are exemplary of the average case, but there can be individual instances in which higher or lower dosages are merited, and such are within the scope of this disclosure. In practice, the physician determines the actual dosing regimen that is most suitable for an individual patient, which can vary with the age, weight, and response of the particular patient.

As stated above, a compound described herein can be administered in combination with one or more other prophylactically or therapeutically active agents. In some embodiments, the other therapeutically active agent (s) is an immune checkpoint inhibitor. An immune checkpoint inhibitor is a type of drug that blocks proteins called checkpoints that are made by some types of immune system cells, such as T cells, and some cancer cells. These checkpoints help keep immune responses from being too strong and sometimes can keep T cells from killing cancer cells. When these checkpoints are blocked, T cells can kill cancer cells better. Examples of immune checkpoint inhibitors include, but are not limited to, PD-1 inhibitors, PD-L1 inhibitors, CTLA-4 inhibitors, LAG-3 inhibitors, TIM-3 inhibitors, CD47 inhibitors, and B7-H1 inhibitors.

In some embodiments, the immune checkpoint inhibitor is a programmed cell death (PD-1) inhibitor. PD-1 is a T-cell coinhibitory receptor that plays a pivotal role in the ability of tumor cells to evade the host's immune system. Blockage of interactions between PD-1 and PD-L1, a ligand of PD-1, enhances immune function and mediates the antitumor activity. Examples of PD-1 inhibitors specifically include, but are not limited to, antibodies that specifically bind to PD-1. The anti-PD-1 antibodies selected from the group consisting of nivolumab, pembrolizumab, STI-A1014, and pidilizumab. The availability, methods of production, mechanism of action, and clinical studies of anti-PD-1 antibodies are described in U.S. Pat. App. Pub. No. 2013/0309250, U.S. Pat. No. 7,595,048. U.S. Pat. No. 8,728,474. U.S. Pat. Nos. 8,779,105, 8,952,136, 8,900,587, 9,073,994, and 9,084,776, all of which are hereby specifically incorporated by reference for their teachings regarding same.

In some embodiments, the immune checkpoint inhibitor is a PD-L1 (also known as B7-H1 or CD274) inhibitor. PD-L1 inhibitors include antibodies that specifically bind to PD-L1. Examples of anti-PD-L1 antibodies include, but are not limited to, avelumab, atezolizumab, durvalumab, and BMS-936559. Anti-PD-L1 antibodies's availability, methods of production, mechanism of action, and clinical studies are described in U.S. Pat. No. 8,217,149, U.S. Pat. App. Pub. No. 2014/0341917. U.S. Pat. App. Pub. No. 2013/0071403, Int'l Pat. App. Pub. No. WO 2015036499, and Naido et al., British Journal of Cancer 2014, 777 2214-19, all of which are hereby specifically incorporated by reference for their teachings regarding same.

In some embodiments, the immune checkpoint inhibitor is a CTLA-4 inhibitor. CTLA-4, also known as cytotoxic T-lymphocyte antigen 4, is a protein receptor that down-regulates the immune system. CTLA-4 is characterized as a “brake” that binds costimulatory molecules on antigen-presenting cells, which prevents interaction with CD28 on T cells and also generates an overtly inhibitory signal that constrains T cell activation. Examples of CTLA-4 inhibitors include antibodies that specifically bind to CTLA-4. Particular anti-CTLA-4 antibodies include, but are not limited to, ipilimumab and tremelimumab. The availability, methods of production, mechanism of action, and clinical studies of CTLA-4 antibodies are described in U.S. Pat. Nos. 6,984,720, 6,207,156, and Naido et al., British Journal of Cancer 2014, 777 2214-19, all of which are hereby specifically incorporated by reference for their teachings regarding same.

In some embodiments, the immune checkpoint inhibitor is a LAG-3 inhibitor. LAG-3, Lymphocyte Activation Gene 3, is a negative co-stimulatory receptor that modulates T cell homeostatis, proliferation, and activation. In addition, LAG-3 has been reported to participate in regulatory T cells (Tregs) suppressive function. A large proportion of LAG-3 molecules are retained in the cell close to the microtubule organizing center and only induced following antigen-specific T cell activation. (see U.S. Pat. App. Pub. No. 2014/0286935). LAG-3 inhibitors include antibodies that specifically bind to LAG-3. Examples of anti-LAG-3 antibodies include, but are not limited to, GSK2831781. For a general discussion of the availability, methods of production, mechanism of action, and studies, see U.S. Pat. App. Pub. No. 2011/0150892, U.S. Pat. App. Pub. No. 2014/0093511, U.S. Pat. App. Pub. No. 2015/0259420, and Huang et al., Immunity, 2004, 21, 503-13, all of which are hereby specifically incorporated by reference for their teachings regarding same.

In some embodiments, the immune checkpoint inhibitor is a TIM-3 inhibitor. TIM-3, T-cell immunoglobulin and mucin domain 3, is an immune checkpoint receptor that functions to limit the duration and magnitude of TH1 and TC1 T-cell responses. The TIM-3 pathway is considered a target for anticancer immunotherapy due to its expression on dysfunctional CD8⁺ T cells and Tregs, which are two reported immune cell populations that constitute immunosuppression in tumor tissue (Anderson, Cancer Immunology Research 2014, 2, 393-98). Examples of TIM3 inhibitors include antibodies that specifically bind to TIM-3. For a general discussion of the availability, methods of production, mechanism of action, and studies of TIM-3 inhibitors, see U.S. Pat. App. Pub. No. 2015/0225457, U.S. Pat. App. Pub. No. 2013/0022623, U.S. Pat. No. 8,522,156, Ngiow et al., Cancer Res 201, 7, 6567-71, Ngiow, et al., Cancer Res 201, 7, 3540-51, and Anderson, Cancer Immunology Res., 2014, 2, 393-98, all of which are hereby specifically incorporated by reference for their teachings regarding same.

In some embodiment, the immune checkpoint inhibitor is a CD47 inhibitor (see Unanue, E. R., PNAS 2013, 110:10886-87, which is specifically incorporated herein by reference for its teachings regarding same).

The term “antibody” refers to intact monoclonal antibodies, polyclonal antibodies, multispecific antibodies formed from at least two intact antibodies, and antibody fragments, so long as they exhibit the desired biological activity. In some embodiments, “antibody” refers to include soluble receptors that do not possess the Fc portion of the antibody. In some embodiments, the antibodies are humanized monoclonal antibodies and fragments thereof made by means of recombinant genetic engineering.

Another class of immune checkpoint inhibitors includes polypeptides that bind to and block PD-1 receptors on T-cells without triggering inhibitor signal transduction. U.S. Pat. No. 8,114,845 (specifically incorporated herein by reference for its teachings regarding same) describes such peptides, including B7-DC polypeptides. B7-H1 polypeptides. B7-1 polypeptides, and B7-2 polypeptides, and soluble fragments thereof.

Another class of immune checkpoint inhibitors includes compounds with peptide moieties that inhibit PD-1 signaling as disclosed in U.S. Pat. No. 8,907,053, which is specifically incorporated herein by reference for its teachings regarding same.

Another class of immune checkpoint inhibitors includes inhibitors of certain metabolic enzymes, such as indoleamine 2,3 dioxygenase (IDO), which is expressed by infiltrating myeloid cells and tumor cells. The IDO enzyme inhibits immune responses by depleting amino acids that are necessary for anabolic functions in T cells or through the synthesis of particular natural ligands for cytosolic receptors that are able to alter lymphocyte functions (Lob, Cancer Immunol Immunother, 2009, 58.T53-57). Particular IDO blocking agents include, but are not limited to, levo-1-methyl tryptophan (L-1MT) and 1-methyl-tryptophan (1MT) (Lob et al., Cancer Immunol Immunother 2009, 58.T53-7; specifically incorporated herein by reference for its teachings regarding same).

In some embodiments, the immune checkpoint inhibitor is nivolumab, pembrolizumab, pidilizumab, STI-A1110, avelumab, atezolizumab, durvalumab, STI-A1014, ipilimumab, tremelimumab, GSK2831781, BMS-936559 or MED14736.

The above-described other prophylactically or therapeutically active agents, one or more of which can be used in combination with a compound of formula (I), (II), or (III), are prepared and administered as described in the art.

The term “a disease or condition wherein inhibition or degradation of PTP1B/TC-PTP provides a benefit” and the like pertains to a disease or condition in which PTP1B/TC-PTP is important or necessary, e.g., for the onset, progress, expression of that disease or condition, or a disease or a condition which is known to be treated by an PTP1B/TC-PTP inhibitor or degrader.

The term “additional prophylactic or therapeutic agent” refers to a prophylactic or therapeutic agent different from a compound of the disclosure and that is known to treat the disease or condition of interest.

The term “disease” or “condition” denotes disturbances and/or anomalies that as a rule are regarded as being pathological conditions or functions, and that can manifest themselves in the form of particular signs, symptoms, and/or malfunctions.

As used herein, the terms “treat,” “treating,” “treatment,” and the like refer to eliminating, reducing, or ameliorating a disease or condition and/or symptoms associated therewith. Although not precluded, treating a disease or condition does not require that the disease, condition, or symptoms associated therewith be completely eliminated. The term “treat” and synonyms contemplate administering a prophylactic or therapeutically effective amount of a compound described herein to a subject in need of such treatment. The treatment can be orientated symptomatically, for example, to suppress symptoms. It can be effected over a short period, be oriented over a medium term, or can be a long-term treatment, for example within the context of maintenance therapy.

The terms “prevent,” “preventing,” and “prevention” refer to a method of preventing the onset of a disease or condition and/or its attendant symptoms or barring a subject from acquiring a disease. As used herein. “prevent,” “preventing,” and “prevention” also include delaying the onset of a disease and/or its attendant symptoms and reducing a subject's risk of acquiring a disease. The terms “prevent,” “preventing” and “prevention” may include “prophylactic treatment,” which refers to reducing the probability of redeveloping a disease or condition, or of a recurrence of a previously-controlled disease or condition, in a subject who does not have, but is at risk of or is susceptible to, redeveloping a disease or condition or a recurrence of the disease or condition.

It will be appreciated by persons skilled in the art that the present disclosure is not limited by what has been particularly shown and described herein above. Rather the scope of the present disclosure includes both combinations and sub-combinations of the various features described hereinabove as well as variations and modifications which would occur to persons skilled in the art upon reading the specification and which are not in the prior art.

### EXAMPLES

Unless otherwise noted, all reagents were purchased from commercial suppliers and used without further purification. The protected nonhydrolyzable phosphotyrosine mimetic 4-(Difluorophosphonomethyl)-N-(9-fluorenylmethyloxycarbonyl)-L-phenylalanine)(FmocF2Pmp-OH) that serve as starting point for the inhibitor development was prepared using the procedure which is well known in the art (M. F. Gordeev et al., Tetrahedron Letters, 1994, 35, 7585-7588), which is hereby specifically incorporated by reference for its teachings regarding the same.

Thin-layer chromatography was performed using glass-precoated Merck silica gel 60 F254 plates. Column chromatography was performed using KP-SIL silica gel (Biotage, USA), and flash column chromatography was performed on Biotage prepacked columns using the automated flash chromatography system Biotage Isolera One.

The ¹H- and ¹³C NMR spectra were recorded on a Bruker AVANCE 500 MHz spectrometer using dimethyl sulfoxide (DMSO-d6) as the solvent. Chemical shifts are expressed in ppm (S scale) and referenced to the residual protonated solvent. Peak multiplicities are reported using the following abbreviations: s (singlet), d (doublet), t (triplet), q (quartet), m (multiplet), or br (broad singlet).

Mass spectra and purity data were obtained using an Agilent Technologies 6470 series, triple quadrupole LC-MS. The purity of all final tested compounds was determined to be >95% (UV, λ=254 nm). High-resolution mass analysis was performed on an Agilent 6550 iFunnel Q-TOF mass LC-MS.

Recombinant mouse IFN-γ was purchased from PeproTech Inc. Anti-ERK1/2 (catalog #4696), antiphospho-ERK1/2 (catalog #9101), anti-p38 (catalog #9212), antiphospho-p38 (catalog #921Is), anti-AKT (catalog #2920s), and antiphospho Akt473 (catalog #9271s) antibodies were purchased from Cell Signaling. Anti-HA (catalog #SC-7392) and anti-GAPDH (catalog #SC-59541) antibodies were purchased from Santa Cruz, pNPP was purchased from Thermo Scientific (catalog #PI34045).

### ABBREVIATIONS USED

r.t.—room temperature; HPLC—high-performance liquid chromatography; LC/MS—liquid chromatography-mass spectrometry;
DCM—dichloromethane; DMF—dimethylformamide; NMM—N-methyl morpholine; DIPEA—N,N-diisopropylethylamine; EDTA—ethylenediaminetetraacetic acid; DTT—dithiothreitol; BSA—bis(trimethylsilyl)acetamide; HBTU-(2-(1H-benzotriazol-1-yl)-1,1,3,3-tetramethyluronium hexafluorophosphate; HOBt-hydroxybenzotriazole; TFA-trifluoroacetic acid; TIS—triisopropylsilane; DMSO—dimethyl sulfoxide; AcOH—acetic acid; 5(6)-FAM SE—5-(and-6)-carboxyfluorescein, succinimidyl ester mixed isomers; JAK—Janus kinase; STAT—signal transducer and activator of transcription.

### Example 1

### General Methods for Solid-Phase Peptide Synthesis

### General Procedure A: Rink Amide Resin Activation.

Rink amide resin was mixed with DCM (1 mL per 100 mg resin) and then shaken for 30 min. After activation, resin was washed three times with DMF (1 mL per 100 mg resin).

General Procedure B for the Removal of the Fmoc Group from the Rink Amide Resin. Rink amide resin was mixed with 30% piperidine in DMF, shaken for 30 min, and then washed with DMF (1 mL per 100 mg resin, 3 times), isopropanol (1 mL per 100 mg resin, 3 times), and DCM (1 mL per 100 mg resin, 3 times) sequentially. The removal of the Fmoc group was confirmed by the ninhydrin test.

General Procedure C for the Removal the Alloc Group from the Rink Amide Resin. The resin was washed with DCM (1 mL per 100 mg resin, 5×) and shaken under N₂ overnight with a solution of tetrakis(triphenylphosphine)-palladium(0) (10 mg), AcOH (0.5 mL), and NMM (0.2 mL) in DCM (10 mL). The resin was then washed with DMF (1 mL per 100 mg resin, 3 times), isopropanol (1 mL per 100 mg resin, 3 times), and DCM (1 mL per 100 mg resin, 3 times). The removal of the Alloc group was confirmed by the ninhydrin test.

General Procedure D for the Coupling of Carboxylic Acids to the Rink Amide Resin. Carboxylic acids (5 equiv, 0.5 M in DMF) were first mixed with HBTU (5 equiv, 0.5 M in DMF), HOBt (5 equiv, 0.5 M in DMF), and NMM (15 equiv, 1.5 M in DMF). The mixed solution was then added to the resin and shaken for 2 h. The resin was then washed with DMF (1 mL per 100 mg resin, 3 times), isopropanol (1 mL per 100 mg resin, 3 times), and DCM (1 mL per 100 mg resin, 3 times). The completion of the coupling reaction was confirmed by the ninhydrin test.

General Procedure E for Peptide Cleavage from the Rink Amide Resin.

The resin was washed with DCM (1 mL per 100 mg resin, 5 times) and subsequently shaken with 95% TFA, 2.5% TIS, and 2.5% H₂O (1 mL per 100 mg resin). The resin was removed by filtration, and the TFA was evaporated under vacuum. The crude peptide was obtained after trituration with diethyl ether (5 mL per 100 mg resin, 2 times).

### Example 2

### Library Assembly

The library was prepared on a Freedom EVO workstation (Tecan) with a 96-channel MCA tip block using disposable tips. The detailed procedure is as follows:

The 576 different carboxylic acids (40 mM, 10 μL) in DMF were placed in six 96-well microplates. HBTU (35 mM, 10 μL), HOBt (50 mM, 10 μL), and NMM (200 mM, 10 μL) were sequentially added to each well of these plates. The library precursor 1, 2, or 3 (for library gen 1, 2, and 3, respectively. 2 mM in DMF, 10 μL) was then added to each well. The reactions were quenched with cyclohexylamine (87 mM in DMF, 10 μL) after 1 h. Finally, 190 μL of DMSO were added to each well to create the ready-for-screening format. The library was stored in a −20° C. freezer.

The fluorescein-labeled precursors for each generation of library were synthesized via solid phase peptide synthesis as shown.

### Synthesis of Intermediate 1 for the First Generation Library

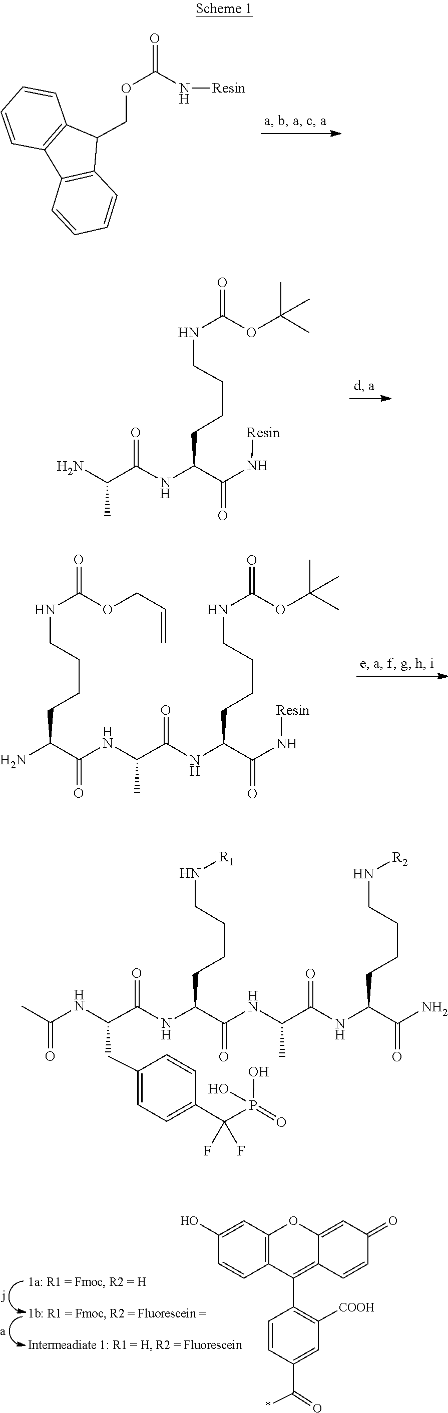

Synthesis of Intermediate 1. (a) 30% piperidine/DMF; (b) Fmoc-Lys(Boc)-OH/HBTU/HOBt/NMM; (c) Fmoc-Ala-OH/HBTU/HOBt/NMM; (d) Fmoc-Lys(Alloc)-OH/HBTU/HOBt/NMM; (e) Fmoc-F2Pmp-OH/HBTU/HOBt/NMM; (f) AcOH/HBTU/HOBt/NMM; (g) Tetrakis(triphenylphosphine)-palladium(0), AcOH/NMM/CH₂Cl₂; (h) Fmoc-C/NMM; (i) TFA/H₂O/TIS (95:2.5:2.5); (j) 5-(and-6)carboxyfluorescein succinimidyl ester/NMM.

### Synthesis of Compound 1a

Compound 1a was synthesized using standard Fmoc chemistry on the Rink amide resin. The resin (200 mg, 0.5 mmol/g loading) was first activated using general procedure A. The Fmoc group was removed with 30% piperidine in DMF using general procedure B. The resin was then coupled with Fmoc-Lys(Boc)-OH using general procedure D, followed by the removal of the Fmoc group using general procedure B. The resin was then sequentially coupled with Fmoc-Ala-OH, Fmoc-Lys(Alloc)-OH, Fmoc-F2Pmp-OH, and AcOH. The Alloc group was then removed using general procedure C. The resin was shaken with FmocCl (0.2 M in DMF, 2.5 mL) and NMM (1.5 M in DMF, 0.5 mL) for 2 h. Compound 1a was cleaved from the resin using general procedure E. Crude peptide was purified by HPLC to afford compound 1a (31.5 mg, 35% yield). Mass calculated for [M] 884.36. found [M+H]⁺ 885.41.

### Synthesis of Compound 1b

Compound 1a (31.5 mg) was treated with 5(6)-FAM SE (20 mg) and NMM (0.1 mL) in DMF (5 mL) overnight. After evaporation of the solvent, the crude product was purified by reversed-phase HPLC to afford 1b (16.39 mg, 37% yield). Mass calculated for [M] 1243.41. found [M+H]+ 1244.39.

### Synthesis of Intermediate 1

Compound 1b (16.39 mg) was treated with 30% piperidine in DMF (10 mL) for 30 min. After evaporation of the solvent, the crude product was purified by reversed-phase HPLC to afford Intermediate 1 (6.9 mg, 51% yield). Mass calculated for [M] 1021.34. found [M+H]+ 1022.37.

### Example 3

### Synthesis of Intermediate 2 for the Second Generation Library

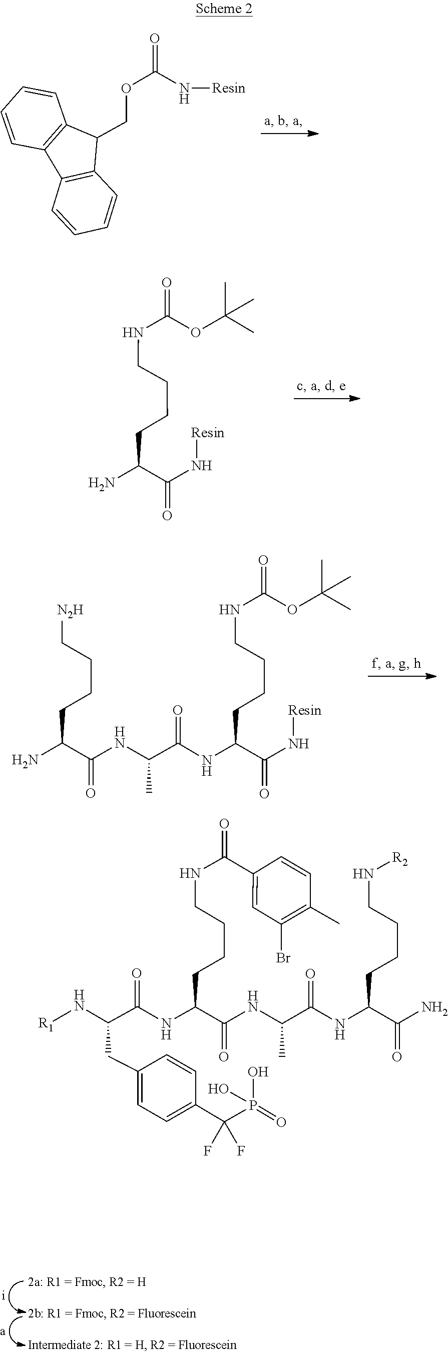

Synthesis of Intermediate 2. (a) 30% piperidine/DMF; (b) Fmoc-Lys(Boc)-OH/HBTU/HOBt/NMM; (c) Fmoc-Ala-OH/HBTU/HOBt/NMM; (d) Fmoc-Lys(Alloc)-OH/HBTU/HOBt/NMM; (e) Tetrakis(triphenylphosphine)-palladium(0), AcOH/NMM/CH₂Cl₂; (f) 3-bromo-4-methylbenzoic acid/HBTU/HOBt/NMM; (g) Fmoc-F2Pmp-OH/HBTU/HOBt/NMM; (h) TFA/H₂O/TIS (95:2.5:2.5); (i) 5-(and-6)-carboxyfluorescein succinimidyl ester/NMM.

### Synthesis of Compound 2a

Compound 2a was synthesized using standard Fmoc chemistry on the Rink amide resin. The resin (200 mg, 0.5 mmol/g loading) was first activated (general procedure A), followed by the removal of the Fmoc group (general procedure B). The resin was sequentially coupled with Fmoc-Lys(Boc)-OH, Fmoc-Ala-OH, and Fmoc-Lys(Alloc)-OH (general procedure D). The Alloc group was then removed (general procedure C), and the exposed amine was coupled with 3-bromo-4-methylbenzoic acid. The Fmoc group was removed, and the exposed amine was coupled with Fmoc-F2Pmp-OH. Compound 2a was then cleaved from resin (general procedure E). The crude product was purified by HPLC to afford compound 2a (33.3 mg, 34% yield). Mass calculated for [M] 1039.31. found [M+H]+ 1040.32.

### Synthesis of Compound 2b

Compound 2a (33.3 mg) was treated with 5(6)-FAM SE (20 mg) and NMM (0.1 mL) in DMF (5 mL) overnight. After evaporation of the solvent, the crude product was purified by reversed-phase HPLC to afford compound 2b (13.9 mg, 31% yield). Mass calculated for [M] 1397.35. found [M+H]+ 1398.31.

### Synthesis of Intermediate 2

Compound 2b (13.9 mg) was treated with 30% piperidine in DMF (10 mL) for 30 min. After evaporation of the solvent, the crude product was purified by reversed-phase HPLC to afford Intermediate 2 (6.4 mg, 55% yield). Mass calculated for [M] 1175.29. found [M+H]+ 1176.33.

### Example 4

### Synthesis of Intermediate 3 for the Third Generation Library

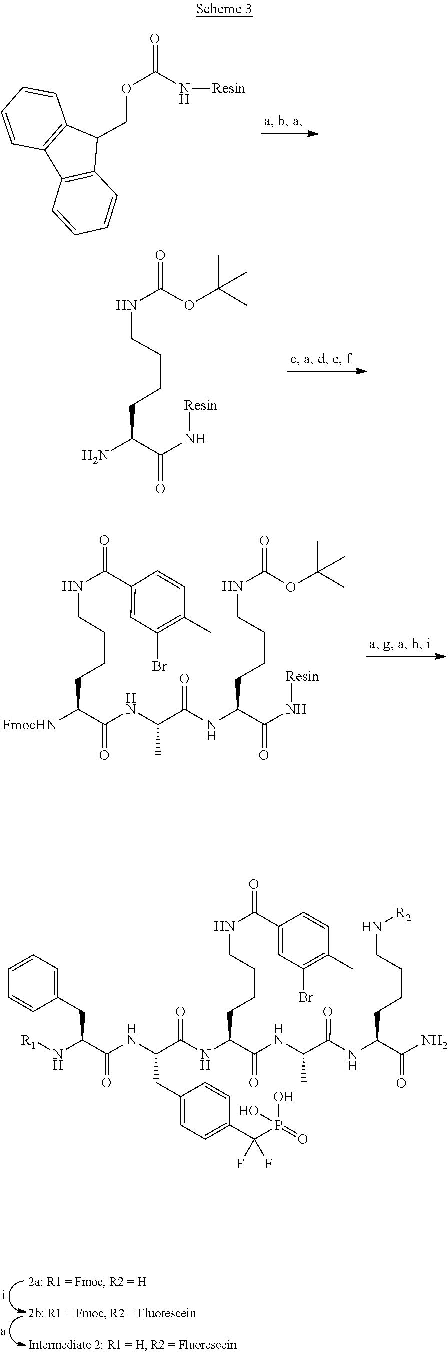

Synthesis of Intermediate 3. (a) 30% piperidine/DMF; (b) Fmoc-Lys(Boc)-OH/HBTU/HOBt/NMM; (c) Fmoc-Ala-OH/HBTU/HOBt/NMM; (d) Fmoc-Lys(Alloc)-OH/HBTU/HOBt/NMM; (e) Tetrakis(triphenylphosphine)-palladium(0), AcOH/NMM/CH₂Cl₂; (f) 3-bromo-4-methylbenzoic acid/HBTU/HOBt/NMM; (g) Fmoc-F2Pmp-OH/HBTU/HOBt/NMM; (h) Fmoc-Phe-OH/HBTU/HOBt/NMM; (i) TFA/H₂O/TIS (95:2.5:2.5); (j) 5-(and-6)-carboxyfluorescein succinimidyl ester/NMM.

### Synthesis of Compound 3a.

Compound 3a was synthesized using standard Fmoc chemistry on the Rink amide resin. The resin (200 mg, 0.5 mmol/g loading) was first activated (general procedure A), followed by the removal of Fmoc group (general procedure B). The exposed amine was sequentially coupled with Fmoc-Lys(Boc)-OH, Fmoc-Ala-OH, and Fmoc-Lys(Alloc)OH. The Alloc group was removed (general procedure C), and the exposed amine was coupled with 3-bromo-4-methylbenzoic acid. After that, the Fmoc group was removed and the resin was sequentially coupled with Fmoc-F2Pmp-OH and Fmoc-Phe-OH. Compound 3a was then cleaved from resin (general procedure E). The crude product was purified by HPLC to afford compound 3a (43.6 mg, 39% yield). Mass calculated for [M] 1186.37. found [M+H]+ 1187.34.

### Synthesis of Compound 3b.

Compound 3a (43.6 mg) was treated with 5(6)-FAM SE (20 mg) and NMM (0.1 mL) in DMF (5 mL) overnight. After evaporation of the solvent, the crude product was purified by reversed-phase HPLC to afford compound 3b (17.6 mg, 31% yield). Mass calculated for [M] 1544.42. found [M+H]+ 1545.40.

### Synthesis of Intermediate 3.

Compound 3b (17.6 mg) was treated with 30% piperidine in DMF (10 mL) for 30 min. After evaporation of the solvent, the crude product was purified by reversed-phase HPLC to afford Intermediate 3 (9.5 mg, 63% yield). Mass calculated for [M] 1322.35. found [M+H]+ 1323.39.

### Example 5

### Synthesis of ((4-((S)-2-acetamido-3-(((S)-1-amino-6-(3-bromo-4-methylbenzamido)-1-oxohexan-2-yl)amino)-3-oxopropyl)phenyl)difluoromethyl)phosphonic Acid

((4-((S)-2-acetamido-3-(((S)-1-amino-6-(3-bromo-4-methylbenzamido)-1-oxohexan-2-yl)amino)-3-oxopropyl)phenyl)difluoromethyl)phosphonic acid was synthesized on the Rink amide resin using standard Fmoc chemistry. The resin (200 mg, 0.5 mmol/g loading) was first activated (general procedure A) and subsequently treated with 30% piperidine to remove the Fmoc group (general procedure B). The exposed amine was coupled with Fmoc-Lys(Alloc)-OH (general procedure D). The Alloc group was removed (general procedure C), and the exposed amine was coupled with 3-bromo-4-methylbenzoic acid. After removal of the Fmoc group, the resin was sequentially coupled with Fmoc-F2Pmp-OH and AcOH. ((4-((S)-2-acetamido-3-(((S)-1-amino-6-(3-bromo-4-methylbenzamido)-1-oxohexan-2-yl)amino)-3-oxopropyl)phenyl)difluoromethyl)phosphonic acid was then cleaved from resin (general procedure E). The crude product was purified by HPLC to afford ((4-((S)-2-acetamido-3-(((S)-1-amino-6-(3-bromo-4-methylbenzamido)-1-oxohexan-2-yl)amino)-3-oxopropyl)phenyl)difluoromethyl)phosphonic acid (23.1 mg, 35% yield). Mass calculated for [M]660.12. found [M+H]+ 661.17. ¹H NMR (500 MHz, DMSO) δ 8.49 (t, J=5.5 Hz, 1H), 8.14 (d, J=8.0 Hz, 1H), 8.03 (d, J=1.8 Hz, 1H), 7.97 (d, J=8.2 Hz, 1H), 7.74 (dd, J=7.9, 1.8 Hz, 1H), 7.40 (d, J=7.8 Hz, 2H), 7.42-7.38 (m, 3H), 7.24 (s, 1H), 7.05 (s, 1H), 4.55-4.47 (m, 1H), 4.15 (m, 1H), 3.20 (q, J=6.6 Hz, 2H), 3.00 (dd, J=13.8, 4.8 Hz, 1H), 2.78 (dd, J=13.9, 9.7 Hz, 1H), 2.35 (s, 3H), 1.75 (s, 3H), 1.67 (ddt, J=15.2, 11.0, 5.5 Hz, 1H), 1.57-1.43 (m, 3H), 1.34-1.24 (m, 2H).

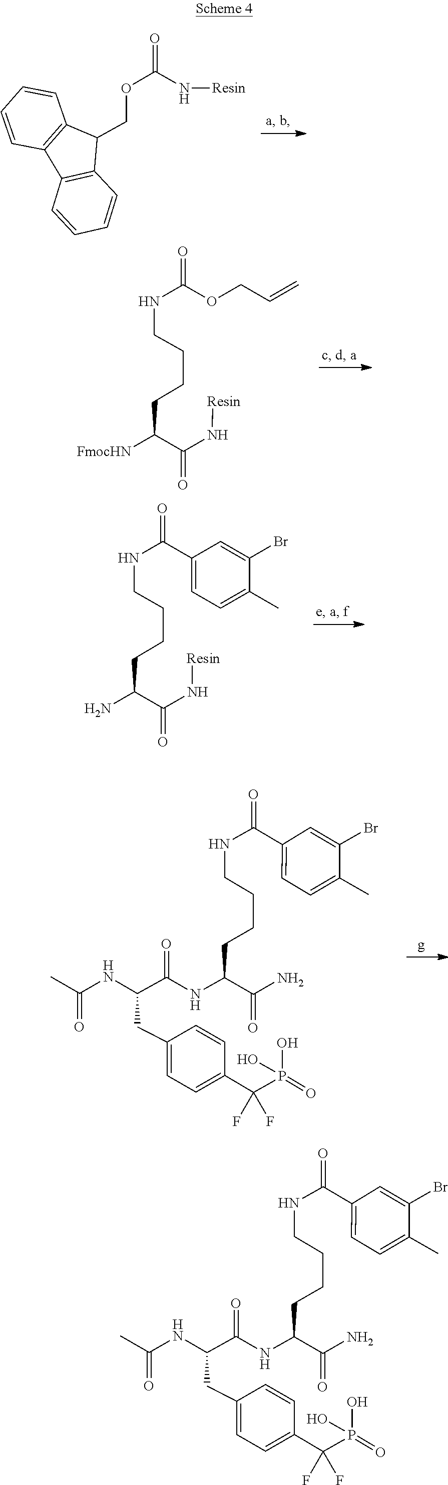

Synthesis of ((4-((S)-2-acetamido-3-(((S)-1-amino-6-(3-bromo-4-methylbenzamido)-1-oxohexan-2-yl)amino)-3-oxopropyl)phenyl)difluoromethyl)phosphonic acid. (a) 30% piperidine/DMF; (b) Fmoc-Lys(Alloc)-OH/HBTU/HOBt/NMM; (c) Tetrakis(triphenylphosphine)-palladium(0), AcOH/NMM/CH₂Cl₂; (d) 3-bromo-4-methylbenzoic acid/HBTU/HOBt/NMM; (e) Fmoc-F2Pmp-OH/HBTU/HOBt/NMM; (f) AcOH/HBTU/HOBt/NMM; (g) TFA/H₂O/TIS (95:2.5:2.5).

### Example 6

### Synthesis of ((4-((S)-2-((S)-2-acetamido-3-phenylpropanamido)-3-(((S)-1-amino-6-(3-bromo-4-methylbenzamido)-1-oxohexan-2-yl)amino)-3-oxopropyl)phenyl)difluoromethyl)phosphonic Acid

((4-((S)-2-((S)-2-acetamido-3-phenylpropanamido)-3-(((S)-1-amino-6-(3-bromo-4-methylbenzamido)-1-oxohexan-2-yl)amino)-3-oxopropyl)phenyl)difluoromethyl)phosphonic acid was synthesized on the Rink amide resin using standard Fmoc chemistry. The resin (200 mg, 0.5 mmol/g loading) was activated (general procedure A) and treated with 30% piperidine to remove the Fmoc group (general procedure B). The exposed amine was coupled with Fmoc-Lys(Alloc)-OH (general procedure D). The Alloc group was removed (general procedure C), and the exposed amine was coupled with 3-bromo-4-methylbenzoic acid. The Fmoc group was removed, and the resin was sequentially coupled with Fmoc-F2Pmp-OH, Fmoc-PheOH, and AcOH. ((4-((S)-2-((S)-2-acetamido-3-phenylpropanamido)-3-(((S)-1-amino-6-(3-bromo-4-methylbenzamido)-1-oxohexan-2-yl)amino)-3-oxopropyl)phenyl)difluoromethyl)phosphonic acid was then cleaved from the resin (general procedure E). The crude product was purified by HPLC to afford ((4-((S)-2-((S)-2-acetamido-3-phenylpropanamido)-3-(((S)-1-amino-6-(3-bromo-4-methylbenzamido)-1-oxohexan-2-yl)amino)-3-oxopropyl)phenyl)difluoromethyl)phosphonic acid (22.6 mg, 28% yield). Mass calculated for [M]807.18. found [M+H]+ 808.17. ¹H NMR (500 MHz, DMSO) δ 8.49 (t, J=5.5 Hz, 1H), 8.18 (d, J=8.0 Hz, 1H), 8.10 (d, J=8.3 Hz, 1H), 8.02 (d, J=1.8 Hz, 1H), 7.96 (d, J=8.1 Hz, 1H), 7.73 (dd, J=7.9, 1.8 Hz, 1H), 7.42-7.38 (m, 3H), 7.34-7.25 (m, 3H), 7.25-7.16 (m, 4H), 7.16-7.10 (m, 1H), 7.07 (s, 1H), 4.53 (td, J=8.4, 4.8 Hz, 1H), 4.44 (ddd, J=10.2, 8.3, 4.2 Hz, 1H), 4.18 (td, J=8.3, 5.2 Hz, 1H), 3.21 (q, J=6.7 Hz, 2H), 3.07 (dd, J=13.9, 4.8 Hz, 1H), 2.94 (dd, J=14.0, 4.2 Hz, 1H), 2.90-2.81 (m, 1H), 2.73-2.62 (m, 1H), 2.35 (s, 3H), 1.70 (s, 3H), 1.70-1.63 (m, 1H), 1.59-1.45 (m, 3H), 1.35-1.25 (m, 2H).

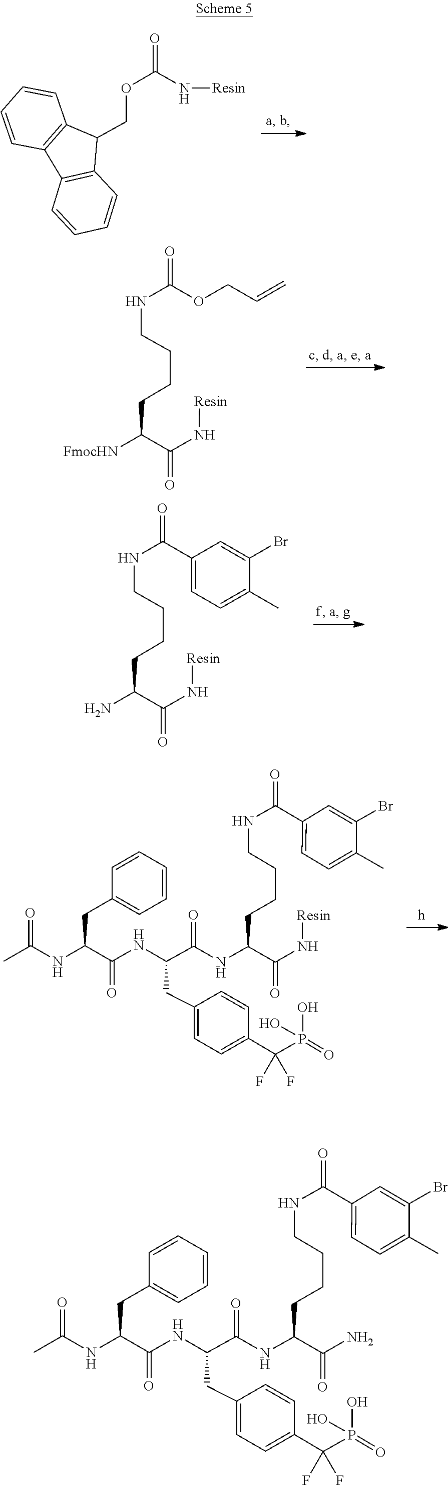

Synthesis of ((4-((S)-2-((S)-2-acetamido-3-phenylpropanamido)-3-(((S)-1-amino-6-(3-bromo-4-methylbenzamido)-1-oxohexan-2-yl)amino)-3-oxopropyl)phenyl)difluoromethyl)phosphonic acid. (a) 30% piperidine/DMF; (b) Fmoc-Lys(Alloc)-OH/HBTU/HOBt/NMM; (c) Tetrakis(triphenylphosphine)-palladium(0), AcOH/NMM/DCM; (d) 3-bromo-4-methylbenzoic acid/HBTU/HOBt/NMM; (e) Fmoc-F2Pmp-OH/HBTU/HOBt/NMM; (f) Fmoc-Phe-OH/HBTU/HOBt/NMM; (g) AcOH/HBTU/HOBt/NMM; (h) TFA/H₂O/MIS (95:2.5:2.5).

### Example 7

### Synthesis of ((4-((S)-3-(((S)-1-amino-6-(3-bromo-4-methylbenzamido)-1-oxohexan-2-yl)amino)-2-((S)-2-(2-(4-hydroxy-3-methoxyphenyl)acetamido)-3-phenylpropanamido)-3-oxopropyl)phenyl)difluoromethyl)phosphonic Acid

((4-((S)-3-(((S)-1-amino-6-(3-bromo-4-methylbenzamido)-1-oxohexan-2-yl)amino)-2-((S)-2-(2-(4-hydroxy-3-methoxyphenyl)acetamido)-3-phenylpropanamido)-3-oxopropyl)phenyl)difluoromethyl)phosphonic acid was synthesized on the Rink amide resin using standard Fmoc chemistry. The resin (200 mg, 0.5 mmol/g loading) was activated (general procedure A) and subsequently treated with 30% piperidine to remove the Fmoc group (general procedure B). The exposed amine was coupled with Fmoc-Lys(Alloc)-OH (general procedure D). The Alloc group was removed (general procedure C), and the exposed amine was coupled with 3-bromo-4-methylbenzoic acid. The Fmoc group was removed, and the resin was sequentially coupled with Fmoc-F2Pmp-OH, Fmoc-Phe-OH, and Homovanillic acid. ((4-((S)-3-(((S)-1-amino-6-(3-bromo-4-methylbenzamido)-1-oxohexan-2-yl)amino)-2-((S)-2-(2-(4-hydroxy-3-methoxyphenyl)acetamido)-3-phenylpropanamido)-3-oxopropyl)phenyl)difluoromethyl)phosphonic acid was then cleaved from resin (general procedure E). The crude product was purified by HPLC to afford ((4-((S)-3-(((S)-1-amino-6-(3-bromo-4-methylbenzamido)-1-oxohexan-2-yl)amino)-2-((S)-2-(2-(4-hydroxy-3-methoxyphenyl)acetamido)-3-phenylpropanamido)-3-oxopropyl)phenyl)difluoromethyl)phosphonic acid (13.0 mg, 14% yield). Mass calculated for [M]929.22. found [M+H]+ 930.24. ¹H NMR (500 MHz, DMSO) δ 8.49 (t, J=5.6 Hz, 1H), 8.21 (d, J=7.9 Hz, 1H), 8.09 (d, J=8.3 Hz, 1H), 8.06-7.97 (m, 2H), 7.73 (dd, J=7.9, 1.8 Hz, 1H), 7.39 (dd, J=8.1, 3.0 Hz, 3H), 7.30 (d, J=8.0 Hz, 2H), 7.25 (s, 1H), 7.17-7.08 (m, 6H), 6.67 (d, J=2.0 Hz, 1H), 6.57 (d, J=8.0 Hz, 1H), 6.41 (dd, J=8.0, 2.0 Hz, 1H), 4.57-4.52 (m, 1H), 4.48-4.43 (m, 1H), 4.21-4.13 (m, 1H), 3.65 (s, 3H), 3.29-3.20 (m, 3H), 3.18 (d, J=14.0 Hz, 2H), 3.03 (dd, J=13.9, 5.3 Hz, 1H), 2.96 (dd, J=14.0, 4.0 Hz, 1H), 2.90-2.82 (m, 1H), 2.74-2.66 (m, 1H), 2.35 (s, 3H), 1.74-1.61 (m, 1H), 1.57-1.44 (m, 3H), 1.37-1.21 (m, 2H).

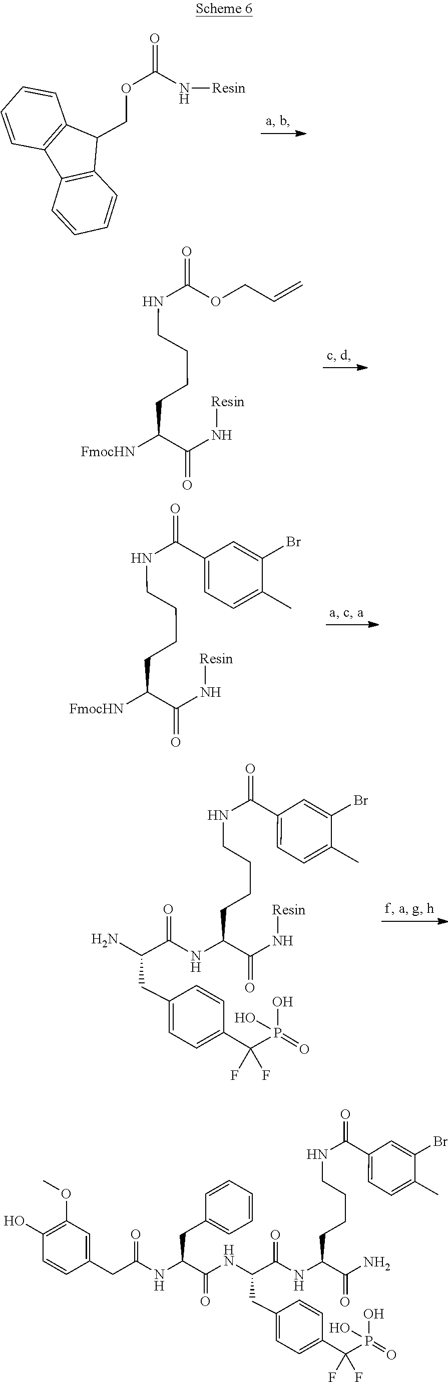

Synthesis of ((4-((S)-3-(((S)-1-amino-6-(3-bromo-4-methylbenzamido)-1-oxohexan-2-yl)amino)-2-((S)-2-(2-(4-hydroxy-3-methoxyphenyl)acetamido)-3-phenylpropanamido)-3-oxopropyl)phenyl)difluoromethyl)phosphonic acid. (a) 30% piperidine/DMF; (b) Fmoc-Lys(Alloc)-OH/HBTU/HOBt/NMM; (c) Tetrakis(triphenylphosphine)-palladium(0), AcOH/NMM/CH₂Cl₂; (d) 3-bromo-4-ethylbenzoic acid/HBTU/HOBt/NMM; (e) Fmoc-F2Pmp-OH/HBTU/HOBt/NMM; (f) Fmoc-Phe-OH/HBTU/HOBt/NMM; (g) Homovanillic acid/HBTU/HOBt/NMM (h) TFA/H₂O/TIS (95:2.5:2.5).

### Example 8

### Synthesis of Intermediate 6

### Benzyl (S)-2-((tert-butoxycarbonyl)amino)-3-(4-iodophenyl)propanoate (Intermediate 4)

To a stirred mixture of (S)-2-((tert-butoxycarbonyl)amino)-3-(4-iodophenyl)propanoic acid (10.00 g, 25.6 mmol, 1.0 eq) and K₂CO₃ (5.31 g, 38.4 mmol, 1.5 eq) in DMF (100 ml) was added Benzyl bromide (6.57 g, 38.4 mmol, 1.5 eq). Then the mixture was stirred at room temperature (r.t.) for 4 hours. After completion, the reaction mixture was diluted with EtOAc (500 ml) and washed with brine (3×500 ml). The organic layer was dried over anhydrous Na₂SO₄ and evaporated in vacuo. The product was then purified by flash chromatography (EtOAc/n-Hexane, 0%→10%). Yield: 8.80 g (72%). ¹H NMR (500 MHz, DMSO) δ 7.63-7.57 (m, 2H), 7.37-7.29 (m, 4H), 7.29-7.22 (m, 2H), 7.07-7.02 (m, 2H), 5.11-5.00 (m, 2H), 4.18 (ddd, J=9.8, 8.0, 5.61 Hz, 1H), 2.94 (dd, J=13.7, 5.6 Hz, 1H), 2.83 (dd, J=13.7, 9.8 Hz, 1H), 1.30 (s, 9H). LC/MS m/z calculated [M+H]⁺ 482.08. found 482.18.

Benzyl (S)-2-((tert-butoxycarbonyl)amino)-3-(4-((diethoxyphosphoryl)difluoromethyl)phenyl)propanoate (Intermediate 5) Intermediate 4 (10.00 g, 20.8 mmol, 1.0 eq) was dissolved in anhydrous DMF (100 mL), then CuBr (5.97 g, 41.6 mmol, 2 equiv) and half of the supernatant solution of the Cd reagent 7 (ca. 1.7 equiv, prepared according to method described in C. Meyer, M. Kohn, New York, 2011, 6) was added in a dropwise manner. After 3 hours, further CuBr (2.98 g, 20.8 mmol, 1 equiv) and the other half of the Cd reagent solution were added. The reaction was allowed to stir at r.t. for 19 hours in total. The progress of the reaction was monitored by LC-MS. Upon completion, the reaction mixture was diluted with EtOAc (500 mL), filtered through Celite, and extracted with aq NH4Cl (2×500 mL) and brine (500 mL). The organic layer was dried over anhydrous Na₂SO₄ and evaporated in vacuo. The product was then purified by flash chromatography (EtOAc/n-Hexane, 0%→10%). Yield 11.86 g (57%). ¹H NMR (500 MHz, DMSO) δ 7.45 (d, J=7.9 Hz, 2H), 7.41-7.26 (m, 8H), 5.09 (s, 2H), 4.26 (ddd, J=9.8, 8.1, 5.3 Hz, 1H), 4.16-3.99 (m, 4H), 3.09 (dd, J=13.8, 5.4 Hz, 1H), 2.95 (dd, J=13.8, 10.0 Hz, 1H), 1.29 (s, 9H), 1.19 (td, J=7.1, 1.5 Hz, 6H). LC/MS m/z calculated [M+H]542.21. found 542.28.

### (S)-2-((tert-butoxycarbonyl)amino)-3-(4-((diethoxyphosphoryl)difluoromethyl)phenyl)propanoic Acid (Intermediate 6)

To a solution of intermediate 5 (8.98 g, 16.6 mmol, 1.0 eq) in EtOAc (50 mL) was added Pd/C (10 wt. % loading, 750.0 mg). The reaction mixture was evacuated and filled with H₂ for three times. The mixture was then stirred at room temperature for 12 hours. Upon completion, the reaction mixture was concentrated in vacuo. The product was then purified by flash chromatography (MeOH/DCM, 0%→5%). Yield 6.66 g (89%). ¹H NMR (500 MHz, DMSO) δ 7.45 (d, J=8.0 Hz, 2H), 7.39 (d, J=8.1 Hz, 2H), 7.12 (d, J=8.5 Hz, 1H), 4.17-3.98 (m, 4H), 3.07 (dd, J=13.8, 4.7 Hz, 2H), 2.91-2.83 (m, 1H), 1.29 (s, 9H), 1.19 (td, J=7.0, 1.9 Hz, 6H). LC/MS m/z calculated [M+H]⁺ 452.16. found 452.23.

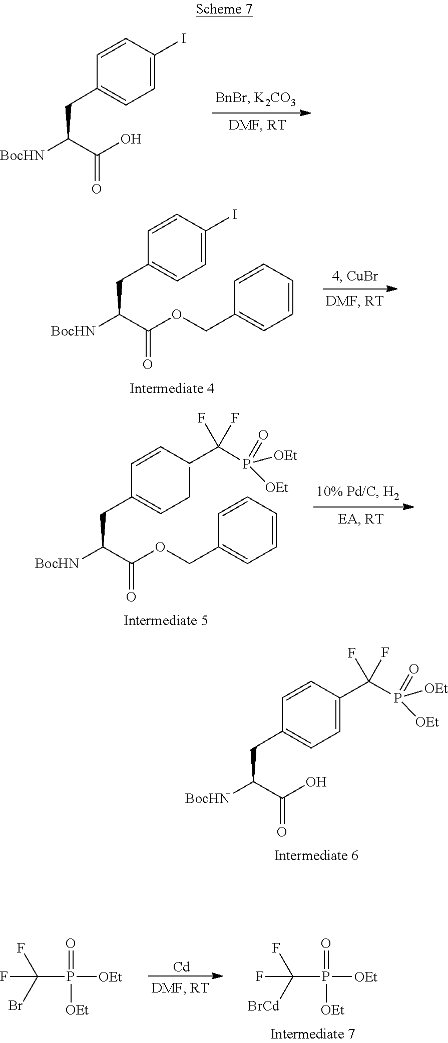

### Synthesis of Intermediate 6

### Example 9

### Synthesis of Intermediate 10

### Benzyl N2-(((9H-fluoren-9-yl)methoxy)carbonyl)-N6-(tert-butoxycarbonyl)-L-lysinate (Intermediate 8)

To a mixture of N2-(((9H-fluoren-9-yl)methoxy)carbonyl)-N6-(tert-butoxycarbonyl)-L-lysine (10.00 g, 21.34 mmol, 1.0 eq) and K₂CO₃ (4.42 g, 32.01 mmol, 1.5 eq) in DMF (100 ml) was added Benzyl bromide (5.47 g, 32.01 mmol, 1.5 eq). Then the mixture was stirred at r.t. for 4 hours. After completion, the reaction mixture was diluted with EtOAc (500 ml) and washed with brine (3×500 ml). The organic layer was dried over anhydrous Na₂SO₄ and evaporated in vacuo. The product was then purified by flash chromatography (EtOAc/n-Hexane, 0%→30%). Yield 9.06 g (76%). ¹H NMR (500 MHz, DMSO) δ 7.90-7.82 (m, 2H), 7.78 (d, J=7.7 Hz, 1H), 7.69 (d, J=7.5 Hz, 2H), 7.43-7.36 (m, 2H), 7.35-7.26 (m, 7H), 6.74 (t, J=5.8 Hz, 1H), 5.10 (d, J=1.3 Hz, 2H), 4.33-4.23 (m, 2H), 4.23-4.17 (m, 1H), 4.06-3.99 (m, 1H), 2.88-2.81 (m, 2H), 1.74-1.57 (m, 2H), 1.36-1.27 (m, 13H). LC/MS m/z calculated [M+H]⁺ 559.28. found 559.39.

### Benzyl N2-(((9H-fluoren-9-yl)methoxy)carbonyl)-N6-(3-bromo-4-methylbenzoyl)-L-lysinate (Intermediate 9)

To a solution of Intermediate 8 (7.50 g, 13.42 mmol, 1.0 eq) in DCM (80 mL), trifluoroacetic acid (20 mL) was added and stirred for 4 hours at r. t. Then the excess reagent and solvent were evaporated under reduced pressure to give the deprotected amine, which was used in the next step without further purification.

The deprotected amine, 3-bromo-4-methyl benzoic acid (3.17 g, 14.76 mmol, 1.1 eq), HOAt (2.19 g, 16.10 mmol, 1.2 eq) and DIPEA (12.14 g, 93.94 mmol, 7.0 eq) were then dissolved in DMF (100 ml) and stirred at 0° C. To the stirred solution HATU (7.65 g 20.13 mmol, 1.5 eq) was added slowly. After 15 minutes, the reaction mixture was diluted with EtOAc (800 ml) and washed with brine (3×500 ml). The organic layer was dried over anhydrous Na₂SO₄ and evaporated in vacuo. The product was then purified by flash chromatography (EtOAc/n-Hexane, 0%→40%). 2 steps yield 7.21 g (82%). ¹H NMR (500 MHz, DMSO) δ 8.50 (t, J=5.6 Hz, 1H), 8.04 (d, J=1.8 Hz, 1H), 7.86 (d, J=7.6 Hz, 2H), 7.81 (d, J=7.8 Hz, 1H), 7.74 (dd, J=7.8, 1.8 Hz, 1H), 7.68 (d, J=7.5 Hz, 2H), 7.44-7.36 (m, 3H), 7.36-7.23 (m, 7H), 5.11 (s, 2H), 4.33-4.15 (m, 3H), 4.12-3.93 (m, 1H), 3.22 (q, J=6.6 Hz, 2H), 2.34 (s, 3H), 1.80-1.61 (m, 2H), 1.55-1.44 (m, 6.7 Hz, 2H), 1.41-1.27 (m, 2H). LC/MS m/z calculated [M+H]⁺ 655.18. found 655.22.

### Benzyl N6-(3-bromo-4-methylbenzoyl)-N2-((S)-2-((tert-butoxycarbonyl)amino)-3-(4-((diethoxyphosphoryl)difluoromethyl)phenyl)propanoyl)-L-lysinate (Intermediate 10)

To a solution of Intermediate 9 (7.10 g, 10.82 mmol, 1.0 eq) in DCM (80 mL), diethyl amine (20 mL) was added and stirred for 4 hours at r. t. Then the excess reagent and solvent were evaporated under reduced pressure to give the crude deprotected amine, which was then purified by flash chromatography (MeOH/DCM, 0%→10%).

The deprotected amine, intermediate 6 (5.37 g, 11.90 mmol, 1.1 eq), HOAt (1.77 g, 12.98 mmol, 1.2 eq), and DIPEA (5.59, 43.28 mmol, 4.0 eq) were then dissolved in DMF (100 ml) and stirred at 0° C. To the stirred solution HATU (6.17 g, 16.23 mmol, 1.5 eq) was added slowly. After 15 minutes, the reaction mixture was diluted with EtOAc (800 ml) and washed with brine (3×500 ml). The organic layer was dried over anhydrous Na₂SO₄ and evaporated in vacuo. The product was then purified by flash chromatography (cyclohexane-EtOAc, 6:1→2:1). 2 steps yield 7.22 g (77%). ¹H NMR (500 MHz, DMSO) δ 8.51-8.46 (m, J=5.5 Hz, 1H), 8.35 (d, J=7.4 Hz, 1H), 8.02 (d, J=1.8 Hz, 1H), 7.73 (dd, J=7.8, 1.8 Hz, 1H), 7.47-7.34 (m, 6H), 7.34-7.25 (m, 4H), 6.92 (d, J=8.7 Hz, 1H), 5.10 (s, 2H), 4.34-4.27 (m, 1H), 4.23 (ddd, J=10.7, 8.7, 3.8 Hz, 1H), 4.14-3.98 (m, 4H), 3.21 (q, J=6.5 Hz, 2H), 2.95 (dd, J=13.9, 3.7 Hz, 1H), 2.71 (dd, J=13.9, 10.9 Hz, 1H), 2.35 (s, 3H), 1.82-1.72 (m, 1H), 1.72-1.62 (m, 1H), 1.55-1.34 (m, 2H), 1.39-1.30 (m, 2H), 1.24 (s, 9H), 1.20-1.17 (m, 6H). LC/MS m/z calculated [M+H]⁺ 866.26. found 866.28.

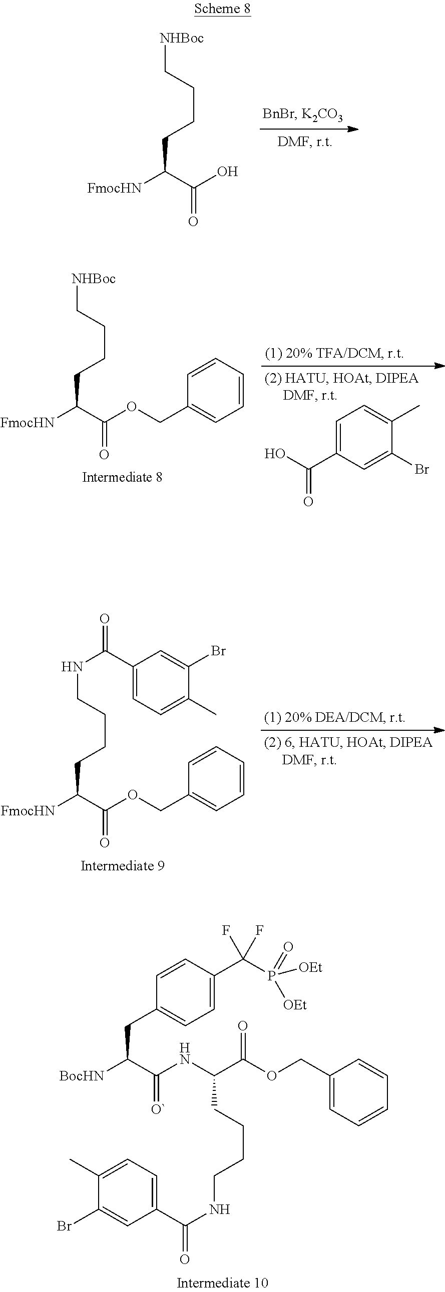

### Synthesis of Intermediate 10

### Example 10

### Synthesis of Intermediate 12

Methyl (2-(4-hydroxy-3-methoxyphenyl)acetyl)-L-phenylalaninate (Intermediate 11) Methyl L-phenylalaninate (5.00 g, 27.90 mmol, 1.0 eq), 2-(4-hydroxy-3-methoxyphenyl)acetic acid (5.59 g, 30.69 mmol, 1.1 eq), HOAt (4.56 g, 33.48 mmol, 1.2 eq) and DIPEA (14.42, 111.60 mmol, 4.0 eq) were then dissolved in DMF (100 ml) and stirred at 0° C. To the stirred solution HATU (15.91 g, 41.85 mmol, 1.5 eq) was added slowly. After 15 minutes, the reaction mixture was diluted with EtOAc (600 ml) and washed with brine (3×500 ml). The organic layer was dried over anhydrous Na₂SO₄ and evaporated in vacuo. The product was then purified by flash chromatography (cyclohexane-EtOAc, 6:1→2:1). Yield 7.76 g (81%). ¹H NMR (500 MHz, DMSO) δ 8.76 (s, 1H), 8.39 (d, J=7.8 Hz, 1H), 7.26-7.12 (m, 5H), 6.73 (d, J=2.0 Hz, 1H), 6.62 (d, J=8.0 Hz, 1H), 6.50 (dd, J=8.0, 2.0 Hz, 1H), 4.45 (ddd, J=9.3, 7.8, 5.3 Hz, 1H), 3.68 (s, 3H), 3.58 (s, 3H), 3.28 (s, 2H), 3.16 (d, J=4.7 Hz, 1H), 3.03-2.97 (m, 1H), 2.92-2.85 (m, 1H). LC/MS m/z calculated [M+H]⁺ 344.15. found 344.24.

### (2-(4-hydroxy-3-methoxyphenyl)acetyl)-L-phenylalanine (Intermediate 12)

To a solution of intermediate 11 (5.22 g, 15.20 mmol) in THF (50 ml), 0.5 M LiOH (aq.) was added. The mixture was stirred at r.t. for 4 hours. Then 50 ml 1.0 M HCl (aq.) was added to the reaction mixture. The mixture was extracted with EtOAc (3×250 ml) and washed with brine (2×250 ml). The organic layer was combined, dried over anhydrous Na₂SO₄, and evaporated in vacuo to give crude intermediate 8. This crude product was used in the following synthesis without further purification.

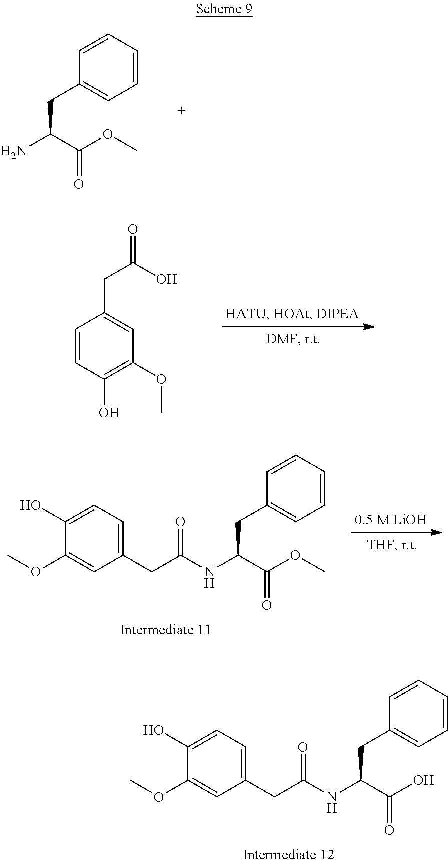 [SMILES: COC(=O)[C@@H](N)Cc1ccccc1 ; COc1cc(CC(=O)O)ccc1O ; COC(=O)[C@H](Cc1ccccc1)NC(=O)Cc1ccc(O)c(OC)c1 ; COc1cc(CC(=O)N[C@@H](Cc2ccccc2)C(=O)O)ccc1O]

### Synthesis of Intermediate 12

### Example 11

### Synthesis of Intermediate 14 (PTP1B/TC-PTP Dual Ligand)

### Benzyl N6-(3-bromo-4-methylbenzoyl)-N2-((S)-3-(4-((diethoxyphosphoryl) difluoromethyl) phenyl)-2-((S)-2-(2-(4-hydroxy-3-methoxyphenyl)acetamido)-3-phenylpropanamido)propanoyl)-L-lysinate (Intermediate 13)

To a solution of Intermediate 10 (5.79 g, 6.68 mmol, 1.0 eq) in DCM (80 mL), trifluoroacetic acid (20 mL) was added and stirred for 6 hours at r. t. Then the excess reagent and solvent were evaporated under reduced pressure to give the crude deprotected amine, which was used in the next step without further purification.

The deprotected amine, intermediate 12 (2.42 g, 7.35 mmol, 1.1 eq). HOAt (1.09 g, 8.02 mmol, 1.2 eq) and DIPEA (6.04 g, 46.76 mmol, 7.0 eq) were then dissolved in DMF (50 ml) and stirred at 0° C. To the stirred solution HATU (3.81 g, 10.02 mmol, 1.5 eq) was added slowly. After 15 minutes, the reaction mixture was diluted with EtOAc (300 ml) and washed with brine (3×300 ml). The organic layer was dried over anhydrous Na₂SO₄ and evaporated in vacuo. The product was then purified by flash chromatography (EtOAc/n-Hexane, 50%→100%). 2 steps yield 4.89 g (69%). ¹H NMR (500 MHz, DMSO) δ 8.70 (s, 1H), 8.54-8.44 (m, 2H), 8.15 (d, J=8.2 Hz, 1H), 8.02 (d, J=1.8 Hz, 1H), 7.96 (d, J=8.1 Hz, 1H), 7.73 (dd, J=7.8, 1.8 Hz, 1H), 7.44-7.37 (m, 3H), 7.37-7.32 (m, 5H), 7.32-7.25 (m, 1H), 7.18-7.07 (m, 5H), 6.66 (d, J=2.0 Hz, 1H), 6.61-6.53 (m, 1H), 6.44-6.37 (m, 1H), 5.11 (s, 2H), 4.60 (td, J=8.7, 4.3 Hz, 1H), 4.43 (ddd, J=9.8, 8.3, 4.1 Hz, 1H), 4.29 (ddd, J=8.8, 7.2, 5.3 Hz, 1H), 4.13-3.95 (m, 4H), 3.64 (s, 3H), 3.29-3.13 (m, 4H), 3.02 (dd, J=14.0, 4.3 Hz, 1H), 2.89 (dd, J=14.0, 4.1 Hz, 1H), 2.80 (dd, J=14.0, 9.3 Hz, 1H), 2.67 (dd, J=14.0, 9.9 Hz, 1H), 2.35 (s, 3H), 1.82-1.72 (m, 1H), 1.71-1.61 (m, 1H), 1.50 (p, J=7.3 Hz, 2H), 1.35 (q, J=7.3 Hz, 2H), 1.24-1.09 (m, 6H). LC/MS m/z calculated [M+H]⁺1077.32. found 1077.38.

### N6-(3-bromo-4-methylbenzoyl)-N2-((S)-3-(4-(difluoro(phosphono)methyl)phenyl)-2-((S)-2-(2-(4-hydroxy-3-methoxyphenyl)acetamido)-3-phenylpropanamido)propanoyl)-L-lysine (Intermediate 14)

To a solution of intermediate 13 (2.50 g, 2.32 mmol, 1.0 eq) in EtOAc (50 mL) was added Pd/C (10 wt. % loading, 250.0 mg). The reaction mixture was evacuated and filled with H₂ for three times. The mixture was then stirred at r.t. for 12 hours. Upon completion, the reaction mixture was concentrated in vacuo to give crude deprotected carboxylic acid, which was directly used in the next step without further purification.

The solution of the deprotected carboxylic acid in anhydrous DCM (25 ml) was cooled to 0° C. and stirred vigorously. Then trimethylsilyl iodide (3.25 g, 16.24 mmol, 7.0 eq) was added to the solution dropwise. The reaction was kept at 0° C., and monitored with LC-MS. Upon completion, the reaction mixture was added to a 50% MeCN/H₂O mixture dropwise and stirred at r.t. for 30 minutes. Then the water and organic solvent was evaporated in vacuo to give crude intermediate 10, which was further purified by prep HPLC (MeOH/H₂O, 50%→90%). 2 steps yield 1.09 g (47%). ¹H NMR (500 MHz, DMSO) δ 8.52 (t, J=5.5 Hz, 1H), 8.29 (d, J=7.6 Hz, 1H), 8.16 (d, J=8.1 Hz, 1H), 8.10-7.98 (m, 2H), 7.74 (dd, J=8.0, 1.8 Hz, 1H), 7.44-7.37 (m, 3H), 7.35 (d, J=8.0 Hz, 2H), 7.17-7.08 (m, 5H), 6.67 (d, J=2.0 Hz, 1H), 6.58 (d, J=8.0 Hz, 1H), 6.43-6.38 (dd, J=8.0, 1.9 Hz, 1H), 4.65-4.55 (m, 1H), 4.50-4.41 (m, 1H), 4.25-4.14 (m, 1H), 3.65 (s, 3H), 3.30-3.15 (m, 4H), 3.09 (dd, J=14.2, 4.4 Hz, 1H), 2.96 (dd, J=14.1, 4.0 Hz, 1H), 2.89-2.81 (m, 1H), 2.70 (dd, J=13.9, 10.0 Hz, 1H), 2.35 (s, 3H), 1.84-1.72 (m, 1H), 1.68-1.58 (m, 1H), 1.57-1.45 (m, 2H), 1.42-1.33 (m, 2H). LC/MS m/z calculated [M−H]⁻ 931.20. found 931.35.

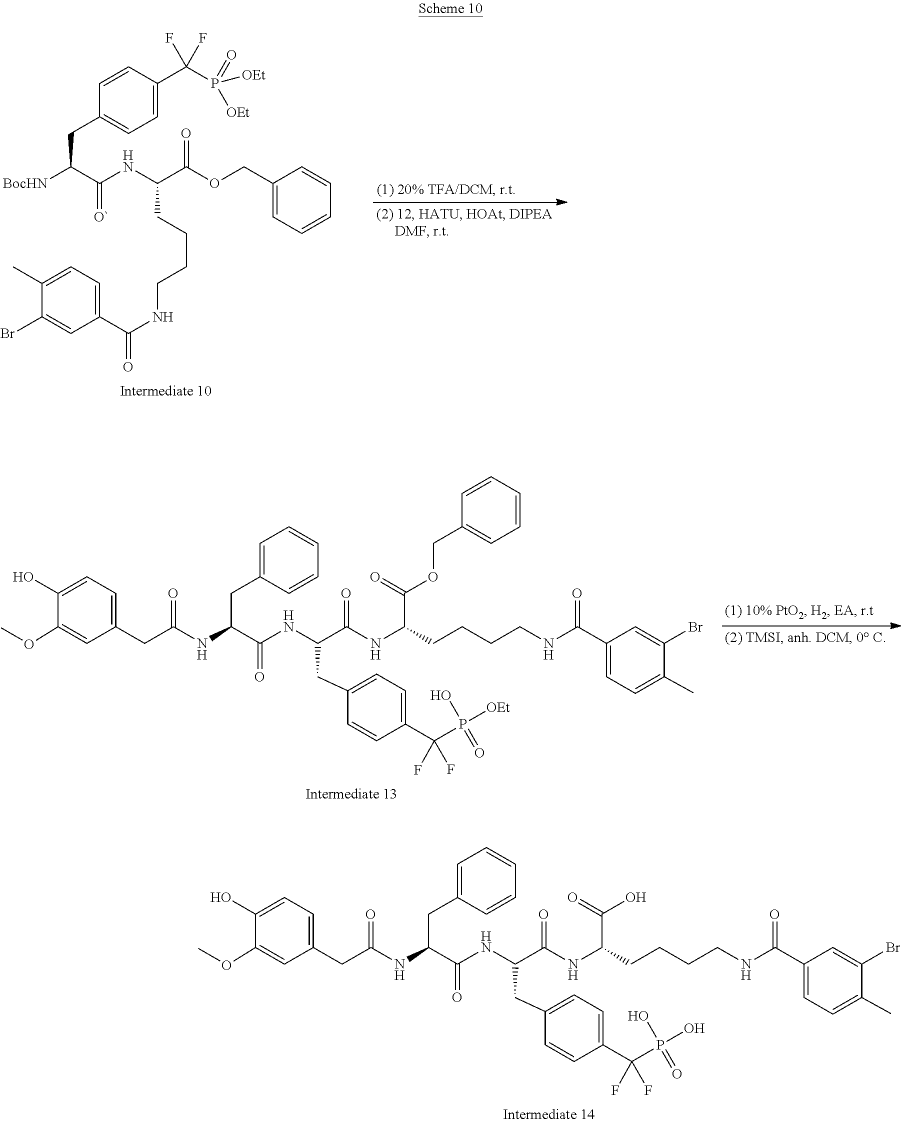

### Synthesis of Intermediate 14 (PTP1B/TC-PTP Dual Ligand).

### Example 12

### General Synthesis Method for Linker-VHL Ligand Tethering

Linker-VHL ligand tethering was conducted based on the known method (Q. Zhao et al., J. Med. Chem. 2019, 62, 9281-9298; X. Han, et al., J. Med. Chem. 2019, 62, 941-964). In specific, to the solution of Boc-protected linker (0.50 mmol, 1.0 eq, commercially available), (S,R,S)-APLC/(S,R,S)-AHPC-Me (0.55 mmol, 1.1 eq. synthesized with known methods (X. Han, et al., J. Med. Chem. 2019, 62, 941-964; K. Raina et al., Proc Natl Acad Sci USA 2016, 113, 7124-7129) HOAt (0.60 mmol, 1.2 eq), and DIPEA (2.00 mmol, 4.0 eq) in DMF was added HATU (0.75 mmol, 1.5 eq). After 15 minutes, the reaction was quenched with D.I. water, and the mixture was purified by reverse phase flash column to give indicated products (MeOH/H₂O, 40%→100%). Yield ranging between 75% and 90%.

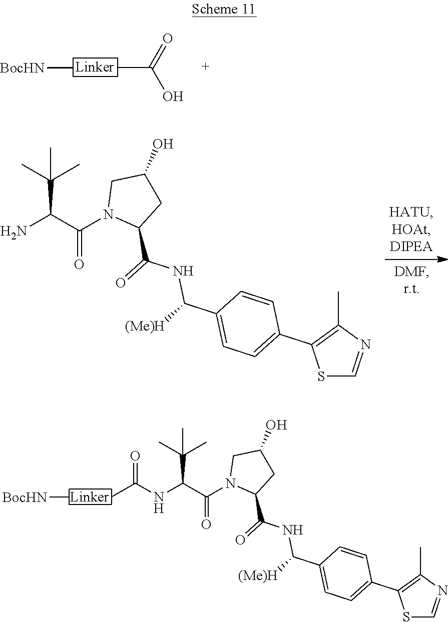

General synthesis method for linker-VHL ligand tethering for synthesis of intermediates 15-22.

### Synthesis of Intermediate 15

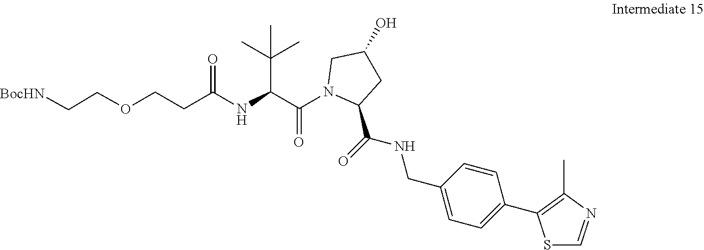 [SMILES: Cc1ncsc1-c1ccc(CNC(=O)[C@@H]2C[C@@H](O)CN2C(=O)[C@@H](NC(=O)CCOCCNC(=O)OC(C)(C)C)C(C)(C)C)cc1]

tert-butyl (2-(3-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-3-oxopropoxy)ethyl)carbamate (Intermediate 15). ¹H NMR (500 MHz, DMSO) δ 8.96 (s, 1H), 8.54 (t, J=6.1 Hz, 1H), 7.91 (d, J=9.3 Hz, 1H), 7.45-7.34 (m, 4H), 6.68 (t, J=5.8 Hz, 1H), 5.11 (d, J=3.5 Hz, 1H), 4.54 (d, J=9.4 Hz, 1H), 4.46-4.38 (m, 2H), 4.37-4.30 (m, 1H), 4.20 (dd, J=15.8, 5.4 Hz, 1H), 3.69-3.50 (m, 4H), 3.37-3.27 (m, 2H), 3.03 (q, J=6.1 Hz, 2H), 2.57-2.45 (m, 1H), 2.43 (s, 3H), 2.36-2.28 (m, 1H), 2.06-1.99 (m, 1H), 1.89 (ddd, J=12.9, 8.6, 4.6 Hz, 1H), 1.35 (s, 9H), 0.92 (s, 9H). LC/MS m/z calculated [M+H]⁺ 646.31. found 646.47.

### Synthesis of Intermediate 16

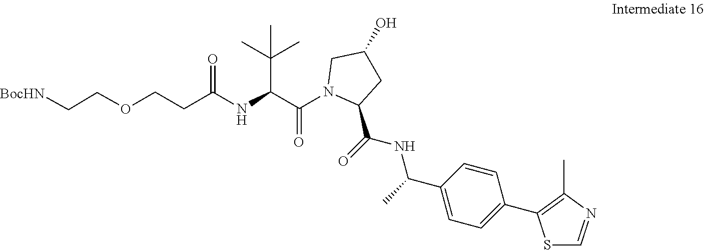 [SMILES: Cc1ncsc1-c1ccc([C@H](C)NC(=O)[C@@H]2C[C@@H](O)CN2C(=O)[C@@H](NC(=O)CCOCCNC(=O)OC(C)(C)C)C(C)(C)C)cc1]

tert-butyl (2-(3-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-3-oxopropoxy)ethyl)carbamate (Intermediate 16). ¹H NMR (500 MHz, DMSO) δ 8.96 (s, 1H), 8.36 (d, J=7.8 Hz, 1H), 7.85 (d, J=9.3 Hz, 1H), 7.45-7.39 (m, 2H), 7.39-7.31 (m, 2H), 6.69 (t, J=5.8 Hz, 1H), 5.09 (d, J=3.6 Hz, 1H), 4.96-4.86 (m, 1H), 4.52 (d, J=9.4 Hz, 1H), 4.41 (t, J=8.0 Hz, 1H), 4.28-4.24 (m, 1H), 3.62-3.51 (m, 4H), 3.39-3.27 (m, 3H), 3.04 (p, J=6.0 Hz, 2H), 2.44 (s, 3H), 2.33 (dt, J=14.6, 6.0 Hz, 1H), 2.03-1.96 (m, 1H), 1.78 (ddd, J=12.9, 8.5, 4.7 Hz, 1H), 1.39-1.33 (m, 12H), 0.92 (s, 9H). LC/MS m/z calculated [M+H]⁺ 660.31. found 660.44.

### Synthesis of Intermediate 17

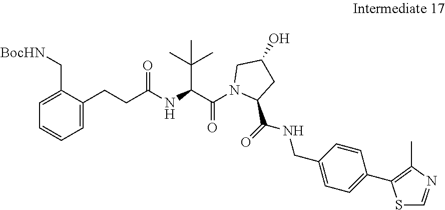 [SMILES: Cc1ncsc1-c1ccc(CNC(=O)[C@@H]2C[C@@H](O)CN2C(=O)[C@@H](NC(=O)CCc2ccccc2CNC(=O)OC(C)(C)C)C(C)(C)C)cc1]

tert-butyl (2-(3-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-3-oxopropyl)benzyl)carbamate (Intermediate 17). ¹H NMR (500 MHz, DMSO) δ 8.96 (s, 1H), 8.55 (t, J=6.1 Hz, 1H), 7.93 (d, J=9.3 Hz, 1H), 7.46-7.34 (m, 4H), 7.27 (t, J=6.1 Hz, 1H), 7.21-7.09 (m, 4H), 5.14 (d, J=3.5 Hz, 1H), 4.54 (d, J=9.4 Hz, 1H), 4.42 (ddd, J=10.7, 6.7, 3.2 Hz, 2H), 4.35 (s, 1H), 4.24-4.13 (m, 3H), 3.70-3.61 (m, 2H), 2.81 (dddd, J=23.2, 19.9, 9.0, 6.2 Hz, 2H), 2.54 (ddd, J=14.3, 9.2, 7.2 Hz, 1H), 2.43 (s, 3H), 2.45-2.33 (m, 1H), 2.08-1.97 (m, 2H), 1.37 (s, 9H), 0.88 (s, 9H). LC/MS m/z calculated [M+H]⁺ 646.33. found 646.39.

### Synthesis of Intermediate 18

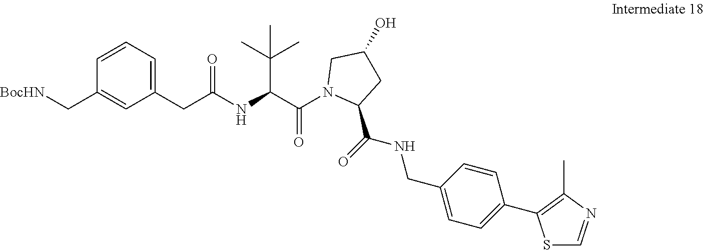 [SMILES: Cc1ncsc1-c1ccc(CNC(=O)[C@@H]2C[C@@H](O)CN2C(=O)[C@@H](NC(=O)Cc2cccc(CNC(=O)OC(C)(C)C)c2)C(C)(C)C)cc1]

tert-butyl (3-(2-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-2-oxoethyl)benzyl)carbamate (Intermediate 18). ¹H NMR (500 MHz, DMSO) δ 8.97 (s, 1H), 8.56 (t, J=6.1 Hz, 1H), 8.09 (d, J=9.3 Hz, 1H), 7.44-7.29 (m, 5H), 7.20 (t, J=7.5 Hz, 1H), 7.14-7.08 (m, 2H), 7.05 (d, J=7.4, 1H), 5.10 (d, J=3.5 Hz, 1H), 4.50 (d, J=9.3 Hz, 1H), 4.45-4.39 (m, 2H), 4.35-4.30 (m, 1H), 4.20 (dd, J=15.8, 5.4 Hz, 1H), 4.08 (d, J=6.2 Hz, 2H), 3.68-3.56 (m, 3H), 3.41 (d, J=13.9 Hz, 1H), 2.43 (s, 3H), 2.04-1.98 (m, 1H), 1.88 (ddd, J=12.9.8.6, 4.6 Hz, 1H), 1.37 (s, 9H), 0.91 (s, 9H). LC/MS m/z calculated [M+H]⁺ 678.33. found 678.42.

### Synthesis of Intermediate 19

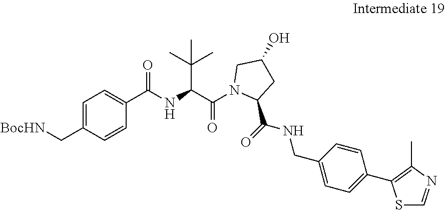 [SMILES: Cc1ncsc1-c1ccc(CNC(=O)[C@@H]2C[C@@H](O)CN2C(=O)[C@@H](NC(=O)c2ccc(CNC(=O)OC(C)(C)C)cc2)C(C)(C)C)cc1]

tert-butyl (4-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)carbamoyl)benzyl)carbamate (Intermediate 19). ¹H NMR (500 MHz, DMSO) δ 8.96 (s, 1H), 8.57 (t, J=6.0 Hz, 1H), 7.89 (d, J=9.1 Hz, 1H), 7.81 (d, J=8.0 Hz, 2H), 7.45-7.34 (m, 5H), 7.29 (d, J=8.0 Hz, 2H), 5.15 (d, J=3.6 Hz, 1H), 4.75 (d, J=9.1 Hz, 1H), 4.48-4.33 (m, 3H), 4.22 (dd, J=15.8, 5.5 Hz, 1H), 4.15 (d, J=6.2 Hz, 2H), 3.71 (d, J=3.1 Hz, 2H), 2.43 (s, 3H), 2.06-2.01 (m, 1H), 1.93-1.88 (m, 1H), 1.37 (s, 9H), 1.01 (s, 9H). LC/MS m/z calculated [M+H]⁻ 664.32. found 664.48.

### Synthesis of Intermediate 20

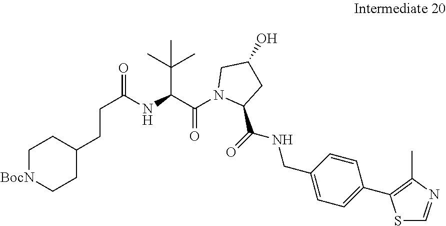 [SMILES: Cc1ncsc1-c1ccc(CNC(=O)[C@@H]2C[C@@H](O)CN2C(=O)[C@@H](NC(=O)CCC2CCN(C(=O)OC(C)(C)C)CC2)C(C)(C)C)cc1]

tert-butyl 4-(3-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-3-oxopropyl)piperidine-1-carboxylate (Intermediate 20). ¹H NMR (500 MHz, DMSO) δ 8.96 s, 1H), 8.54 (t, J=6.1 Hz, 1H), 7.87 (d, J=9.3 Hz, 1H), 7.45-7.34 (m, 4H), 5.11 (d, J=3.6 Hz, 1H), 4.52 (d, J=9.4 Hz, 1H), 4.46-4.37 (m, 2H), 4.33 (s, 1H), 4.20 (dd, J=15.9, 5.5 Hz, 1H), 3.88 (d, J=12.5 Hz, 2H), 3.69-3.59 (m, 2H), 3.40-3.35 (m, 2H), 2.42 (s, 3H), 2.32-2.20 (m, 1H), 2.14 (ddd, J=14.3, 8.6, 6.1 Hz, 1H), 2.08-1.97 (m, 2H), 1.88 (ddd, J=12.9, 8.6, 4.6 Hz, 1H), 1.64-1.55 (m, 2H), 1.48-1.27 (m, 4H), 1.36 (s, 9H), 0.91 (s, 9H). LC/MS m/z calculated [M+H]⁺ 670.36. found 670.43.

### Synthesis of Intermediate 21

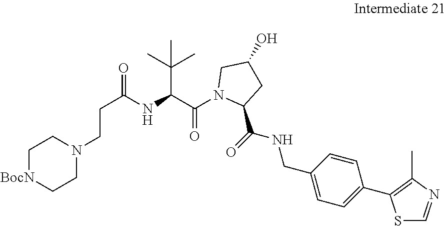 [SMILES: Cc1ncsc1-c1ccc(CNC(=O)[C@@H]2C[C@@H](O)CN2C(=O)[C@@H](NC(=O)CCN2CCN(C(=O)OC(C)(C)C)CC2)C(C)(C)C)cc1]

tert-butyl 4-(3-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-3-oxopropyl)piperazine-1-carboxylate (Intermediate 21). ¹H NMR (500 MHz, DMSO) δ 6 8.95 (s, 1H), 8.58 (t, J=6.1 Hz, 1H), 8.53 (d, J=9.5 Hz, 1H), 7.45-7.32 (m, 4H), 5.13 (s, 1H), 4.53 (d, J=9.5 Hz, 1H), 4.42 (dt, J=12.1, 7.3 Hz, 2H), 4.34 (s, 1H), 4.19 (dd, J=15.9, 5.4 Hz, 1H), 3.68-3.56 (m, 2H), 3.34-3.24 (m, 6H, 2.42 (s, 3H), 2.40-2.35 (m, 2H), 2.32-2.21 (m, 3H), 2.08-1.98 (m, 2H), 1.88 (ddd, J=13.0, 8.7, 4.6 Hz, 1H), 1.32 (s, 9H), 0.92 (s, 9H). LC/MS m/z calculated [M+H]⁺ 671.36. found 671.45.

### Synthesis of Intermediate 22

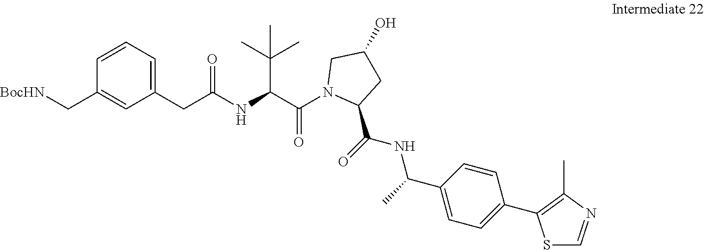 [SMILES: Cc1ncsc1-c1ccc([C@H](C)NC(=O)[C@@H]2C[C@@H](O)CN2C(=O)[C@@H](NC(=O)Cc2cccc(CNC(=O)OC(C)(C)C)c2)C(C)(C)C)cc1]

tert-butyl (3-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethyl)benzyl)carbamate (Intermediate 22). ¹H NMR (500 MHz, DMSO) δ 8.96 (s, 1H), 8.37 (d, J=7.8 Hz, 1H), 8.01 (d, J=9.2 Hz, 1H), 7.45-7.39 (m, 2H), 7.39-7.27 (m, 3H), 7.21 (t, J=7.5 Hz, 1H), 7.14-7.09 (m, 2H), 7.06 (dt, J=7.6, 1.5 Hz, 1H), 5.07 (d, J=3.5 Hz, 1H), 4.95-4.87 (m, 1H), 4.48 (d, J=9.3 Hz, 1H), 4.42 (t, J=8.0 Hz, 1H), 4.28-4.22 (m, 1H), 4.08 (d, J=6.2 Hz, 2H), 3.64-3.53 (m, 3H), 3.41 (d, J=13.9 Hz, 1H), 2.44 (s, 3H), 2.03-1.95 (m, 1H), 1.77 (ddd, J=12.9, 8.5, 4.6 Hz, 1H), 1.41-1.34 (m, 12H), 0.91 (s, 9H). LC/MS m/z calculated [M+H]^{+ 692.35}. found 692.46.

### Example 13

### General Synthesis Method for PROTAC Molecules

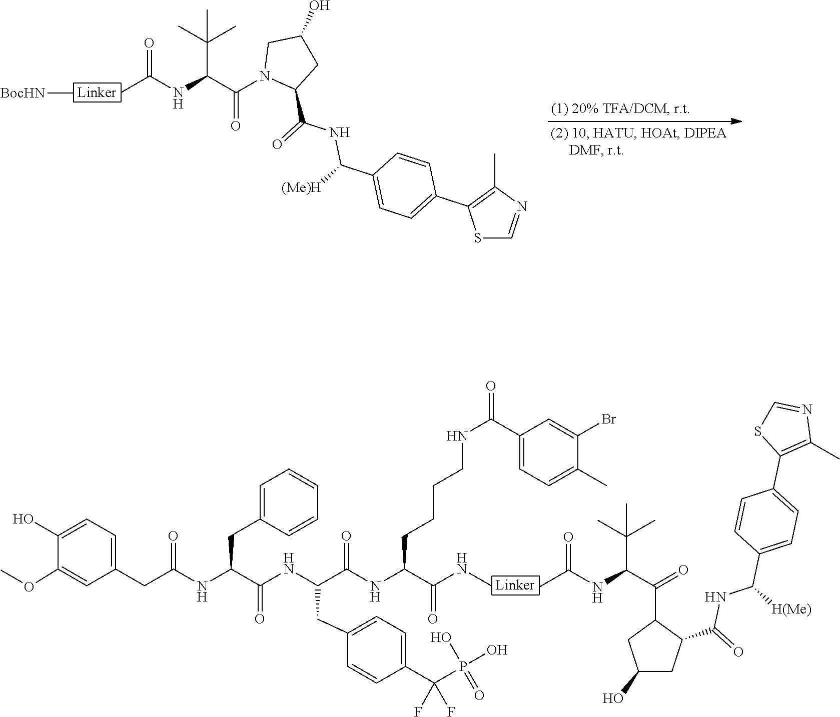

To a solution of the Boc-protected Linker-E3 Ligase ligand complex (1.0 eq) in DCM (4 mL), trifluoroacetic acid (1 mL) was added and stirred for 4 hours at rt. Then the excess reagent and solvent were evaporated under reduced pressure to give the crude deprotected amine, which was used in the next step without further purification.

The deprotected amine, intermediate 10 (1 eq), HOAt (1.2 eq) and DIPEA (7.0 eq) were then dissolved in DMF (50 ml) and stirred at 0° C. To the stirred solution HATU (1.5 eq) was added slowly. After 15 minutes, the reaction mixture was quenched with D.I. water. The mixture was then purified by prep HPLC to give indicated products (MeOH/H₂O, 50%→90%). Yield ranging between 55% and 70%.

Linker-E3 ligand complex from Example 14 to Example 27 was synthesized with a known method (see, e.g., X. Han, et al., J. Med. Chem. 2019, 62, 941-964, which is specifically incorporated herein by reference for its teachings regarding same).

### Synthesis of PROTAC Compound 6

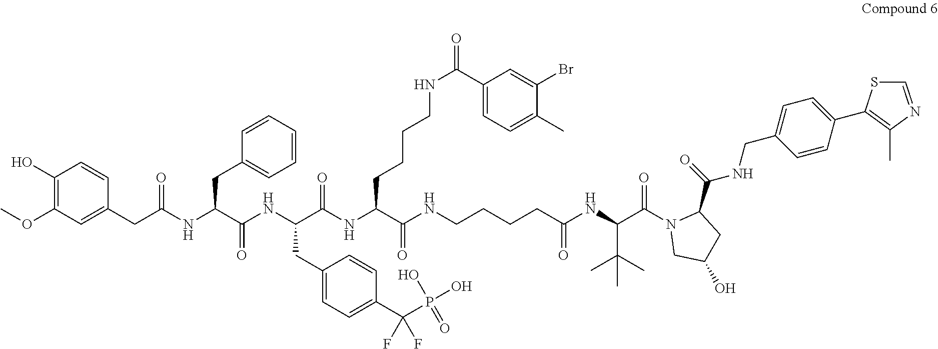 [SMILES: COc1cc(CC(=O)N[C@@H](Cc2ccccc2)C(=O)N[C@@H](Cc2ccc(C(F)(F)P(=O)(O)O)cc2)C(=O)N[C@@H](CCCCNC(=O)c2ccc(C)c(Br)c2)C(=O)NCCCCC(=O)N[C@@H](C(=O)N2C[C@@H](O)C[C@@H]2C(=O)NCc2ccc(-c3scnc3C)cc2)C(C)(C)C)ccc1O]

### ((4-((S)-3-(((S)-6-(3-bromo-4-methylbenzamido)-1-((5-(((R)-1-((2R,4S)-4-hydroxy-2-((4-(4-methylthiazol-5-yl)benzyl)carbamoyl)pyrrolidin-1-yl)-3,3-dimethyl-1-oxobutan-2-yl)amino)-5-oxopentyl)amino)-1-oxohexan-2-yl)amino)-2-((S)-2-(2-(4-hydroxy-3-methoxyphenyl)acetamido)-3-phenylpropanamido)-3-oxopropyl)phenyl)difluoromethyl)phosphonic Acid (Compound 6)

Linker-E3 ligand complex was synthesized with a known method (X. Han, et al., J. Med. Chem. 2019, 62, 941-964). NMR characterization matches the reported result. ¹H NMR (500 MHz, DMSO) δ 8.98-8.93 (m, 1H), 8.60-8.54 (m, 2H), 8.13-8.00 (m, 3H), 7.99-7.68 (m, 4H), 7.46-7.34 (m, 8H), 7.25-7.07 (m, 6H), 6.66 (d, J=2.0 Hz, 1H), 6.57 (d, J=8.1 Hz, 1H), 6.44-6.38 (m, 1H), 4.57-4.47 (m, 2H), 4.47-4.35 (m, 3H), 4.35-4.29 (m, 1H), 4.23-4.13 (m, 2H), 3.67-3.54 (m, 5H), 3.28-3.13 (m, 4H), 3.08-2.98 (m, 2H), 2.98-2.90 (m, 2H), 2.88-2.78 (m, 1H), 2.73-2.66 (m, 1H), 2.42 (s, 3H), 2.35 (s, 3H), 2.27-2.18 (m, 1H), 2.14-1.97 (m, 3H), 1.89 (ddd, J=13.0, 8.5, 4.6 Hz, 1H), 1.67-1.58 (m, 1H), 1.57-1.28 (m, 8H), 0.91 (s 9H). LC/MS m/z calculated [M−H]⁻ 1442.46. found 1442.58.

### Example 14

### Synthesis of PROTAC Compound 8

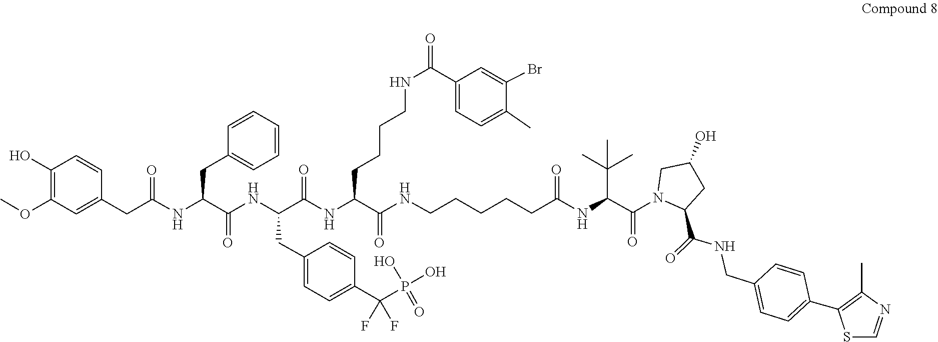 [SMILES: COc1cc(CC(=O)N[C@@H](Cc2ccccc2)C(=O)N[C@@H](Cc2ccc(C(F)(F)P(=O)(O)O)cc2)C(=O)N[C@@H](CCCCNC(=O)c2ccc(C)c(Br)c2)C(=O)NCCCCCC(=O)N[C@H](C(=O)N2C[C@H](O)C[C@H]2C(=O)NCc2ccc(-c3scnc3C)cc2)C(C)(C)C)ccc1O]

### ((4-((S)-3-(((S)-6-(3-bromo-4-methylbenzamido)-1-((6-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-6-oxohexyl)amino)-1-oxohexan-2-yl)amino)-2-((S)-2-(2-(4-hydroxy-3-methoxyphenyl)acetamido)-3-phenylpropanamido)-3-oxopropyl)phenyl)difluoromethyl)phosphonic Acid (Compound 8)

Linker-E3 ligand complex was synthesized with a known method (X. Han, et al., J. Med. Chem. 2019, 62, 941-964). NMR characterization matches the reported result. ¹H NMR (500 MHz, DMSO) δ 8.97 (s, 1H), 8.57-8.45 (m, 2H), 8.19 (d, J=8.1 Hz, 1H), 8.12-7.98 (m, 3H), 7.86-7.80 (m, 2H), 7.76-7.70 (m, 1H), 7.42-7.27 (m, 9H), 7.17-7.08 (m, 5H), 6.65 (d, J=2.0 Hz, 1H), 6.56 (d, J=8.1 Hz, 1H), 6.43-6.37 (m, 1H), 4.58-4.48 (m, 2H), 4.47-4.35 (m, 3H), 4.35-4.29 (m, 1H), 4.23-4.13 (m, 2H), 3.67-3.54 (m, 5H), 3.29-3.13 (m, 4H), 3.09-2.99 (m, 2H), 2.99-2.91 (m, 2H), 2.89-2.79 (m, 1H), 2.69 (dd, J=13.5, 9.6 Hz, 1H), 2.42 (s, 3H), 2.34 (s, 3H), 2.26-2.15 (m, 1H), 2.13-1.98 (m, 3H), 1.88 (ddd, J=13.0, 8.6, 4.6 Hz, 1H), 1.68-1.59 (m, 1H), 1.56-1.25 (m, 10H), 0.90 (s, 9H). LC/MS m/z calculated [M−H]⁻ 1456.47. found 1456.60.

### Example 15

### Synthesis of PROTAC Compound 10

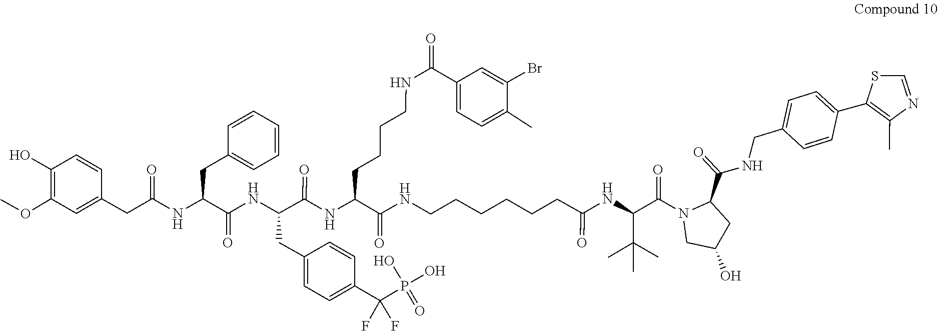 [SMILES: COc1cc(CC(=O)N[C@@H](Cc2ccccc2)C(=O)N[C@@H](Cc2ccc(C(F)(F)P(=O)(O)O)cc2)C(=O)N[C@@H](CCCCNC(=O)c2ccc(C)c(Br)c2)C(=O)NCCCCCCC(=O)N[C@@H](C(=O)N2C[C@@H](O)C[C@@H]2C(=O)NCc2ccc(-c3scnc3C)cc2)C(C)(C)C)ccc1O]

### ((4-((S)-3-(((S)-6-(3-bromo-4-methylbenzamido)-1-((7-(((R)-1-((2R,4S)-4-hydroxy-2-((4-(4-methylthiazol-5-yl)benzyl)carbamoyl)pyrrolidin-1-yl)-3,3-dimethyl-1-oxobutan-2-yl)amino)-7-oxoheptyl)amino)-1-oxohexan-2-yl)amino)-2-((S)-2-(2-(4-hydroxy-3-methoxyphenyl)acetamido)-3-phenylpropanamido)-3-oxopropyl)phenyl)difluoromethyl)phosphonic Acid (Compound 10)

Linker-E3 ligand complex was synthesized with a known method (X. Han, et al., J. Med. Chem. 2019, 62, 941-964). NMR characterization matches the reported result. ¹H NMR (500 MHz, DMSO) δ 8.96 (d, J=3.7 Hz, 1H), 8.62-8.51 (m, 2H), 8.12-8.00 (m, 4H), 7.88-7.70 (m, 3H), 7.45-7.32 (m, 8H), 7.16-7.08 (m, 6H), 6.67 (d, J=2.0 Hz, 1H), 6.58 (d, J=8.1 Hz, 1H), 6.41 (dd, J=8.2, 2.1 Hz, 1H), 4.55-4.48 (m, 2H), 4.47-4.35 (m, 3H), 4.35-4.28 (m, 1H), 4.23-4.13 (m, 2H), 3.69-3.53 (m, 5H), 3.29-3.13 (m, 4H), 3.03-2.90 (m, 4H), 2.89-2.78 (m, 1H), 2.75-2.67 (m, 1H), 2.42 (s, 3H), 2.35 (s, 3H), 2.26-2.17 (m, 1H), 2.13-1.98 (m, 3H), 1.91-1.84 (m, 1H), 1.67-1.57 (m, 1H), 1.57-1.26 (m, 12H), 0.90 (s, 9H). LC/MS m/z calculated [M−H]⁻ 1470.49. found 1470.61.

### Example 16

### Synthesis of PROTAC Compound 83

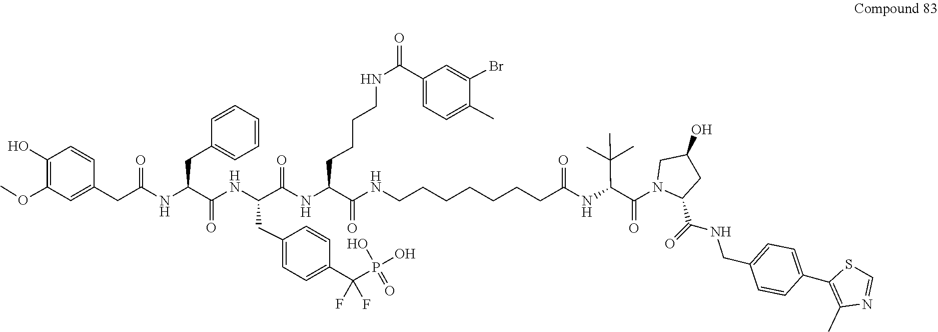 [SMILES: COc1cc(CC(=O)N[C@@H](Cc2ccccc2)C(=O)N[C@@H](Cc2ccc(C(F)(F)P(=O)(O)O)cc2)C(=O)N[C@@H](CCCCNC(=O)c2ccc(C)c(Br)c2)C(=O)NCCCCCCCC(=O)N[C@@H](C(=O)N2C[C@@H](O)C[C@@H]2C(=O)NCc2ccc(-c3scnc3C)cc2)C(C)(C)C)ccc1O]

### ((4-((S)-3-(((S)-6-(3-bromo-4-methylbenzamido)-1-((8-(((R)-1-((2R,4S)-4-hydroxy-2-((4-(4-methylthiazol-5-yl)benzyl)carbamoyl)pyrrolidin-1-yl)-3,3-dimethyl-1-oxobutan-2-yl)amino)-8-oxooctyl)amino)-1-oxohexan-2-yl)amino)-2-((S)-2-(2-(4-hydroxy-3-methoxyphenyl)acetamido)-3-phenylpropanamido)-3-oxopropyl)phenyl)difluoromethyl)phosphonic Acid (Compound 83)

Linker-E3 ligand complex was synthesized with a known method (X. Han, et al., J. Med. Chem. 2019, 62, 941-964). NMR characterization matches the reported result. ¹H NMR (500 MHz, DMSO) δ 8.97 (d, J=3.7 Hz, 1H), 8.63-8.52 (m, 2H), 8.13-8.01 (m, 4H), 7.89-7.71 (m, 3H), 7.45-7.30 (m, 8H), 7.18-7.09 (m, 6H), 6.68 (d, J=2.0 Hz, 1H), 6.59 (d, J=8.1 Hz, 1H), 6.42 (dd, J=8.2, 2.1 Hz, 1H), 4.56-4.49 (m, 2H), 4.48-4.36 (m, 3H), 4.35-4.29 (m, 1H), 4.24-4.14 (m, 2H), 3.69-3.54 (m, 5H), 3.30-3.14 (m, 4H), 3.04-2.91 (m, 4H), 2.89-2.79 (m, 1H), 2.76-2.68 (m, 1H), 2.42 (s, 3H), 2.35 (s, 3H), 2.27-2.17 (m, 1H), 2.13-1.98 (m, 3H), 1.91-1.84 (m, 1H), 1.67-1.57 (m, 1H), 1.58-1.25 (m, 14H), 0.90 (s, 9H). LC/MS m/z calculated [M−H]⁻ 1484.50. found 1485.60.

### Example 17

### Synthesis of PROTAC Compound 14

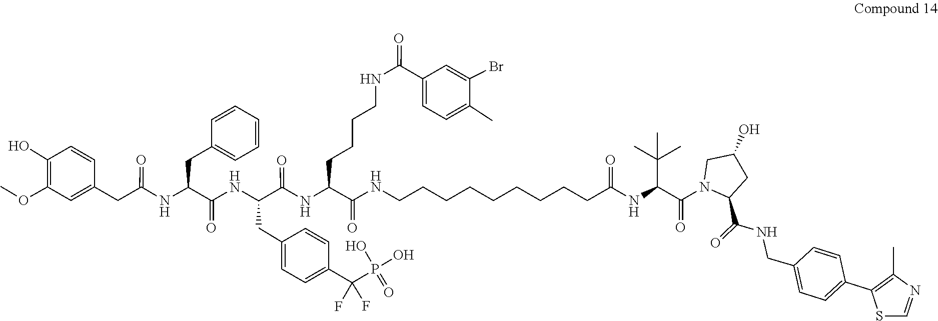 [SMILES: COc1cc(CC(=O)N[C@@H](Cc2ccccc2)C(=O)N[C@@H](Cc2ccc(C(F)(F)P(=O)(O)O)cc2)C(=O)N[C@@H](CCCCNC(=O)c2ccc(C)c(Br)c2)C(=O)NCCCCCCCCCC(=O)N[C@H](C(=O)N2C[C@H](O)C[C@H]2C(=O)NCc2ccc(-c3scnc3C)cc2)C(C)(C)C)ccc1O]

### ((4-((S)-3-(((S)-6-(3-bromo-4-methylbenzamido)-1-((10-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-10-oxodecyl)amino)-1-oxohexan-2-yl)amino)-2-((S)-2-(2-(4-hydroxy-3-methoxyphenyl)acetamido)-3-phenylpropanamido)-3-oxopropyl)phenyl)difluoromethyl)phosphonic Acid (Compound 14)

Linker-E3 ligand complex was synthesized with a known method (X. Han, et al., J. Med. Chem. 2019, 62, 941-964). NMR characterization matches the reported result. ¹H NMR (500 MHz, DMSO) δ 8.99 (s, 1H), 8.54 (t, J=6.1 Hz, 1H), 8.47 (t, J=5.6 Hz, 1H), 8.11-7.94 (m, 3H), 7.91-7.76 (m, 3H), 7.73 (d, J=8.0 Hz, 1H), 7.45-7.23 (m, 8H), 7.20-7.03 (m, 6H), 6.69-6.49 (m, 2H), 6.44-6.37 (m, 1H), 4.59-4.48 (m, 2H), 4.48-4.36 (m, 3H), 4.35-4.29 (m, 1H), 4.24-4.13 (m, 2H), 3.69-3.53 (m, 5H), 3.30-3.12 (m, 4H), 3.10-2.78 (m, 5H), 2.75-2.65 (m, 1H), 2.42 (s, 3H), 2.34 (s, 3H), 2.23 (dt, J=14.7, 7.7 Hz, 1H), 2.13-1.94 (m, 3H), 1.88 (ddd, J=12.9, 8.6, 4.6 Hz, 1H), 1.68-1.58 (m, 1H), 1.58-1.16 (m, 18H), 0.91 (s, 9H). LC/MS m/z calculated [M−H]⁻ 1512.54. found 1512.61.

### Example 18

### Synthesis of PROTAC Compound 18

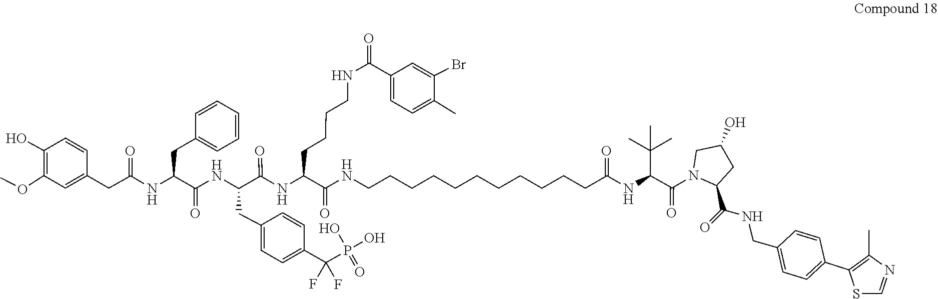 [SMILES: COc1cc(CC(=O)N[C@@H](Cc2ccccc2)C(=O)N[C@@H](Cc2ccc(C(F)(F)P(=O)(O)O)cc2)C(=O)N[C@@H](CCCCNC(=O)c2ccc(C)c(Br)c2)C(=O)NCCCCCCCCCCCC(=O)N[C@H](C(=O)N2C[C@H](O)C[C@H]2C(=O)NCc2ccc(-c3scnc3C)cc2)C(C)(C)C)ccc1O]

### ((4-((S)-3-(((S)-6-(3-bromo-4-methylbenzamido)-1-((12-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-12-oxododecyl)amino)-1-oxohexan-2-yl)amino)-2-((S)-2-(2-(4-hydroxy-3-methoxyphenyl)acetamido)-3-phenylpropanamido)-3-oxopropyl)phenyl)difluoromethyl)phosphonic Acid (Compound 18)

Linker-E3 ligand complex was synthesized with a known method (X. Han, et al., J. Med. Chem. 2019, 62, 941-964). NMR characterization matches the reported result. ¹H NMR (500 MHz, DMSO) δ 8.97 (s, 1H), 8.77-8.67 (m, 1H), 8.58-8.43 (m, 2H), 8.12-7.94 (m, 3H), 7.92-7.76 (m, 2H), 7.73 (d, J=7.8 Hz, 1H), 7.51-7.25 (m, 8H), 7.24-6.99 (m, 6H), 6.67-6.47 (m, 2H), 6.42-6.35 (m, 1H), 4.59-4.49 (m, 2H), 4.48-4.36 (m, 3H), 4.35-4.29 (m, 1H), 4.27-4.13 (m, 2H), 3.66-3.49 (m, 5H), 3.28-3.11 (m, 4H), 3.10-2.78 (m, 5H), 2.73-2.64 (m, 1H), 2.42 (s, 3H), 2.34 (s, 3H), 2.28-2.20 (m, 1H), 2.10-1.85 (m, 3H), 1.92-1.84 (m, 1H), 1.67-1.58 (m, 1H), 1.58-1.09 (m, 22H), 0.91 (s, 9H). LC/MS m/z calculated [M−H]⁻ 1540.57. found 1540.70.

### Example 19

### Synthesis of PROTAC Compound 28

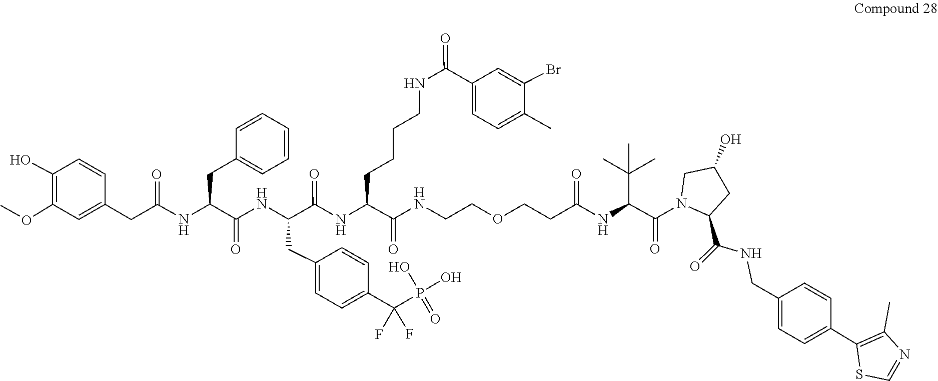 [SMILES: COc1cc(CC(=O)N[C@@H](Cc2ccccc2)C(=O)N[C@@H](Cc2ccc(C(F)(F)P(=O)(O)O)cc2)C(=O)N[C@@H](CCCCNC(=O)c2ccc(C)c(Br)c2)C(=O)NCCOCCC(=O)N[C@H](C(=O)N2C[C@H](O)C[C@H]2C(=O)NCc2ccc(-c3scnc3C)cc2)C(C)(C)C)ccc1O]

### ((4-((2S,5S,15S)-5-(4-(3-bromo-4-methylbenzamido)butyl)-15-((2S,4R)-4-hydroxy-2-((4-(4-methylthiazol-5-yl)benzyl)carbamoyl)pyrrolidine-1-carbonyl)-2-((S)-2-(2-(4-hydroxy-3-methoxyphenyl)acetamido)-3-phenylpropanamido)-16,16-dimethyl-3,6,13-trioxo-10-oxa-4,7,14-triazaheptadecyl)phenyl)difluoromethyl)phosphonic Acid (Compound 28)

¹H NMR (500 MHz, DMSO) δ 9.01 (s, 1H), 8.58-8.53 (m, 1H), 8.52-8.46 (m, 1H), 8.19 (d, J=8.0 Hz, 1H), 8.10-8.02 (m, 2H), 8.02 (d, J=1.8 Hz, 1H), 8.00-7.91 (m, 2H), 7.73 (dd, J=7.8, 1.9 Hz, 1H), 7.44-7.29 (m, 9H), 7.20-7.08 (m, 5H), 6.65 (d, J=2.0 Hz, 1H), 6.60-6.54 (m, 1H), 6.44-6.36 (m, 1H), 4.60-4.51 (m, 2H), 4.48-4.36 (m, 3H), 4.35-4.30 (m, 1H), 4.26-4.16 (m, 2H), 3.68-3.51 (m, 7H), 3.40-3.29 (m, 2H), 3.28-3.10 (m, 6H), 3.06 (dd, J=14.2, 4.4 Hz, 1H), 2.95 (dd, J=14.0, 3.9 Hz, 1H), 2.89-2.78 (m, 1H), 2.73-2.65 (m, 1H), 2.57-2.50 (m, 1H), 2.42 (s, 3H), 2.38-2.28 (m, 4H), 2.05-1.98 (m, 1H), 1.88 (ddd, J=12.9, 8.7, 4.6 Hz, 1H), 1.69-1.42 (m, 4H), 1.37-1.19 (m, 2H), 0.91 (s, 9H). LC/MS m/z calculated [M−H]⁻ 1458.45. found 1458.54.

### Example 20

### Synthesis of PROTAC Compound 27

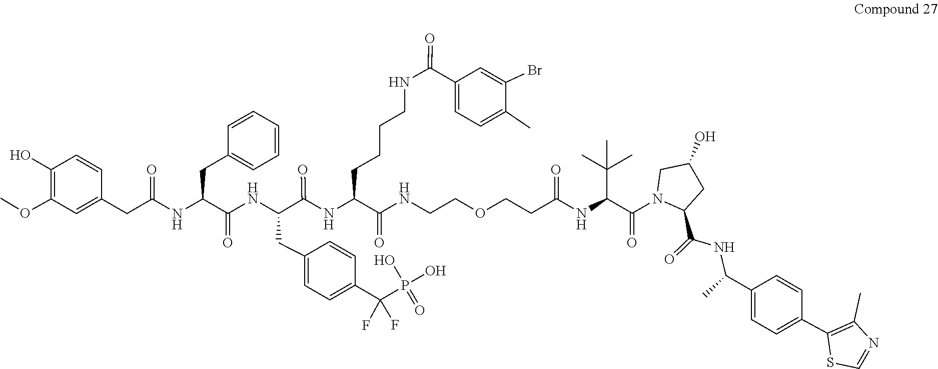 [SMILES: COc1cc(CC(=O)N[C@@H](Cc2ccccc2)C(=O)N[C@@H](Cc2ccc(C(F)(F)P(=O)(O)O)cc2)C(=O)N[C@@H](CCCCNC(=O)c2ccc(C)c(Br)c2)C(=O)NCCOCCC(=O)N[C@H](C(=O)N2C[C@H](O)C[C@H]2C(=O)N[C@@H](C)c2ccc(-c3scnc3C)cc2)C(C)(C)C)ccc1O]

### ((4-((2S,5S,15S)-5-(4-(3-bromo-4-methylbenzamido)butyl)-15-((2S,4R)-4-hydroxy-2-(((S)-1-(4-(4-methylthiazol-5-yl)phenyl)ethyl)carbamoyl)pyrrolidine-1-carbonyl)-2-((S)-2-(2-(4-hydroxy-3-methoxyphenyl)acetamido)-3-phenylpropanamido)-16,16-dimethyl-3,6,13-trioxo-10-oxa-4,7,14-triazaheptadecyl)phenyl)difluoromethyl)phosphonic Acid (Compound 27)

¹H NMR (500 MHz, DMSO) δ 9.01 (s, 1H), 8.53-8.47 (m, 1H), 8.37 (d, J=7.7 Hz, 1H), 8.19 (d, J=8.0 Hz, 1H), 8.12-8.04 (m, 2H), 8.03 (d, J=1.8 Hz, 1H), 8.00-7.84 (m, 1H), 7.87 (d, J=9.3 Hz, 1H), 7.73 (dd, J=7.9, 1.9 Hz, 1H), 7.44-7.29 (m, 9H), 7.17-7.08 (m, 5H), 6.65 (d, J=2.0 Hz, 1H), 6.56 (d, J=7.9 Hz, 1H), 6.39 (dd, J=8.1, 2.0 Hz, 1H), 4.93-4.84 (m, 1H), 4.61-4.49 (m, 2H), 4.49-4.38 (m, 2H), 4.29-4.20 (m, 2H), 3.66-3.52 (m, 7H), 3.41-3.30 (m, 2H), 3.29-3.10 (m, 6H), 3.07 (dd, J=14.1, 4.2 Hz, 1H), 2.96 (dd, J=14.0, 3.9 Hz, 1H), 2.89-2.79 (m, 1H), 2.73-2.65 (m, 1H), 2.52 (dd, J=14.4, 6.9 Hz, 1H), 2.43 (s, 3H), 2.38-2.28 (m, 4H), 2.04-1.96 (m, 1H), 1.77 (ddd, J=12.9, 8.5, 4.6 Hz, 1H), 1.69-1.41 (m, 4H), 1.37-1.19 (m, 5H), 0.91 (s, 9H). LC/MS m/z calculated [M−H]⁻ 1472.47. found 1472.57.

### Example 21

### Synthesis of PROTAC Compound 40

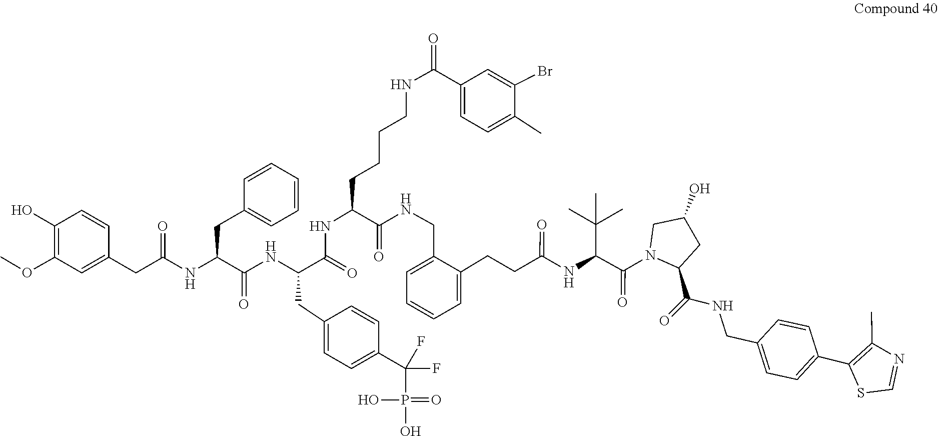 [SMILES: COc1cc(CC(=O)N[C@@H](Cc2ccccc2)C(=O)N[C@@H](Cc2ccc(C(F)(F)P(=O)(O)O)cc2)C(=O)N[C@@H](CCCCNC(=O)c2ccc(C)c(Br)c2)C(=O)NCc2ccccc2CCC(=O)N[C@H](C(=O)N2C[C@H](O)C[C@H]2C(=O)NCc2ccc(-c3scnc3C)cc2)C(C)(C)C)ccc1O]

### ((4-((S)-3-(((S)-6-(3-bromo-4-methylbenzamido)-1-((2-(3-(((S)-1-((2S,4R)-4-hydroxy-2-((4-(4-methylthiazol-5-yl)benzyl)carbamoyl)pyrrolidin-1-yl)-3,3-dimenthyl-1-oxobutan-2-yl)amino)-3-oxopropyl)benzyl)amino)-1-oxohexan-2-yl)amino)-2-((S)-2-(2-(4-hydroxy-3-methoxyphenyl)acetamido)-3-phenylpropanamido)-3-oxopropyl)phenyl)difluoromethyl)phosphonic Acid (Compound 40)

¹H NMR (500 MHz, DMSO) δ 9.00 (s, 1H), 8.52 (dt, J=21.6, 5.8 Hz, 2H), 8.37 (t, J=5.8 Hz, 1H), 8.17 (dd, J=14.4, 7.9 Hz, 1H), 8.09-8.00 (m, 2H), 7.96 (d, J=9.3 Hz, 1H), 7.76-7.69 (m, 1H), 7.42-7.26 (m, 9H), 7.18-7.04 (m, 10H), 6.65 (d, J=1.9 Hz, 1H), 6.56 (d, J=8.0 Hz, 1H), 6.39 (dd, J=8.1, 2.0 Hz, 1H), 4.62-4.50 (m, 2H), 4.47-4.36 (m, 3H), 4.35-4.28 (m, 4H), 4.19 (dd, J=15.9, 5.4 Hz, 1H), 3.69-3.53 (m, 6H), 3.28-3.13 (m, 4H), 3.06 (d, J=13.3 Hz, 1H), 2.94 (dd, J=13.8, 3.7 Hz, 1H), 2.88-2.73 (m, 3H), 2.62-2.49 (m, 2H), 2.43-2.35 (m, 4H), 2.34 (d, J=5.3 Hz, 3H), 2.01-1.95 (m, 1H), 1.93-1.83 (m, 1H), 1.75-1.65 (m, 1H), 1.64-1.55 (m, 1H), 1.54-1.44 (m, 2H), 1.37-1.18 (m, 2H), 0.87 (s, 9H). LC/MS m/z calculated [M−H]⁻ 1504.47. found 1504.55.

### Example 22

### Synthesis of PROTAC Compound 48

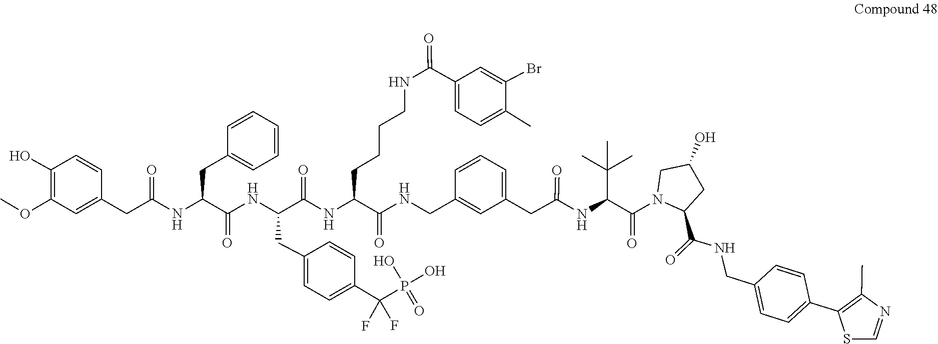 [SMILES: COc1cc(CC(=O)N[C@@H](Cc2ccccc2)C(=O)N[C@@H](Cc2ccc(C(F)(F)P(=O)(O)O)cc2)C(=O)N[C@@H](CCCCNC(=O)c2ccc(C)c(Br)c2)C(=O)NCc2cccc(CC(=O)N[C@H](C(=O)N3C[C@H](O)C[C@H]3C(=O)NCc3ccc(-c4scnc4C)cc3)C(C)(C)C)c2)ccc1O]

### ((4-((S)-3-(((S)-6-(3-bromo-4-methylbenzamido)-1-((3-(2-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-2-oxoethyl)benzyl)amino)-1-oxohexan-2-yl)amino)-2-((S)-2-(2-(4-hydroxy-3-methoxyphenyl)acetamido)-3-phenylpropanamido)-3-oxopropyl)phenyl)difluoromethyl)phosphonic Acid (Compound 48)

¹H NMR (500 MHz, DMSO) δ 8.99 (s, 1H), 8.56 (t, J=6.1 Hz, 1H), 8.50 (t, J=5.5 Hz, 1H), 8.42 (t, J=6.1 Hz, 1H), 8.20 (d, J=8.0 Hz, 1H), 8.16-8.00 (m, 4H), 7.73 (dd, J=7.8, 1.8 Hz, 1H), 7.38 (dd, J=10.5, 7.6 Hz, 6H), 7.31 (d, J=8.0 Hz, 2H), 7.23-7.00 (m, 10H), 6.65 (d, J=2.0 Hz, 1H), 6.56 (d, J=8.0 Hz, 1H), 6.39 (dd, J=8.1, 1.9 Hz, 1H), 4.60-4.54 (m, 1H), 4.51-4.37 (m, 4H), 4.33-4.14 (m, 5H), 3.66-3.55 (m, 6H), 3.42 (d, J=13.9 Hz, 1H), 3.29-3.12 (m, 4H), 3.11-3.05 (m, 1H), 2.99-2.91 (m, 1H), 2.89-2.79 (m, 1H), 2.73-2.65 (m, 1H), 2.42 (s, 3H), 2.35 (s, 3H), 2.04-1.97 (m, 1H), 1.87 (ddd, J=12.9, 8.6, 4.6 Hz, 1H), 1.75-1.65 (m, 1H), 1.64-1.44 (m, 3H), 1.38-1.25 (m, 2H), 0.90 (s, 9H). LC/MS m/z calculated [M−H]⁻ 1490.46. found 1490.54.

### Example 23

### Synthesis of PROTAC Compound 54

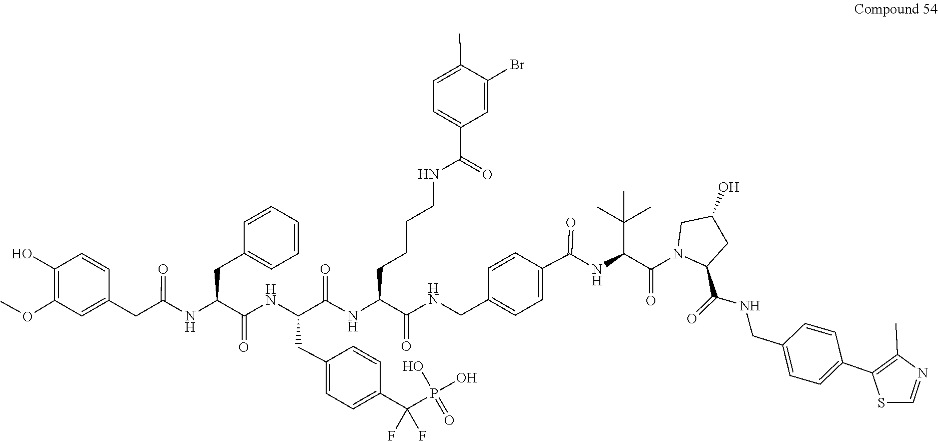 [SMILES: COc1cc(CC(=O)N[C@@H](Cc2ccccc2)C(=O)N[C@@H](Cc2ccc(C(F)(F)P(=O)(O)O)cc2)C(=O)N[C@@H](CCCCNC(=O)c2ccc(C)c(Br)c2)C(=O)NCc2ccc(C(=O)N[C@H](C(=O)N3C[C@H](O)C[C@H]3C(=O)NCc3ccc(-c4scnc4C)cc3)C(C)(C)C)cc2)ccc1O]

### ((4-((S)-3-(((S)-6-(3-bromo-4-methylbenzamido)-1-((4-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)carbamoyl)benzyl)amino)-1-oxohexan-2-yl)amino)-2-((S)-2-(2-(4-hydroxy-3-methoxyphenyl)acetamido)-3-phenylpropanamido)-3-oxopropyl)phenyl)difluoromethyl)phosphonic Acid (Compound 54)

¹H NMR (500 MHz, DMSO) δ 8.98 (s, 1H), 8.59-8.53 (m, 1H), 8.53-8.44 (m, 2H), 8.19 (dd, J=24.3, 7.9 Hz, 2H), 8.09-8.01 (m, 2H), 7.91 (d, J=9.0 Hz, 1H), 7.81 (d, J=7.9 Hz, 2H), 7.75-7.68 (m, 1H), 7.42-7.35 (m, 7H), 7.35-7.27 (m, 4H), 7.17-7.08 (m, 5H), 6.65 (d, J=2.0 Hz, 1H), 6.56 (d, J=8.0 Hz, 1H), 6.39 (dd, J=8.1, 2.0 Hz, 1H), 4.75 (d, J=9.0 Hz, 1H), 4.58 (td, J=8.6, 4.3 Hz, 1H), 4.48-4.24 (m, 7H), 4.24-4.19 (m, 1H), 3.73-3.67 (m, 2H), 3.64 (s, 3H), 3.62-3.57 (m, 1H), 3.28-3.18 (m, 3H), 3.16 (d, J=13.6 Hz, 1H), 3.12-3.04 (m, 1H), 2.95 (dd, J=13.8, 4.1 Hz, 1H), 2.84 (dt, J=14.0, 6.8 Hz, 1H), 2.69 (dd, J=14.1, 10.2 Hz, 1H), 2.42 (s, 3H), 2.34 (s, 3H), 2.05-1.98 (m, 1H), 1.90 (ddd, J=12.9, 8.6, 4.6 Hz, 1H), 1.76-1.66 (m, 1H), 1.64-1.45 (m, 3H), 1.28-1.19 (m, 2H), 1.00 (s, 9H). LC/MS m/z calculated [M−H]⁻ 1476.44. found 1476.59.

### Example 24

### Synthesis of PROTAC Compound 58

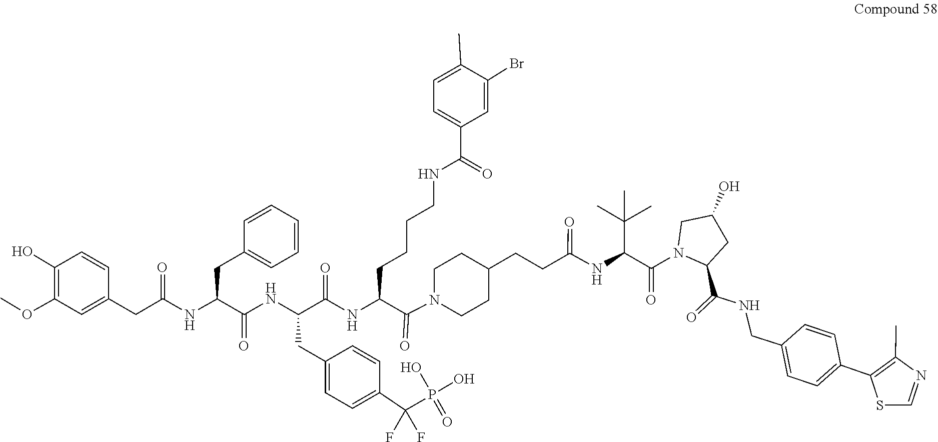 [SMILES: COc1cc(CC(=O)N[C@@H](Cc2ccccc2)C(=O)N[C@@H](Cc2ccc(C(F)(F)P(=O)(O)O)cc2)C(=O)N[C@@H](CCCCNC(=O)c2ccc(C)c(Br)c2)C(=O)N2CCC(CCC(=O)N[C@H](C(=O)N3C[C@H](O)C[C@H]3C(=O)NCc3ccc(-c4scnc4C)cc3)C(C)(C)C)CC2)ccc1O]

### ((4-((S)-3-(((S)-6-(3-bromo-4-methylbenzamido)-1-(4-(3-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-3-oxopropyl)piperidin-1-yl)-1-oxohexan-2-yl)amino)-2-((S)-2-(2-(4-hydroxy-3-methoxyphenyl)acetamido)-3-phenylpropanamido)-3-oxopropyl)phenyl)difluoromethyl)phosphonic Acid (Compound 58)

¹H NMR (500 MHz, DMSO) δ 8.99 (s, 1H), 8.55 (t, J=6.1 Hz, 1H), 8.49 (p, J=4.5 Hz, 1H), 8.24-8.17 (m, 2H), 8.07 (dd, J=8.4, 3.0 Hz, 1H), 8.02 (d, J=1.8 Hz, 1H), 7.95-7.86 (m, 2H), 7.73 (dt, J=8.1, 2.2 Hz, 1H), 7.40 (d, J=7.9 Hz, 4H), 7.38-7.32 (m, 3H), 7.31 (dd, J=8.3, 3.2 Hz, 1H), 7.19-7.08 (m, 5H), 6.69-6.63 (m, 1H), 6.56 (dd, J=8.0, 2.9 Hz, 1H), 6.39 (dd, J=8.2, 2.2 Hz, 1H), 4.75-4.62 (m, 1H), 4.58-4.49 (m, 2H), 4.48-4.37 (m, 3H), 4.37-4.27 (m, 2H), 4.20 (dd, J=15.9, 5.5 Hz, 1H), 3.88 (dd, J=29.1, 12.5 Hz, 1H), 3.68-3.54 (m, 5H), 3.28-3.13 (m, 4H), 3.07-2.91 (m, 2H), 2.87-2.77 (m, 1H), 2.72-2.64 (m, 1H), 2.42 (s, 3H), 2.34 (s, 3H), 2.32-2.20 (m, 1H), 2.18-2.05 (m, 3H), 2.05-1.97 (m, 1H), 1.88 (ddd, J=12.9, 8.6, 4.5 Hz, 1H), 1.73-1.58 (m, 3H), 1.53-1.19 (m, 9H), 0.91 (m, 9H). LC/MS m/z calculated [M−H]⁻ 1482.49. found 1482.61.

### Example 25

### Synthesis of PROTAC Compound 56

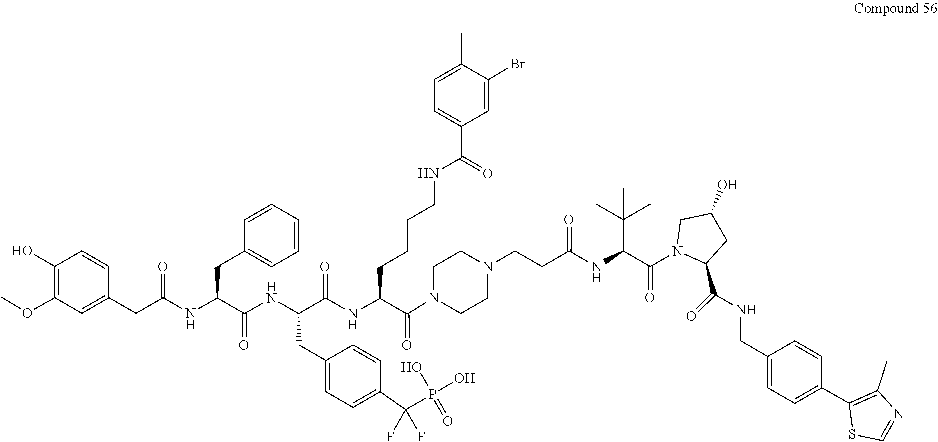 [SMILES: COc1cc(CC(=O)N[C@@H](Cc2ccccc2)C(=O)N[C@@H](Cc2ccc(C(F)(F)P(=O)(O)O)cc2)C(=O)N[C@@H](CCCCNC(=O)c2ccc(C)c(Br)c2)C(=O)N2CCN(CCC(=O)N[C@H](C(=O)N3C[C@H](O)C[C@H]3C(=O)NCc3ccc(-c4scnc4C)cc3)C(C)(C)C)CC2)ccc1O]

### ((4-((S)-3-(((S)-6-(3-bromo-4-methylbenzamido)-1-(4-(3-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-3-oxopropyl)piperazin-1-yl)-1-oxohexan-2-yl)amino)-2-((S)-2-(2-(4-hydroxy-3-methoxyphenyl)acetamido)-3-phenylpropanamido)-3-oxopropyl)phenyl)difluoromethyl)phosphonic Acid (Compound 56)

¹H NMR (500 MHz, DMSO) δ 8.97 (s, 1H), 8.61 (s, 1H), 8.50-8.42 (m, 1H), 8.36-8.26 (m, 2H), 8.09 (d, J=8.3 Hz, 1H), 8.00 (d, J=1.8 Hz, 1H), 7.75-7.68 (m, 1H), 7.47-7.32 (m, 8H), 7.25-7.19 (m, 2H), 7.19-7.09 (m, 5H), 6.72-6.67 (m, 1H), 6.57 (d, J=8.0 Hz, 1H), 6.44 (d, J=8.1 Hz, 1H), 4.66-4.38 (m, 7H), 4.37-4.31 (m, 1H), 4.22 (dd, J=16.1, 5.4 Hz, 1H), 3.71-3.56 (m, 6H), 3.30-3.13 (m, 9H), 3.02-2.97 (m, 1H), 2.94 (dd, J=13.8, 4.1 Hz, 1H), 2.86 (s, 1H), 2.74 (m, 1H), 2.48-1.44 (m, 2H), 2.42 (s, 3H), 2.40 (m, 2H), 2.35 (s, 3H), 2.12-1.99 (m, 3H), 1.93-1.84 (m, 1H), 1.70-1.59 (m, 1H), 1.55-1.41 (m, 3H), 1.27-1.18 (m, 2H), 0.94 (s, 9H). LC/MS m/z calculated [M−H]⁻ 1483.48. found 1483.62.

### Example 26

### Synthesis of PROTAC Cis Compound 47

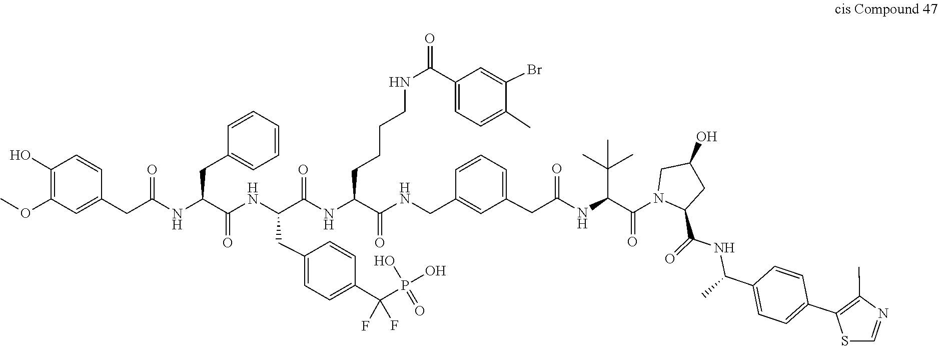 [SMILES: COc1cc(CC(=O)N[C@@H](Cc2ccccc2)C(=O)N[C@@H](Cc2ccc(C(F)(F)P(=O)(O)O)cc2)C(=O)N[C@@H](CCCCNC(=O)c2ccc(C)c(Br)c2)C(=O)NCc2cccc(CC(=O)N[C@H](C(=O)N3C[C@@H](O)C[C@H]3C(=O)N[C@@H](C)c3ccc(-c4scnc4C)cc3)C(C)(C)C)c2)ccc1O]

¹H NMR (500 MHz, DMSO) δ 8.97 (d, J=3.2 Hz, 1H), 8.70 (brs, 1H), 8.53-8.47 (m, 1H), 8.35-8.28 (m, 1H), 8.16 (dd, J=23.7, 7.9 Hz, 1H), 0.8.09-8.00 (m, 2H), 1H), 7.73 (dd, J=7.9, 1.9 Hz, 1H), 7.43-7.33 (m, 8H), 7.31 (d, J=7.9 Hz, 2H), 7.22-7.07 (m, 9H), 7.07 (d, J=7.6 Hz, 2H), 6.65 (d, J=2.0 Hz, 1H), 6.56 (d, J=8.0 Hz, 1H), 6.39 (dd, J=8.2, 1.9 Hz, 1H), 5.30 (s, 1H), 4.95-4.87 (m, 1H), 4.61-4.54 (m, 1H), 4.48-4.37 (m, 2H), 4.34-4.21 (m, 5H), 4.15 (q, J=5.7 Hz, 1H), 3.86-3.79 (m, 1H), 3.65-3.54 (m, 5H), 3.41 (d, J=13.9 Hz, 1H), 3.34 (dd, J=10.1, 5.3 Hz, 1H), 3.28-3.13 (m, 3H), 3.07 (d, J=12.5 Hz, 1H), 2.95 (dd, J=13.9, 4.0 Hz, 1H), 2.89-2.79 (m, 1H), 2.73-2.65 (m, 1H), 2.44 (s, 3H), 2.35 (s, 3H), 2.31-2.23 (m, 1H), 1.75-1.41 (m, 5H), 1.55-1.47 (m, 2H), 1.39-1.20 (m, 5H), 0.92 (s, 9H).

### Example 27

### Synthesis of PROTAC Compound 47

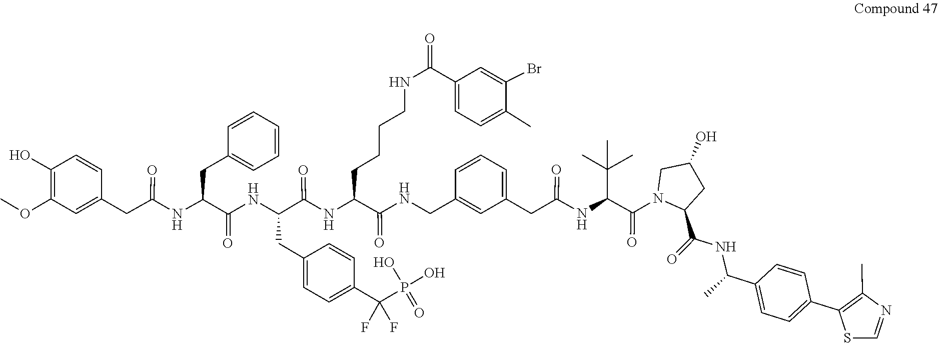 [SMILES: COc1cc(CC(=O)N[C@@H](Cc2ccccc2)C(=O)N[C@@H](Cc2ccc(C(F)(F)P(=O)(O)O)cc2)C(=O)N[C@@H](CCCCNC(=O)c2ccc(C)c(Br)c2)C(=O)NCc2cccc(CC(=O)N[C@H](C(=O)N3C[C@H](O)C[C@H]3C(=O)N[C@@H](C)c3ccc(-c4scnc4C)cc3)C(C)(C)C)c2)ccc1O]

### ((4-((S)-3-(((S)-6-(3-bromo-4-methylbenzamido)-1-((3-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethyl)benzyl)amino)-1-oxohexan-2-yl)amino)-2-((S)-2-(2-(4-hydroxy-3-methoxyphenyl)acetamido)-3-phenylpropanamido)-3-oxopropyl)phenyl)difluoromethyl)phosphonic Acid (Compound 47)

¹H NMR (500 MHz, DMSO) δ 8.99 (s, 1H), 8.51 (t, J=5.2 Hz, 1H), 8.41 (dd, J=26.6, 6.9 Hz, 2H), 8.23-7.96 (m, 5H), 7.74 (d, J=8.0 Hz, 1H), 7.44-7.35 (m, 6H), 7.32 (d, J=8.1 Hz, 2H), 7.23-7.15 (m, 2H), 7.15-7.10 (m, 6H), 7.07 (d, J=7.9 Hz, 2H), 6.65 (s, 1H), 6.57 (d, J=7.9 Hz, 1H), 6.39 (d, J=7.9 Hz, 1H), 4.90 (p, J=7.2 Hz, 1H), 4.62-4.53 (m, 1H), 4.50-4.38 (m, 3H), 4.32-4.17 (m, 4H), 3.65-3.54 (m, 6H), 3.45-3.39 (m, 1H), 3.29-3.12 (m, 5H), 3.08 (d, J=12.3 Hz, 1H), 2.95 (dd, J=14.0.4.0 Hz, 1H), 2.89-2.80 (m, 1H), 2.70 (dt, J=13.9, 6.3 Hz, 1H), 2.43 (s, 3H), 2.34 (s, 3H), 2.03-1.95 (m, 1H), 1.80-1.68 (m, 2H), 1.60 (d, J=9.8 Hz, 1H), 1.52-1.44 (m, 2H), 1.39-1.18 (m, 5H), 0.89 (s, 9H). ¹³C NMR (126 MHz, DMSO) δ 171.91 (2C), 171.77, 171.07 (2C), 170.30, 169.88, 164.92, 158.36, 152.05, 147.62, 145.43, 145.14, 140.89, 140.43, 139.56, 138.22, 136.99, 134.60, 131.38, 131.13, 130.16, 129.64 (2C), 129.53, 129.30 (3C), 128.66, 128.57, 128.33 (2C), 127.98, 127.63, 127.16, 126.96, 126.69, 126.84 (3C), 126.57, 126.28, 125.40, 124.47, 121.68, 115.57, 113.61, 69.23, 59.04, 57.04, 56.76, 55.90, 54.10, 54.08, 53.20, 49.06, 48.17, 42.49, 42.17, 42.04, 38.19, 37.74, 37.52, 35.87, 32.48, 29.24, 26.86 (3C), 23.36, 22.90, 22.81, 16.44. LC/MS m/z calculated [M−H]⁻ 1504.47. found 1504.55.

### Example 28

### FP-Based Library Screening

### Screening of Library 1

The library screening assay was carried out on a Tecan Genesis workstation with a 96-channel tip block with fixed tips. Before the screening, the library compounds were diluted from the DMSO stock solution into 3,3-dimethylglutarate buffer (50 mM 3,3-dimethylglutarate buffer, pH 7.0, containing 1 mM EDTA with an ionic strength of 0.15 M adjusted by addition of NaCl), resulting in a set of six daughter plates with an ~75 nM concentration of each compound in each well. In the first screen, PTP1B or TCPTP (2 μM in 50 mM 3,3-dimethylglutarate buffer, 50 μL, pH 7.0, containing 1 mM EDTA with an ionic strength of 0.15 M adjusted by addition of NaCl) was dispensed into each well of a 384-well plate, and then 2 μL of the fluorescein-tagged library compounds were transferred from four 96-well intermediate plates to the 384-well plate (final compound concentration ~3 nM). The fluorescence polarization values (A1) were recorded on an Envision 2021 Multilabel Microplate Reader (Perkin-Elmer). In the second screen, 50 μL of a mixture of 2 μM PTP1B or TCPTP and 500 μM Fmoc-F2Pmp-OH (as a competitive ligand) in 3,3-dimethylglutarate buffer (50 mM 3,3-dimethylglutarate buffer, pH 7.0, containing 1 mM EDTA with an ionic strength of 0.15 M adjusted by addition of NaCl) were dispensed into each well of another 384-well plate, followed by the addition of 2 μL of the fluorescein tagged library compounds (75 nM in 3,3-dimethylglutarate buffer, pH 7.0, containing 1 mM EDTA with an ionic strength of 0.15 M adjusted by addition of NaCl). The fluorescence polarization values (A2) were again measured. A displacement percentage was calculated for each library compound as (A1−A2)/(A1−A0)×100%, where A1 and A2 are the fluorescence anisotropy values of each sample as described, and A0 is the fluorescence anisotropy of free library compounds in 3,3-dimethylglutarate buffer. To simplify the calculation, A0 was set to 30. The binding affinity ranking of each compound was determined on the displacement percentage: the smaller the displacement percentage, the higher the binding affinity. The best hits were selected based on affinity and are listed in Table 1.

Table 1 shows top 5 hits from library 1 (compounds show high and comparable binding affinity on PTP1B and TC-PTP were selected).^{a}

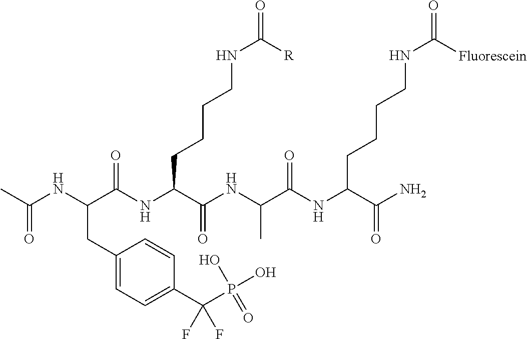

| | | Displacement %, | Displacement %, | |
|---|---|---|---|---|
| Acid Name | Acid Structure | PTP1B | TC-PTP | Rank |
| 3-bromo-4- methylbenzoic acid | 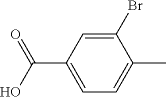 | 34 | 39 | 1 |
| 3-methoxy-4- nitrobenzoic acid | 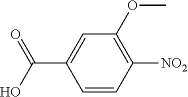 | 41 | 40 | 2 |
| 4-fluorobenzoic acid | 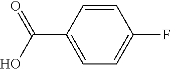 | 44 | 46 | 3 |
| 3-thiophenecarboxylic acid | 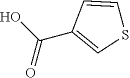 | 45 | 51 | 4 |
| 3-fluoro-4- methoxybenzoic acid | 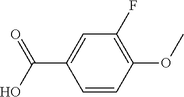 | 57 | 54 | 5 |

^{a}The displacement percentage was calculated for each library compound as (A1 − A2)/(A1 − A0) × 100%.
In this equation, A1 and A2 are the fluorescence polarization values generated by the binding between library compound and target enzyme in the absence and presence of the competitive ligand (Fmoc-F2Pmp-OH), respectively. A0 is the fluorescence anisotropy of free library compounds. The binding affinity ranking of each compound was determined on the displacement percentage: the smaller the displacement percentage, the higher the binding affinity.

### Screening of Library 2.

The library was screened using the same protocol as the library 1, except that 0.5 μM PTP1BB/TC-PTP and 1.5 mM competitive ligand Fmoc-F2Pmp-OH were used. The best hits were selected based on affinity and are listed in Table 2.

Table 2 shows top 5 Hits from library 2 (compounds show high and comparable binding affinity on PTP1B and TC-PTP were selected).^{a}

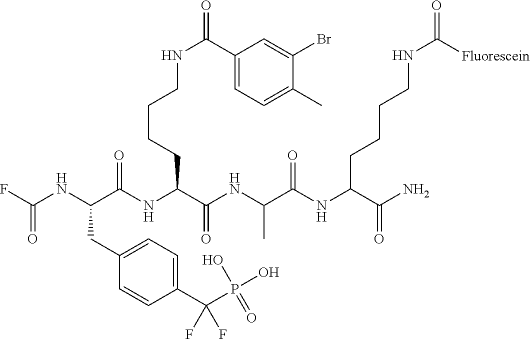

| | | Displacement %, | Displacement %, | |
|---|---|---|---|---|
| Acid Name | Acid Structure | PTP1B | TC-PTP | Rank |
| acetyl-L-phenylalanine | 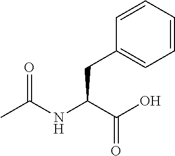 | 37% | 41% | 1 |
| (ethoxycarbonyl)-L- phenylalanine | 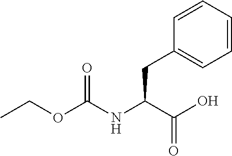 [SMILES: CCOC(=O)N[C@@H](Cc1ccccc1)C(=O)O] | 44% | 41% | 2 |
| (S)-2-acetamido-3-(4- fluorophenyl)propanoic acid | 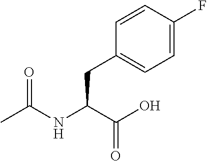 [SMILES: CC(=O)N[C@@H](Cc1ccc(F)cc1)C(=O)O] | 45% | 45% | 3 |
| 3-(p-tolyl)propanoic acid | 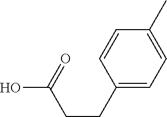 | 47% | 53% | 4 |
| 2-(4-chloro-2- methylphenoxy)acetic acid | 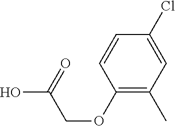 | 57% | 55% | 5 |

^{a}The displacement percentage was calculated for each library compound as (A1 − A2)/(A1 − A0) × 100%.
In this equation, A1 and A2 are the fluorescence polarization values generated by the binding between library compound and target enzyme in the absence and presence of the competitive ligand (Fmoc-F2Pmp-OH), respectively. A0 is the fluorescence anisotropy of free library compounds. The binding affinity ranking of each compound was determined on the displacement percentage: the smaller the displacement percentage, the higher the binding affinity.

### Screening of Library 3

The library was screened using the same protocol as the library 1, except that 0.5 μM PTP1B/TC-PTP and 5 μM ((4-((S)-2-((S)-2-acetamido-3-phenylpropanamido)-3-(((S)-1-amino-6-(3-bromo-4-methylbenzamido)-1-oxohexan-2-yl)amino)-3-oxopropyl)phenyl)difluoromethyl)phosphonic acid (as a competitor) were used. The best hits were selected based on affinity and are listed in Table 3.

Table 3 shows top 5 hits from library 3 (compounds show high and comparable binding affinity on PTP1B and TC-PTP were selected).^{a}

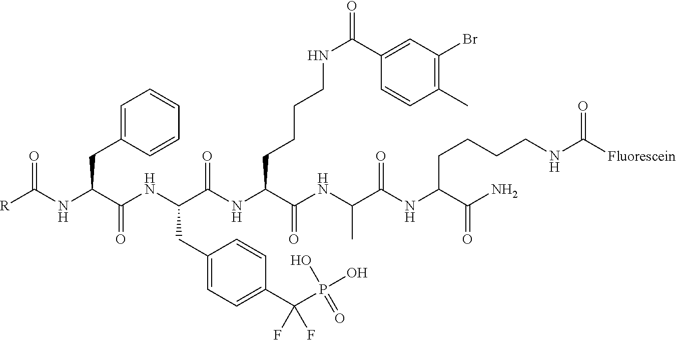

| | | Displacement %, | Displacement %, | |
|---|---|---|---|---|
| Acid Name | Acid Structure | PTP1B | TC-PTP | Rank |
| 2-(4-hydroxy-3- methoxyphenyl)acetic acid | 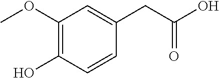 | 51 | 54 | 1 |
| 2-(4- hydroxyphenyl)acetic acid | 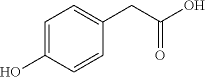 [SMILES: O=C(O)Cc1ccc(O)cc1] | 66 | 62 | 2 |
| 2-(3-fluoro-4- hydroxyphenyl)acetic acid | 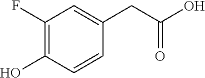 [SMILES: O=C(O)Cc1ccc(O)c(F)c1] | 72 | 75 | 3 |
| 2-(4-bromophenyl)acetic acid | 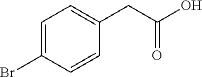 [SMILES: O=C(O)Cc1ccc(Br)cc1] | 77 | 77 | 4 |
| 2-(4- (dimethylamino)phenyl) acetic acid | 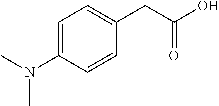 | 81 | 79 | 5 |

^{a}The displacement percentage was calculated for each library compound as (A1 − A2)/(A1 − A0) × 100%.
In this equation, Al and A2 are the fluorescence polarization values generated by the binding between library compound and target enzyme in the absence and presence of the competitive ligand (Fmoc-F2Pmp-OH), respectively. A0 is the fluorescence anisotropy of free library compounds. The binding affinity ranking of each compound was determined on the displacement percentage: the smaller the displacement percentage, the higher the binding affinity.

### Example 29

Determination of Inhibition Constant (Kᵢ) and IC₅₀ Value

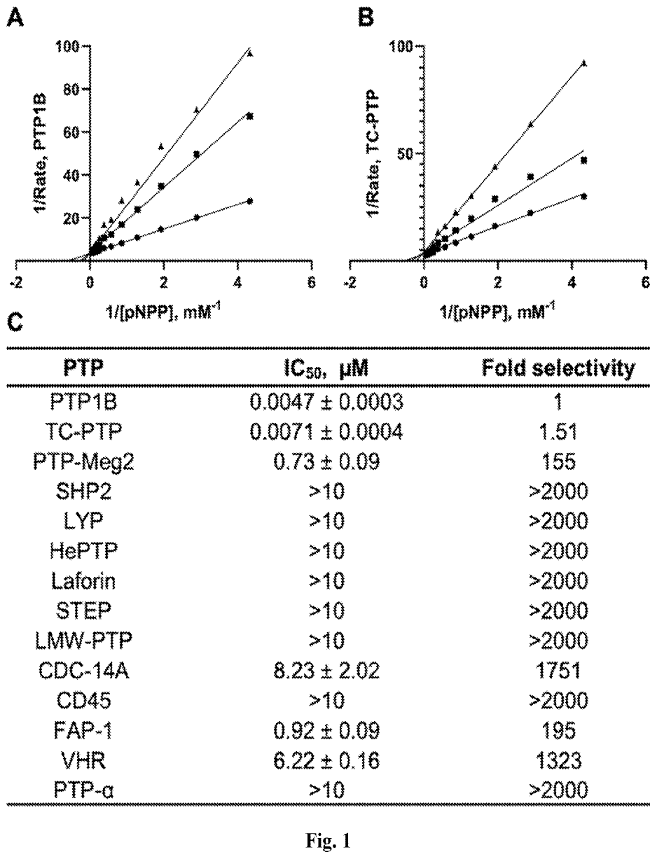

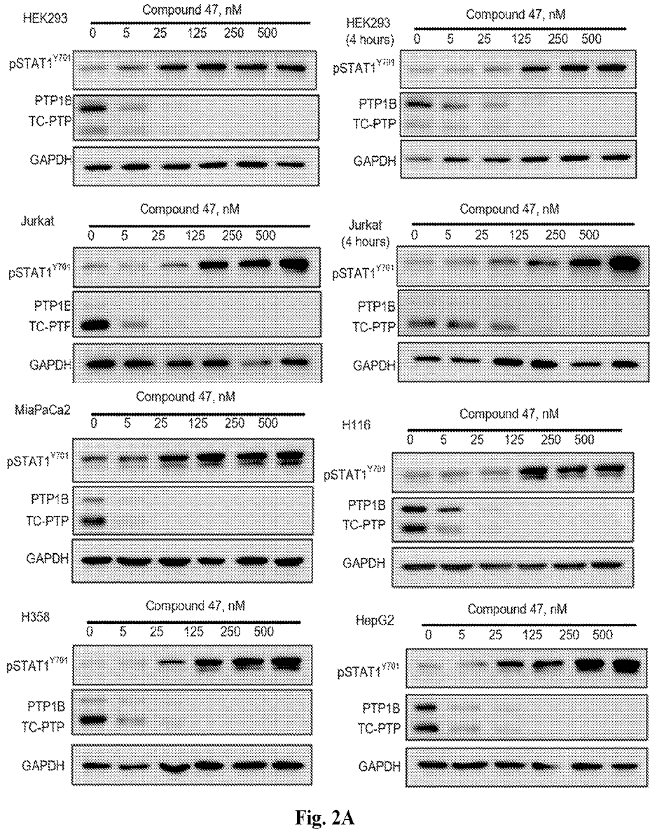

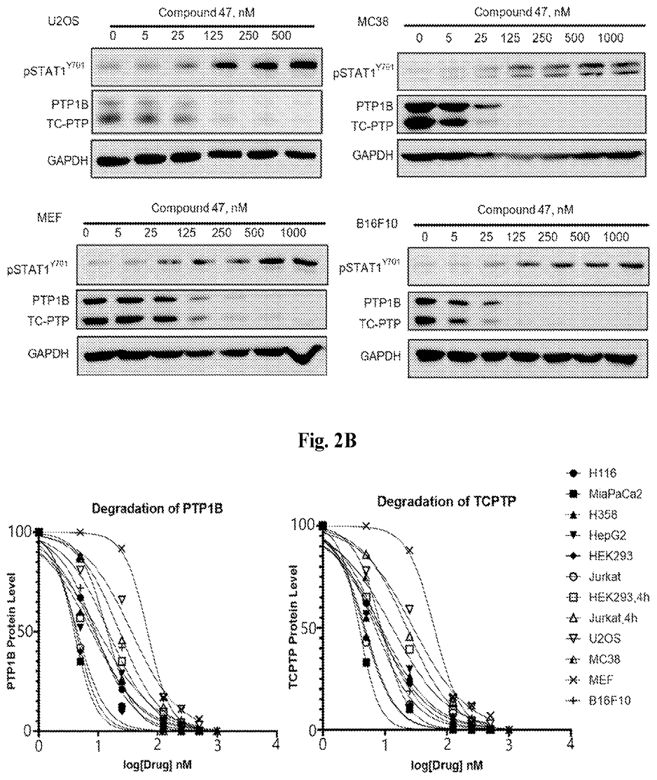

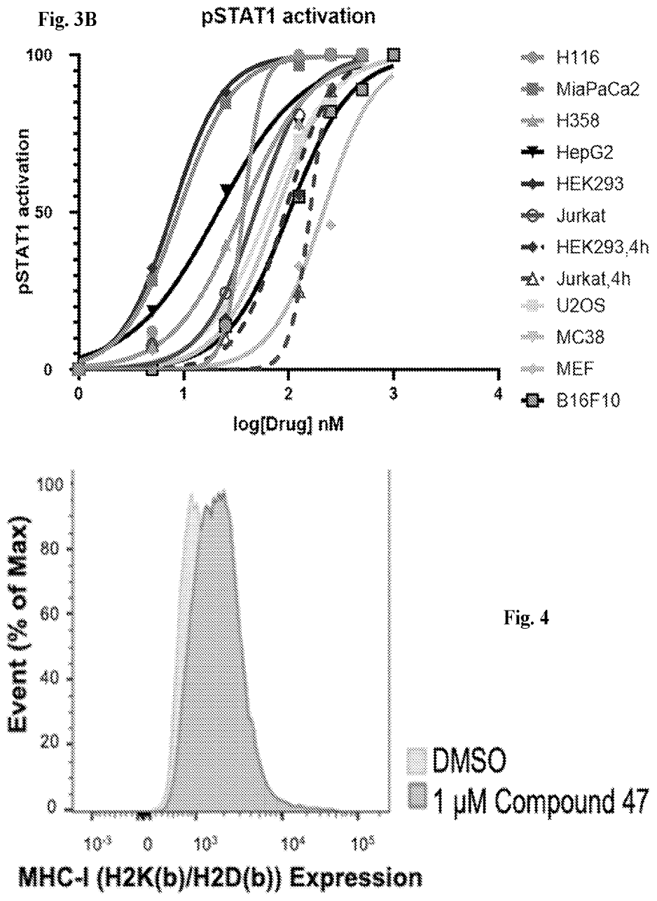

PTP activity was assayed using p-nitrophenyl phosphate (pNPP) as a substrate in DMG buffer (50 mM DMG. pH 7.0, 1 mM EDTA, 150 mM NaCl, 2 mM DTT, 0.1 mg/mL BSA) at 25° C. The assays were performed in 96-well plates. Normally, to determine the IC₅₀ values, the reaction was initiated by the addition of enzyme (for PTP1B and TCPTP, the final concentration was 0.4 nM, for other PTPs, the final concentration was 10 nM) to a reaction mixture (0.2 mL) containing 2 mM (Km for the substrate) pNPP with various concentrations of inhibitors (see FIG. 1). The reaction rate was measured using a SpectraMax Plus 384 Microplate Spectrophotometer (Molecular Devices). Data were fitted using SigmaPlot Enzyme Kinetics Module (Systat Software, Inc.). To determine the mode of inhibition, the reactions were initiated by the addition of enzyme (final concentration for TC-PTP and PTP1B was 0.4 nM) to the reaction mixtures (0.2 mL) containing various concentrations of pNPP with different concentrations of the inhibitor. Data were fitted using SigmaPlot Enzyme Kinetics Module (Systat Software. Inc.).

### Example 30

### Cell Culture

HEK293. MIAPaCa-2, HepG2, U2OS, H116, MEF, B16F10, and MC38 cells, were grown in DMEM, while Jurkat and H358 cells were grown in RPMI 1640 supplemented with 10% fetal bovine serum, penicillin (50 units/mL), and streptomycin (50 μg/mL) in a 37° C. incubator containing 5% CO₂. 20 ng/ml human (Biolegend #713906) or mouse (Biolegend #714006) Interferon-γ were used for JAK/STAT pathway stimulation.

### Immunoblotting

Tissues or cultured cells were lysed with ice-cold lysis buffer (50 mM Tris (pH 8.0, 150 mM NaCl, 10% Glycerol, 1% Triton-X-100) supplied with phosphatase inhibitor (Bimake) and protease inhibitor mixture (Roche Applied Science). Equal amounts of protein were resolved by SDS-PAGE, transferred to nitrocellulose membrane, and subjected to immunoblotting. Antibodies used in this study include anti-following proteins: pSTAT1^{Tyr701} (Cell Signaling Technology #9167, 1:3000), GAPDH (Cell Signaling Technology #97166, 1:5000), pJAK1^{Tyr1034/1035} (Cell Signaling Technology #74129, 1:1000), pJAK2^{Tyr1007/1008} (Cell Signaling Technology #3776, 1:1000), TC-PTP (Abcam #ab180764, 1:3000). PTP1B (Abcam #ab244207, 1:1500).

### Evaluation of Degradation Efficiency.

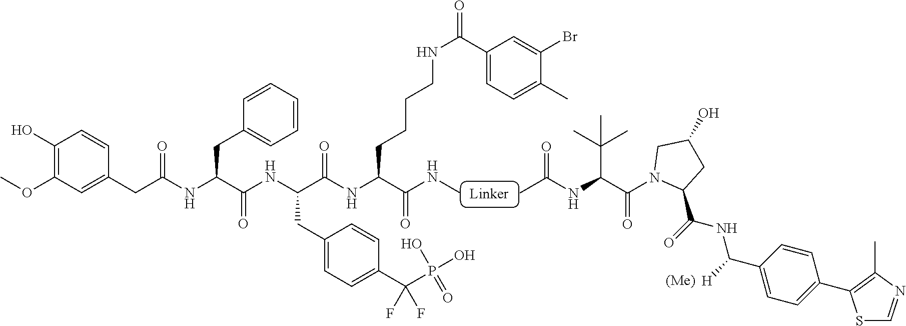

Table 4 shows structure and degradation results of an initial set of degraders. Degradation assay was conducted in HEK293 cells with 1 μM degrader and 24 hours incubation.

| | | % Protein Degradation | |
|---|---|---|---|
| Compound | Linker Structure | PTP1B | TC-PTP |
| DMSO | N/A | 0 | 0 |
| Example 7 | N/A | 0 | 0 |
| 6 | 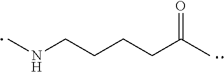 | 53 | 58 |
| 8 | 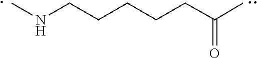 | 81 | 83 |
| 10 | 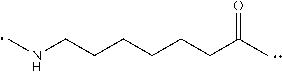 | 61 | 69 |
| 83 | 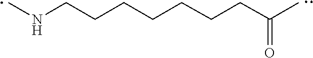 | 58 | 66 |
| 14 | 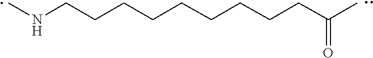 | 38 | 62 |
| 18 | 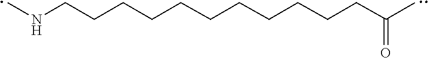 | 29 | 30 |

Table 5 shows the structure and degradation results of the second set of degraders. The degradation assay was conducted in HEK293 cells with 0.2 μM degrader and 16 hours incubation.

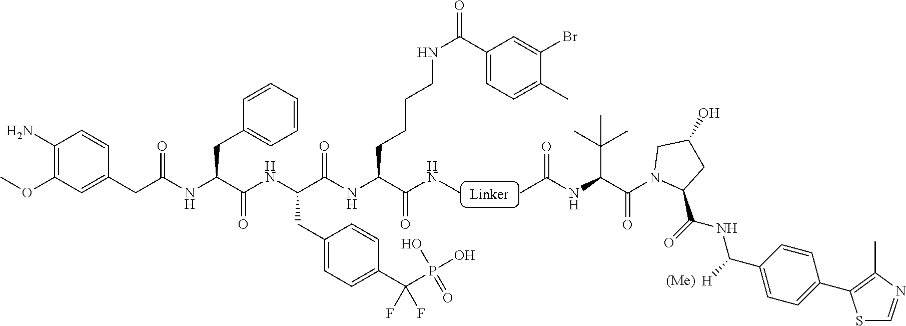

| | | | % Protein Degradation | |
|---|---|---|---|---|
| Compound | Linker Structure | E3 Ligand | PTP1B | TC-PTP |
| DMSO | N/A | N/A | 0 | 0 |
| Example 7 | N/A | N/A | 0 | 0 |
| 8 | 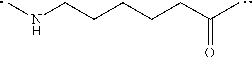 | (S,R,S)-AHPC | 61 | 58 |
| 28 | 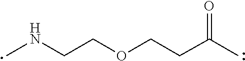 | (S,R,S)-AHPC | 45 | 47 |
| 27 | 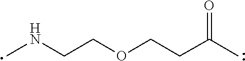 | (S,R,S)-AHPC- Me | 75 | 63 |
| 40 | 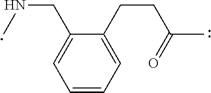 | (S,R,S)-AHPC | 16 | 0 |
| 48 | 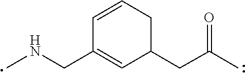 | (S,R,S)-AHPC | 75 | 70 |
| 54 | 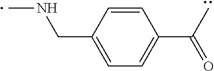 | (S,R,S)-AHPC | 24 | 37 |
| 58 | 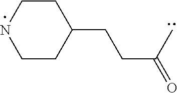 | (S,R,S)-AHPC | 26 | 32 |
| 56 | 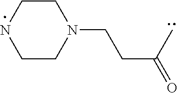 | (S,R,S)-AHPC | 30 | 14 |
| 47 | 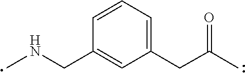 | (S,R,S)-AHPC- Me | 95 | 92 |

### Degradation Mechanism Verification

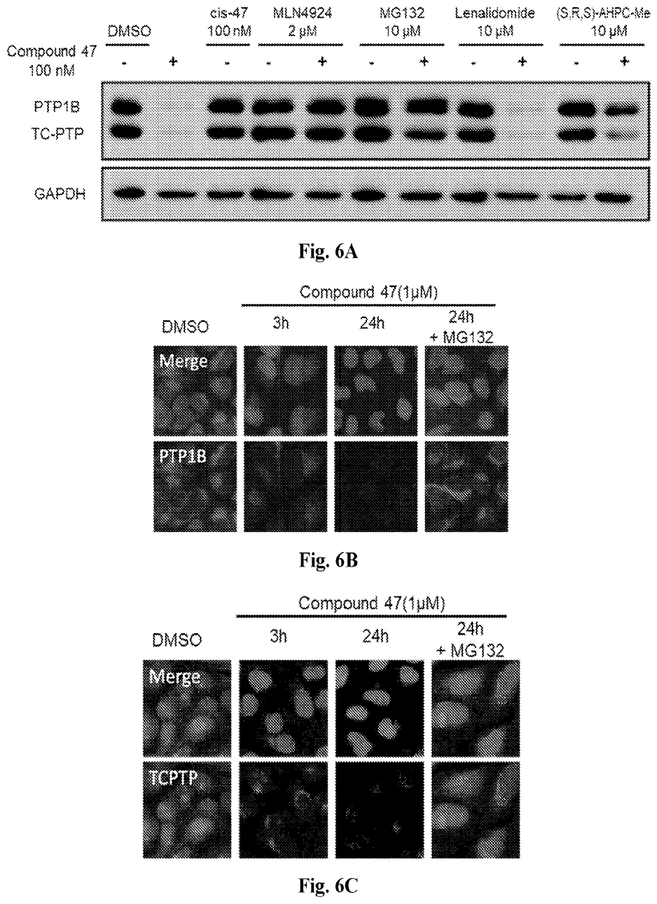

PROTAC-mediated protein degradation requires the obligatory formation of afunctional ternary complex of target protein-PROTAC-E3 ligase to enable target protein ubiquitination by the E3 ligase, followed by proteasomal degradation. The VHL E3 ligase dependency for compound 47 induced PTP1B and TC-PTP degradation was confirmed by preparing cis-isomer of compound 47 (cis-47) in which the VHL E3 ligand was replaced with (S,S,S)-AHPC-Me, an epimer of (S,R,S)-AHPC-Me with diminished affinity for VHL. It was established that the IC₅₀ values of cis-47 for PTP1B and TC-PTP (25.1±1.6 and 29.7±2.1 nM) are similar to those of compound 47. Consistent with the impaired binding of (S,S,S)-AHPC-Me to VHL, cis-47 failed to degrade either PTP1B or TC-PTP in HEK293 cells (FIG. 6A). Moreover, the addition of the VHL ligand (S,R,S)-AHPC-Me reduced the compound 47-mediated degradation of the two proteins (FIG. 6A). In contrast, pretreatment of the cell with lenalidomide, a ligand for cereblon (another E3 ligase commonly employed for PROTAC development), had no effect on compound 47-mediated PTP1B and TC-PTP degradation (FIG. 6A). These observations suggest that compound 47-induced PTP1B and TC-PTP degradation is VHL-dependent. The ubiquitination and proteasome dependency of the compound 47-induced PTP1B and TC-PTP degradation was demonstrated by determining the effect of MLN-4924, an inhibitor of the E1 ubiquitin-activating enzyme, and the proteasome inhibitor MG-132. Pretreatment of the cells for 30 minutes with these inhibitors markedly reduced the extent of PTP1B and TC-PTP degradation by compound 47, indicating that the E1 ubiquitin-activating enzyme and 26S proteasome were indeed required for the compound 47 induced PTP1B and TC-PTP degradation (FIG. 6A). The compound 47 mediated PTP1B and TC-PTP degradation and proteasome dependency were also verified by immunofluorescent imaging (FIGS. 6B and 6C). Taken together, the data demonstrated that compound 47 is a PTP1B and TC-PTP dual-PROTAC degrader with high potency and selectivity.

### Proteomic Analysis for Selectivity

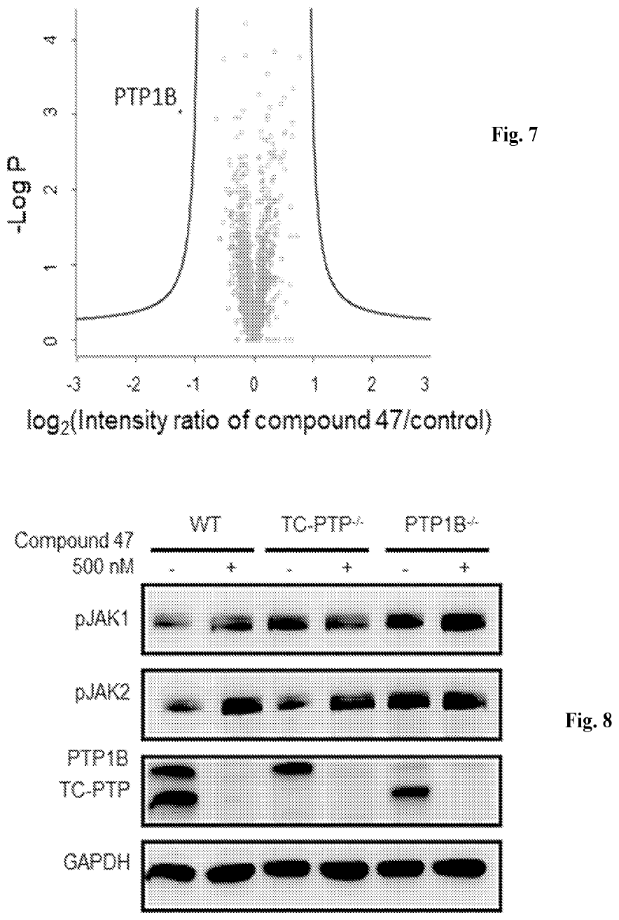

The proteome-wide degradation selectivity of compound 47 was assessed by performing quantitative mass spectrometry-based proteomic experiments to determine the range of targets degraded by compound 47 in HEK293 cells. As shown in FIG. 7, PTP1B was the only protein whose levels were significantly reduced by 100 nM of compound 47. The mass spectrometry measurements under the given conditions did not detect TC-PTP. Taken together, the above results indicated that compound 47 is a potent PTP1B and TC-PTP dual degrader with remarkably high selectivity.

### Flow Cytometry

After incubation, MC38 cells were trypsinized and washed in PBS+2% FBS, stained with Alexa Fluor® 647 anti-mouse H-2Kb/H-2Db Antibody against mouse MHC-I complex and then analyzed on a BD Fortessa LSR flow cytometry cell analyzer. Compound 47 treated MC38 cells exhibited elevated expression of MHC-I. (FIG. 4)

### In Vivo Anti-Tumor Studies

All the in vivo studies were performed under an animal protocol (1511001324) approved by the Institutional Animal Care and Use Committee of the Purdue University, in accordance with the recommendations in the guide for the care and use of laboratory animals of the National Institutes of Health.

### Pharmacokinetic Study

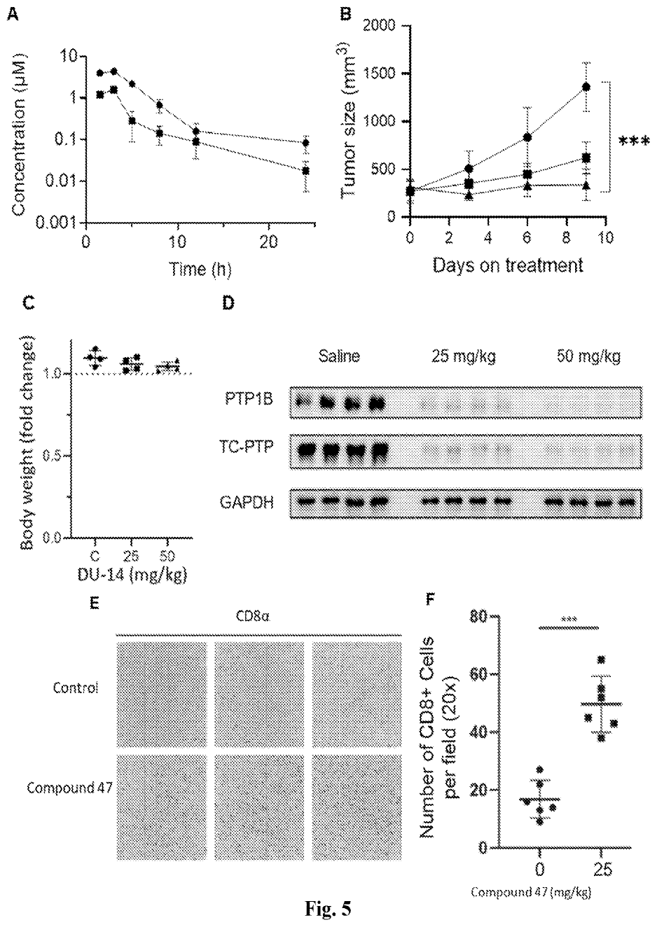

For the PK study, C57BL6 female mice (25-30 g body weight) were injected intraperitoneally with 25 or 50 mg/kg compound 47 dissolved in 0.4 ml saline. Blood samples were collected through the tail vein at indicated time points after injection. Isoflurane was used as an anesthetic. All blood samples were centrifuged at 1,500 g for 5 minutes, and plasma was separated and stored at −80° C. until analysis by a validated method based on reversed-phase liquid chromatography coupled to mass-spectrometric detection (LC/MS) using a reported procedure (Journal of Clinical Investigation, 2018, 128, 816-825), which is hereby specifically incorporated by reference for its teachings regarding same (FIG. 5A).

For MC38 syngeneic tumor study, 12 weeks C57BL6 female mice were injected subcutaneously with 10⁶ MC38 cells for tumor growth. When tumors reached an average volume of 200 mm³, mice were tumor size-matched and randomly assigned to different experimental groups for experiments. Mice were injected intraperitoneally with saline or 25 or 50 mg/kg compound 47 daily. Tumor sizes and animal weights were measured twice per week (FIGS. 5B-5C). Tumor volume (mm³)=(length×width²)/2. At the end of the experiments, mice were euthanized, and tumors were collected for analysis.

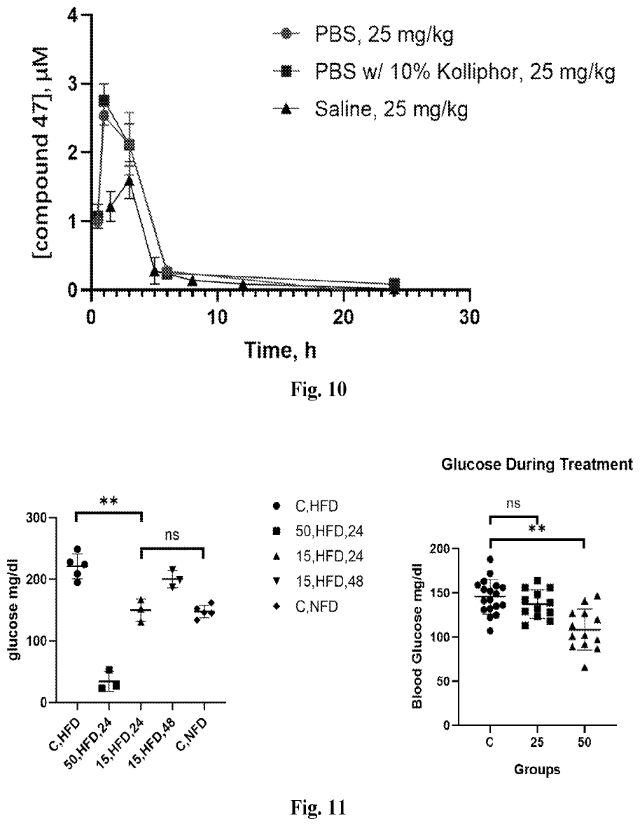

Saline was replaced by PBS buffer in the treatment of compound 47 in mice to improve PK properties (FIG. 10). Both Cₘₐₓ and area under the curve (AUC) were increased compared to the above method with saline. No significant improvement was observed with the addition of the solubilizer Kolliphor.

Compound 47 enhances IFN-γ induced JAK1/2-STAT1 pathway activation and promotes MHC-I expression in tumor cells. Compound 47 also activates CD8⁺ T-cells by augmenting STAT1 and STAT5 phosphorylation. Importantly, compound 47 induces PTP1B and TC-PTP degradation in vivo and suppresses MC38 syngeneic tumor growth by increasing CD8⁺ T-cell mediated immune response (FIGS. 5D-5F).

Biochemical IC₅₀s of the dual PROTACs are listed in Table 6. The compound of example 7 was included as a control compound.

TABLE 6

IC₅₀, nM

| Compound | TC-PTP | PTP1B |
|---|---|---|
| Example 7 | 7.1 ± 0.4 | 4.7 ± 0.3 |
| 6 | 34.3 ± 14.8 | 29.4 ± 2.1 |
| 8 | 12.8 ± 3.7 | 16.7 ± 0.9 |
| 10 | 6.8 ± 0.6 | 7.4 ± 0.6 |
| 83 | 10.6 ± 0.3 | 19.0 ± 1.9 |
| 14 | 6.8 ± 0.3 | 10.3 ± 0.6 |
| 18 | 8.9 ± 0.6 | 22.3 ± 1.1 |
| 28 | 11.2 ± 1.3 | 15.4 ± 3.3 |
| 27 | 6.7 ± 2.0 | 15.5 ± 2.1 |
| 40 | 2.9 ± 0.5 | 3.8 ± 0.4 |
| 48 | 11.5 ± 0.6 | 16.7 ± 3.3 |
| 54 | 21.3 ± 1.7 | 51.2 ± 14.4 |
| 58 | 8.0 ± 0.7 | 8.9 ± 0.5 |
| 56 | 12.0 ± 0.8 | 13.7 ± 1.9 |
| 47 | 30.1 ± 2.3 | 24.2 ± 1.7 |

TC-PTP/PTP1B KO MEF Cells for pJAK1/2 Elevation and 1B/TC Degradation.

The target engagement by compound 47 inside the cell was ascertained. As negative regulators of IFN-γ signaling, PTP1B and TC-PTP dephosphorylate JAK2 at Y1007/Y1008 and JAK1 at Y1034/Y1035, respectively. In addition, TC-PTP can also directly dephosphorylate STAT family members, including STAT1 and STAT3, in the nucleus to comprehensively attenuate IFN signaling. Treatment of MEF cells with 500 nM compound 47 for 16 hours led to the complete removal of both PTP1B and TC-PTP and augmented IFN-γ stimulated JAK2 Y1007N1008 and JAK1 Y1034/Y1035 phosphorylation (FIG. 8). The increase in pJAK1/Y1034/Y1035 and pJAK2Y1007/Y1008 caused by PTP1B/TC-PTP degradation was demonstrated by utilizing MEF cells that either lack PTP1B or TC-PTP as negative controls. As shown in FIG. 8, compound 47 treatment of TC-PTP^{-/-} MEF cells further elevated IFN-γ-mediated PTP1B substrate JAK2/Y1007/Y1008 phosphorylation with no effect on the level of TC-PTP substrate pJAK1/Y1034/Y1035. In contrast, the addition of compound 47 to PTP1B^{-/-} MEF cells further increased the IFN-γ mediated TC-PTP substrate pJAK1/Y1034/Y1035 level with no alteration of PTP1B substrate JAK2/Y1007/Y1008 phosphorylation. These results indicate that compound 47 can block both PTP1B- and TC-PTP-catalyzed substrate dephosphorylation.

Compound 47 Induces PTP1B and TC-PTP Degradation in CD8⁺ Naïve T-Cells, Enhances STAT1 and STAT5 Phosphorylation, and Promotes CD8⁺ T-Cell Activation.

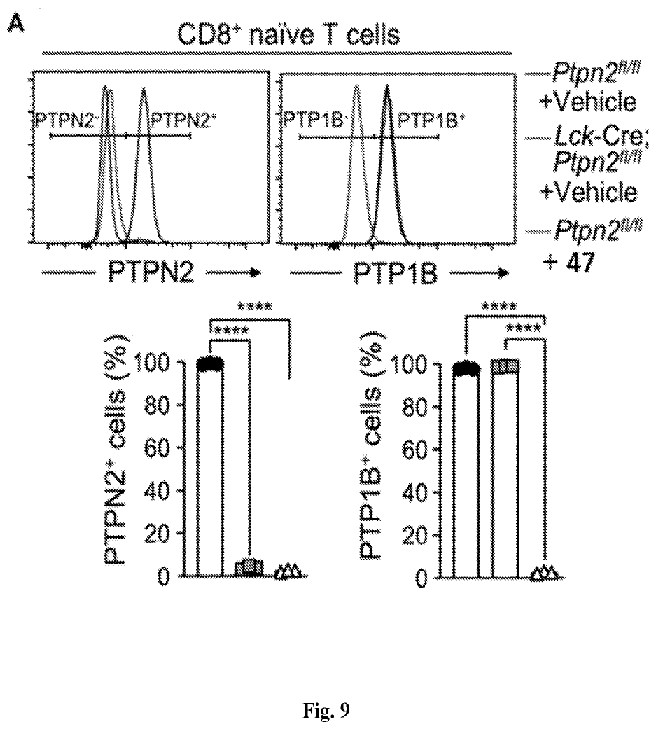

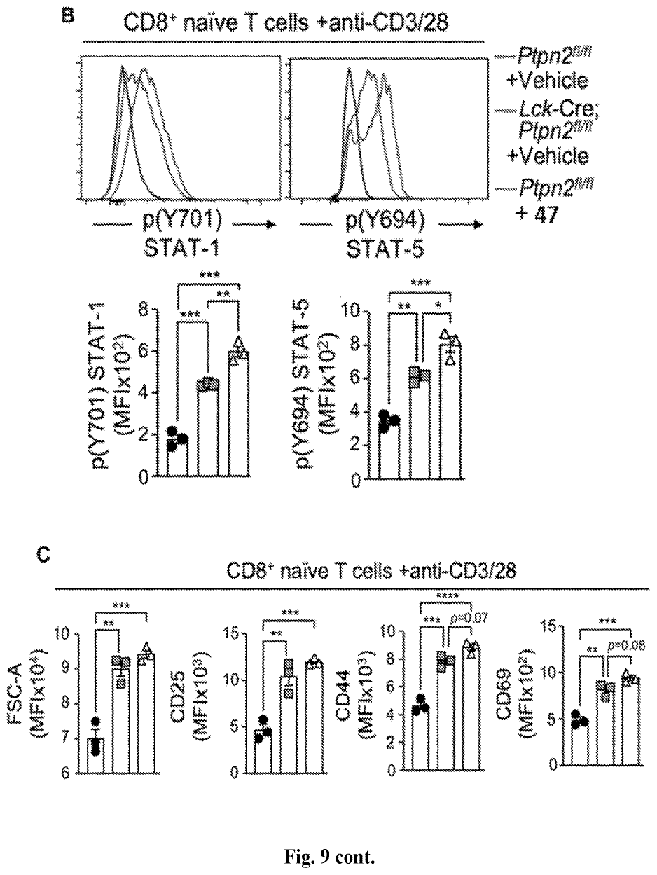

Beyond attenuating JAK/STAT signaling in tumor cells, PTP1B and TC-PTP also have fundamental roles in T-cells, and the deletion of either TC-PTP or PTP1B in T-cells can markedly enhance anti-tumor immunity. TC-PTP attenuates T-cell receptor (TCR) signaling by dephosphorylating and inactivating the Src family kinase LCK. TC-PTP attenuates JAK/STAT1/5 signaling in response to cytokines such as IFNs and IL-2, which are required for the activation, clonal expansion, and differentiation of T-cells. The deletion of TC-PTP in T-cells enhances immunosurveillance and inhibits the growth of syngeneic tumors in mice and the anti-tumor efficacy of adoptively transferred T-cells. PTP1B also negatively regulates IL-2-induced JAK/STAT5 signaling in T-cells, and its deletion or inhibition in vivo can enhance the anti-tumor activity of T-cells. Accordingly, the impact of targeting PTP1B and TC-PTP with compound 47 on JAK/STAT signaling and the activation of T-cells after crosslinking the TCR was assessed. The effects of compound 47 were compared with the genetic deletion of TC-PTP (encoded by Ptpn2) in T-cells (Lck-Cre; Ptpn2^{fl/fl}). First, the impact of compound 47 on TC-PTP and PTP1B protein levels was assessed by flow cytometry using validated antibodies. After 48 hr of treatment, TC-PTP and PTP1B were effectively degraded in T-cells (FIG. 9A). The degradation of TC-PTP and PTP1B was accompanied by a more than 3-fold increase in STAT1 Y701 phosphorylation and a 2-fold increase in STAT5 Y694 phosphorylation detected after crosslinking the TCR with α-CD3/α-CD28 to activate T-cells (FIG. 9B). Importantly, the promotion of pSTAT1/Y701 and pSTAT5/Y694 by compound 47 treatment exceeded that achieved by the genetic deletion of TC-PTP, consistent with compound 47 targeting both TC-PTP and PTP1B to enhance signaling. Furthermore, compound 47 treatment also enhanced the TCR-induced activation of T-cells, as assessed by monitoring for cell size and the expression of cell surface activation markers, including CD44. CD25 (IL-2 receptor a), and CD69 (FIG. 9C). In this instance, compound 47 only moderately, albeit not significantly, increased T-cell activation beyond that achieved by the deletion of TC-PTP. This was not necessarily a surprise since TC-PTP, but not PTP1B, attenuates TCR signaling in naive CD8⁺ T-cells. Nonetheless, these results demonstrate that the combinatorial targeting of PTP1B and TC-PTP with compound 47 can enhance JAK/STAT signaling and the activation of CD8⁺ T-cells that are instrumental in anti-viral and anti-tumor immunity.

### Blood Glucose Levels Study—Implication for Diabetes and Obesity

PTP1B and TC-PTP are known to function in concert in regulating both insulin and leptin-mediated cellular processes. The inhibition of PTP1B and TC-PTP was reported to improve insulin sensitivity and glucose homeostasis in diet-induced obese mice. The effect of compound 47 on the glucose levels in mice was evaluated. The results showed that 15 mg/kg of compound 47 was sufficient to reduce blood glucose level in high-fat diet (HFD) mice to that of normal fat diet fed (NFD) mice, and treatment with 50 mg/kg of compound 47 led to further reduced glucose levels (FIG. 11). In the MC38 synergetic tumor mouse model, 25 mg/kg of compound 47 did not lead to significant reduction in the level of blood glucose, while 50 mg/kg slightly reduced blood glucose level (FIG. 11), suggesting glucose level reduction by compound 47 is minimal in mice with healthy blood glucose levels.

### Comparison of Compound 47 to TC-PTP Inhibitor ABBV-CLS-484 for STAT1 Phosphorylation

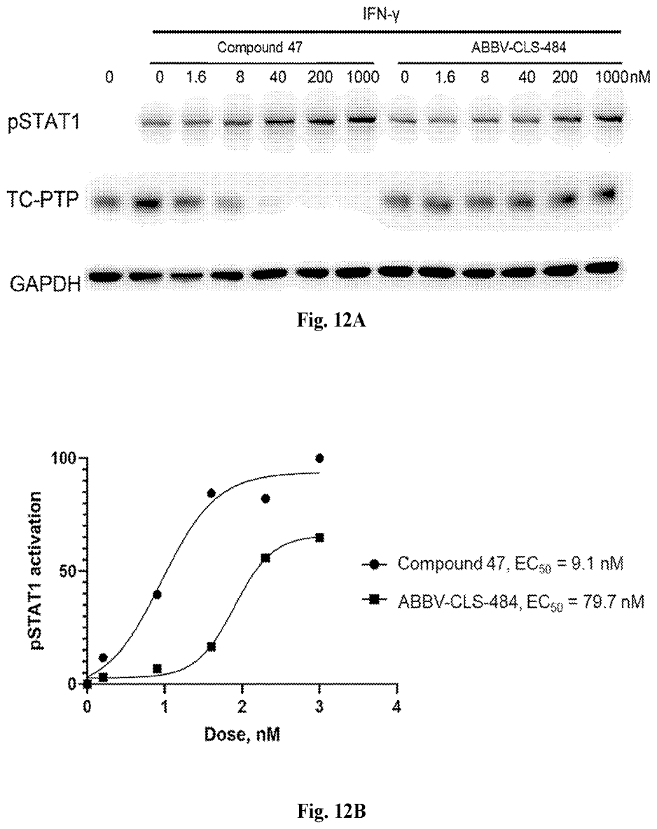

Abbvie and Calico developed a potent PTP1B/TC-PTP inhibitor ABBV-CLS-484 (disclosed in International PCT application WO2019246513), and it is currently being evaluated as a treatment for locally advanced or metastatic tumors (NCT04777994). The effectiveness of compound 47 was demonstrated by comparing its efficiency on the activation of STAT1 phosphorylation with ABBV-CLS-484 (FIG. 12A). In HEK293 cells, compound 47 dose-dependently induced degradation of TC-PTP, and activated pSTAT1 with an EC₅₀ of 9.1 nM, which is superior to that of ABBV-CLS-484 (79.7 nM).

As used herein, the following terms and phrases shall have the meanings set forth below. Unless defined otherwise, all technical and scientific terms used herein have the same meaning as commonly understood to one of ordinary skill in the art.

The term “about” can allow for a degree of variability in a value or range, for example, within 10%, within 5%, or within 1% of a stated value or of a stated limit of a range.

The term “substantially” can allow for a degree of variability in a value or range, for example, within 90%, within 95%, or within 99% of a stated value or of a stated limit of a range.

The terms “a,” “an,” or “the” are used to include one or more than one unless the context clearly dictates otherwise. The term “or” is used to refer to a nonexclusive “or” unless otherwise indicated. In addition, the phraseology or terminology employed herein, and not otherwise defined, is for the purpose of description only and not of limitation. Any use of section headings is intended to aid the reading of the document and is not to be interpreted as limiting. Further, information that is relevant to a section heading may occur within or outside of that particular section. The terms “including” and “having” are defined as comprising (i.e., open language).

All patents, patent application publications, journal articles, textbooks, and other publications mentioned in the specification are indicative of the level of skill of those in the art to which the disclosure pertains. All such publications are incorporated herein by reference to the same extent as if each individual publication were specifically and individually indicated to be incorporated by reference.

1. A compound of formula (I), or a pharmaceutically acceptable salt, hydrate, tautomer, or stereoisomer thereof: 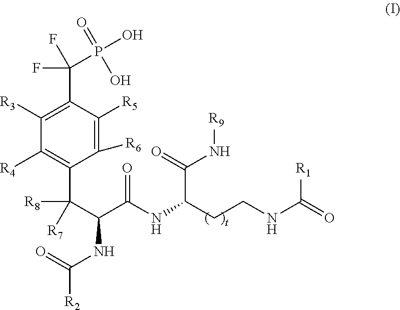
wherein
each R₁ and R₂ independently is a residue of a carboxylic acid, or a pharmaceutically acceptable salt thereof, wherein R₁ and R₂ can be the same or different;
R₃, R₄, R₅, R₆, R₇, and R₈ each independently are selected from the group consisting of hydrogen, deuterium, halogen, hydroxy, C₁-C₆ alkyl, C₃-C₆ cycloalkyl, —C₁-C₆ alkylene, 4-6 membered heterocyclyl, and —C₁-C₆ alkylene-4-6 membered heterocyclyl, wherein C₁-C₆ alkyl, C₃-C₆cycloalkyl, and C₁-C₆ alkylene-4-6 membered heterocyclyl group optionally substituted on one or more available carbons by one or more substituents each independently selected from deuterium, halogen, hydroxy, C═O, C₁-C₆ alkyl, C₃-C₆ alkenyl, C₃-C₆ alkynyl, C₃-C₆ cycloalkyl, —C₁-C₆-alkylene-C₃-C₆ cycloalkyl, C₁-C₆ alkyl-S(O)₂—, C₃-C₆ cycloalkyl-S(O)₂—, C₁-C₆ alkyl-C(O)—, C₁-C₆ alkoxy-C(O)—, —NH—C(O)—Rₐ, and —C(O)—NH—Rₐ, wherein Rₐ is C₁-C₆ alkyl, C₃-C₆ cycloalkyl and —C₁-C₆ alkylene-4-6 membered heterocyclyl optionally substituted on one or more available carbons by one, or more substituents each independently selected from deuterium, halogen, hydroxy, C═O, C₁-C₆ alkyl, C₃-C₆ alkenyl, C₃-C₆ alkynyl, C₃-C₆ cycloalkyl, —C₁-C₆-alkylene-C₃-C₆ cycloalkyl, C₁-C₆ alkyl-S(O)₂—, C₁-C₆ cycloalkyl-S(O)₂—, C₁-C₆ alkyl-C(O)—, C₁-C₆ alkoxy-C(O)—, —NH—C(O)—R_{b}, and —C(O)—NH—R_{b}, wherein R_{b} is independently selected from deuterium, halogen, hydroxy, C═O, C₁-C₆ alkyl, C₃-C₆ alkenyl, C₃-C₆ alkynyl, C₃-C₆ cycloalkyl, —C₁-C₆-alkylene-C₃-C₆ cycloalkyl, C₁-C₆ alkyl-S(O)₂—, C₃-C₆ cycloalkyl-S(O)₂—, C₁-C₆ alkyl-C(O)—, and C₁-C₆ alkoxy-C(O)—;
t is 0 to 6;
R₉ is H or a group represented by formula L-B, wherein L is a linker selected from the group consisting of: 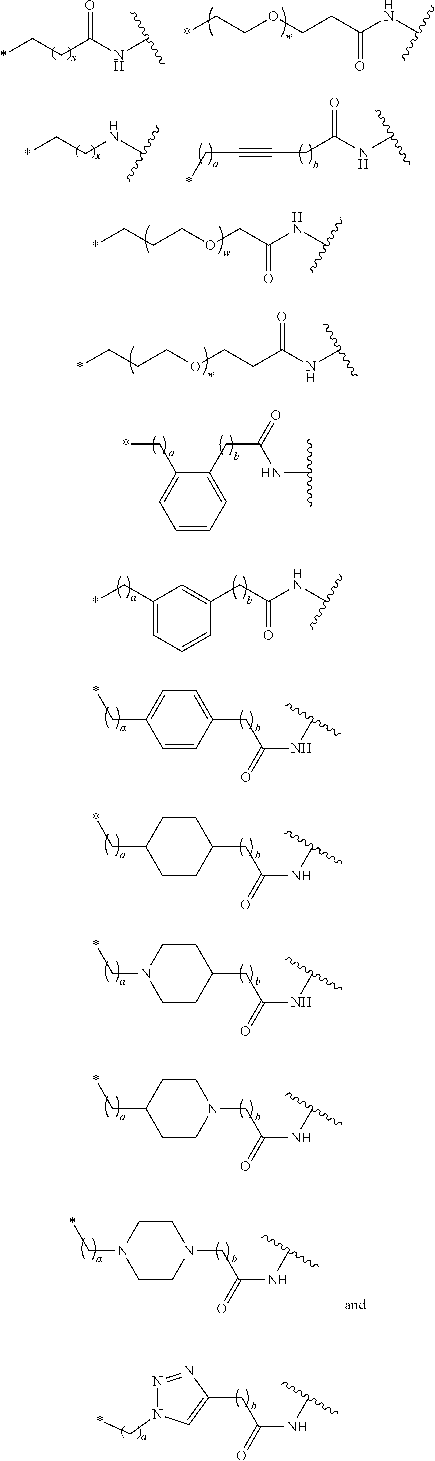
wherein w is 1-5, x is 1-15, and each a and b is independently 0-3;
the bond designated with an “ [symbol] ” is attached to B;
the bond designated with “*” represents the point of attachment of R₉; and
B is selected from the group B₁, B₂, and B₃, wherein B₁ is represented by a structure: 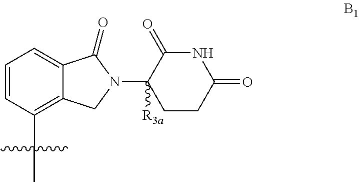
wherein R₃ₐ is fluoro, hydrogen, or deuterium;
B₂ is represented by a structure: 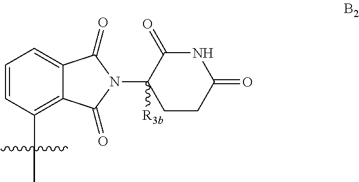
wherein R_{3b} is fluoro, hydrogen, or deuterium; and
B₃ is represented by a structure: 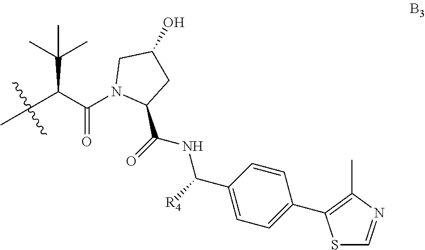
wherein R₄ is methyl, hydrogen, or deuterium.

2. The compound of claim 1, wherein the carboxylic acid is represented by a formula RCOOH, wherein R is selected from a group consisting of hydrogen, an aliphatic group, or an aromatic group, wherein the aliphatic group is saturated or unsaturated, and wherein the aliphatic group or the aromatic group is substituted with C₁-C₂₄ alkyl, C₁-C₂₄ alkenyl, C₁-C₂₄ alkynyl, haloalkyl, haloalkenyl, heteroalkyl, cycloalkyl, cycloalkenyl, heterocycloalkyl, heterocycloalkenyl, aryl, heteroaryl, cycloalkylalkyl, heterocycloalkylalkyl, arylalkyl, heteroarylalkyl, arylalkenyl, cycloalkylheteroalkyl, heterocycloalkylheteroalkyl, heteroarylheteroalkyl, arylheteroalkyl, and acyl, wherein R group is optionally substituted with at least one of the groups selected from C₁-C₂₄ alkyl, hydroxy, alkoxy, cyano, halo, nitro, aryl, amino, C₁-C₂₄ alkenyl, C₁-C₂₄ alkynyl, haloalkyl, haloalkenyl, heteroalkyl, cycloalkyl, cycloalkenyl, heterocycloalkyl, heterocycloalkenyl, aryl, heteroaryl, cycloalkylalkyl, heterocycloalkylalkyl, arylalkyl, heteroarylalkyl, arylalkenyl, cycloalkylheteroalkyl, heterocycloalkylheteroalkyl, heteroarylheteroalkyl, arylheteroalkyl, and acyl.

3. The compound of claim 1, wherein the carboxylic acid is selected from the group consisting of 3-dimethylaminobenzoic acid, 2-(2-cyanophenylthio)benzoic acid, 2-(4-chlorobenzoyl)benzoic acid, (−)-2-oxo-4-thiazolidine-carboxylic acid, (−)-N-acetylneuraminic acid, (+)-6-methoxy-α-methyl-2-naphthaleneacetic acid, (+)-carbobenzyloxy-D-proline, (+)-menthoxyacetic acid, (±)-2-(2-chlorophenoxy)propionic acid, (±)-1-methyl-2-cyclohexene-1-carboxylic acid, (1-naphthoxy)acetic acid, (IR)-(Ia,2b,3a)-(+)-3-methyl-2-nitromethyl-5-oxocyclopentaneacetic acid, (1R,4R)-7,7-dimethyl-2-oxobicyclo[2.2.1]heptane-1-carboxylic acid, (1S)-(+)-camphanic acid, (1S,3R,4S,5R)-1,3,4,5-tetrahydroxycyclohexanecarboxylic acid, (2,4-di-tert-pentylphenoxyl)acetic acid, (2-naphthoxy)acetic acid, (2-pyrimidylthio)acetic acid, (4-carboxybutyl)triphenyl-phosphonium bromide, (4-chlorophenylthio)acetic acid, (4-methylphenoxy)acetic acid, (α, α, α-trofluoro-m-tolyl)acetic acid, (E)-2-((4-hydroxyphenyl)diazenyl)benzoic acid, (E)-2-methyl-3-(2,4,5-trimethoxyphenyl)acrylic acid, (methylthio)acetic acid, (R)-(−)-2-hydroxy-4-phenylbutyric acid, (R)-(−)-3-chloromandelic acid, (R)-(−)-hexahydromandelic acid, (R)-(+)-2-pyrrolidone-5-carboxylic acid, (R)-(+)-citronellic acid, (R)-2-(1-phenylethylcarbamoyl)benzoic acid, (R)-2-hydroxy-2-phenylacetic acid, (R)-3,3,3-trifluoro-2-methoxy-2-phenylpropanoic acid, (R)-6-hydroxy-2,5,7,8-tetramethylchroman-2-carboxylic acid, (S)-(−)-indoline-2-carboxylic acid, (S)-(+)-2-oxo-4-phenyl-3-oxazolidineacetic acid, (S)-(+)-5-oxo-2-tetrahydro-furancarboxylic acid, (S)-(+)-hexahydromandelic acid, (S)-(+)-N-[1-(1-naphthyl)-ethyl]-phthalamic acid, (S)-(+)-O-acetylmandelic acid, (S)-2-(1-phenylethylcarbamoyl)benzoic acid, (S)-2-(4-isobutylphenyl)propanoic acid, (S)-2-(phenylcarbamoyloxy)propanoic acid, (S)-3-(benzyloxycarbonyl)-2-oxoimidazolidine-4-carboxylic acid, (S)-3,3,3-trifluoro-2-methoxy-2-phenylpropanoic acid, (S)-3,3,3-trifluoro-2-methoxy-2-phenylpropanoic acid, (S)-6-methoxy-2,5,7,8-tetramethylchroman-2-carboxylic acid, (trimethylsilyl)acetic acid, (z)-2-cyano-3-(3-hydroxyphenyl)acrylic acid, 1-(4-chlorophenyl)-1-cyclopentanecarboxylic acid, 1-(tert-butyl)hydrocinnamic acid, 1,2-phenylenedioxydiacetic acid, 1,4-dihydro-2-methylbenzoic acid, 1,4-dihydroxy-2-naphthoic acid, 10-hydroxydecanoic acid, 10-undecynoic acid, 1-admantanecarboxylic acid, 1-cyano-1-cyclopropane-carboxylic acid, 1-hydroxy-2-naphthoic acid, 1-isoquinolinecarboxylic acid, 1-methyl-(1S,2R)-(+)-cis-1,2,3,6-tetrahydrophthalate, 1-methyl-1-cyclohexane-carboxylic acid, 1-methyl-1H-indole-2-carboxylic acid, 1-methyl-2-pyrrolecarboxylic acid, 1-methylcyclopropane-carboxylic acid, 1-naphthoic acid, 1-phenyl-1-cyclopentane-carboxylic acid, 1-phenyl-1-cyclopropane-carboxylic acid, 1-pyreneacetic acid, 1-pyrenebutyric acid, 1-pyrenecarboxylic acid, 2-((1R,2R,3R,4S)-3-hydroxy-4,7,7-trimethylbicyclo[2.2.1]heptan-2-yl)acetic acid, 2-((benzyloxycarbonyl)(methyl)amino)-2-methylpropanoic acid, 2-(2, (trifluoromethyl)phenyl)acetic acid, 2-(2,4,5-trichlorophenoxy)-propionic acid, 2-(2,4-dichlorophenoxy)-propionic acid, 2-(3,5-dinitrobenzamido)-2-phenylacetic acid, 2-(3,5-dinitrobenzamido)-4-methylpentanoic acid, 2-(3-chlorophenoxy)propionic acid, 2-(4-(trifluoromethyl)phenyl)acetic acid, 2-(4-chloro-3-nitrobenzoyl)-benzoic acid, 2-(4-chlorophenoxy)-2-methyl-propionic acid, 2-(4-chlorophenoxy)propionic acid, 2-(4-fluorobenzoyl)benzoic acid, 2-(4-hydroxy-3-methoxyphenyl)acetic acid, 2-(4-hydroxyphenoxy)-propionic acid, 2-(4-isobutylphenyl)propanoic acid, 2-(4-nitrophenyl)propionic acid, 2-(benzyloxycarbonylamino)-3-(1 H-indol-3-yl)propanoic acid, 2-(trifluoromethyl)acrylic acid, 2-(trifluoromethyl)benzoic acid, 2-(trifluoromethyl)cinnamic acid, 2,2,3,3-tetramethyl-cyclopropanecarboxylic acid, 2,2-bis(hydroxymethyl)-propionic acid, 2,3,4,5,6-pentafluoro-cinnamic acid, 2,3,4,5,6-pentafluorophenoxy acetic acid, 2,3,4,5,6-pentafluorophenyl-acetic acid, 2,3,4,5-tetrafluorobenzoic acid, 2,3,4-trifluorocinnamic acid, 2,3,4-trihydroxybenzoic acid, 2,3,4-trimethoxybenzoic acid, 2,3,5,6-tetrafluoro-4-hydroxy-benzoic acid hydrate, 2,3,5,6-tetrafluorobenzoic acid, 2,3,5,6-tetrafluoro-p-toluic acid, 2,3,5-triiodobenzoic acid, 2,3,6-trifluorobenzoic acid, 2,3-dichlorobenzoic acid, 2,3-difluorobenzoic acid, 2,3-dihydroxybenzoic acid, 2,3-dimethylbenzoic acid, 2,4,5-trichlorophenoxyacetic acid, 2,4,5-trimethoxybenzoic acid, 2,4,6-trichlorobenzoic acid, 2,4,6-trifluorobenzoic acid, 2,4,6-trihydroxybenzoic acid monohydrate, 2,4,6-trimethylbenzoic acid, 2,4-bis(trifluoromethyl)-benzoic acid, 2,4-dichloro-5-fluorobenzoic acid, 2,4-dichloro-5-sulfamoyl-benzoic acid, 2,4-dichlorobenzoic acid, 2,4-dichlorophenylacetic acid, 2,4-difluorobenzoic acid, 2,4-difluorophenylacetic acid, 2,4-dihydroxybenzoic acid, 2,4-dimethylbenzoic acid, 2,4-dinitrobenzoic acid, 2,4-dinitrophenylacetic acid, 2,4-hexadienoic acid, 2,5-bis(trifluoromethyl)-benzoic acid, 2,5-dichlorobenzoic acid, 2,5-difluorobenzoic acid, 2,5-difluorophenylacetic acid, 2,5-dihydroxybenzoic acid, 2,5-dihydroxyphenylacetic acid, 2,5-dimethoxybenzoic acid, 2,5-dimethoxycinnamic acid, 2,6-dichloro-3-nitrobenzoic acid, 2,6-difluorobenzoic acid, 2,6-difluorophenylacetic acid, 2,6-dihydroxybenzoic acid, 2,6-dimethoxynicotinic acid, 2,6-dimethylbenzoic acid, 2,6-heptadienoic acid, 2-[4-(dibutylamino)-2-hydroxy-benzoyl]benzoic acid, 2-bibenzylcarboxylic acid, 2-biphenylcarboxylic acid, 2-bromo-3-nitrobenzoic acid, 2-bromo-4,5-dimethoxybenzoic acid, 2-bromo-5-methoxybenzoic acid, 2-bromo-5-nitrobenzoic acid, 2-bromoacrylic acid, 2-bromophenylacetic acid, 2-chloro-3-nitrobenzoic acid, 2-chloro-4,5-difluorobenzoic acid, 2-chloro-4-fluorobenzoic acid, 2-chloro-5-(methylthio)-benzoic acid, 2-chloro-5-(trifluoro-methyl)benzoic acid, 2-chloro-5-nitrobenzoic acid, 2-chloro-5-nitrocinnamic acid, 2-chloro-6-fluorobenzoic acid, 2-chloro-6-fluorophenylacetic acid, 2-chloro-6-methylnicotinic acid, 2-chlorobenzoic acid, 2-chloronicotinic acid, 2-chlorophenylacetic acid, 2-chloropropionic acid, 2-ethoxy-1-naphthoic acid, 2-ethoxybenzoic acid, 2-ethyl-2-hydroxybutyric acid, 2-ethylbutyric acid, 2-ethylhexanoic acid, 2-ethylthio-2,2-diphenyl-acetic acid, 2-fluoro-3-(trifluoromethyl)-benzoic acid, 2-fluoro-4-(trifluoromethyl)-benzoic acid, 2-fluoro-5-methylbenzoic acid, 2-fluoro-5-nitrobenzoic acid, 2-fluoro-6-(trifluoromethyl)-benzoic acid, 2-fluorobenzoic acid, 2-fluorocinnamic acid, 2-fluorophenylacetic acid, 2-hydroxy-3-isopropyl-6-methylbenzoic acid, 2-hydroxy-3-isopropylbenzoic acid, 2-hydroxy-3-methylbutyric acid, 2-hydroxy-6-isopropyl-3-methylbenzoic acid, 2-hydroxycaproic acid, 2-hydroxyhippuric acid, 2-hydroxyisobutyric acid, 2-hydroxyisobutyric acid, 2-hydroxynicotinic acid, 2-hydroxyphenylacetic acid, 2-iodobenzoic acid, 2-mercaptonicotinic acid, 2-methoxy-2-phenylacetic acid, 2-methoxy-4-(methylthio)-benzoic acid, 2-methoxy-4-nitrobenzoic acid, 2-methoxyphenylacetic acid, 2-methyl-1-cyclohexane-carboxylic acid (cis and trans), 2-methyl-3-nitrobenzoic acid, 2-methyl-3-phenylpropanoic acid, 2-methyl-4-oxo-4-phenylbutyric acid, 2-methyl-6-nitrobenzoic acid, 2-methylbutyric acid, 2-methylcinnamic acid, 2-methylcyclopropane-carboxylic acid (cis and trans), 2-methylhexanoic acid, 2-methylhippuric acid, 2-methylhydrocinnamic acid, 2-methylvaleric acid, 2-naphthoic acid, 2-naphthylacetic acid, 2-nitro-4-(trifluoromethyl)benzoic acid, 2-nitrobenzoic acid, 2-norbornaneacetic acid, 2-oxo-6-pentyl-2H-pyran-3-carboxylic acid, 2-phenoxybenzoic acid, 2-phenoxybutyric acid, 2-phenoxypropionic acid, 2-propylpentanoic acid, 2-quinoxalinecarboxylic acid, 2-thiopheneacetic acid, 2-thiopheneacetic acid, 2-thiopheneglyoxylic acid, 3-(2-hydroxyphenyl)propionic acid, 3-(2-thienyl)acrylic acid, 3-(3,4,5-trimethoxyphenyl)-propionic acid, 3-(3,4-dimethoxyphenyl)-propionic acid, 3-(3-hydroxy-2,4,6-triiodophenyl)pentanoic acid, 3-(3-hydroxyphenyl)-propionic acid, 3-(3-methoxyphenyl)propionic acid, 3-(4-chlorobenzoyl)propionic acid, 3-(4-fluorobenzoyl)propionic acid, 3-(4-hydroxyphenyl)propionic acid, 3-(phenylsulfonyl)propionic acid, 3-(trifluoromethyl)cinnamic acid, 3-(trimethylsilyl)propynoic acid, 3,3,3-triphenylpropionic acid, 3,4-(methylenedioxy)cinnamic acid, 3,4-(methylenedioxy)phenyl-acetic acid, 3,4-dichlorobenzoic acid, 3,4-dichlorophenoxyacetic acid, 3,4-diethoxybenzoic acid, 3,4-difluorobenzoic acid, 3,4-dihydroxybenzoic acid, 3,4-dihydroxyhydrocinnamic acid, 3,4-dihydroxyphenylacetic acid, 3,5,6-trichlorosalicylic acid, 3,5-bis(trifluoromethyl)-phenyl acetic acid, 3,5-dibromobenzoic acid, 3,5-dichlorosalicyclic acid, 3,5-difluorocinnamic acid, 3,5-dihydroxy-2-naphthoic acid, 3,5-dinitrobenzoic acid, 3,5-dinitro-o-tuluic acid, 3,5-dinitro-p-toluic acid, 3,5-dinitrosalicyclic acid, 3,5-di-tert-butyl-4-hydroxy-benzoic acid, 3,5-di-tert-butylbenzoic acid, 3,7-dihydroxy-2-naphthoic acid, 3, thiopheneacetic acid, 3-benzoyl-2-pyridine-carboxylic acid, 3-benzoylbenzoic acid, 3-bromo-4-fluorobenzoic acid (95%), 3-bromo-4-methylbenzoic acid, 3-bromo-5-iodobenzoic acid, 3-bromobenzoic acid, 3-bromocinnamic acid, 3-carboxy-proxyl, 3-chloro-2-nitrobenzoic acid, 3-chloro-4-fluorobenzoic acid, 3-chloro-4-hydroxyphenyl-acetic acid, 3-chlorosalicylic acid, 3-cyanobenzoic acid, 3-fluoro-2-methylbenzoic acid, 3-fluoro-4-hydroxy-phenylacetic acid, 3-fluoro-4-methoxybenzoic acid, 3-fluorophenylacetic acid, 3-furoic acid, 3-hydroxy-2-naphthoic acid, 3-hydroxy-2-quinoxaline-carboxylic acid, 3-hydroxy-4-methoxybenxoic acid, 3-hydroxy-4-methoxy-cinnamic acid, 3-hydroxy-4-nitrobenzoic acid, 3-hydroxybenzoic acid, 3-hydroxybutyric acid, 3-hydroxyphenylacetic acid, 3-indolebutyric acid, 3-indoleglyoxylic acid, 3-indolepropionic acid, 3-iodo-4-methylbenzoic acid, 3-iodobenzoic acid, 3-isoquinolinecarboxylic acid hydrate, 3-methoxy-4-nitrobenzoic acid, 3-methoxycyclohexane-carboxylic acid (cis and trans), 3-methyl-2-phenyvaleric acid, 3-methylhippuric acid, 3-methylindene-2-carboxylic acid, 3-methylsalicylic acid, 3-methyl valeric acid, 3-nitreobenzoic acid, 3-nitrophenylacetic acid, 3-nitropropionic acid, 3-noradamantanecarboxylic acid, 3-oxo-1-indancarboxylic acid, 3-phenoxybenzoic acid, 3-phenylbutyric acid, 3-p-tolylpropanoic acid, 3-thiophenecarboxylic acid, 4-(1,3-dioxoisoindolin-2-yl)-2-hydroxybutanoic acid, 4-(2,4,5-trichlorophenoxy)-butyric acid, 4-(2,4-dichlorophenoxy)-butyric acid, 4-(2,4-di-tert-pentylphenoxy)butyric acid, 4-(2-phenoxyethoxy)benzoic acid, 4-(3,4-dimethoxyphenyl)-butyric acid, 4-(4-methoxyphenyl)butyric acid, 4-(4-nitrophenyl)butyric acid, 4-(diethylamino)benzoic acid, 4-(dimethylamino)cinnamic acid, 4-(dimethylamino)phenyl-acetic acid, 4-(ethylthio)benzoic acid, 4-(hydroxymethyl)benzoic acid, 4-(methylsulfonyl)benzoic acid, 4-(methylthio)benzoic acid, 4-(methylthio)phenylacetic acid, 4-(trifluoromethoxy)benzoic acid, 4′-(trifluoromethyl)biphenyl-2-carboxylic acid, 4-(trifluoromethyl)mandelic acid, 4,4,4-trifluoro-3-methyl-2-butenoic acid, 4,4-bis(4-hydroxyphenyl)-valeric acid, 4,5-dimethoxy-2-nitrobenzoic acid, 4,6-dioxoheptanoic acid, 4-[4-(2-carboxybenzoyl)-phenyl]butyric acid, 4-acetamidobenzoic acid, α-acetylbenzoic acid, 4-acetylphenoxyacetic acid, 4-benzyloxy-3-methoxyphenyl-acetic acid, 4-biphenylacetic acid, 4-bromo-3,5-dihydroxy-benzoic acid, 4-bromobenzoic acid, 4-bromocinnamic acid, 4-bromophenylacetic acid, 4-butoxybenzoic acid, α-butoxyphenylacetic acid, 4-butylbenzoic acid, 4-chloro-2,5-difluorobenzic acid, 4-chloro-3-sulfamoylbenzoic acid, 4-chlorobenzoic acid, 4-chloro-o-tolyloxyacetic acid, α-chlorophenylacetic acid, 4-chlorosalicylic acid, 4-ethoxycarbonyloxy-3,5-dimethoxybenzoic acid, 4-ethoxyphenylacetic acid, 4-ethylbenzoic acid, 4′-ethylbiphenyl-4-carboxylic acid, 4-fluorenecarboxylic acid, 4-fluoro-1-naphthoic acid, a-fluoro-2-(trifluoromethyl)-benzoic acid, 4-fluoro-3-nitrobenzoic acid, 4-fluorobenzoic acid, 4-fluorobenzoic acid, 4-fluorocinnamic acid, 4-fluorophenoxyacetic acid, α-heptyloxybenzoic acid, 4-hexylbenzoic acid, 4-hexyloxybenzoic acid, 4-hydroxy-3-(morpholino-methyl)benzoic acid hydrate, 4-hydroxy-3,5-dinitrobenzoic acid, 4-hydroxy-3-methoxy-benzoic acid, 4-hydroxy-3-methoxy-mandelic acid, 4-hydroxy-3-nitrobenzoic acid, 4-hydroxy-3-nitrophenylacetic acid, 4-hydroxybenzoic acid, 4′-hydroxybiphenyl-4-carboxylic acid, 4-hydroxyphenylacetic acid, 4-hydroxyphenylacetic acid, α-hydroxyphenylpyruvic acid, 4-iodobenzoic acid, 4-isopropoxybenzoic acid, 4-methoxy-3-nitrobenzoic acid, 4-methoxycyclohexane-carboxylic acid, 4-methoxysalicylic acid, 1-methyl-1-cyclohexane-carboxylic acid (cis and trans), 4-methyl-3-nitrobenzoic acid, a-methylhippuric acid, 4-methylsalicylic acid, 4-methylvaleric acid, 4-nitro-3-pyrazolecarboxylic acid, 4-nitrohippuric acid, 4-nonyloxybenzoic acid, 4-octylbenoic acid, 4-oxo-4H-1-benzopyran-2-carboxylic acid, 4-oxo-6-phenyl-5-hexenoic acid, α-pentenoic acid, 4-pentylbenzoic acid, 4-pentylbicyclo[2.2.2]octane-1-carboxylic acid, α-pentyloxybenzoic acid, 4-pentynoic acid, 4-phenylbutyric acid, 4-propoxybenzoic acid, 4-propylbenzoic acid, 4-pyrazolecarboxylic acid, 4-tert-butylbenzoic acid, 4-tert-butylcyclohexanecarboxylic acid, 4-vinylbenzoic acid, 5-(4-chlorophenyl)-2-furoic acid, 5,6-dichloronicotinic acid, 5-bromo-2,4-dihydroxybenzoic acid, 5-fluoro-2-methylbenzoic acid, 5-fluoroindole-2-carboxylic acid, 5-fluorosalicylic acid, 5-hydantoinacetic acid, 5-hydroxy-2-indole-carboxylic acid, 5-methoxy-1-indanone-3-acetic acid, 5-methoxy-2-methyl-3-indoleacetic acid, 5-methoxy-2-nitrobenzoic acid, 5-methoxysalicylic acid, 5-methyl-2-nitrobenzoic acid, 5-methyl-2-pyrazine-carboxylic acid, 5-nitro-2-furoic acid, 5-nitro-3-pyrazolecarboxylic acid, 5-phenyl valeric acid, 6-(carbobenzyloxyamino)-caproic acid, 6-acetamidohexnoic acid, 6-bromohexanoic acid, 6-chloronicotinic acid, 6-hydroxy-2,5,7,8-tetramethylchroman-2-carboxylic acid, 6-methylchromone-2-carboxylic acid, 6-methylnicotinic acid, 6-nitrocaproic acid, 6-oxoheptaoic acid, 6-phenylhexanoic acid, 7-(carboxymethyoxy)-4-methylcoumarin, 7-hydroxycoumarin-4-acetic acid, 7-methoxy-2-benzofuran-carboxylic acid, 7-methoxycoumarin-4-acetic acid, 7-oxoctanoic acid, 9-anthracenecarboxylic acid, 9-fluoreneacetic acid, 9-fluorenone-1-carboxylic acid, α, α, α-trifluoro-m-toluic acid, α-acetamidocinnamic acid, abietic acid, acetic acid, acetyl-L-asparagine, acetylsalicyclic acid, α-cyano-4-hydroxycinnamic acid, adipic acid monoethyl ester, α-hydroxyhippuric acid, anthranilic acid, anti-3-oxotricyclo[2.2.1.02,6]heptane-7-carboxylic acid, α-phenylcyclopentaneacetic acid, α-phenyl-o-toluic acid, Atrolactic acid, benzilic acid, benzotriazole-5-carboxylic acid, benzoylformic acid, bis(4-chlorophenyl)acetic acid, carbobenzyloxy-DL-alanine, carbobenzyloxy-L-alanine, carbobenzyloxy-1-glutamine, carbobenzyloxy-L-valine, cis-2-methoxycinnamic acid, crotonic acid, cyclohexanebutyric acid, cyclohexanecarboxylic acid, cyclohexanepentanoic acid, cyclohexanepropionic acid, cyclopentylacetic acid, D,L-3,4-dihydroxymandelic acid, D-3-phenyllactic acid, decanoic acid, dicyclohexylacetic acid, diethylphosphonoacetic acid, dikegulac hydrate, diphenylacetic acid, fumaric acid monoethyl ester, fusaric acid, gallic acid, geranic acid, glycolic acid, heptadecafluorononanoic acid, heptanoic acid, hexanoic acid, hippuric acid, hydrocinnamic acid, indole-3-carboxylic acid, indole-4-carboxylic acid, isovaleric acid, L-3-phenyl lactic acid, lauric acid, L-lactic acid (85%), maleamic acid, methoxyacetic acid, mono-(1R)-(−)-menthyl phthalate, mono-(1S)-(+)-menthyl phthalate, mono-methyl cis-5-norbornene-endo-2,3-dicarboxylate, mono-methyl phthalate, mono-methylterephthalate, N-(2-furoyl)glycine, n-(3,5-dinitrobenzoyl)-DL-a-phenylglycine, N-(3-indolylacetyl)-L-alanine, N-(3-indolylacetyl)-L-isoleucine, N-(3-indolylacetyl)-L-leucine, N-(3-indolylacetyl)-L-phenylalanine, N-(3-indolylacetyl)-L-valine, N-(carbobenzyloxy)-1-phenyl-alanine, N,N-diethyl-3,6-difluoro-phthalamic acid, N—[(R)—I-(1-naphthyl)ethyl]-phthalamic acid, N-[5-(trifluoromethyl)-2-pyridyl]-L-valine, N-acetyl-4-fluoro-DL-phenylalanine, N-acetyl-DL-tryptophan, N-acetyl-1-leucine, N-acetyl-L-methionine, N-acetyl-L-phenylalanine, N-acetyl-1-tyrosine, N-benzoyl-(2R,3S)-3-phenyl-isoserine, N-benzoyl-L-threonine, N-carbobenzyloxy-2-methyl-alanine, N-carbobenzyloxy-L-glutamic acid 1-methyl ester, N-carbobenzyloxy-L-isoleucine, N-carbobenzyloxy-L-leucine, N-carbobenzyloxy-1-threonine, N-ethoxycarbonyl-1-phenylalanine, nonanoic acid, N-p-tosylglycine, N-p-tosyl-L-phenylalanine, o-anisic acid, p-anisic acid, pentafluorobenzoic acid, phenoxyacetic acid, phenylacetic acid, podocarpic acid, pyruvic acid, rhodanine-3-acetic acid, S-(thiobenzoyl)thioglycolic acid, s-benzyl-N-carbobenzyloxy-1-cysteine, sebacic acid monomethyl ester, succinamic acid, succinic 2,2-dimethyl-hydrazide, tetrahydro-2-furoic acid, trans-1-acetyl-4-hydroxy-L-proline, trans-2,3-dimethoxycinnamic acid, trans-2,4-dichlorocinnamic acid, trans-2,4-difluorocinnamic acid, trans-2,5-difluorocinnamic acid, trans-2,6-difluorocinnamic acid, trans-2-chloro-6-fluro-cinnamic acid, trans-2-hexenoic acid, trans-3-(2,3,5,6-tetramethyl-benzoyl)acrylic acid, trans-3-(2,5-dimethylbenzo-yl)-acrylic acid, trans-3-(4-ethoxy-benzoyl)acrylic acid, trans-3-(4-methoxybenzoyl)-acrylic acid, trans-3-(4-methylbenzoyl)-acrylic acid, trans-3,4-difluorocinnamic acid, trans-3-fluorocinnamic acid, trans-3-furanacrylic acid, trans-3-hexenoic acid, trans-4-chloro-3-nitrocinnamic acid, trans-4-hydroxy-3-methoxy-cinnamic acid, trans-4-methyl-1-cyclohexane carboxylic acid, trans-4-pentylcyclohexane carboxylic acid, trans-5-bromo-2-methoxy cinnamic acid, trans-styrylacetic acid, tridecafluoroheptanoic acid, trimethylacetic acid, triphenylacetic acid, valeric acid, and yyohimbinic acid, mono-hydrate; wherein R₁ and R₂ can be the same or different.

4. A compound of formula (II), or a pharmaceutically acceptable salt, hydrate, tautomer, and stereoisomer thereof: 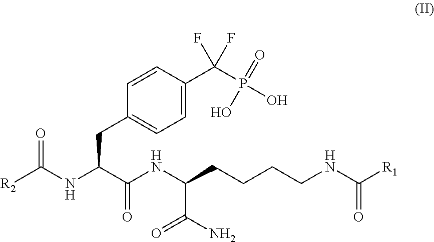
wherein
each R₁ and R₂ independently is a residue of a carboxylic acid, or a pharmaceutically acceptable salt thereof, wherein R₁ and R₂ can be the same or different.

5. The compound of claim 4, wherein the carboxylic acid is represented by a formula RCOOH, wherein R is selected from a group consisting of hydrogen, an aliphatic group, or an aromatic group, wherein the aliphatic group is saturated or unsaturated, and wherein the aliphatic group or the aromatic group is substituted with C₁-C₂₄ alkyl, C₁-C₂₄ alkenyl, C₁-C₂₄ alkynyl, haloalkyl, haloalkenyl, heteroalkyl, cycloalkyl, cycloalkenyl, heterocycloalkyl, heterocycloalkenyl, aryl, heteroaryl, cycloalkylalkyl, heterocycloalkylalkyl, arylalkyl, heteroarylalkyl, arylalkenyl, cycloalkylheteroalkyl, heterocycloalkylheteroalkyl, heteroarylheteroalkyl, arylheteroalkyl, and acyl, wherein R group is optionally substituted with at least one of the groups selected from C₁-C₂₄ alkyl, hydroxy, alkoxy, cyano, halo, nitro, aryl, amino, C₁-C₂₄ alkenyl, C₁-C₂₄ alkynyl, haloalkyl, haloalkenyl, heteroalkyl, cycloalkyl, cycloalkenyl, heterocycloalkyl, heterocycloalkenyl, aryl, heteroaryl, cycloalkylalkyl, heterocycloalkylalkyl, arylalkyl, heteroarylalkyl, arylalkenyl, cycloalkylheteroalkyl, heterocycloalkylheteroalkyl, heteroarylheteroalkyl, arylheteroalkyl, and acyl.

6. The compound of claim 4, wherein R₁ or R₂ is:

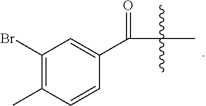 [SMILES: *C(=O)c1ccc(C)c(Br)c1]

7. The compound of claim 4, wherein R₁ or R₂ is:

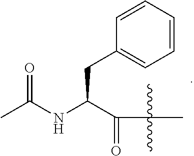 [SMILES: *C(=O)[C@H](Cc1ccccc1)NC(C)=O]

8. The compound of claim 4, wherein R₁ or R₂ is:

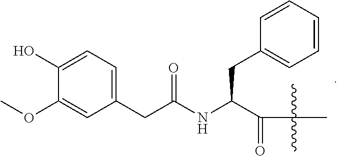 [SMILES: *C(=O)[C@H](Cc1ccccc1)NC(=O)Cc1ccc(O)c(OC)c1]

9. The compound of claim 4 or 5, wherein the compound is 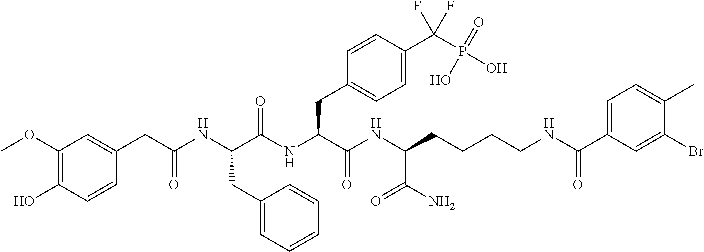
or a pharmaceutically acceptable salt, hydrate, tautomer, or stereoisomer thereof.

10. A compound of formula (III), or a pharmaceutically acceptable salt, hydrate, tautomer, or stereoisomer thereof: 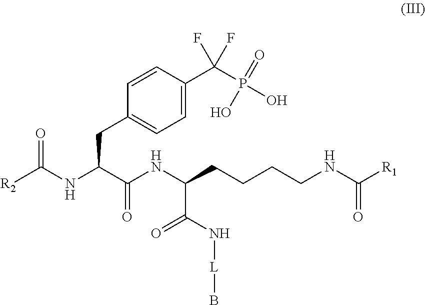
wherein
each R₁ and R₂ is independently a residue of a carboxylic acid, or a pharmaceutically acceptable salt thereof, wherein R₁ and R₂ can be the same or different;
L is a linker selected from the group consisting of: 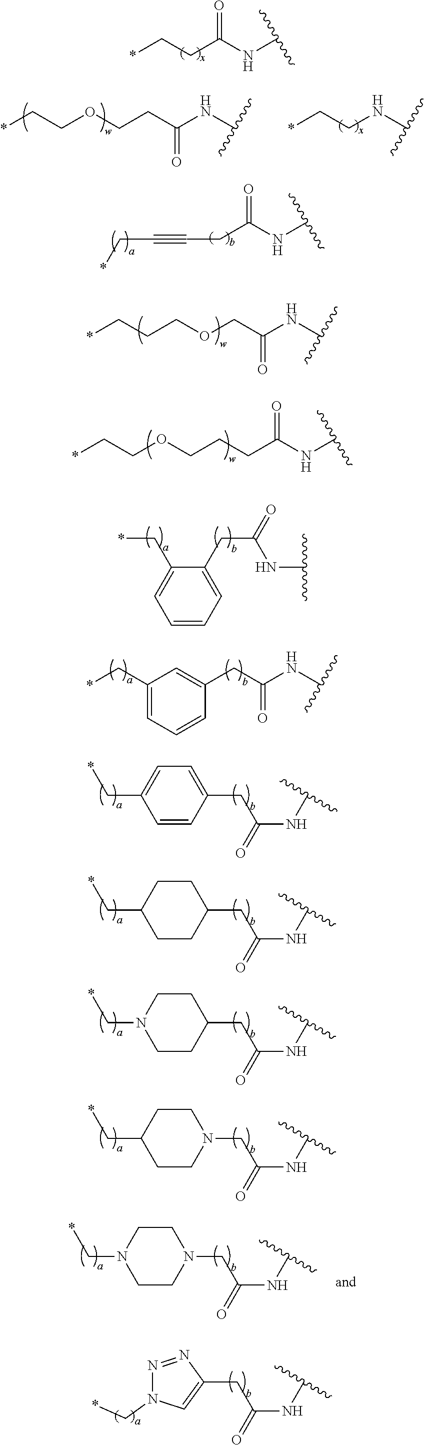
wherein, w is 1-5;
x is 1-15;
each a and b is independently 0-3;
the bond designated with an [symbol] is attached to B;
the bond designated with “*” represents the point of attachment of R₉; and
B is represented by a structure selected from B₁, B₂, and B₃, wherein
B₁ is represented by a structure: 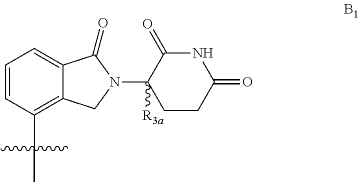
wherein R₃ₐ is fluoro, hydrogen, or deuterium;
B₂ is represented by a structure: 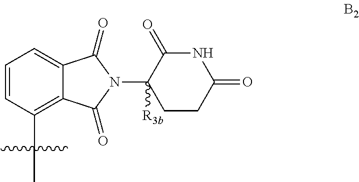
wherein R_{3b} is fluoro, hydrogen, or deuterium; and
B₃ is represented by a structure: 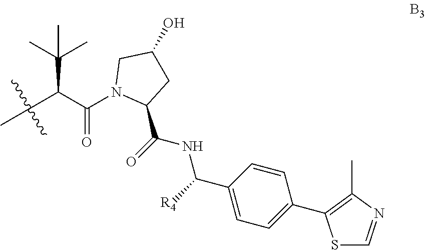
wherein R₄ is methyl, hydrogen, or deuterium.

11. The compound of claim 10, wherein the carboxylic acid is represented by a formula RCOOH, wherein R is selected from a group consisting of hydrogen, an aliphatic group, or an aromatic group, wherein the aliphatic group is saturated or unsaturated, and wherein the aliphatic group or the aromatic group is substituted with C₁-C₂₄ alkyl, C₁-C₂₄ alkenyl, C₁-C₂₄ alkynyl, haloalkyl, haloalkenyl, heteroalkyl, cycloalkyl, cycloalkenyl, heterocycloalkyl, heterocycloalkenyl, aryl, heteroaryl, cycloalkylalkyl, heterocycloalkylalkyl, arylalkyl, heteroarylalkyl, arylalkenyl, cycloalkylheteroalkyl, heterocycloalkylheteroalkyl, heteroarylheteroalkyl, arylheteroalkyl, and acyl, wherein R group is optionally substituted with at least one of the groups selected from C₁-C₂₄ alkyl, hydroxy, alkoxy, cyano, halo, nitro, aryl, amino, C₁-C₂₄ alkenyl, C₁-C₂₄ alkynyl, haloalkyl, haloalkenyl, heteroalkyl, cycloalkyl, cycloalkenyl, heterocycloalkyl, heterocycloalkenyl, aryl, heteroaryl, cycloalkylalkyl, heterocycloalkylalkyl, arylalkyl, heteroarylalkyl, arylalkenyl, cycloalkylheteroalkyl, heterocycloalkylheteroalkyl, heteroarylheteroalkyl, arylheteroalkyl, and acyl.

12. The compound of claim 10, wherein B is B₃ 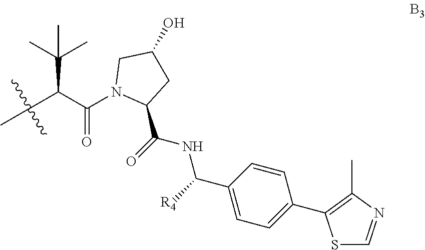
wherein R₄ is methyl, hydrogen, or deuterium.

13. The compound of claim 10, wherein R₁ or R₂ is selected from:

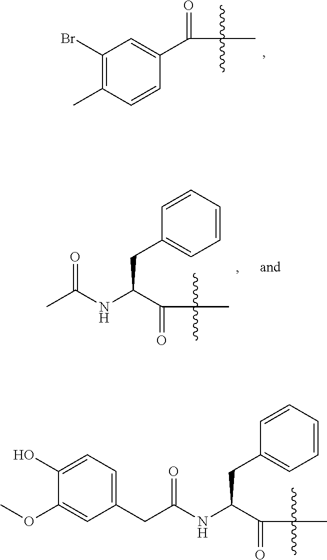 [SMILES: *C(=O)c1ccc(C)c(Br)c1 ; *C(=O)[C@H](Cc1ccccc1)NC(C)=O ; *C(=O)[C@H](Cc1ccccc1)NC(=O)Cc1ccc(O)c(OC)c1]

14. The compound of claim 10, wherein linker L is selected from:

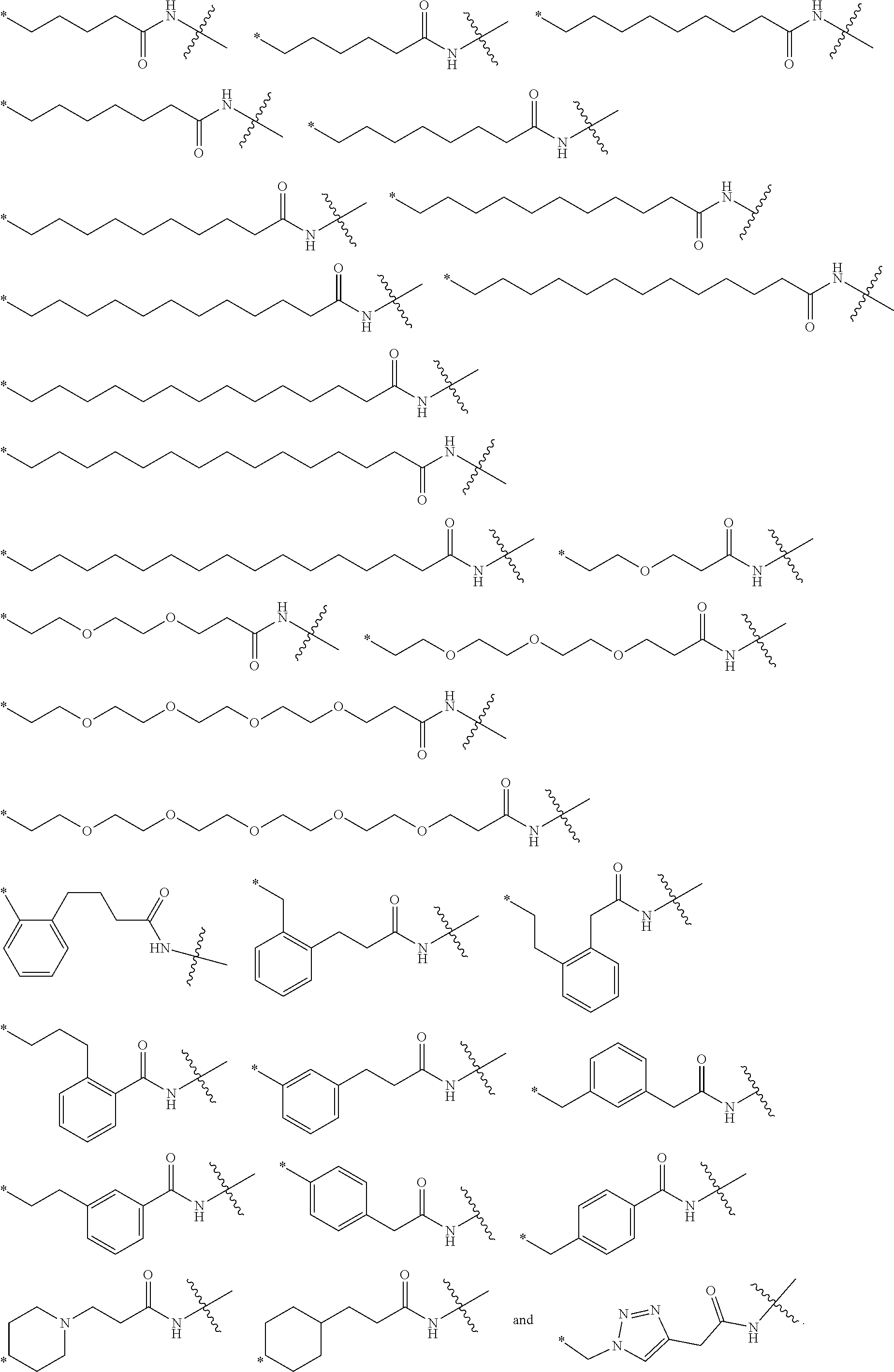

15. The compound of any one of claims 10-14, wherein the compound is: 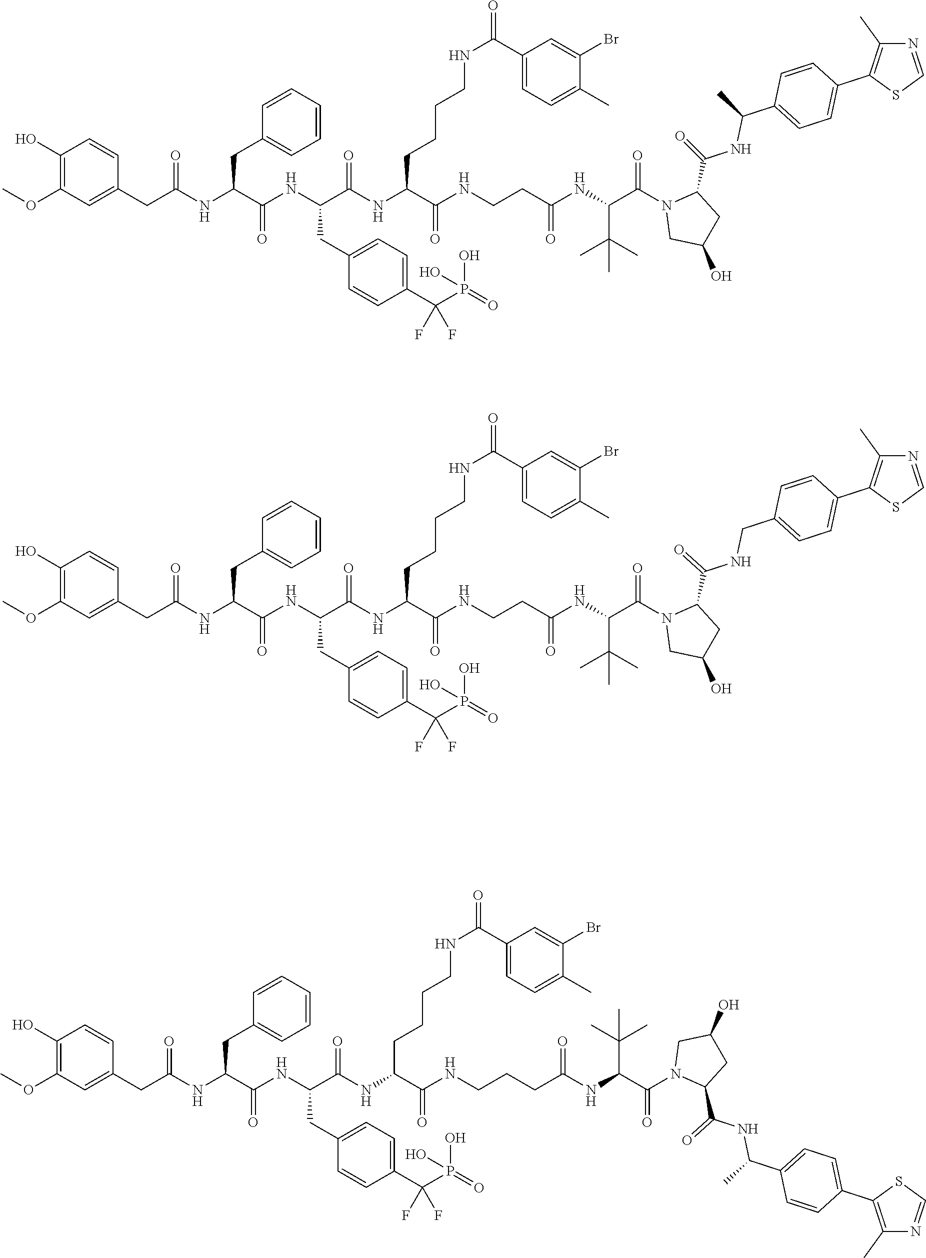 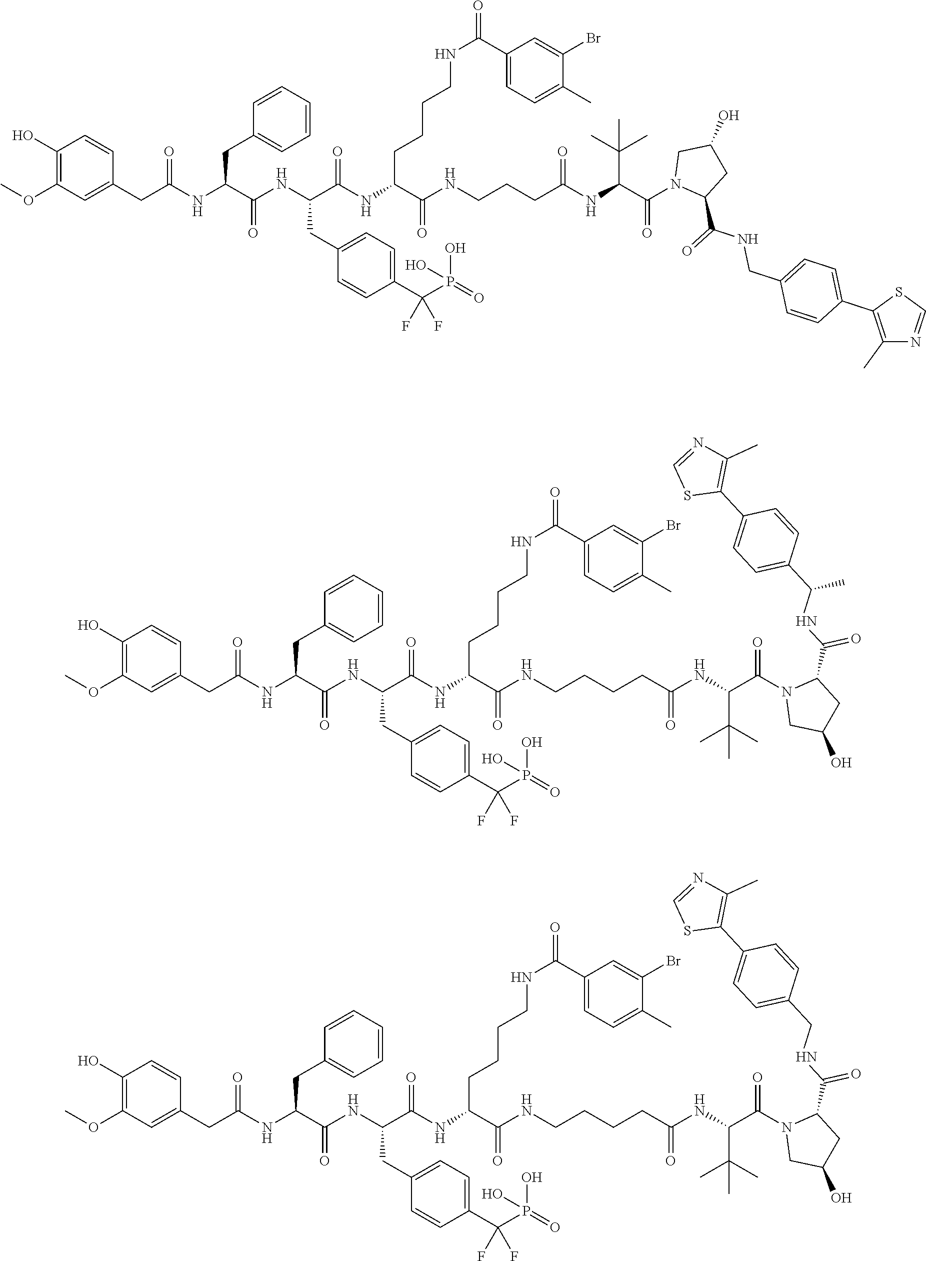 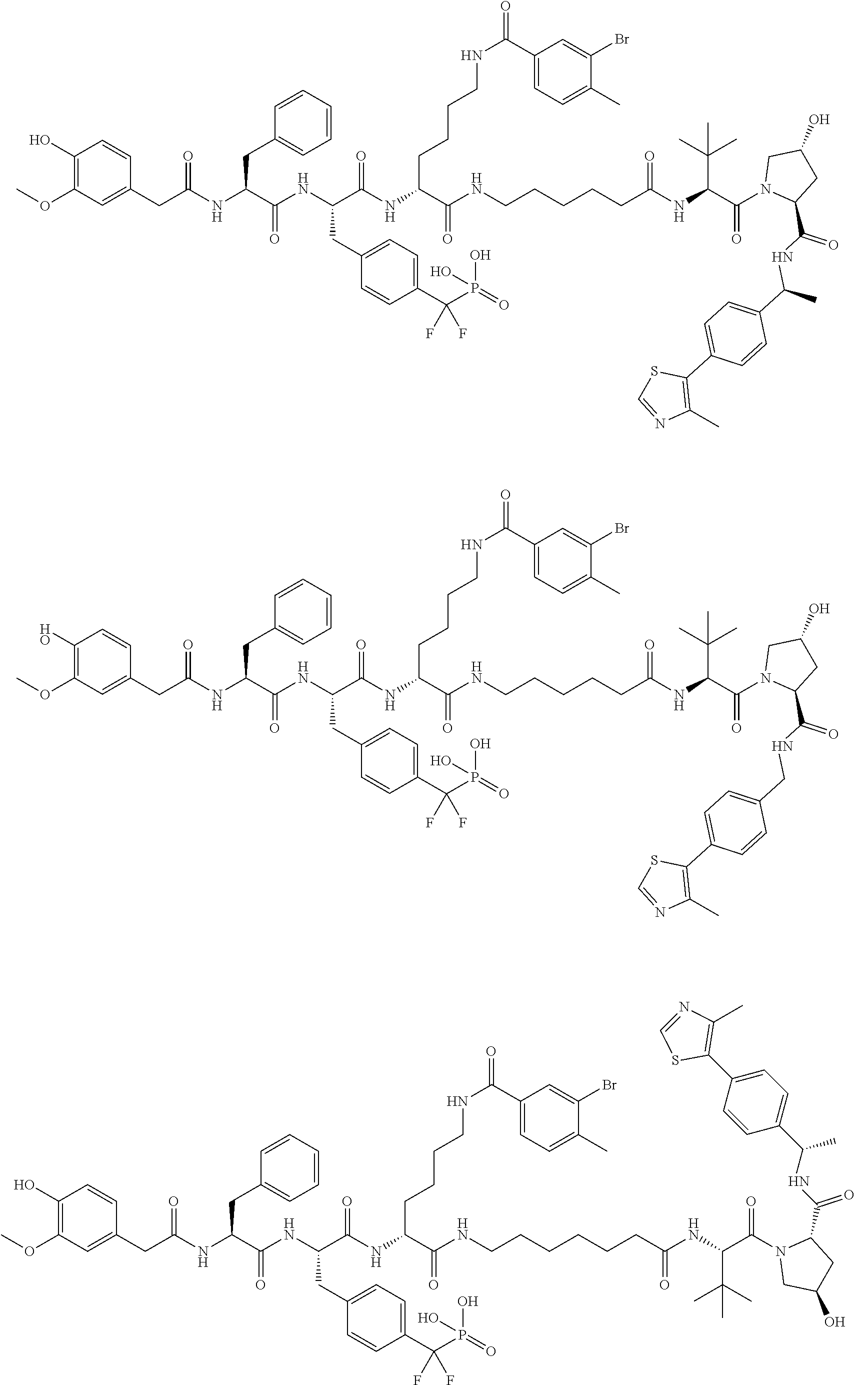 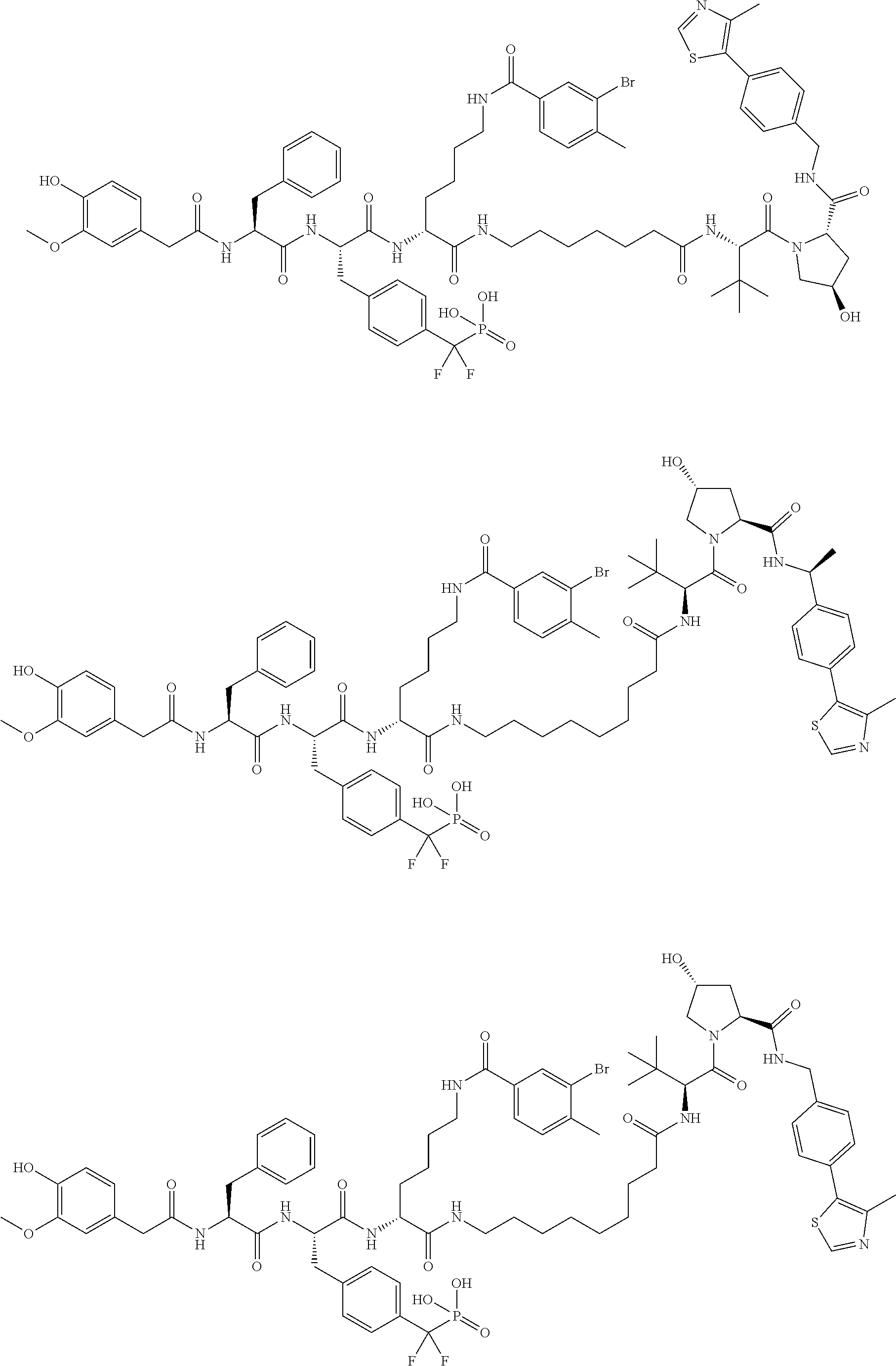 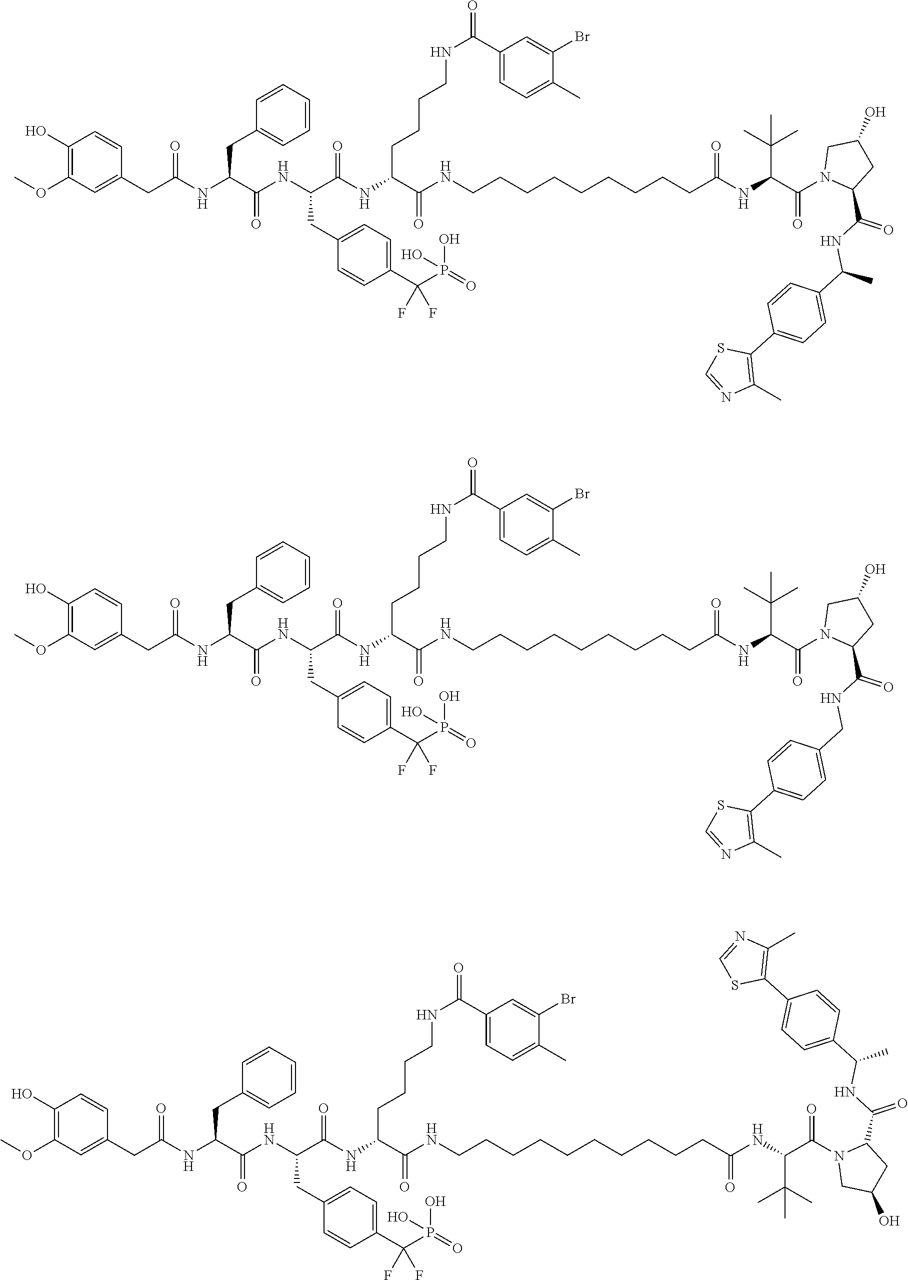 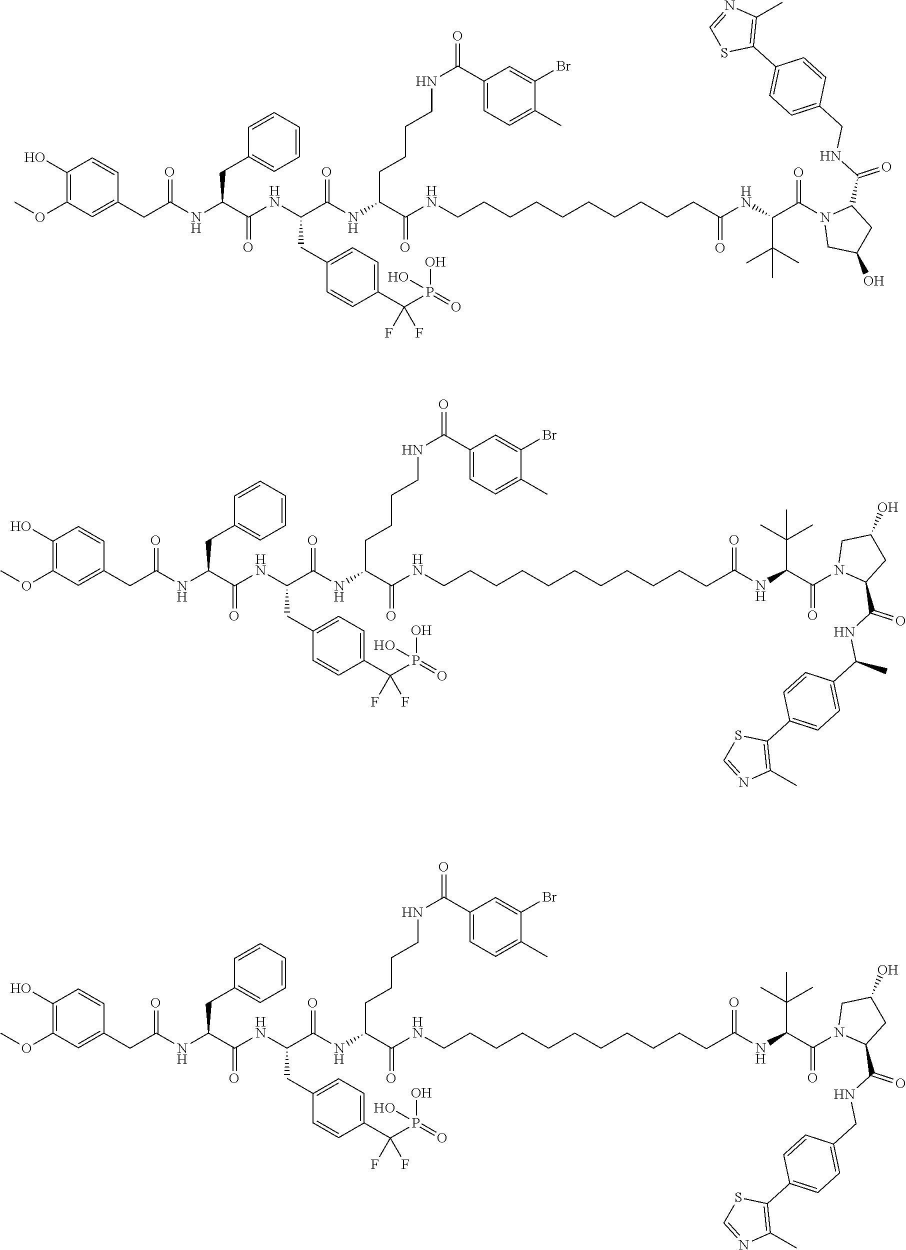 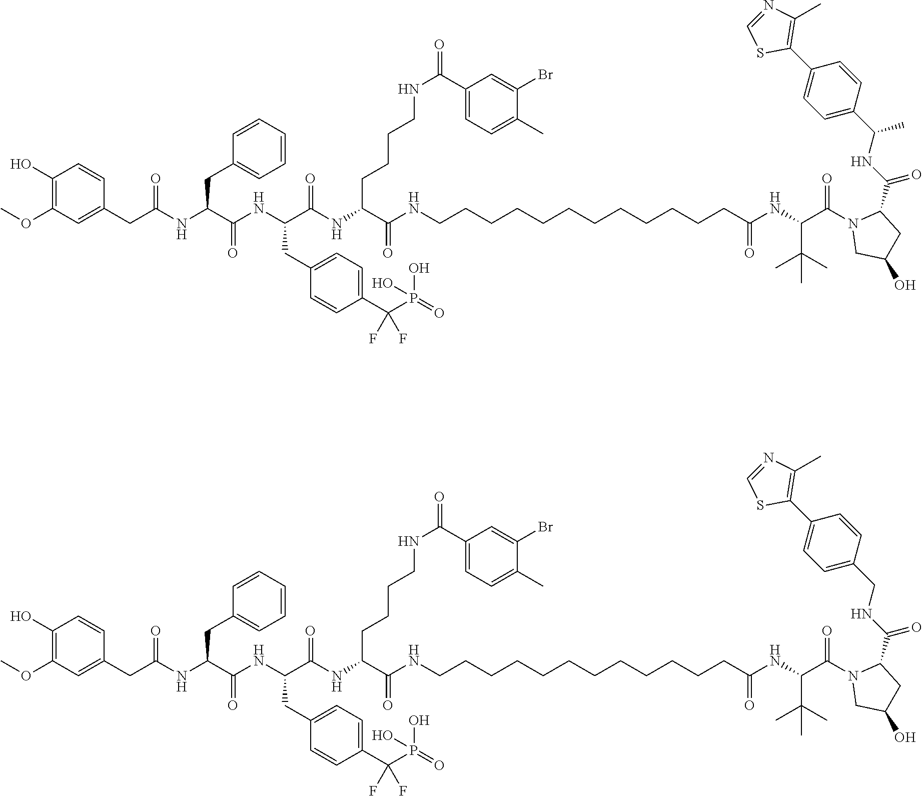 [SMILES: COc1cc(CC(=O)N[C@@H](Cc2ccccc2)C(=O)N[C@@H](Cc2ccc(C(F)(F)P(=O)(O)O)cc2)C(=O)N[C@H](CCCCNC(=O)c2ccc(C)c(Br)c2)C(=O)NCCCCCCCCCCCCC(=O)N[C@H](C(=O)N2C[C@H](O)C[C@H]2C(=O)N[C@@H](C)c2ccc(-c3scnc3C)cc2)C(C)(C)C)ccc1O ; COc1cc(CC(=O)N[C@@H](Cc2ccccc2)C(=O)N[C@@H](Cc2ccc(C(F)(F)P(=O)(O)O)cc2)C(=O)N[C@H](CCCCNC(=O)c2ccc(C)c(Br)c2)C(=O)NCCCCCCCCCCCCC(=O)N[C@H](C(=O)N2C[C@H](O)C[C@H]2C(=O)NCc2ccc(-c3scnc3C)cc2)C(C)(C)C)ccc1O] 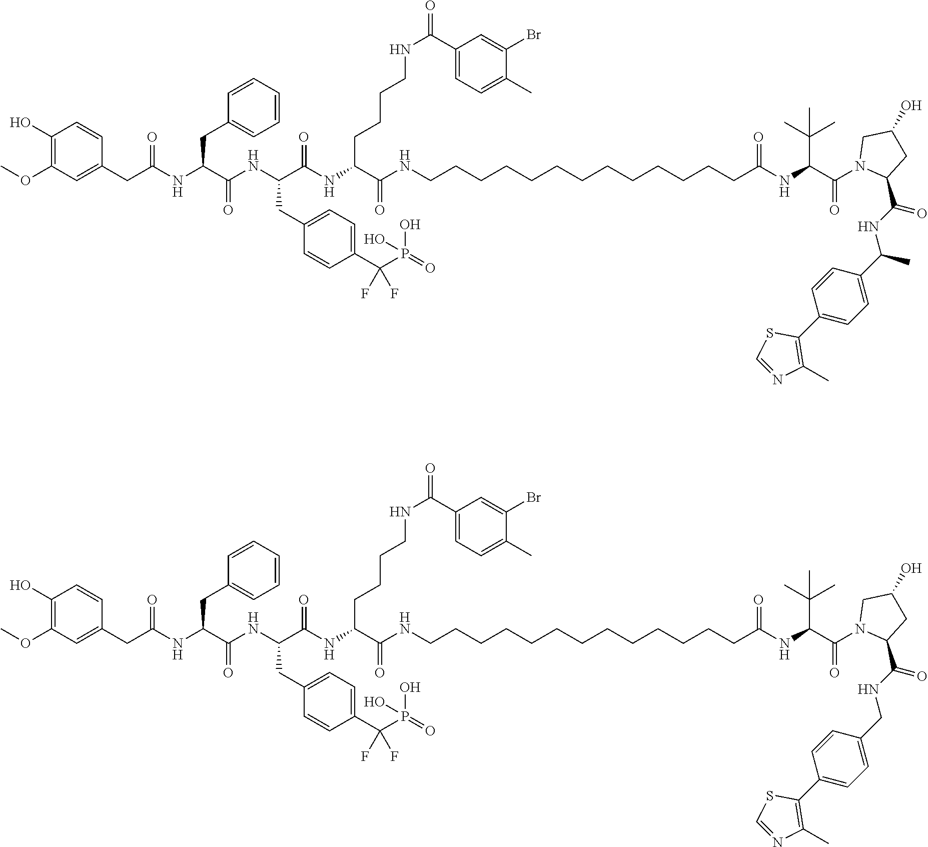 [SMILES: COc1cc(CC(=O)N[C@@H](Cc2ccccc2)C(=O)N[C@@H](Cc2ccc(C(F)(F)P(=O)(O)O)cc2)C(=O)N[C@H](CCCCNC(=O)c2ccc(C)c(Br)c2)C(=O)NCCCCCCCCCCCCCC(=O)N[C@H](C(=O)N2C[C@H](O)C[C@H]2C(=O)N[C@@H](C)c2ccc(-c3scnc3C)cc2)C(C)(C)C)ccc1O ; COc1cc(CC(=O)N[C@@H](Cc2ccccc2)C(=O)N[C@@H](Cc2ccc(C(F)(F)P(=O)(O)O)cc2)C(=O)N[C@H](CCCCNC(=O)c2ccc(C)c(Br)c2)C(=O)NCCCCCCCCCCCCCC(=O)N[C@H](C(=O)N2C[C@H](O)C[C@H]2C(=O)NCc2ccc(-c3scnc3C)cc2)C(C)(C)C)ccc1O] 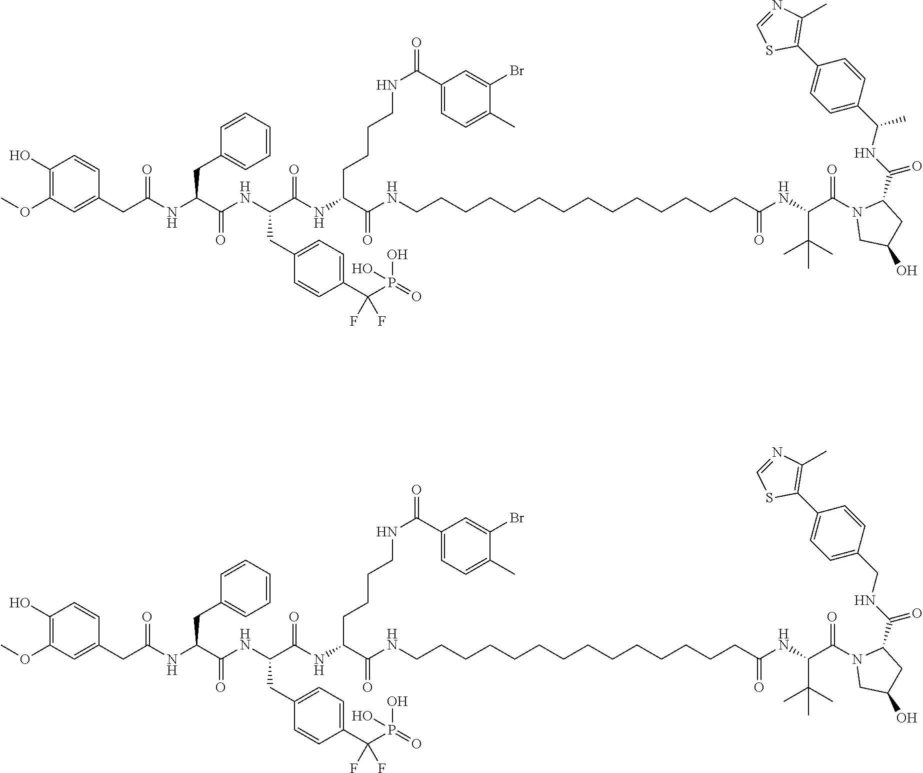 [SMILES: COc1cc(CC(=O)N[C@@H](Cc2ccccc2)C(=O)N[C@@H](Cc2ccc(C(F)(F)P(=O)(O)O)cc2)C(=O)N[C@H](CCCCNC(=O)c2ccc(C)c(Br)c2)C(=O)NCCCCCCCCCCCCCCC(=O)N[C@H](C(=O)N2C[C@H](O)C[C@H]2C(=O)N[C@@H](C)c2ccc(-c3scnc3C)cc2)C(C)(C)C)ccc1O ; COc1cc(CC(=O)N[C@@H](Cc2ccccc2)C(=O)N[C@@H](Cc2ccc(C(F)(F)P(=O)(O)O)cc2)C(=O)N[C@H](CCCCNC(=O)c2ccc(C)c(Br)c2)C(=O)NCCCCCCCCCCCCCCC(=O)N[C@H](C(=O)N2C[C@H](O)C[C@H]2C(=O)NCc2ccc(-c3scnc3C)cc2)C(C)(C)C)ccc1O] 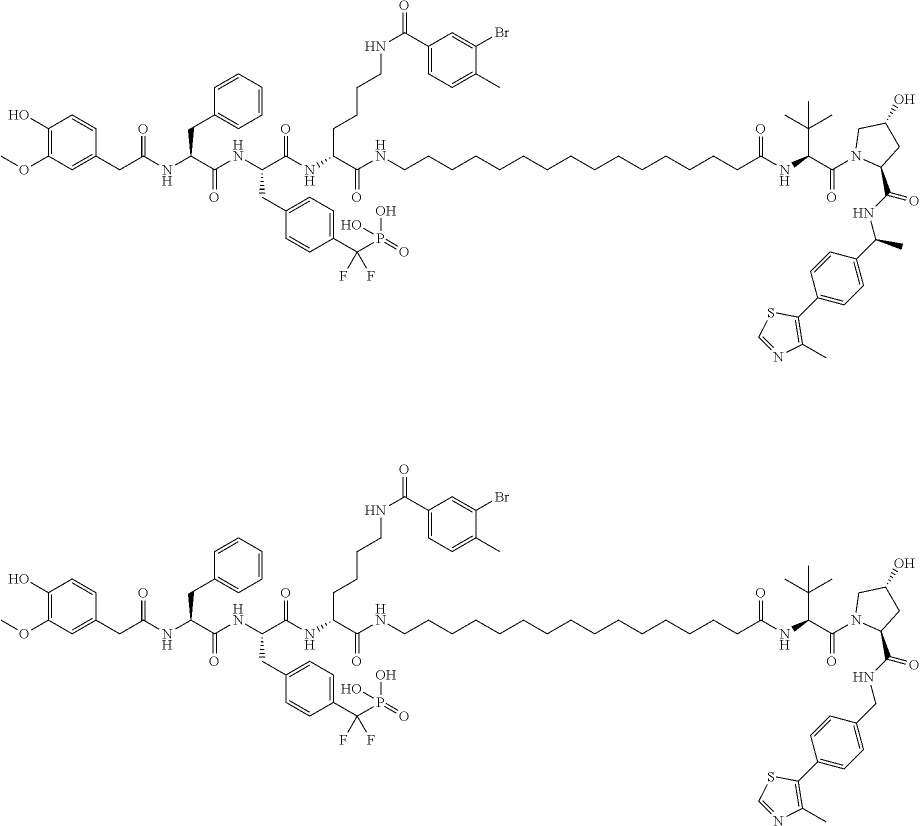 [SMILES: COc1cc(CC(=O)N[C@@H](Cc2ccccc2)C(=O)N[C@@H](Cc2ccc(C(F)(F)P(=O)(O)O)cc2)C(=O)N[C@H](CCCCNC(=O)c2ccc(C)c(Br)c2)C(=O)NCCCCCCCCCCCCCCCC(=O)N[C@H](C(=O)N2C[C@H](O)C[C@H]2C(=O)N[C@@H](C)c2ccc(-c3scnc3C)cc2)C(C)(C)C)ccc1O ; COc1cc(CC(=O)N[C@@H](Cc2ccccc2)C(=O)N[C@@H](Cc2ccc(C(F)(F)P(=O)(O)O)cc2)C(=O)N[C@H](CCCCNC(=O)c2ccc(C)c(Br)c2)C(=O)NCCCCCCCCCCCCCCCC(=O)N[C@H](C(=O)N2C[C@H](O)C[C@H]2C(=O)NCc2ccc(-c3scnc3C)cc2)C(C)(C)C)ccc1O] 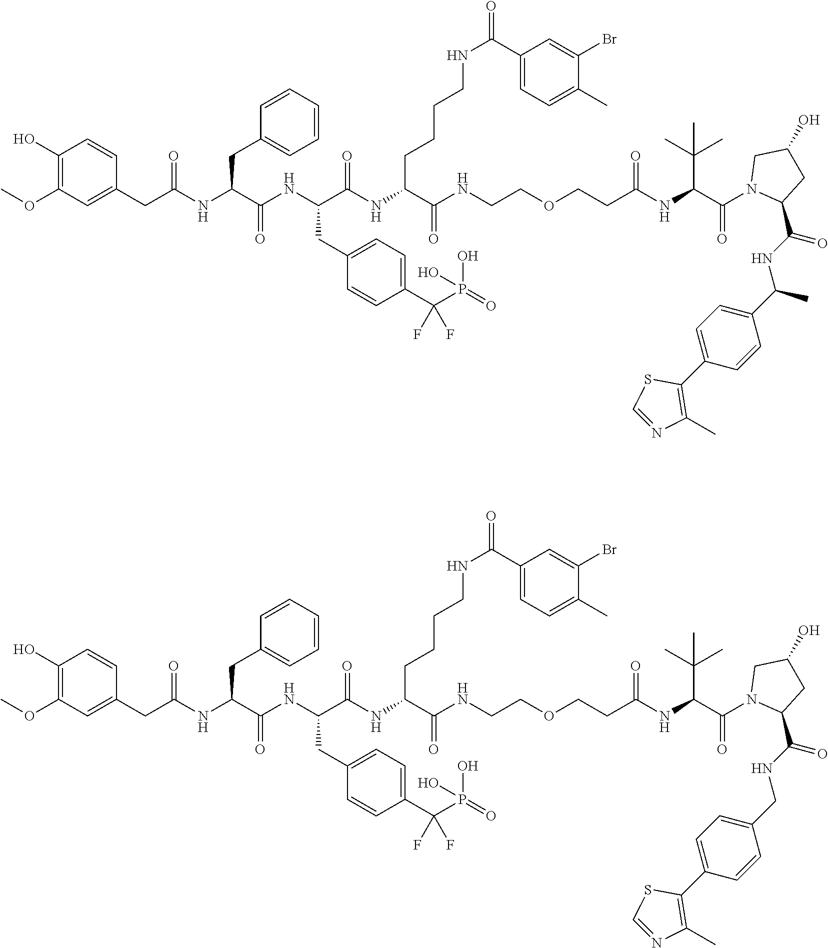 [SMILES: COc1cc(CC(=O)N[C@@H](Cc2ccccc2)C(=O)N[C@@H](Cc2ccc(C(F)(F)P(=O)(O)O)cc2)C(=O)N[C@H](CCCCNC(=O)c2ccc(C)c(Br)c2)C(=O)NCCOCCC(=O)N[C@H](C(=O)N2C[C@H](O)C[C@H]2C(=O)N[C@@H](C)c2ccc(-c3scnc3C)cc2)C(C)(C)C)ccc1O ; COc1cc(CC(=O)N[C@@H](Cc2ccccc2)C(=O)N[C@@H](Cc2ccc(C(F)(F)P(=O)(O)O)cc2)C(=O)N[C@H](CCCCNC(=O)c2ccc(C)c(Br)c2)C(=O)NCCOCCC(=O)N[C@H](C(=O)N2C[C@H](O)C[C@H]2C(=O)NCc2ccc(-c3scnc3C)cc2)C(C)(C)C)ccc1O] 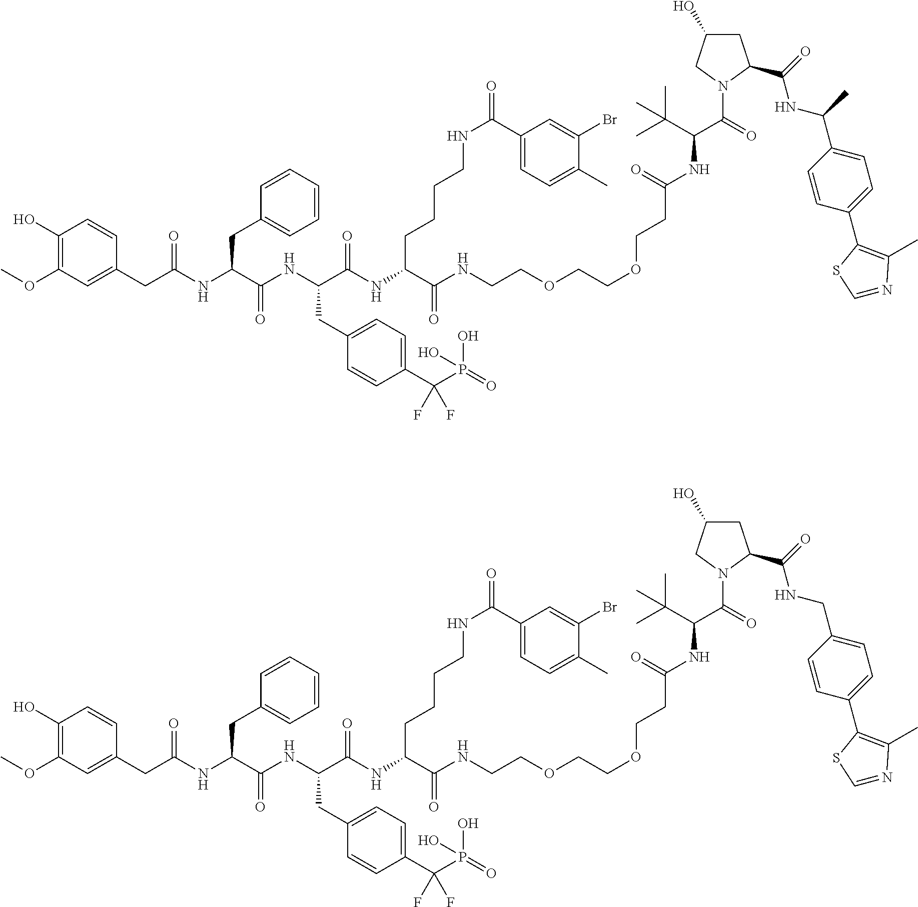 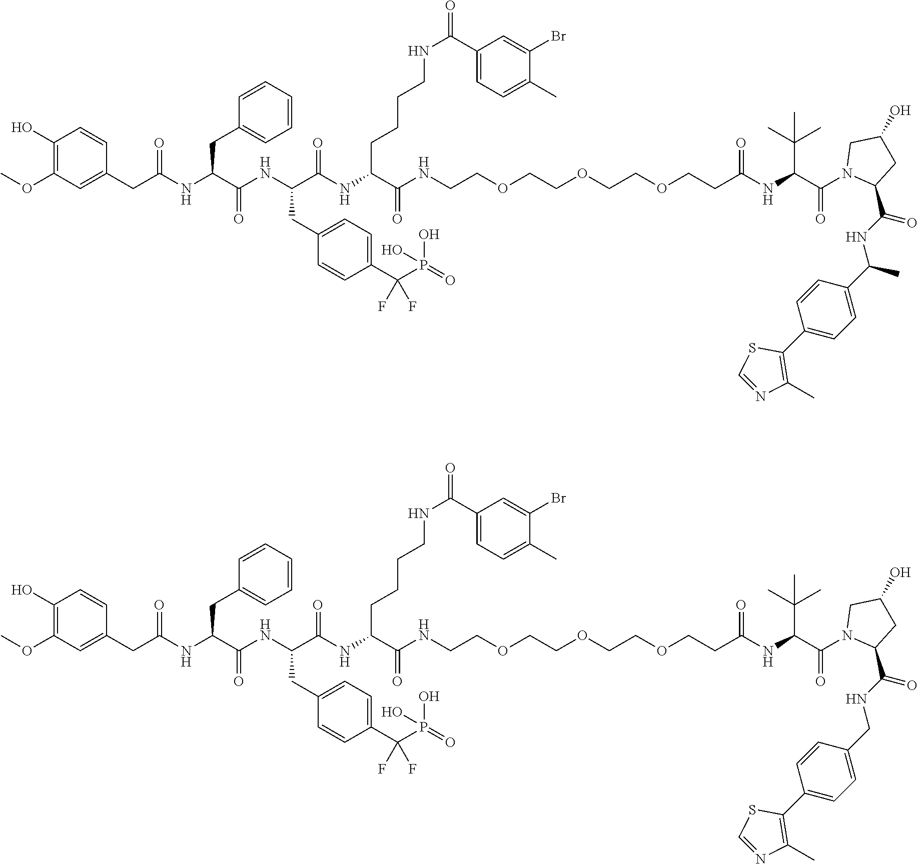 [SMILES: COc1cc(CC(=O)N[C@@H](Cc2ccccc2)C(=O)N[C@@H](Cc2ccc(C(F)(F)P(=O)(O)O)cc2)C(=O)N[C@H](CCCCNC(=O)c2ccc(C)c(Br)c2)C(=O)NCCOCCOCCOCCC(=O)N[C@H](C(=O)N2C[C@H](O)C[C@H]2C(=O)N[C@@H](C)c2ccc(-c3scnc3C)cc2)C(C)(C)C)ccc1O ; COc1cc(CC(=O)N[C@@H](Cc2ccccc2)C(=O)N[C@@H](Cc2ccc(C(F)(F)P(=O)(O)O)cc2)C(=O)N[C@H](CCCCNC(=O)c2ccc(C)c(Br)c2)C(=O)NCCOCCOCCOCCC(=O)N[C@H](C(=O)N2C[C@H](O)C[C@H]2C(=O)NCc2ccc(-c3scnc3C)cc2)C(C)(C)C)ccc1O] 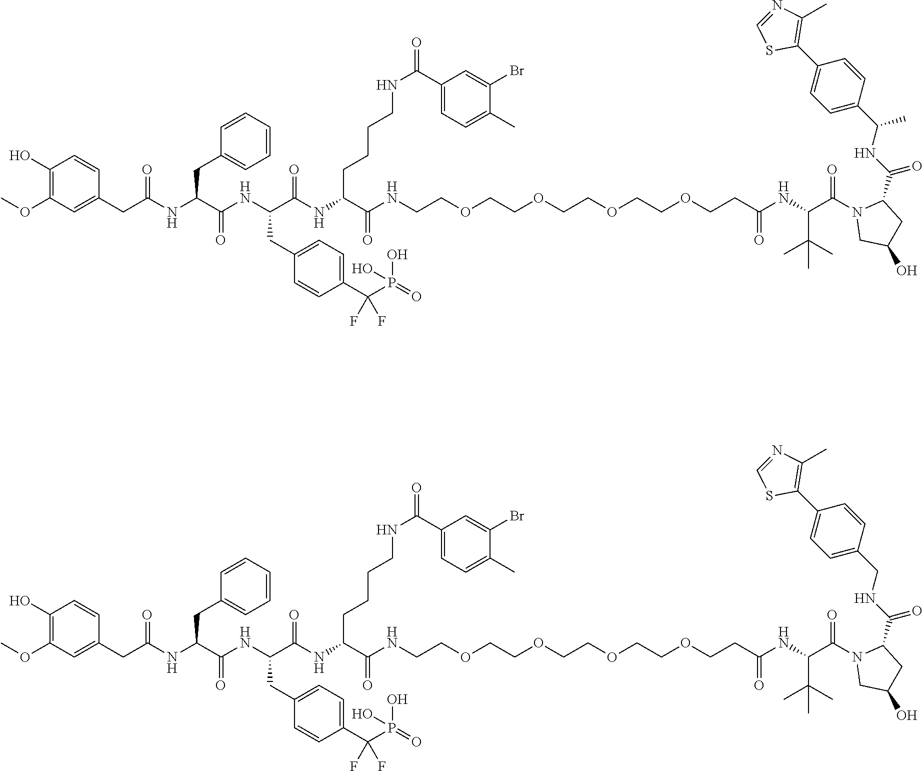 [SMILES: COc1cc(CC(=O)N[C@@H](Cc2ccccc2)C(=O)N[C@@H](Cc2ccc(C(F)(F)P(=O)(O)O)cc2)C(=O)N[C@H](CCCCNC(=O)c2ccc(C)c(Br)c2)C(=O)NCCOCCOCCOCCOCCC(=O)N[C@H](C(=O)N2C[C@H](O)C[C@H]2C(=O)N[C@@H](C)c2ccc(-c3scnc3C)cc2)C(C)(C)C)ccc1O ; COc1cc(CC(=O)N[C@@H](Cc2ccccc2)C(=O)N[C@@H](Cc2ccc(C(F)(F)P(=O)(O)O)cc2)C(=O)N[C@H](CCCCNC(=O)c2ccc(C)c(Br)c2)C(=O)NCCOCCOCCOCCOCCC(=O)N[C@H](C(=O)N2C[C@H](O)C[C@H]2C(=O)NCc2ccc(-c3scnc3C)cc2)C(C)(C)C)ccc1O] 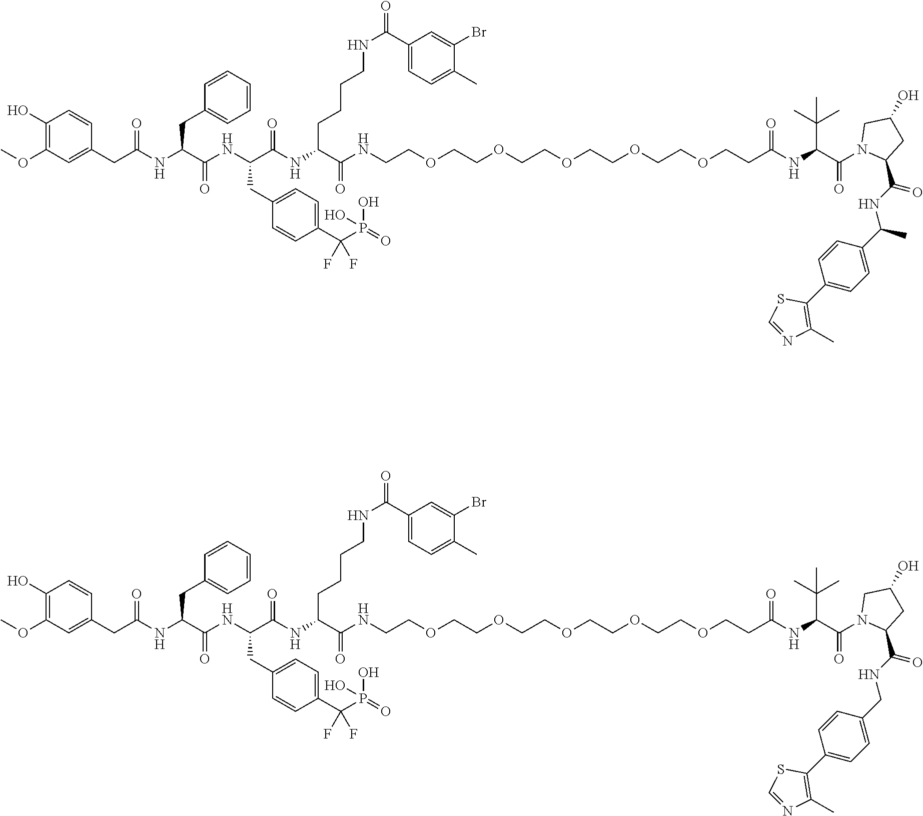 [SMILES: COc1cc(CC(=O)N[C@@H](Cc2ccccc2)C(=O)N[C@@H](Cc2ccc(C(F)(F)P(=O)(O)O)cc2)C(=O)N[C@H](CCCCNC(=O)c2ccc(C)c(Br)c2)C(=O)NCCOCCOCCOCCOCCOCCC(=O)N[C@H](C(=O)N2C[C@H](O)C[C@H]2C(=O)N[C@@H](C)c2ccc(-c3scnc3C)cc2)C(C)(C)C)ccc1O ; COc1cc(CC(=O)N[C@@H](Cc2ccccc2)C(=O)N[C@@H](Cc2ccc(C(F)(F)P(=O)(O)O)cc2)C(=O)N[C@H](CCCCNC(=O)c2ccc(C)c(Br)c2)C(=O)NCCOCCOCCOCCOCCOCCC(=O)N[C@H](C(=O)N2C[C@H](O)C[C@H]2C(=O)NCc2ccc(-c3scnc3C)cc2)C(C)(C)C)ccc1O] 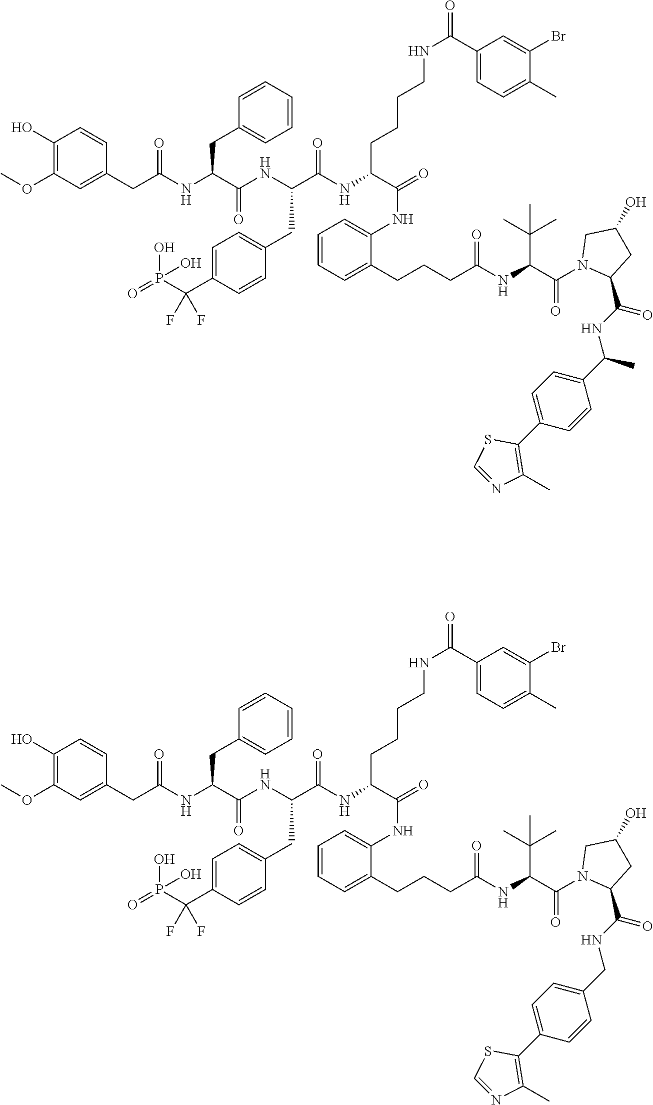 [SMILES: COc1cc(CC(=O)N[C@@H](Cc2ccccc2)C(=O)N[C@@H](Cc2ccc(C(F)(F)P(=O)(O)O)cc2)C(=O)N[C@H](CCCCNC(=O)c2ccc(C)c(Br)c2)C(=O)Nc2ccccc2CCCC(=O)N[C@H](C(=O)N2C[C@H](O)C[C@H]2C(=O)N[C@@H](C)c2ccc(-c3scnc3C)cc2)C(C)(C)C)ccc1O ; COc1cc(CC(=O)N[C@@H](Cc2ccccc2)C(=O)N[C@@H](Cc2ccc(C(F)(F)P(=O)(O)O)cc2)C(=O)N[C@H](CCCCNC(=O)c2ccc(C)c(Br)c2)C(=O)Nc2ccccc2CCCC(=O)N[C@H](C(=O)N2C[C@H](O)C[C@H]2C(=O)NCc2ccc(-c3scnc3C)cc2)C(C)(C)C)ccc1O] 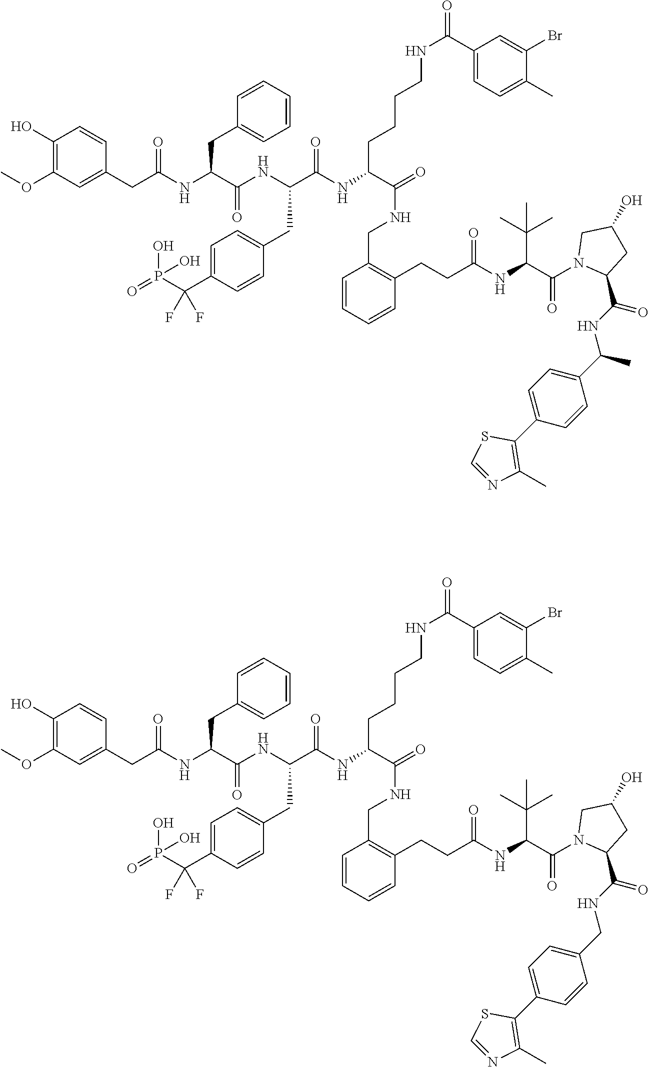 [SMILES: COc1cc(CC(=O)N[C@@H](Cc2ccccc2)C(=O)N[C@@H](Cc2ccc(C(F)(F)P(=O)(O)O)cc2)C(=O)N[C@H](CCCCNC(=O)c2ccc(C)c(Br)c2)C(=O)NCc2ccccc2CCC(=O)N[C@H](C(=O)N2C[C@H](O)C[C@H]2C(=O)N[C@@H](C)c2ccc(-c3scnc3C)cc2)C(C)(C)C)ccc1O ; COc1cc(CC(=O)N[C@@H](Cc2ccccc2)C(=O)N[C@@H](Cc2ccc(C(F)(F)P(=O)(O)O)cc2)C(=O)N[C@H](CCCCNC(=O)c2ccc(C)c(Br)c2)C(=O)NCc2ccccc2CCC(=O)N[C@H](C(=O)N2C[C@H](O)C[C@H]2C(=O)NCc2ccc(-c3scnc3C)cc2)C(C)(C)C)ccc1O] 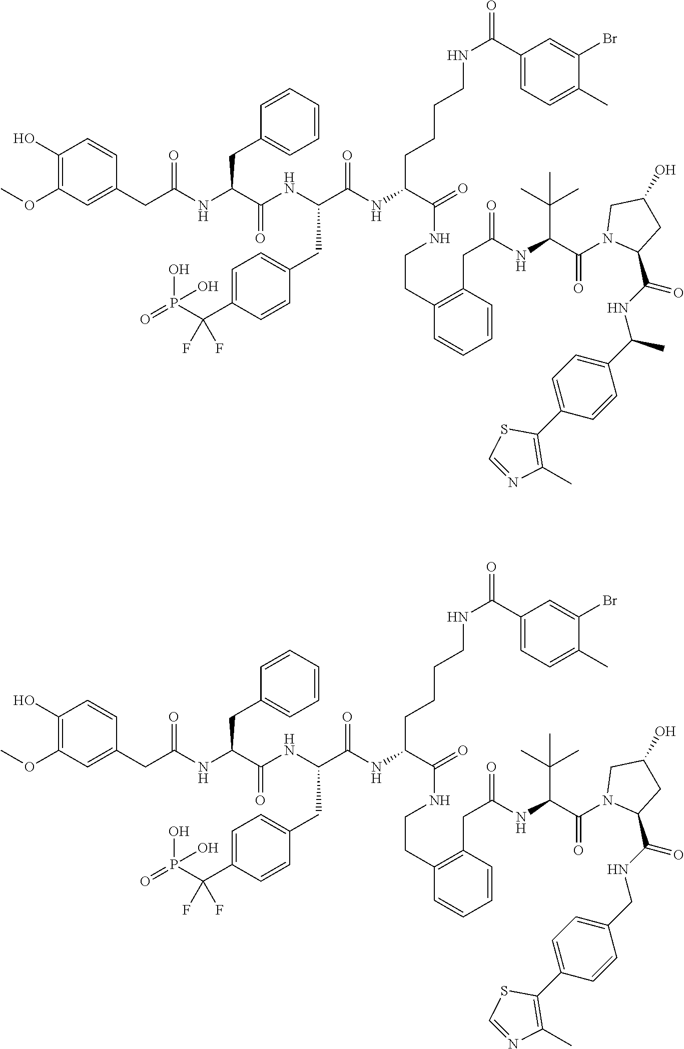 [SMILES: COc1cc(CC(=O)N[C@@H](Cc2ccccc2)C(=O)N[C@@H](Cc2ccc(C(F)(F)P(=O)(O)O)cc2)C(=O)N[C@H](CCCCNC(=O)c2ccc(C)c(Br)c2)C(=O)NCCc2ccccc2CC(=O)N[C@H](C(=O)N2C[C@H](O)C[C@H]2C(=O)N[C@@H](C)c2ccc(-c3scnc3C)cc2)C(C)(C)C)ccc1O ; COc1cc(CC(=O)N[C@@H](Cc2ccccc2)C(=O)N[C@@H](Cc2ccc(C(F)(F)P(=O)(O)O)cc2)C(=O)N[C@H](CCCCNC(=O)c2ccc(C)c(Br)c2)C(=O)NCCc2ccccc2CC(=O)N[C@H](C(=O)N2C[C@H](O)C[C@H]2C(=O)NCc2ccc(-c3scnc3C)cc2)C(C)(C)C)ccc1O] 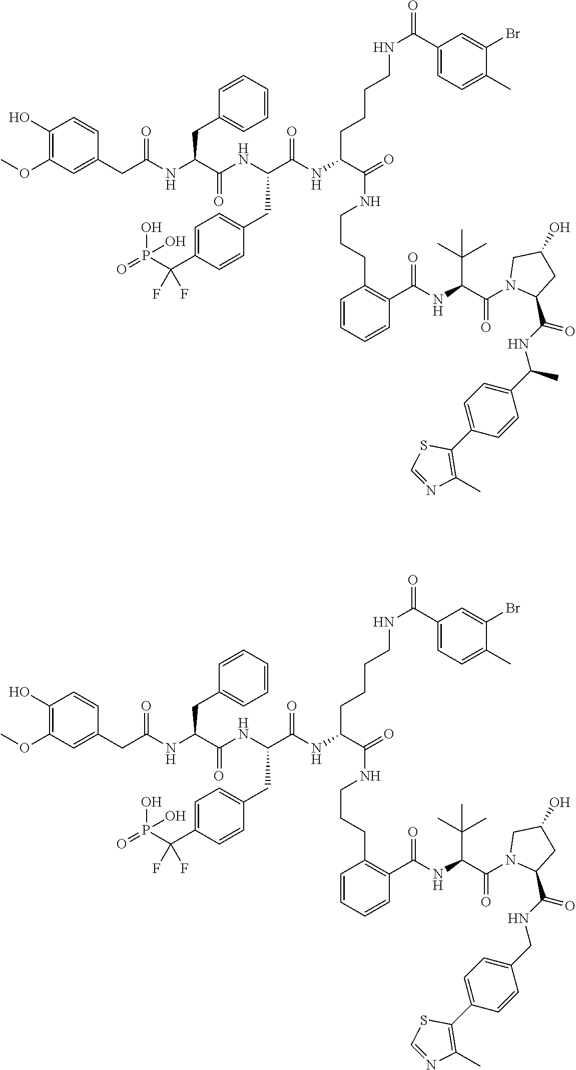 [SMILES: COc1cc(CC(=O)N[C@@H](Cc2ccccc2)C(=O)N[C@@H](Cc2ccc(C(F)(F)P(=O)(O)O)cc2)C(=O)N[C@H](CCCCNC(=O)c2ccc(C)c(Br)c2)C(=O)NCCCc2ccccc2C(=O)N[C@H](C(=O)N2C[C@H](O)C[C@H]2C(=O)N[C@@H](C)c2ccc(-c3scnc3C)cc2)C(C)(C)C)ccc1O ; COc1cc(CC(=O)N[C@@H](Cc2ccccc2)C(=O)N[C@@H](Cc2ccc(C(F)(F)P(=O)(O)O)cc2)C(=O)N[C@H](CCCCNC(=O)c2ccc(C)c(Br)c2)C(=O)NCCCc2ccccc2C(=O)N[C@H](C(=O)N2C[C@H](O)C[C@H]2C(=O)NCc2ccc(-c3scnc3C)cc2)C(C)(C)C)ccc1O] 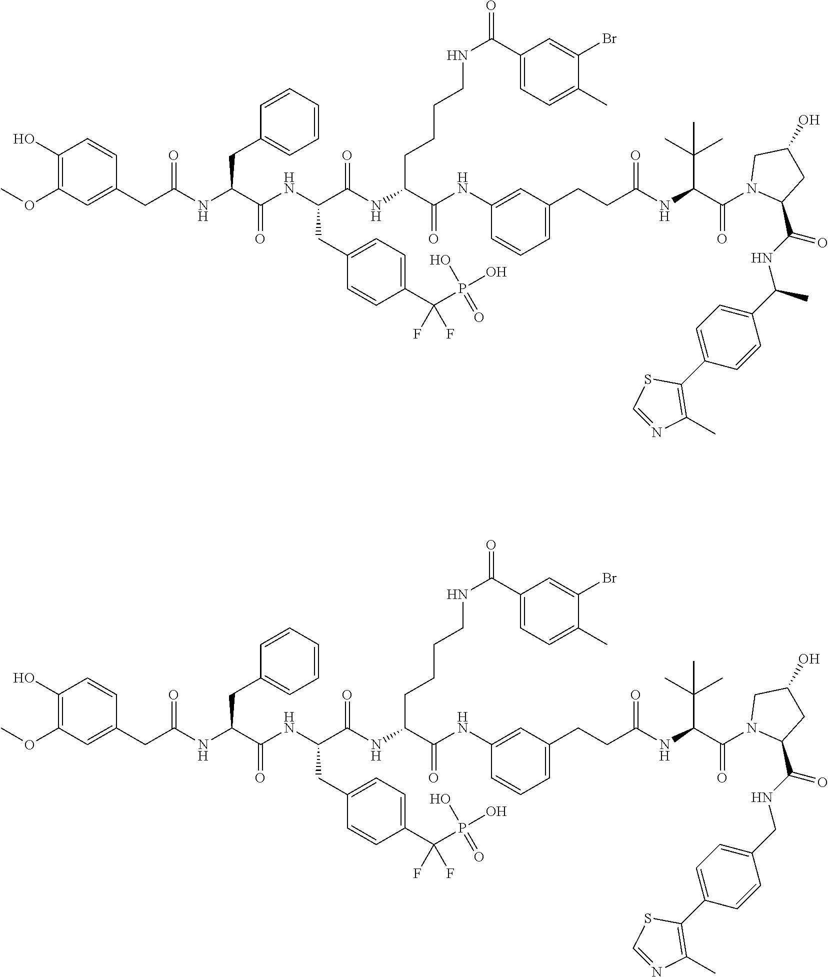 [SMILES: COc1cc(CC(=O)N[C@@H](Cc2ccccc2)C(=O)N[C@@H](Cc2ccc(C(F)(F)P(=O)(O)O)cc2)C(=O)N[C@H](CCCCNC(=O)c2ccc(C)c(Br)c2)C(=O)Nc2cccc(CCC(=O)N[C@H](C(=O)N3C[C@H](O)C[C@H]3C(=O)N[C@@H](C)c3ccc(-c4scnc4C)cc3)C(C)(C)C)c2)ccc1O ; COc1cc(CC(=O)N[C@@H](Cc2ccccc2)C(=O)N[C@@H](Cc2ccc(C(F)(F)P(=O)(O)O)cc2)C(=O)N[C@H](CCCCNC(=O)c2ccc(C)c(Br)c2)C(=O)Nc2cccc(CCC(=O)N[C@H](C(=O)N3C[C@H](O)C[C@H]3C(=O)NCc3ccc(-c4scnc4C)cc3)C(C)(C)C)c2)ccc1O] 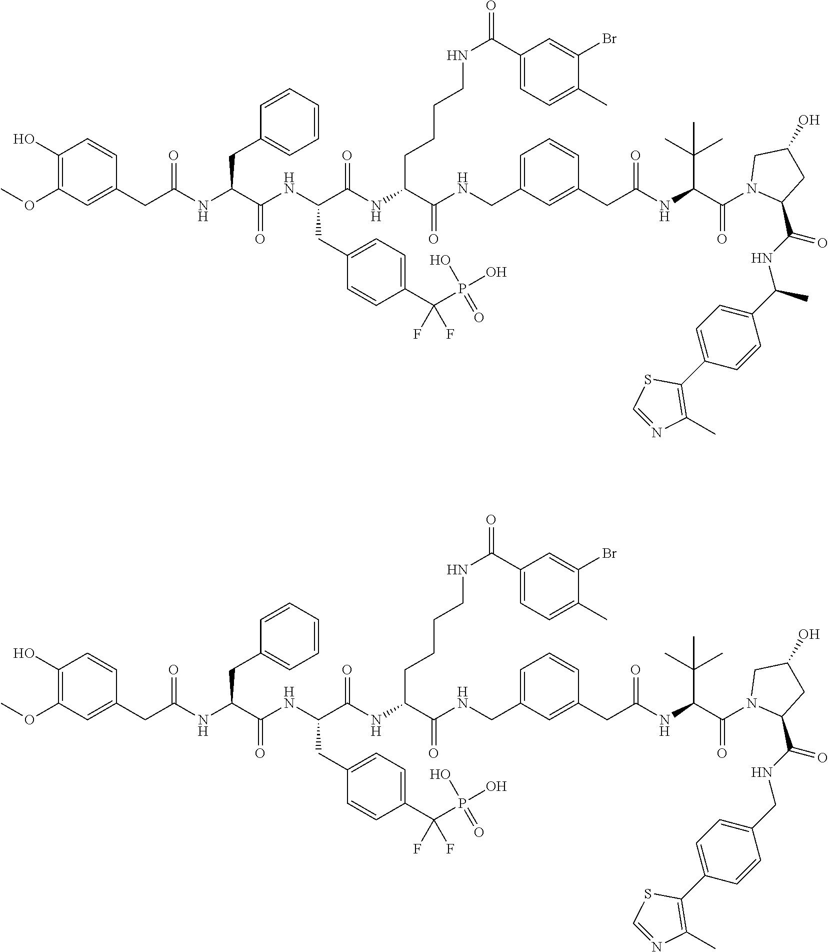 [SMILES: COc1cc(CC(=O)N[C@@H](Cc2ccccc2)C(=O)N[C@@H](Cc2ccc(C(F)(F)P(=O)(O)O)cc2)C(=O)N[C@H](CCCCNC(=O)c2ccc(C)c(Br)c2)C(=O)NCc2cccc(CC(=O)N[C@H](C(=O)N3C[C@H](O)C[C@H]3C(=O)N[C@@H](C)c3ccc(-c4scnc4C)cc3)C(C)(C)C)c2)ccc1O ; COc1cc(CC(=O)N[C@@H](Cc2ccccc2)C(=O)N[C@@H](Cc2ccc(C(F)(F)P(=O)(O)O)cc2)C(=O)N[C@H](CCCCNC(=O)c2ccc(C)c(Br)c2)C(=O)NCc2cccc(CC(=O)N[C@H](C(=O)N3C[C@H](O)C[C@H]3C(=O)NCc3ccc(-c4scnc4C)cc3)C(C)(C)C)c2)ccc1O] 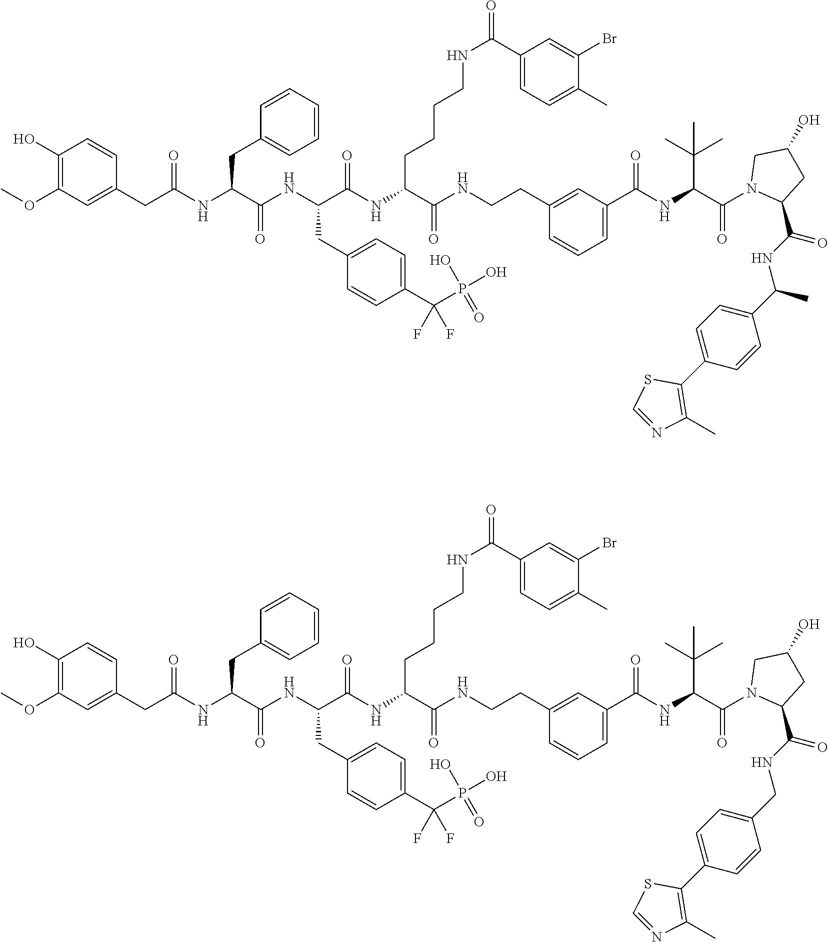 [SMILES: COc1cc(CC(=O)N[C@@H](Cc2ccccc2)C(=O)N[C@@H](Cc2ccc(C(F)(F)P(=O)(O)O)cc2)C(=O)N[C@H](CCCCNC(=O)c2ccc(C)c(Br)c2)C(=O)NCCc2cccc(C(=O)N[C@H](C(=O)N3C[C@H](O)C[C@H]3C(=O)N[C@@H](C)c3ccc(-c4scnc4C)cc3)C(C)(C)C)c2)ccc1O ; COc1cc(CC(=O)N[C@@H](Cc2ccccc2)C(=O)N[C@@H](Cc2ccc(C(F)(F)P(=O)(O)O)cc2)C(=O)N[C@H](CCCCNC(=O)c2ccc(C)c(Br)c2)C(=O)NCCc2cccc(C(=O)N[C@H](C(=O)N3C[C@H](O)C[C@H]3C(=O)NCc3ccc(-c4scnc4C)cc3)C(C)(C)C)c2)ccc1O] 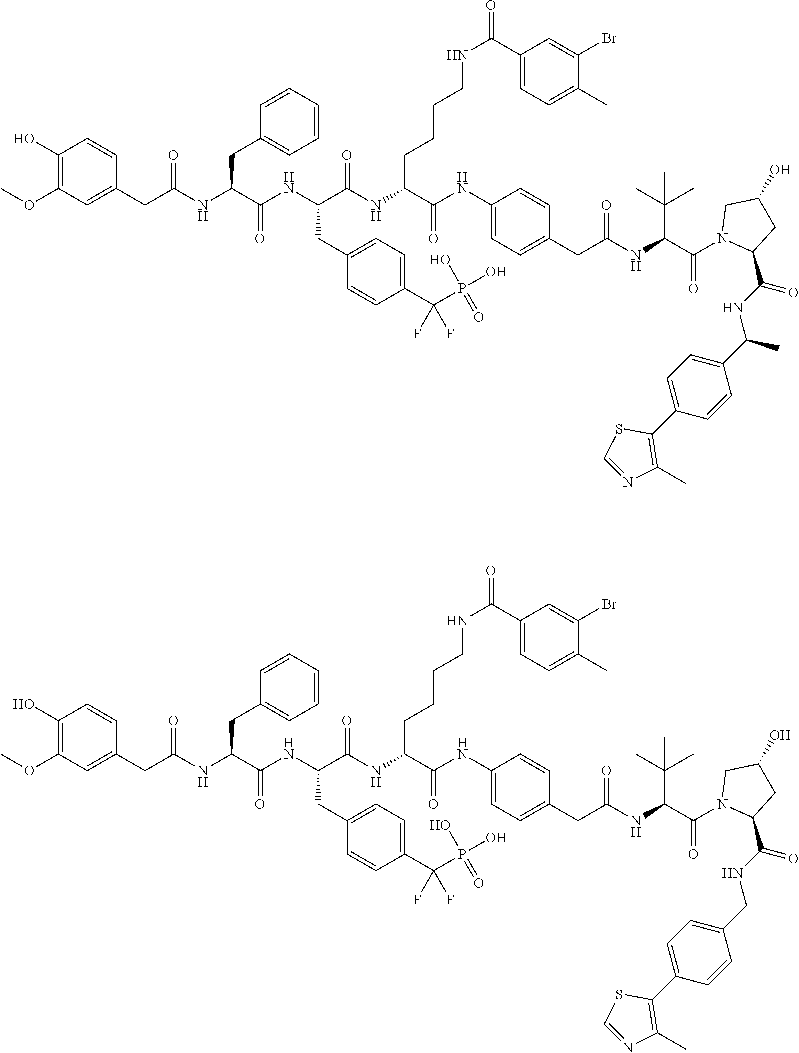 [SMILES: COc1cc(CC(=O)N[C@@H](Cc2ccccc2)C(=O)N[C@@H](Cc2ccc(C(F)(F)P(=O)(O)O)cc2)C(=O)N[C@H](CCCCNC(=O)c2ccc(C)c(Br)c2)C(=O)Nc2ccc(CC(=O)N[C@H](C(=O)N3C[C@H](O)C[C@H]3C(=O)N[C@@H](C)c3ccc(-c4scnc4C)cc3)C(C)(C)C)cc2)ccc1O ; COc1cc(CC(=O)N[C@@H](Cc2ccccc2)C(=O)N[C@@H](Cc2ccc(C(F)(F)P(=O)(O)O)cc2)C(=O)N[C@H](CCCCNC(=O)c2ccc(C)c(Br)c2)C(=O)Nc2ccc(CC(=O)N[C@H](C(=O)N3C[C@H](O)C[C@H]3C(=O)NCc3ccc(-c4scnc4C)cc3)C(C)(C)C)cc2)ccc1O] 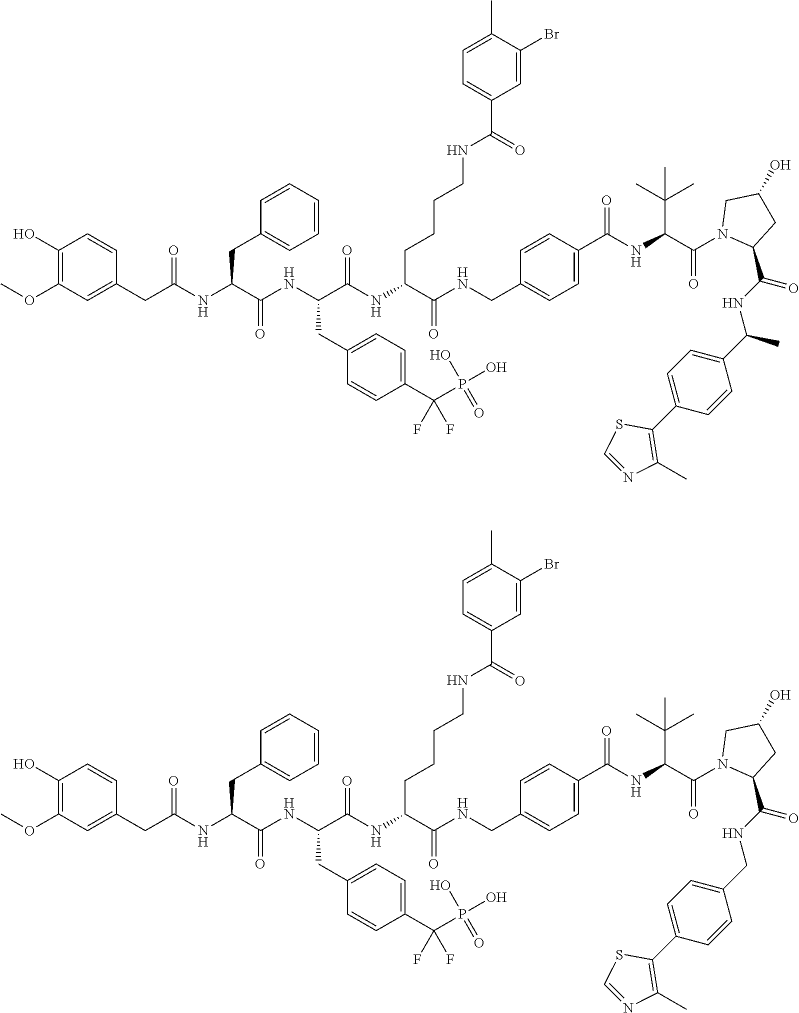 [SMILES: COc1cc(CC(=O)N[C@@H](Cc2ccccc2)C(=O)N[C@@H](Cc2ccc(C(F)(F)P(=O)(O)O)cc2)C(=O)N[C@H](CCCCNC(=O)c2ccc(C)c(Br)c2)C(=O)NCc2ccc(C(=O)N[C@H](C(=O)N3C[C@H](O)C[C@H]3C(=O)N[C@@H](C)c3ccc(-c4scnc4C)cc3)C(C)(C)C)cc2)ccc1O ; COc1cc(CC(=O)N[C@@H](Cc2ccccc2)C(=O)N[C@@H](Cc2ccc(C(F)(F)P(=O)(O)O)cc2)C(=O)N[C@H](CCCCNC(=O)c2ccc(C)c(Br)c2)C(=O)NCc2ccc(C(=O)N[C@H](C(=O)N3C[C@H](O)C[C@H]3C(=O)NCc3ccc(-c4scnc4C)cc3)C(C)(C)C)cc2)ccc1O] 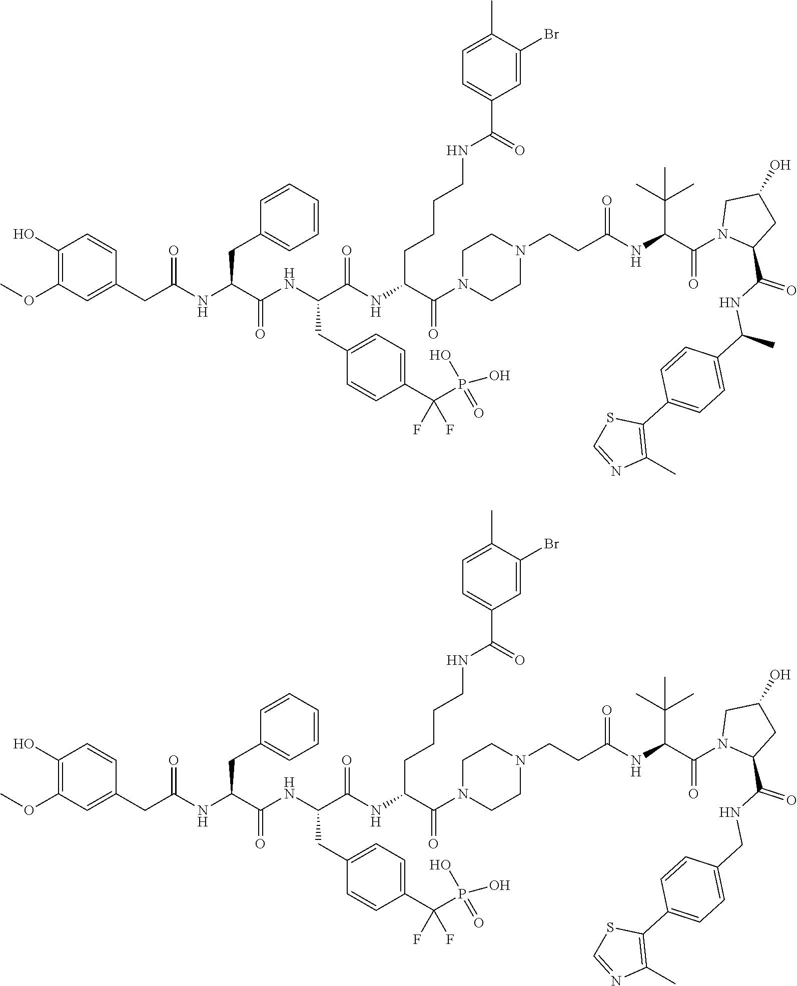 [SMILES: COc1cc(CC(=O)N[C@@H](Cc2ccccc2)C(=O)N[C@@H](Cc2ccc(C(F)(F)P(=O)(O)O)cc2)C(=O)N[C@H](CCCCNC(=O)c2ccc(C)c(Br)c2)C(=O)N2CCN(CCC(=O)N[C@H](C(=O)N3C[C@H](O)C[C@H]3C(=O)N[C@@H](C)c3ccc(-c4scnc4C)cc3)C(C)(C)C)CC2)ccc1O ; COc1cc(CC(=O)N[C@@H](Cc2ccccc2)C(=O)N[C@@H](Cc2ccc(C(F)(F)P(=O)(O)O)cc2)C(=O)N[C@H](CCCCNC(=O)c2ccc(C)c(Br)c2)C(=O)N2CCN(CCC(=O)N[C@H](C(=O)N3C[C@H](O)C[C@H]3C(=O)NCc3ccc(-c4scnc4C)cc3)C(C)(C)C)CC2)ccc1O] 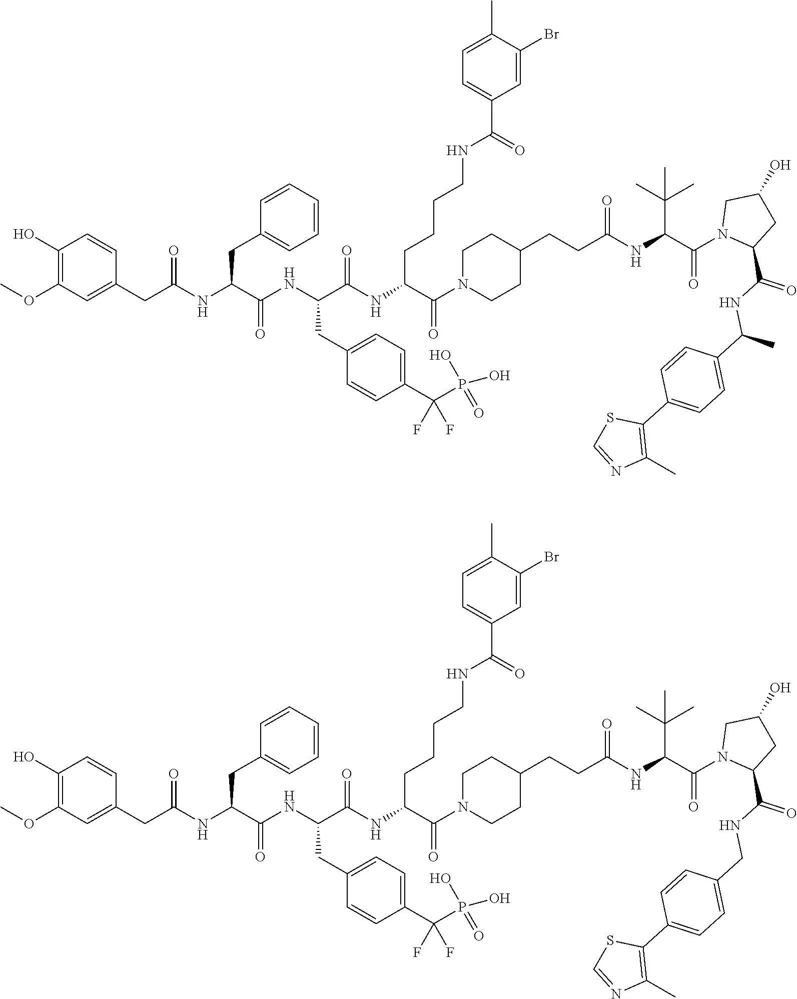 [SMILES: COc1cc(CC(=O)N[C@@H](Cc2ccccc2)C(=O)N[C@@H](Cc2ccc(C(F)(F)P(=O)(O)O)cc2)C(=O)N[C@H](CCCCNC(=O)c2ccc(C)c(Br)c2)C(=O)N2CCC(CCC(=O)N[C@H](C(=O)N3C[C@H](O)C[C@H]3C(=O)N[C@@H](C)c3ccc(-c4scnc4C)cc3)C(C)(C)C)CC2)ccc1O ; COc1cc(CC(=O)N[C@@H](Cc2ccccc2)C(=O)N[C@@H](Cc2ccc(C(F)(F)P(=O)(O)O)cc2)C(=O)N[C@H](CCCCNC(=O)c2ccc(C)c(Br)c2)C(=O)N2CCC(CCC(=O)N[C@H](C(=O)N3C[C@H](O)C[C@H]3C(=O)NCc3ccc(-c4scnc4C)cc3)C(C)(C)C)CC2)ccc1O] 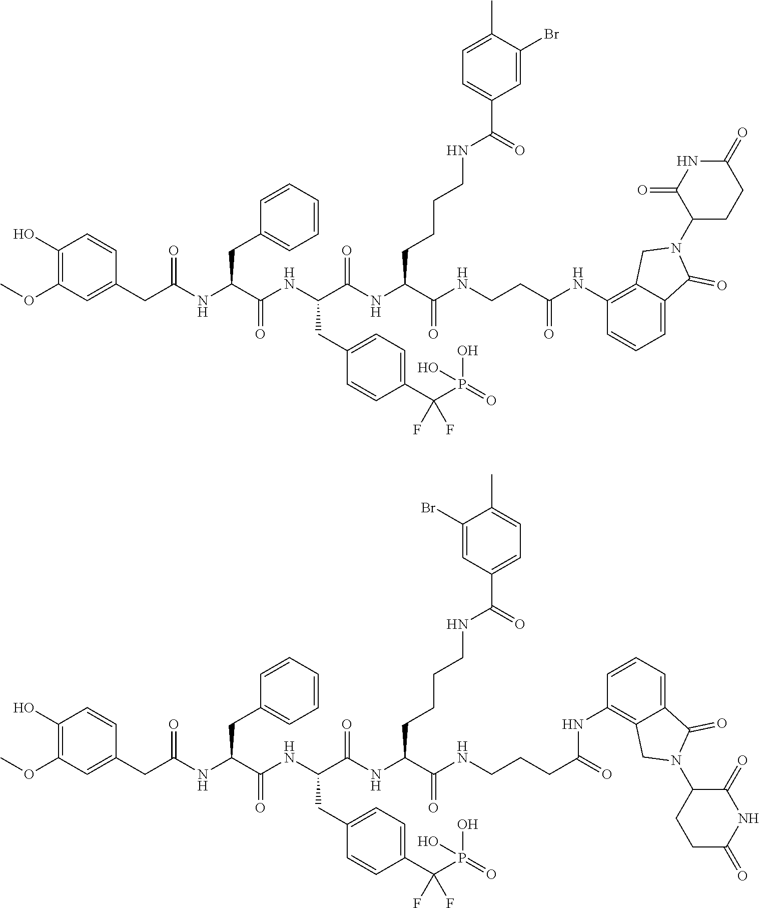 [SMILES: COc1cc(CC(=O)N[C@@H](Cc2ccccc2)C(=O)N[C@@H](Cc2ccc(C(F)(F)P(=O)(O)O)cc2)C(=O)N[C@@H](CCCCNC(=O)c2ccc(C)c(Br)c2)C(=O)NCCC(=O)Nc2cccc3c2CN(C2CCC(=O)NC2=O)C3=O)ccc1O ; COc1cc(CC(=O)N[C@@H](Cc2ccccc2)C(=O)N[C@@H](Cc2ccc(C(F)(F)P(=O)(O)O)cc2)C(=O)N[C@@H](CCCCNC(=O)c2ccc(C)c(Br)c2)C(=O)NCCCC(=O)Nc2cccc3c2CN(C2CCC(=O)NC2=O)C3=O)ccc1O] 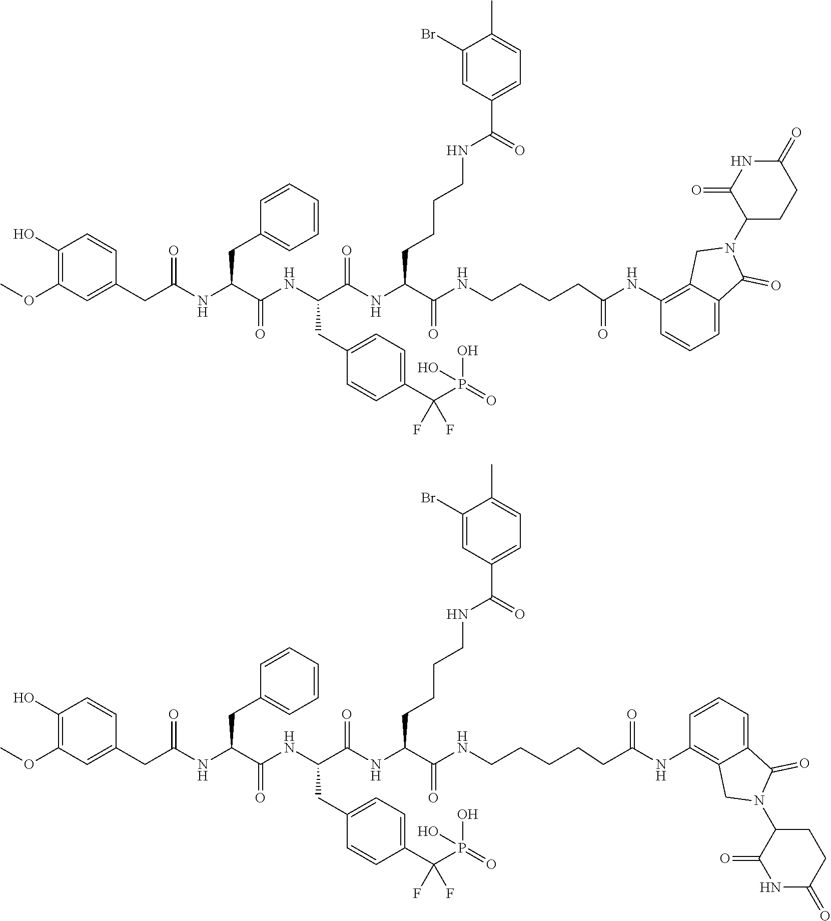 [SMILES: COc1cc(CC(=O)N[C@@H](Cc2ccccc2)C(=O)N[C@@H](Cc2ccc(C(F)(F)P(=O)(O)O)cc2)C(=O)N[C@@H](CCCCNC(=O)c2ccc(C)c(Br)c2)C(=O)NCCCCC(=O)Nc2cccc3c2CN(C2CCC(=O)NC2=O)C3=O)ccc1O ; COc1cc(CC(=O)N[C@@H](Cc2ccccc2)C(=O)N[C@@H](Cc2ccc(C(F)(F)P(=O)(O)O)cc2)C(=O)N[C@@H](CCCCNC(=O)c2ccc(C)c(Br)c2)C(=O)NCCCCCC(=O)Nc2cccc3c2CN(C2CCC(=O)NC2=O)C3=O)ccc1O] 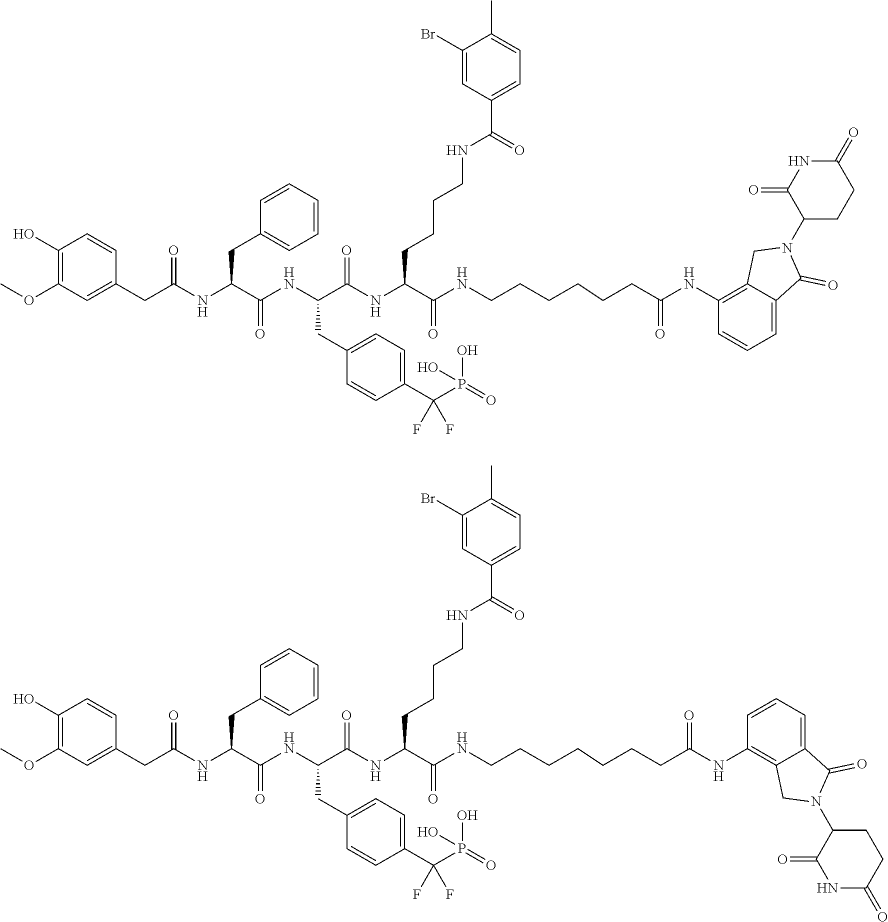 [SMILES: COc1cc(CC(=O)N[C@@H](Cc2ccccc2)C(=O)N[C@@H](Cc2ccc(C(F)(F)P(=O)(O)O)cc2)C(=O)N[C@@H](CCCCNC(=O)c2ccc(C)c(Br)c2)C(=O)NCCCCCCC(=O)Nc2cccc3c2CN(C2CCC(=O)NC2=O)C3=O)ccc1O ; COc1cc(CC(=O)N[C@@H](Cc2ccccc2)C(=O)N[C@@H](Cc2ccc(C(F)(F)P(=O)(O)O)cc2)C(=O)N[C@@H](CCCCNC(=O)c2ccc(C)c(Br)c2)C(=O)NCCCCCCCC(=O)Nc2cccc3c2CN(C2CCC(=O)NC2=O)C3=O)ccc1O] 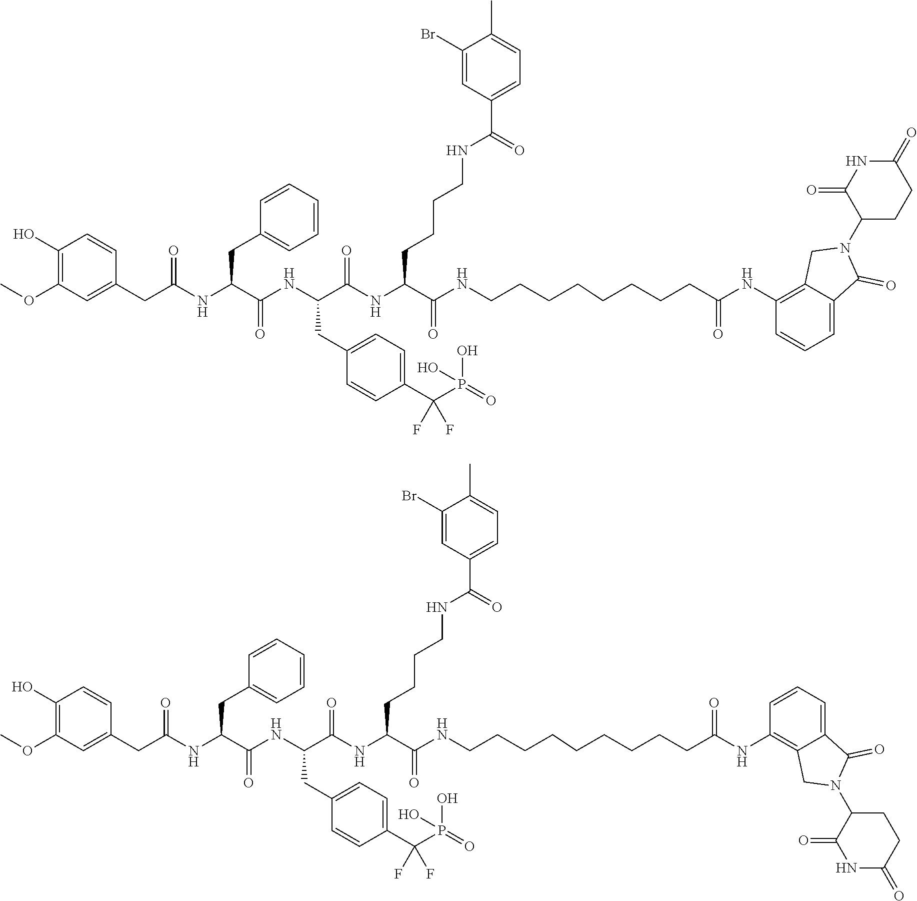 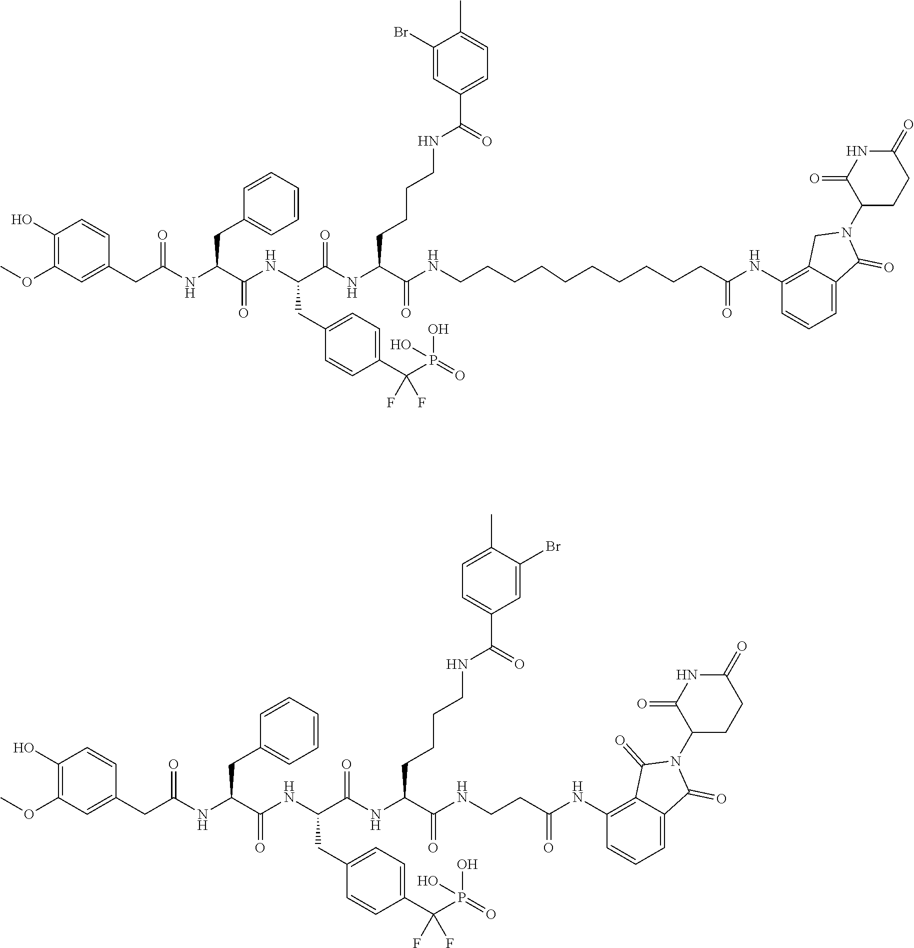 [SMILES: COc1cc(CC(=O)N[C@@H](Cc2ccccc2)C(=O)N[C@@H](Cc2ccc(C(F)(F)P(=O)(O)O)cc2)C(=O)N[C@@H](CCCCNC(=O)c2ccc(C)c(Br)c2)C(=O)NCCCCCCCCCCC(=O)Nc2cccc3c2CN(C2CCC(=O)NC2=O)C3=O)ccc1O ; COc1cc(CC(=O)N[C@@H](Cc2ccccc2)C(=O)N[C@@H](Cc2ccc(C(F)(F)P(=O)(O)O)cc2)C(=O)N[C@@H](CCCCNC(=O)c2ccc(C)c(Br)c2)C(=O)NCCC(=O)Nc2cccc3c2C(=O)N(C2CCC(=O)NC2=O)C3=O)ccc1O] 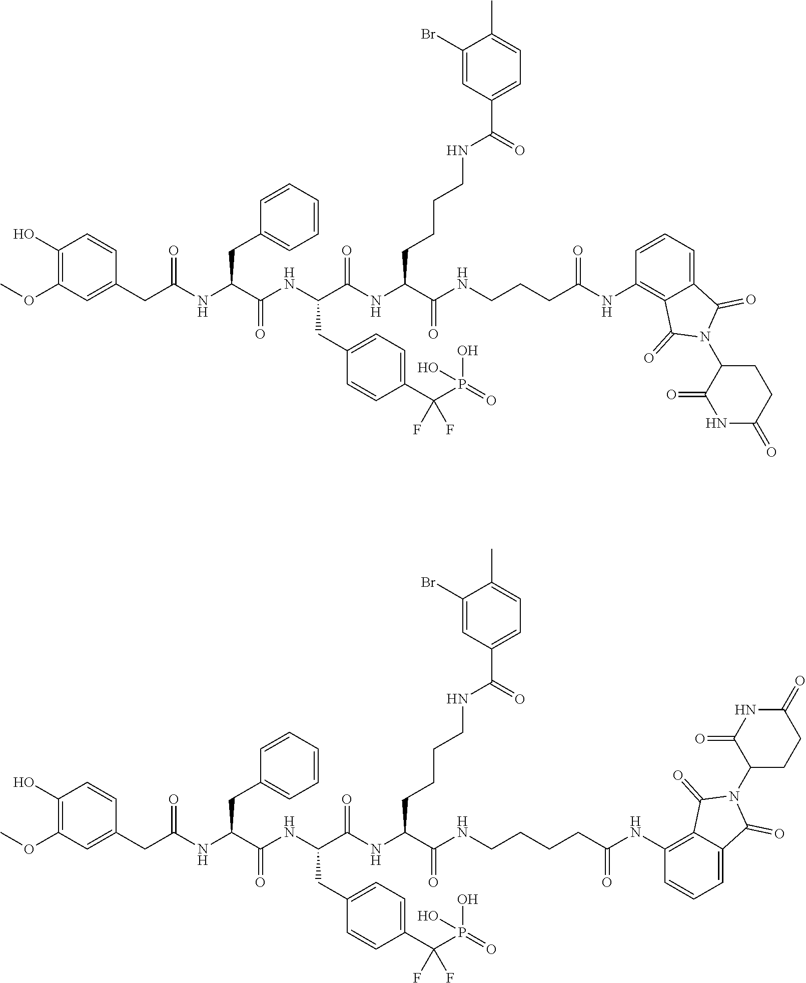 [SMILES: COc1cc(CC(=O)N[C@@H](Cc2ccccc2)C(=O)N[C@@H](Cc2ccc(C(F)(F)P(=O)(O)O)cc2)C(=O)N[C@@H](CCCCNC(=O)c2ccc(C)c(Br)c2)C(=O)NCCCC(=O)Nc2cccc3c2C(=O)N(C2CCC(=O)NC2=O)C3=O)ccc1O ; COc1cc(CC(=O)N[C@@H](Cc2ccccc2)C(=O)N[C@@H](Cc2ccc(C(F)(F)P(=O)(O)O)cc2)C(=O)N[C@@H](CCCCNC(=O)c2ccc(C)c(Br)c2)C(=O)NCCCCC(=O)Nc2cccc3c2C(=O)N(C2CCC(=O)NC2=O)C3=O)ccc1O] 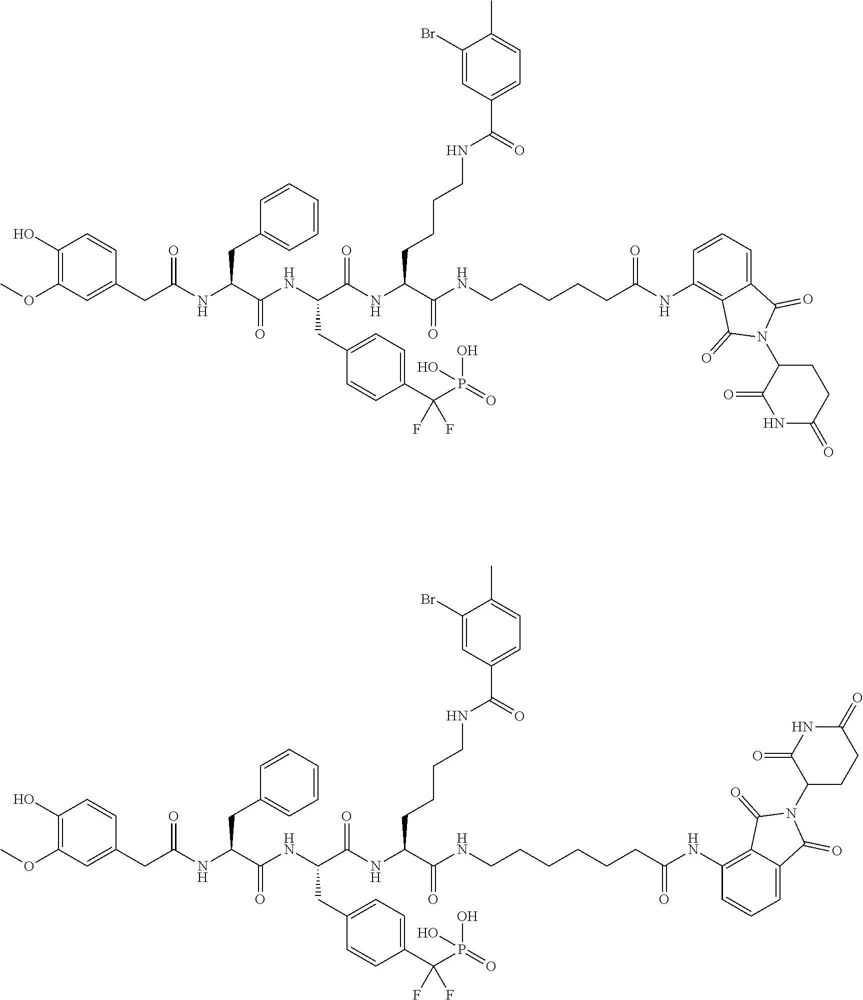 [SMILES: COc1cc(CC(=O)N[C@@H](Cc2ccccc2)C(=O)N[C@@H](Cc2ccc(C(F)(F)P(=O)(O)O)cc2)C(=O)N[C@@H](CCCCNC(=O)c2ccc(C)c(Br)c2)C(=O)NCCCCCC(=O)Nc2cccc3c2C(=O)N(C2CCC(=O)NC2=O)C3=O)ccc1O ; COc1cc(CC(=O)N[C@@H](Cc2ccccc2)C(=O)N[C@@H](Cc2ccc(C(F)(F)P(=O)(O)O)cc2)C(=O)N[C@@H](CCCCNC(=O)c2ccc(C)c(Br)c2)C(=O)NCCCCCCC(=O)Nc2cccc3c2C(=O)N(C2CCC(=O)NC2=O)C3=O)ccc1O] 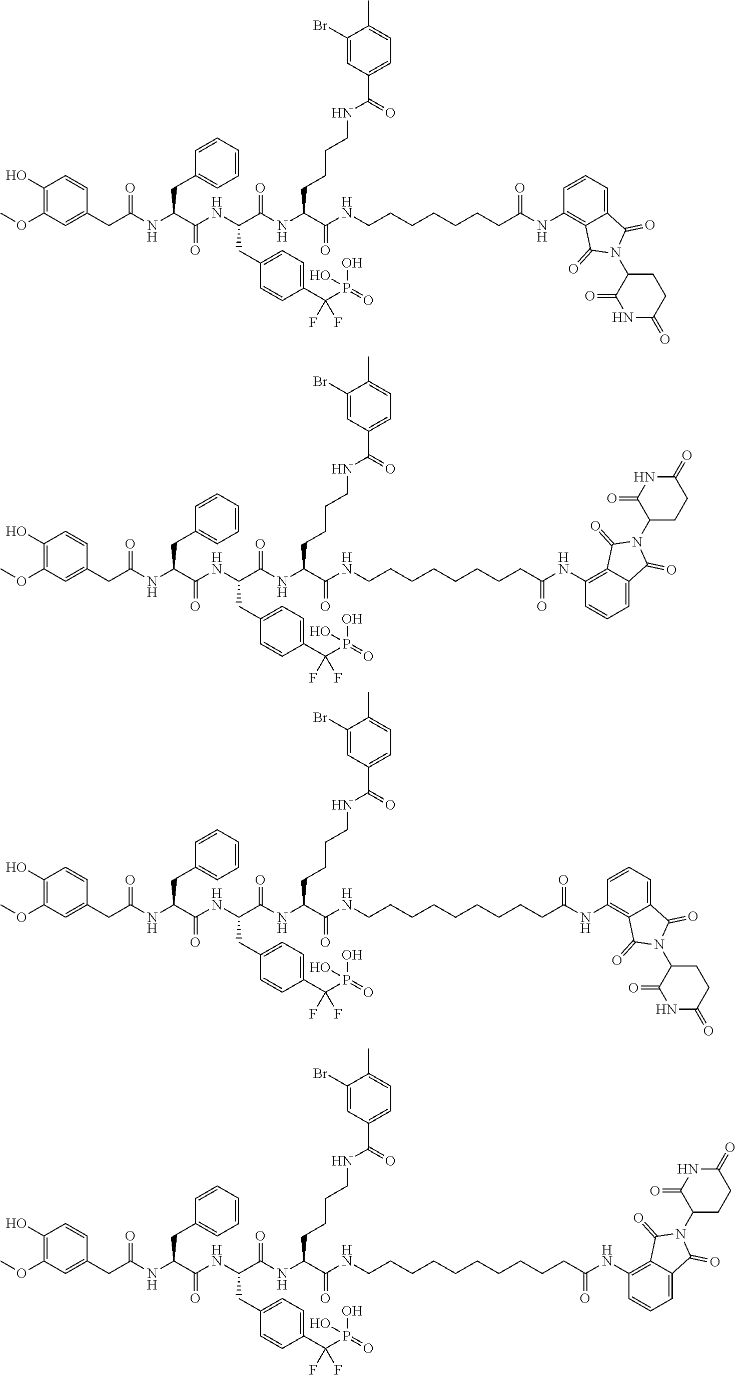 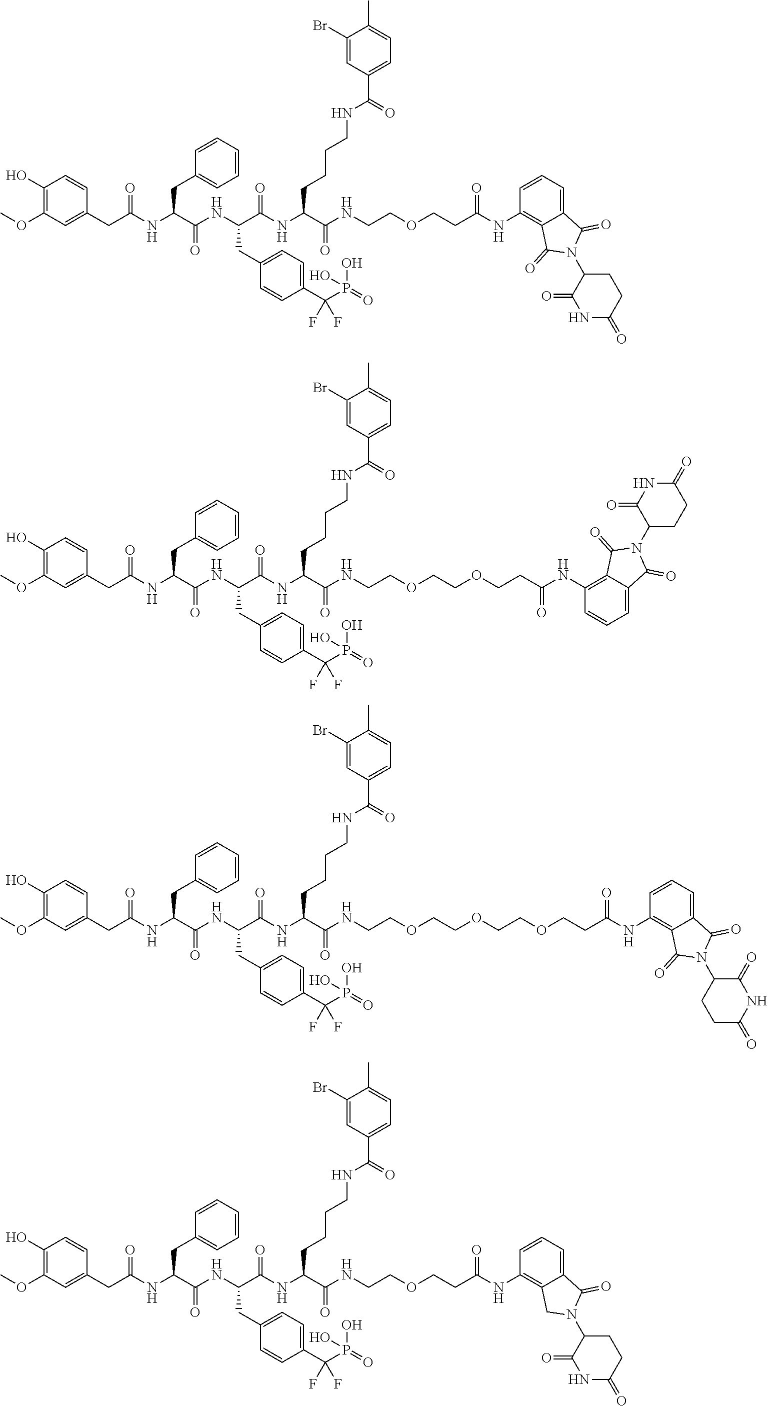 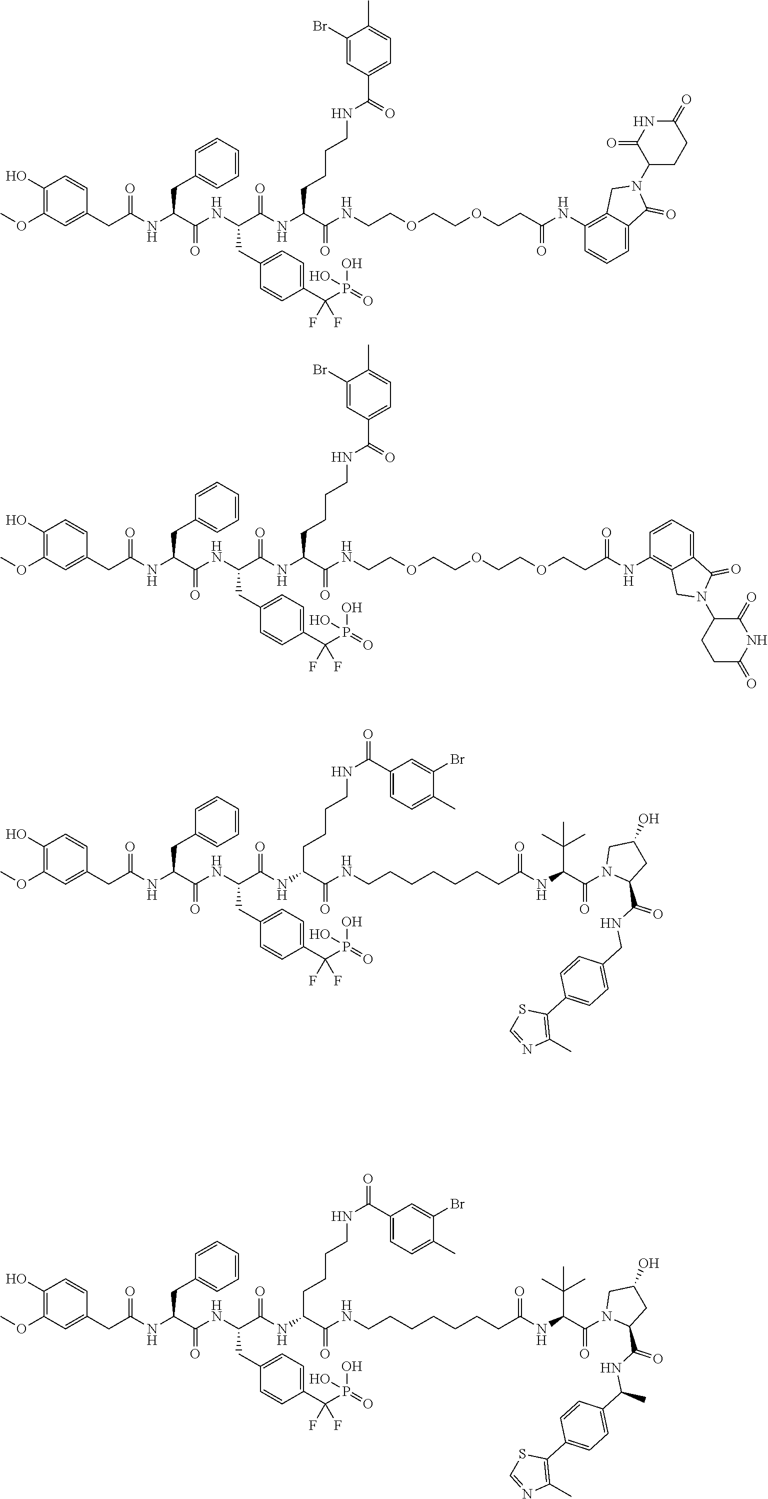
or, pharmaceutically acceptable salt, hydrate, tautomer, and stereoisomer thereof.

16. A pharmaceutical composition comprising one or more compounds of any one of claims 1-3 or a pharmaceutically acceptable salt, hydrate, tautomer, and stereoisomer thereof, and a pharmaceutically acceptable carrier or excipient.

17. A pharmaceutical composition comprising one or more compounds of any one of claims 4-9 or a pharmaceutically acceptable salt, hydrate, tautomer, and stereoisomer thereof, and a pharmaceutically acceptable carrier or excipient.

18. A pharmaceutical composition comprising one or more compounds of any one of claims 10-14 or a pharmaceutically acceptable salt, hydrate, tautomer, and stereoisomer thereof, and a pharmaceutically acceptable carrier or excipient.

19. A method of treating or preventing cancer in a patient, wherein the method comprises inhibiting or degrading dual protein-tyrosine phosphatase 1B (PTP1B) and T-cell protein tyrosine phosphatase (TC-PTP) by administering to the patient in need thereof an effective amount of a compound of any one of claims 1-3 or a pharmaceutical composition of claim 16, whereupon the cancer in the patient is treated or prevented.

20. The method of claim 19, wherein the cancer is colon cancer, lung adenocarcinoma, squamous cell carcinoma, or melanoma.

21. The method of claim 19, wherein the compound is administered orally.

22. A method of treating or preventing cancer in a patient, wherein the method comprises inhibiting dual PTP1B and TC-PTP inhibitors by administering to the patient in need thereof an effective amount of a compound of any one of claims 4-9 or a pharmaceutical composition of claim 17, whereupon the cancer in the patient is treated or prevented.

23. The method of claim 22, wherein the cancer is colon cancer, lung adenocarcinoma, squamous cell carcinoma, or melanoma.

24. The method of claim 22, wherein the compound is administered orally.

25. A method of treating or preventing cancer in a patient, wherein the method comprises degrading dual PTP1B and TC-PTP proteins by administering to the patient in need thereof an effective amount of a compound of any one of claims 10-14 or a pharmaceutical composition of claim 18, whereupon the cancer in the patient is treated or prevented.

26. The method of claim 25, wherein the cancer is colon cancer, lung adenocarcinoma, squamous cell carcinoma, or melanoma.

27. The method of claim 25, wherein the compound is administered orally.

28. A method of treating or preventing type II diabetes in a patient, wherein the method comprises inhibiting or degrading dual PTP1B and TC-PTP by administering to the patient in need thereof an effective amount of a compound of any one of claims 1-3 or a pharmaceutical composition of claim 16, whereupon the type II diabetes in the patient is treated or prevented.

29. A method of treating or preventing type II diabetes in a patient, wherein the method comprises inhibiting dual PTP1B and TC-PTP inhibitors by administering to the patient in need thereof an effective amount of a compound of any one of claims 4-9 or a pharmaceutical composition of claim 17, whereupon the type II diabetes in the patient is treated or prevented.

30. A method of treating or preventing type II diabetes in a patient, wherein the method comprises degrading dual PTP1B and TC-PTP proteins by administering to the patient in need thereof an effective amount of a compound of any one of claims 10-14 or a pharmaceutical composition of claim 18, whereupon the type II diabetes in the patient is treated or prevented.

31. A method of treating or preventing obesity in a patient, wherein the method comprises inhibiting or degrading dual PTP1B and TC-PTP by administering to the patient in need thereof an effective amount of a compound of any one of claims 1-3 or a pharmaceutical composition of claim 16, whereupon the obesity in the patient is treated or prevented.

32. A method of treating or preventing obesity in a patient, wherein the method comprises inhibiting dual PTP1B and TC-PTP inhibitors by administering to the patient in need thereof an effective amount of a compound of any one of claims 4-9 or a pharmaceutical composition of claim 17, whereupon the obesity in the patient is treated or prevented.

33. A method of treating or preventing obesity in a patient, wherein the method comprises degrading dual PTP1B and TC-PTP proteins by administering to the patient in need thereof an effective amount of a compound of any one of claims 10-14 or a pharmaceutical composition of claim 18, whereupon the obesity in the patient is treated or prevented.

34. A method of inhibiting or degrading dual PTP1B and TC-PTP in a patient, wherein the method comprises administering to the patient in need thereof an effective amount of a compound of any one of claims 1-14 or a pharmaceutical composition of any one of claims 16-18, whereupon dual PTP1B and TC-PTP in the patient is inhibited or degraded.

35. The method of claim 34, wherein the patient has colon cancer, lung adenocarcinoma, squamous cell carcinoma, or melanoma.

36. The method of claim 34, wherein the patient has type-II diabetes.

37. The method of claim 34, wherein the patient is obese.

38. Use of a compound of any one of claims 1-9 in the treatment of a disease or condition that can be treated by inhibiting dual PTP1B or TC-PTP proteins.

39. The use of claim 38, wherein the disease or condition is cancer, type-II diabetes, or obesity.

40. Use of a compound of any one of claims 1-3 or 10-14 in the treatment of a disease or condition that can be treated by degrading dual PTP1B or TC-PTP proteins.

41. The use of claim 40, wherein the disease or condition is cancer, type-II diabetes, or obesity.
